(19) United States
(12) Patent Application Publication
(10) Pub. No.: US 2004/0102413 A1
(43) Pub. Date: May 27, 2004

(54) Method and reagent for the inhibition of telomerase enzyme

(75) Inventors: James McSwiggen, Boulder, CO (US); Bharat M. Chowrira, Louisville, CO (US); Dan T. Stinchcomb, Ft. Collins, CO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sirna Therapeutics, Inc., Boulder, CO (US)

(21) Appl. No.: 10/712,672

(22) Filed: Nov. 13, 2003

Related U.S. Application Data
(63) Continuation of application No. 09/653,225, filed on Aug. 31, 2000.
(60) Provisional application No. 60/151,713, filed on Aug. 31, 1999.
(60) Provisional application No. 60/197,769, filed on Apr. 14, 2000.

(51) Int. Cl. A61K 48/00; C07H 21/02

(52) U.S. Cl. 514/44; 536/23.1

(57) ABSTRACT

The present invention relates to nucleic acid molecules which modulate the synthesis, expression and/or stability of an RNA encoding one or more protein subunits of telomerase enzyme.

This application is a continuation of U.S. application Ser. No. 09/653,225 filed Aug. 31, 2000 which claims the benefit of U.S. Provisional Application No. 60/151,713 filed on Aug. 31, 1999 and U.S. Provisional Application No. 60/197,769 filed on Apr. 14, 2000. All of the applications are incorporated by reference herein in their entireties, including the drawings.

The Sequence Listing file named “MBHBOO,882-C SequenceListing.txt” (1,187,852 bytes in size) submitted in duplicate on Compact Disc-Recordable (CD-R) medium (“010913_{—}1300”) in compliance with 37 C.F.R. §1.52(e) is incorporated herein by reference.

### BACKGROUND OF THE INVENTION

The present invention concerns compounds, compositions, and methods for the study, diagnosis, and treatment of conditions and diseases related to the level of telomerase enzyme.

The following is a brief description of the current understanding in the biology of telomerase and its components. The discussion is not meant to be complete and is provided only for understanding of the invention that follows. The summary is not an admission that any of the work described below is prior art to the claimed invention.

The ribonucleoprotein enzyme telomerase consists of an RNA template subunit and one or more protein subunits including telomerase reverse transcriptase (TERT), which function together to direct the synthesis of telomeres. Telomeres exist as non-nucleosome DNA/protein complexes at the physical ends of eukaryotic chromosomes. These capping structures maintain chromosome stability and replicative potential (Zakian, V. A., 1995, Science, 270, 1601-1607). Telomere structure is characterized by tandem repeats of conserved DNA sequences rich in G-C base pairs. Additional conserved telomere elements include a terminal 3′-overhang in the G-rich strand and non-histone structural proteins that are complexed with telomeric DNA in the nucleus. (Blackburn, “E., 1990, JBC., 265, 5919-5921.). Observed shortening of telomeres coincides with the onset of cellular senescence in most somatic cell lines lacking significant levels of telomerase. This finding has had a profound impact on our views concerning the mechanisms of aging, age related disease, and cancer.

Conventional DNA polymerases are unable to fully replicate the ends of linear chromosomes (Watson, J. D., 1972, Nature, 239, 197-201). This inability stems from the 3′ G-rich overhang that is a product of ribonuclease cleavage of the RNA primer used in DNA replication. The overhang prevents DNA polymerase replication since the recessed C-rich parent strand cannot be used as a template. Telomerase overcomes this limitation by extending the 3′ and of the chromosome using deoxyribonucleotides as substrates and a sequence within the telomerase RNA subunit as a template. (Lingner, J., 1995, Science, 269, 1533-1534). As such, telomerase is considered a reverse transcriptase that is responsible for telomere maintenance.

Telomerase was first discovered by in Tetrahymena thermophila in 1985 (Greider, C. W., 1995, Cell, 43, 405-413). The RNA subunits and their respective genes were later discovered and characterized in protozoa, budding yeast, and mammals. Genetic studies of these genes confirmed the role of telomerase RNA (TR) in determining telomere sequence by mutating genes which encode the telomeric RNA (Yu, G. L., 1990, Nature, 344, 126-132), (Singer, M. S., 1994, Science, 266, 404-409), (Blasco, M. A., 1995, Science, 269, 1267-1270). These studies showed that telomerase activity parallels TR expression in protozoa, yeast and mice. However, the expression of human telomerase RNA (hTR) does not correlate well with telomerase activity in mammalian cells. Many human tissues express hTR but are devoid of telomerase activity (Feng, J., 1995, Science, 269, 1236-1241). Knockout mice, in which the mTR gene has been deleted from germline cells, have been shown to be viable for at least six generations. Cells from later generations of these mice showed chromosomal abnormalities consistent with telomere degradation, indicating that mTR is necessary for telomere length maintenance, but is not required for embryonic development, oncogenic transformation, or tumor formation in mice (Blasco, M. A., 1997, Cell, 91, 25-34).

The first catalytically active subunit of telomerase (p123) was isolated from Euplotes aediculatus along with another subunit (p43) and a 66-kD RNA subunit (Linger, J., 1996, Proc. Natl. Acad. Sci., 93, 10712-10717). Subsequent studies revealed telomerase catalytic subunit homologs from fission yeast (Est2p) and human genes (TRT1). The human homolog, TRT1 encoding hTERT, expressed mRNA with a strong correlation to telomerase activity in human cells (Nakamura, T. M., 1997, Science, 277, 955-959). Reconstitution of telomerase activity with in vitro transcribed and translated hTERT and hTR, either co-synthesized or simply mixed, demonstrated that hTERT and hTR represent the minimal components of telomerase. Furthermore, transient expression of hTERT in normal diploid human cells restored telomerase activity, demonstrating that hTERT is the only component necessary to restore telomerase activity in normal human cells (Weinrich, S. L., 1997, Nature Genetics, 17, 498-502). The introduction of telomerase into normal human cells using hTERT expression via transfection has resulted in the extension of life span in these cells. Such findings indicate that telomere loss in the absence of telomerase is the “mitotic clock” that controls the replicative potential of a cell prior to senescence (Bodnar, A. G., 1998, Science, 279, 349-352).

Expression of telomerase is observed in germ cell and most cancer cell lines. These “immortal” cell lines continue to divide without shortening of their telomeres (Kim, N. W., 1994, Science, 266, 2011-2015). A model of tumor progression has evolved from these findings, suggesting a role for telomerase expression in malignant transformation. Successful malignant transformation in human cells was accomplished for the first time by ectopic expression of hTERT in combination with two oncogenes, SV40 large-T and H-ras. Injection of nude mice with cells expressing these oncogenes and hTERT resulted in rapid growth of tumors. These observations indicate that hTERT mediated telomere maintenance is essential for the formation of human tumor cells (Hahn, W. C., 1999, Nature, 400, 464-468).

Various methods have been developed to assay telomerase activity in vitro. The most widely used method to characterize telomerase activity is the telomeric repeat amplification protocol (TRAP). TRAP utilizes RT-PCR of cellular extracts to measure telomerase activity by making the amount of PCR target dependant upon the biochemical activity of the enzyme (Kim, N. W., 1997, Nucleic Acids Research, 25, 2595-2597).

A variety of animal models have been designed to assay telomerase activity in vivo. Inhibition of telomerase activity has been analyzed in rats via cell proliferation studies with MNU (N-methyl-N-nitosurea) induced mammary carcinomas in response to treatment with 4-(hydroxyphenyl)retinamide (4-HPR), a known inhibitor of mammary carcinogenesis in animal models and premenopausal women (Bednarek, A., 1999, Carcinogenesis, 20, 879-883). Additional studies have focused on the up-regulation of telomerase in transformed cell lines from animal and human model systems (Zhang, P. B., 1998, Leuk. Res., 22, 509-516), (Chadeneau, C., 1995, Oncogene, 11, 893-898), (Greenberg, R., 1999, Oncogene, 18, 1219-1226).

Human cell culture studies have been established to assay inhibition of telomerase activity in human carcinomas responding to various therapeutics. A human breast cancer model for studying telomerase inhibitors is described (Raymond, E., 1999, Br. J. Cancer, 80, 1332-1341). Human studies of telomerase expression as related to various other cancers are described including cervical cancer (Nakano, K., 1998, Am. J. Pathol, 153, 857-864), endometrial cancer (Kyo, S., 1999, Int. J. Cancer, 80, 60-63), meningeal carcinoma (Kleinschmidt-DeMasters, B. K., 1998, J. Neurol. Sci., 161, 124-134), lung carcinoma (Yashima, K., 1997, Cancer Reseach, 57, 2372-2377), testicular cancer in response to cisplatin (Burger, A. M., 1997, Eur. J. Cancer, 33, 638-644), and ovarian carcinoma (Counter, C. M., 1994, Proc. Natl. Acad. Sci., 91, 2900-2904).

Particular degenerative and disease states that can be associated with telomerase expression modulation include but are not limited to:

Cancer: Almost all human tumors have detectable telomerase activity (Shay, J. W., 1997, Eur. J. Cancer, 33, 787-791). Treatment with telomerase inhibitors may provide effective cancer therapy with minimal side effects in normal somatic cells that lack telomerase activity. The therapeutic potential exists for the treatment of a wide variety of cancer types.

Restinosis: Telomerase inhibition in vascular smooth muscle cells may inhibit restinosis by limiting proliferation of these cells.

Infectious disease: Telomerase inhibition in infectious cell types that express telomerase activity may provide selective anti-infectious agent activity. Such treatment may prove especially effective in protozoan-based infection such as Giardia and Lesh Meniesis.

Transplant rejection: Telomerase inhibition in endothelial cell types may demonstrate selective immunnosuppressant activity. Activation of telomerase in transplant cells could benefit grafting success through increased proliferative potential.

Autoimmune disease: Telomerase modulation in various immune cells may prove beneficial in treating diseases such as multiple sclerosis, lupus, and AIDS.

Age related disease: Activation of telomerase expression in cells at or nearing senescence as a result of advanced age or premature aging could benefit conditions such as macular degeneration, skin ulceration, and rheumatoid arthritis.

The present body of knowledge in telomerase research indicates the need for methods to assay telomerase activity and for compounds that can regulate telomerase expression for research, diagnostic, trait alteration, animal health and therapeutic use.

Gaeta et al., U.S. Pat. Nos. 5,760,062; 5,767,278; 5,770,613 have described small molecule inhibitors of human telomerase RNA (hTR) subunit.

Blasco et al., 1995, Science, 269, 1267-1270 describe the synthesis and testing of antisense oligonucleotides targeted against a specific region of the mouse telomerase RNA (mTR) subunit and reported reduction in telomerase activity in mice.

Bisoffi et al., 1998, Eur. J. Cancer, 34, 1242-1249 have studied the down regulation of human telomerase activity by a retrovirus vector expressing antisense RNA targeted against the hTR RNA.

Norton et al., 1996, Nature Biotechnology, 14, 615-619 have reported the use of a peptide nucleic acid (PNA) molecule targeting hTR RNA to down regulate telomerase activity in human immortal breast epithelial cells.

Yokoyama et al., 1998, Cancer Research, 58, 5406-5410 have reported the synthesis and testing of hammerhead ribozyme constructs targeting hTR RNA resulting in a decrease in the telomerase activity in Ishikawa cells.

Henderson, European Patent Application No. 666,313-A2 describes methods of identifying and cloning hTR gene for use in gene therapy approaches for creating aberrant telomeric sequences in transfected human tumor cells. A ribozyme based gene therapy approach to inhibit the expression of hTR gene is described as well. The intended result of such therapies involves incurred genetic instability based on non-native telomeric sequences resulting in rapid cell death of the treated cells.

West et al., U.S. Pat. No. 5,489,508 describe methods for determining telomere length and telomerase activity in cells. Inhibitors of hTR RNA, including oligonucleotides and/or small molecules are described.

These foregoing approaches of targeting the telomerase RNA subunit (TR) may not be very beneficial, because as demonstrated by Feng et al., (Feng, J., 1995, Science, 269, 1236-1241), telomerase activity in humans does not correlate well to hTR concentration.

Collins et al., International PCT publication No. WO 98/01542 describes assays for the detection of telomerase activity. Four human telomerase subunit proteins are described called p140, p105, p48 and p43. In addition, hybridization probes and primers are described as inhibitors of telomerase gene function. Antibody based inhibitors of telomerase protein subunits are described.

A more attractive approach to telomerase regulation would involve the regulation of human telomerase by modulating the expression of the protein subunits of the enzyme, preferably the reverse transcriptase (hTERT) subunit. Based of reconstitution experiments, hTERT and hTR represent the minimal components of telomerase. Since hTR expression does not correlate well with telomerase activity in human cells and since many human cells express hTR without telomerase activity, targeting hTERT may prove more beneficial than targeting hTR. hTERT is the only component necessary to restore telomerase activity in normal human cells. A study in which the three major subunits of telomerase (hTR, TP1, and hTERT were assayed in normal and malignant endometrial tissues determined that hTERT is a rate limiting determinant of enzymatic activity of human telomerase (Kyo, S., 1999, Int. J. Cancer, 80, 60-63). Additional protein subunits that have been isolated most likely serve only a structural role in telomerase activity, but may be important in enhancing the activity of the telomerase enzyme. As such, hTERT is one of the better targets for the ectopic regulation of telomerase activity.

Cech et al., International PCT publication No. WO 98/14593 describe compositions and methods related to hTERT for diagnosis, prognosis and treatment of human diseases, for altering proliferative capacity in cells and organisms, and for screening compounds and treatments with potential use as human therapeutics.

Cech et al., International PCT publication No. WO 98/14592 describe nucleic acid and amino acid sequences encoding various telomerase protein subunits and motifs of Euplotes aediculatus, and related sequences from Schizosaccharomyces, Saccharomyces sequences, and human telomerase. The polypeptides comprising telomeric subunits and functional polypeptides and ribonucleoproteins that contain these subunits are described as well. Cech et al., International PCT Publication No. WO 98/14592, mentions in general terms the the possibility of using antisense and ribozymes to down regulate the expression of human telomerase reverse transcriptase enzyme.

### SUMMARY OF THE INVENTION

The invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups (Cook et al., U.S. Pat. No. 5,359,051)] and methods for their use to down regulate or inhibit the expression of telomerase enzyme.

In a preferred embodiment, the invention features use of one or more of the nucleic acid-based techniques to inhibit the expression of the genes encoding the protein subunits of the telomerase enzyme, preferably the catalytic subunit of the telomerase enzyme. Specifically, the invention features the use of nucleic acid-based techniques to specifically inhibit the expression of telomerase reverse transcriptase (TERT) gene.

In another preferred embodiment, the invention features the use of an enzymatic nucleic acid molecule, preferably in the hammerhead, NCH, G-cleaver and/or DNAzyme motif, to inhibit the expression TERT gene.

In another preferred embodiment, the invention features the inhibition or down regulation of telomerase activity by inhibiting or down regulating the expression of one or more activators of telomerase enzyme, such as protein encoded by ras gene. Such activator gene expression may be regulated by the use of nucleic acid-based techniques, such as enzymatic nucleic acid molecules and antisense oligonucleotides.

By “inhibit” it is meant that the activity of telomerase enzyme or level of RNAs or equivalent RNAs encoding one or more protein subunits of the telomerase enzyme is reduced below that observed in the absence of the nucleic acid. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of for example, an oligonucleotide with scrambled sequence or with mismatches. In another embodiment, inhibition of TERT genes with the nucleic acid molecule of the instant invention is greater than in the presence of the nucleic acid molecule than in its absence. According to the invention, the activity of telomerase enzyme or the level of RNA encoding one or more protein subunits of the telomerase enzyme is inhibited if it is at least 10% less, 20% less, 50% less, 75% less or even not active or present at all, in the presence of a nucleic acid of the invention relative to the level in the absence of such a nucleic acid.

As used herein, the term “telomerase activity” refers to enzyme activity that replicates, for example, the TTAGGG repeats at the ends of linear chromosomes. Telomerase activity is comprised by a ribonucleoprotein enzyme comprising one or more protein subunits and an RNA subunit. The enzymatic activity extends the 5′-recessed end of a linear chromosome using deoxyribonucleotides and an RNA sequence within the RNA subunit as a primer. Telomerase activity may be assayed as follows. Samples to be assayed for telomerase activity are prepared by extraction into CHAPS lysis buffer (10 mM Tris pH 7.5, 1 mM MgCl₂, 1 mM EGTA, 0.1 mM PMSF, 5mM -mercaptoethanol, 1 mM DTT, 0.5% 3-[(3-cholamidopropyl)-dimethyl-amino]-1- propanesulfonate (CHAPS), 10% glycerol and 40 U/ml RNAse inhibitor (Promega, Madison, Wis., U.S.A.). Cells are suspended in CHAPS lysis buffer and incubated on ice for 30 minutes, which allows lysis of 90-100% of cells. Lysate is then transferred to polyallomer centrifuge tubes and spun at 100,000×g for 1 hour at 4 degrees C. The supernatant is the protein extract, and concentration ranges of 4-10 μg/μl are suitable for telomerase assay. Extracts may be concentrated if necessary using a Microcon Microfilter 30 (Amicron, Beverly, Mass. U.S.A.) according to the manufactureris instructions. Extracts may be stored frozen at −80 degrees C. until assayed.

Telomerase may be assayed according to Kim and Wu, Nucl. Acids Res. 25: 2595-2597, incorporated herein by reference. Briefly, for the telomerase assay, 2 μg of protein extract is used. The extract is assayed in 50 μl of reaction mixture containing 0.1 μg TS substrate primer (5′-MTCCGTCGAGCAGAGTT-3′ (SEQ. ID. NO. 5569) end-labeled using alpha-³²P-ATP and T4 polynucleotide kinase)(SEQ. ID. NO. 5570) 0.1 μg ACX return primer (5′-GCGCGG[CTTACC]₃ CTAACC-3′), 0.1 μg NT internal control primer (5′-ATCGCTTCTCGGCCTTTT-3′) (SEQ. ID. NO. 5571) 0.01 micromol TSNT internal control template (5′-MTCCGTCGAGCAGAGTTAAAAGGCCGAGMCGAT-3′) (SEQ. ID. No. 5572) 50 μM each deoxynucleoside triphosphate, 2 U of Taq DNA polymerase, and 2 μl CHAPS protein extract, all in 1×TRAP buffer (20 mM Tris (pH 8.3), 68 mM KCl, 1.5 mM MgCl₂, 1 mM EGTA, 0.05% Tween 20). Each reaction is placed in a thermocycler block preheated to 30 C and incubated at 30 C for 10 minutes, then cycled for 27 cycles of 94 degrees C. for 30 seconds, 60 degrees C. for 30 seconds. Reaction products are separated on a denaturing 8% polyacrylamide gel, followed by drying of the gel and autoradiography. The internal control (to control for possible Taq polymerase inhibition) generates a band of 36 nt. Comparison of radioactive signal integrated (e.g., by phorphorimager analysis) for telomerase-extended bands with the radioactive signal from a reaction performed with a known amount of quantification standard template (termed R8; 5′-AATCCGTCGAGCAGAGTTAG [GGTTAG]₇-3′) (SEQ. ID. NO. 5573) allows expression of telomerase activity as an absolute value. The absolute value=TPG (total product generated)=[(TP-TPi)/Tl]/[(R8-B)/RI)]×100, where TP=telomerase products from test extract, TPi=telomerase products from a heat-inactivated (75° C., 10 minutes) extract reaction, TI=the signal from the internal control, R8=the signal from the R8 qualification standard template reaction, B=signal from a lysis buffer-only blank reaction, and RI=the internal control value for the reaction containing R8 template and NT and TSNT control primers. TPG values of 0-10,000 are possible, with the linear range being from approximately 1 to 1000 TPG. The range of 1 to 1000 TPG encompasses the minimum and maximum levels of telomerase activity in most tumor samples tested, while non-tumor cells most often have no telomerase activity (TPG approximately zero).

An alternative telomerase assay, which does not employ PCR amplification, is described by Raymond et al. 1999, Br. J. Cancer 80: 1332-1341.

By “enzymatic nucleic acid molecule” it is meant an RNA molecule which has complementarity in a substrate binding region to a specified gene target, and also has an enzymatic activity which is active to specifically cleave target RNA. That is, the enzymatic RNA molecule is able to intermolecularly cleave RNA and thereby inactivate a target RNA molecule. This complementary regions allow sufficient hybridization of the enzymatic RNA molecule to the target RNA and thus permit cleavage. One hundred percent complementarity between RNA and the target gene or target RNA is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

By “enzymatic portion” or “catalytic domain” is meant that portion/region of the enzymatic nucleic acid molecule essential for cleavage of a nucleic acid substrate (for example see FIG. 1).

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired. Such arms are shown generally in FIG. 1. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides and of sufficient length to stably interact with the target RNA; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By DNAzyme is meant, an enzymatic nucleic acid molecule lacking a 2′-OH group. In particular embodiments the enzymatic nucleic acid molecule may have an attached linker(s) or other attached or associated groups, moieties, or chains containing one or more nucleotides with 2′-OH groups.

By “sufficient length” is meant an oligonucleotide of greater than or equal to 3 nucleotides, 5 nucleotides, 7 nucleotides, 9 nucleotides or even 12 nucleotides.

By “stably interact” is meant, interaction of the oligonucleotides with target nucleic acid (e.g., by forming hydrogen bonds with complementary nucleotides in the target under physiological conditions).

By “equivalent” RNA to telomerase enzyme is meant to include those naturally occurring RNA molecules having homology (partial or complete) to nucleic acid sequences encoding telomerase proteins or encoding for proteins with similar function as telomerase in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review see Stein and Cheng, 1993 Science 261, 1004). Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “2-5A antisense chimera” it is meant, an antisense oligonucleotide containing a 5′ phosphorylated 2′-5′-linked adenylate residues. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300).

By “triplex DNA” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci.USA 89, 504).

By “gene” it is meant a nucleic acid that encodes an RNA.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix inhibition. Determination of binding free energies for nucleic acid molecules is well known in the art (see, e.g., Turner et al., 1987, CSH Symp. Quant. Biol. Lll pp.123-133; Frier et al., 1986, Proc. Nat. Acad. Sci. USA 83:9373-9377; Turner et al., 1987, J. Am. Chem. Soc. 109:3783-3785. A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

At least seven basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in telomerase-specific RNAs represent a novel therapeutic approach to treat a variety of pathologic indications, including, cancer, tumorigenesis, restenosis and others.

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 3; Beigelman et al., U.S. Ser. No. 09/301,511) and Zinzyme (Beigelman et al., U.S. Ser. No. 09/301,511) can also be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule, e.g., an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

In a preferred embodiment the invention provides a method for producing a class of nucleic acid -based gene inhibiting agents which exhibit a high degree of specificity for the RNA of a desired target. For example, the enzymatic nucleic acid molecule is preferably targeted to a highly conserved sequence region of target RNAs encoding telomerase proteins (specifically TERT gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

By “highly conserved sequence region” is meant a nucleotide sequence of one or more regions in a target gene does not vary significantly from one generation to the other or from one biological system to the other.

The nucleic acid-based inhibitors of telomerase expression are useful for the prevention of the diseases and conditions including cancer, macular degeneration, restenosis, certain infectious diseases, transplant rejection and autoimmune disease such as multiple sclerosis, lupus, and AIDS; Age related disease such as macular degeneration, skin ulceration, and rheumatoid arthritis and any other diseases or conditions that are related to the levels of telomerase in a cell or tissue.

By “related” is meant that the reduction of telomerase expression (specifically TERT gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences which are complementary to the substrate sequences in Tables III-VII. Examples of such enzymatic nucleic acid molecules also are shown in Tables III to VII. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in tables III to VII. Such nucleic acid molecules can include sequences as shown for the binding arms of the enzymatic nucleic acid molecules in Tables III to VII. Similarly, triplex molecules can be provided targeted to the corresponding DNA target regions, and containing the DNA equivalent of a target sequence or a sequence complementary to the specified target (substrate) sequence. Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “consists essentially of” is meant that the active ribozyme contains an enzymatic center or core equivalent to those in the examples, and binding arms able to bind mRNA such that cleavage at the target site occurs. Other sequences may be present which do not interfere with such cleavage. Thus, a core region may, for example, include one or more loop, stem-loop structure, which does not prevent enzymatic activity. The underlined regions in the sequences in Tables III and IV can be such a loop, and can be represented generally as sequence “X”. For example, a core sequence for a hammerhead ribozyme can be a 5′-CUGAUGAG-3′ and 5′-CGAA-3′connected by “X”, where X is 5′-GCCGUUAGGC-3′(SEQ ID NO 5574), or any other Stem II region known in the art.”

In another aspect of the invention, ribozymes or antisense molecules that cleave target RNA molecules and inhibit telomerase enzyme (specifically TERT) activity are expressed from transcription units inserted into DNA or RNA vectors. The recombinant vectors are preferably DNA plasmids or viral vectors. Ribozyme or antisense expressing viral vectors could be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors may be used that provide for transient expression of ribozymes or antisense. Such vectors might be repeatedly administered as necessary. Once expressed, the ribozymes or antisense bind to the target RNA and inhibit its function or expression. Delivery of ribozyme or antisense expressing vectors could be systemic, such as by intravenous or intramuscular administration, by administration to target cells ex-planted from the patient followed by reintroduction into the patient, or by any other means that would allow for introduction into the desired target cell.

By “vectors” is meant any nucleic acid- and/or viral-based technique used to deliver a desired nucleic acid.

By “patient” is meant an organism which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

The nucleic acid molecules of the instant invention, individually, or in combination or in conjunction with other drugs, can be used to treat diseases or conditions discussed above. For example, to treat a disease or condition associated with the levels of telomerase enzyme, the patient may be treated, or other appropriate cells may be treated, as is evident to those skilled in the art, individually or in combination with one or more drugs under conditions suitable for the treatment.

In a further embodiment, the described molecules, such as antisene or ribozymes can be used in combination with other known treatments to treat conditions or diseases discussed above. For example, the described molecules could be used in combination with one or more known therapeutic agents to treat cancer.

In another preferred embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., TERT) capable of progression and/or maintenance of cancer.

In another preferred embodiment, the invention features nucleic acid-based techniques (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of TERT gene expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### BRIEF DESCRIPTION OF THE DRAWINGS

FIG. 1 shows the secondary structure model for seven different classes of enzymatic nucleic acid molecules. Arrow indicates the site of cleavage. —indicate the target sequence. Lines interspersed with dots are meant to indicate tertiary interactions. —is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: I-IV are meant to indicate four stem-loop structures (Been et al., U.S. Pat. No. 5,625,047). Hammerhead Ribozyme: I-II are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “_” refers to a covalent bond. (Burke et al., 1996, Nucleic Acids & Mol. Biol., 10, 129; Chowrira et al., U.S. Pat. No. 5,631,359).

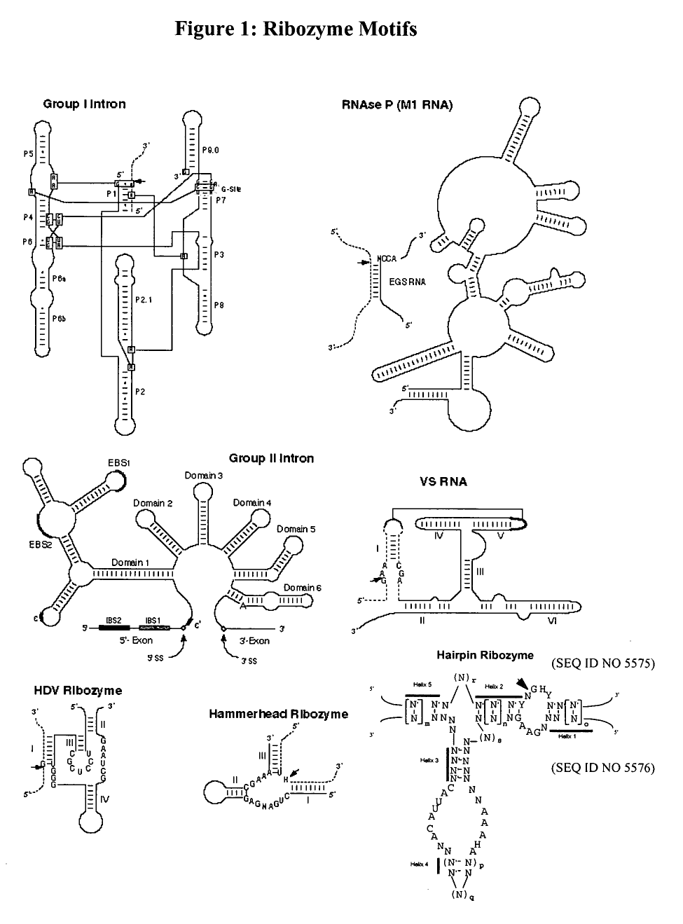

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which may be same or different and have complementarity to each other; rI, represents riboInosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

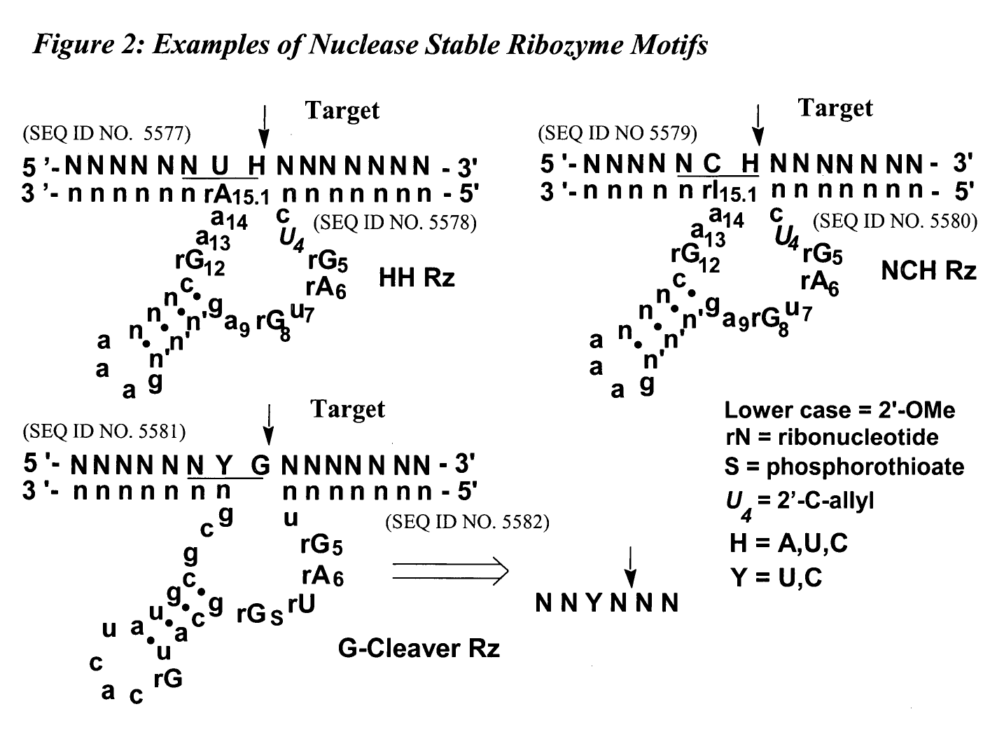

FIG. 3 shows an example of the Amberzyme ribozyme motif that is chemically stabilized (see for example Beigelman et al., WO 99/55857; also referred to as Class I Motif).

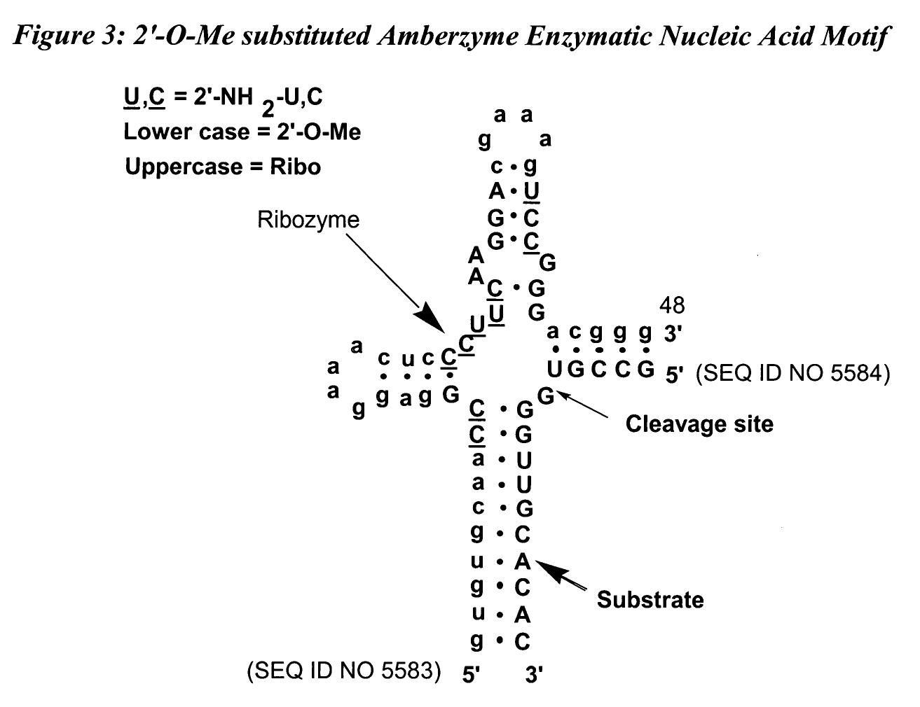

FIG. 4 shows an example of the Zinzyme A ribozyme motif that is chemically stabilized (see for example Beigelman et al., WO 99/55857; also referred to as Class A Motif).

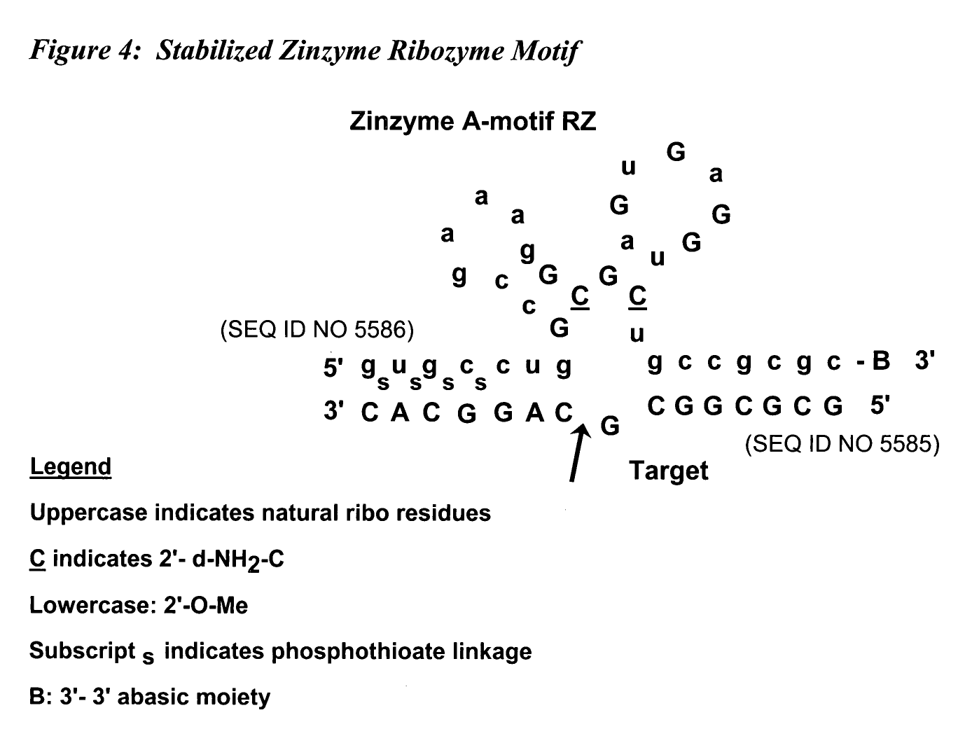

### DETAILED DESCRIPTION OF THE INVENTION

Mechanism of Action of Nucleic Acid Molecules of the Invention

Antisense: Antisense molecules may be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates and phosphorodithioates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. S No. 60/082,404 which was filed on Apr. 20, 1998; Hartmann et al., U.S. S No. 60/101,174 which was filed on Sep. 21, 1998) all of these are incorporated by reference herein in their entirety.

Triplex Forming Oligonucleotides (TFO): Single stranded DNA may be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism may result in gene expression or cell death since binding may be irreversible (Mukhopadhyay & Roth, supra)

2-5A Antisense Chimera: The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures may be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid: Seven basic varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent telomerase protein expression (specifically TERT) and can be used to treat disease or diagnose disease associated with the levels of telomerase enzyme.

The enzymatic nature of a ribozyme has significant advantages, such as the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieved efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving ribozymes show promise as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited.

Target Sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art. Ribozymes and antisense to such targets are designed as described in those applications and synthesized to be tested in vitro and in vivo, as also described. The sequence of human TERT RNAs were screened for optimal enzymatic nucleic acid and antisense target sites using a computer folding algorithm. Antisense, hammerhead, DNAzyme, NCH, or G-Cleaver ribozyme binding/cleavage sites were identified. These sites are shown in Tables III to VII (all sequences are 5′ to 3′ in the tables; the underlined region can be any base-paired sequence, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH, or G-Cleaver ribozyme binding/cleavage sites were identified. The nucleic acid molecules were individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core were eliminated from consideration. Varying binding arm lengths can be chosen to optimize activity.

Antisense, hammerhead, DNAzyme, NCH, or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., antisense oligonucleotides, hammerhead or the hairpin ribozymes) are preferably used for exogenous delivery. The simple structure of these molecules increases the ability of the nucleic acid to invade targeted regions of RNA structure. Exemplary molecules of the instant invention were chemically synthesized, and others can similarly be synthesized. Oligodeoxyribonucleotides were synthesized using standard protocols as described in Caruthers et al., 1992, Methods in Enzymology 211,3-19, and is incorporated herein by reference.

The method of synthesis used for normal RNA including certain enzymatic nucleic acid molecules follows the procedure as described in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses were conducted on a 394 Applied Biosystems, Inc. synthesizer using a 0.2 μmol scale protocol with a 7.75 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 15-fold excess (31 μL of 0.1 M=3.1 μmol) of phosphoramidite and a 38.7-fold excess of S-ethyl tetrazole (31 μL of 0.25 M=7.75 μmol) relative to polymer-bound 5′-hydroxyl was used in each coupling cycle. Average coupling yields on the 394 Applied Biosystems, Inc. synthesizer, determined by colorimetric quantitation of the trityl fractions, were 97.5-99%. Other oligonucleotide synthesis reagents for the 394 Applied Biosystems, Inc. synthesizer; detritylation solution was 3% TCA in methylene chloride (ABI); capping was performed with 16% N-methyl imidazole in THF (ABI) and 10% acetic anhydride/10% 2,6-lutidine in THF (ABI); oxidation solution was 16.9 mM I₂, 49 mM pyridine, 9% water in THF (PERSEPTIVE™). Burdick & Jackson Synthesis Grade acetonitrile was used directly from the reagent bottle. S-Ethyltetrazole solution (0.25 M in acetonitrile) was made up from the solid obtained from American International Chemical, Inc.

Deprotection of the RNA was performed using either a two-pot or one-pot protocol. For the two-pot protocol, the polymer-bound trityl-on oligoribonucleotide was transferred to a 4 mL glass screw top vial and suspended in a solution of 40% aq. methylamine (1 mL) at 65° C. for 10 min. After cooling to −20° C., the supernatant was removed from the polymer support. The support was washed three times with 1.0 mL of EtOH:MeCN:H20/3:1:1, vortexed and the supernatant was then added to the first supernatant. The combined supernatants, containing the oligoribonucleotide, were dried to a white powder. The base deprotected oligoribonucleotide was resuspended in anhydrous TEA/HF/NMP solution (300 μL of a solution of 1.5 mL N-methylpyrrolidinone, 750 μL TEA and 1 mL TEA-3HF to provide a 1.4 M HF concentration) and heated to 65° C. After 1.5 h, the oligomer was quenched with 1.5 M NH₄HCO₃.

Alternatively, for the one-pot protocol, the polymer-bound trityl-on oligoribonucleotide was transferred to a 4 mL glass screw top vial and suspended in a solution of 33% ethanolic methylamine/DMSO:1/1 (0.8 mL) at 65° C. for 15 min. The vial was brought to r.t. TEA•3HF (0.1 mL) was added and the vial was heated at 65° C. for 15 min. The sample was cooled at −20° C. and then quenched with 1.5 M NH₄HCO₃.

For purification of the trityl-on oligomers, the quenched NH₄HCO₃ solution was loaded onto a C-18 containing cartridge that had been prewashed with acetonitrile followed by 50 mM TEAA. After washing the loaded cartridge with water, the RNA was detritylated with 0.5% TFA for 13 min. The cartridge was then washed again with water, salt exchanged with 1 M NaCl and washed with water again. The oligonucleotide was then eluted with 30% acetonitrile.

Inactive hammerhead ribozymes or binding attenuated control (BAC) oligonucleotides) were synthesized by substituting a U for G₅ and a U for A14 (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields were >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997 Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992 TIBS 17, 34; Usman et al., 1994 Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

The sequences of the ribozymes that are chemically synthesized, useful in this study, are shown in Tables III to VII. Those in the art will recognize that these sequences are representative only of many more such sequences where the enzymatic portion of the ribozyme (all but the binding arms) is altered to affect activity. The ribozyme sequences listed in Tables III to V and VII may be formed of ribonucleotides or other nucleotides or non-nucleotides. Such ribozymes with enzymatic activity are equivalent to the ribozymes described specifically in the Tables.

Optimizing Activity of the Nucleic Acid Molecule of the Invention.

Chemically synthesizing synthesizing nucleic acid molecules with modifications (base, sugar and/or phosphate) that prevent their degradation by serum ribonucleases may increase their potency (see e.g., Eckstein et al., International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992 TIBS 17, 34; Usman et al., 1994 Nucleic Acids Symp. Ser. 31, 163; Burgin et al., 1996 Biochemistry 35, 14090). Sugar modification of nucleic acid molecules have been extensively described in the art (see Eckstein et al., International Publication PCT No. WO 92/07065; Perrault et al. Nature 1990, 344, 565-568; Pieken et al. Science 1991, 253, 314-317; Usman and Cedergren, Trends in Biochem. Sci. 1992, 17, 334-339; Usman et al. International Publication PCT No. WO 93/15187; Sproat, U.S. Pat. No. 5,334,711 and Beigelman et al., 1995 J. Biol. Chem. 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. S. No. 60/082,404 which was filed on Apr. 20, 1998; Karpeisky et al., 1998 Tetrahedron Lett. 39, 1131; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19) incorporated by reference herein) have expanded the ability to modify nucleic acid molecules by introducing nucleotide modifications to enhance their nuclease stability as described above.

Use of these the nucleic acid-based molecules of the invention will lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules)). The treatment of patients with nucleic acid molecules may also include combinations of different types of nucleic acid molecules.

Therapeutic nucleic acid molecules (e.g., enzymatic nucleic acid molecules and antisense nucleic acid molecules) delivered exogenously must optimally be stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

By “enhanced enzymatic activity” is meant to include activity measured in cells and/or in vivo where the activity is a reflection of both catalytic activity and ribozyme stability. In this invention, the product of these properties is increased or not significantly (less that 10 fold) decreased in vivo compared to an all RNA ribozyme.

In yet another preferred embodiment, nucleic acid catalysts having chemical modifications which maintain or enhance enzymatic activity is provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity on all RNA ribozyme.

In another aspect the nucleic acid molecules comprise a 5′ and/or a 3′-cap structure.

By “cap structure” is meant chemical modifications, which have been incorporated at the terminus of the oligonucleotide (see for example Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both terminus. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Beigelman et al., International PCT publication No. WO 97/26270, incorporated by reference herein). In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moeity; 5′-5′-inverted abasic moeity; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moeities (for more details see Beaucage and Iyer, 1993, Tetrahedron 49, 1925; incorporated by reference herein). By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group may be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups may also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above. Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra) all are hereby incorporated by reference herein). There are several examples of modified nucleic acid bases known in the art and has recently been summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

By “abasic” is meant sugar moieties lacking a base or having other chemical groups in place of a base at the 1′ position.

By “ribonucleotide” is meant a nucleotide with one of the bases adenine, cytosine, guanine, or uracil joined to the 1′ carbon of β-D-ribo-furanose.

By “unmodified nucleoside” is meant one of the bases adenine, cytosine, guanine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which may be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols may be utilized for the delivery of virtually any nucleic acid molecule. Nucleic acid molecules may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules may be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra and Draper et al., PCT WO93/23569 which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as the cancer cells.

The invention also features the use of the composition comprising surface-modified liposomes containing poly (ethylene glycol) lipids (PEG-modified, or long-circulating liposomes or stealth liposomes). These formulations offer an method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention (e.g., formula IV) can be expressed within cells from eukaryotic promoters (e.g., Izant and Weintraub, 1985 Science 229, 345; McGarry and Lindquist, 1986 Proc. Natl. Acad. Sci. USA 83, 399; Scanlon et al., 1991, Proc. Natl. Acad. Sci. USA, 88, 10591-5; Kashani-Sabet et al., 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad. Sci. USA 89, 10802-6; Chen et al., 1992 Nucleic Acids Res., 20, 4581-9; Sarver et al., 1990 Science 247, 1222-1225; Thompson et al., 1995 Nucleic Acids Res. 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992 Nucleic Acids Symp. Ser., 27, 15-6; Taira et al., 1991, Nucleic Acids Res., 19, 5125-30; Ventura et al., 1993 Nucleic Acids Res., 21, 3249-55; Chowrira et al., 1994 J. Biol. Chem. 269, 25856; all of the references are hereby incorporated in their totality by reference herein).

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intramuscular administration, by administration to target cells explanted from the patient followed by reintroduction into the patient, or by any other means that would allow for introduction into the desired target cell (for a review see Couture et al., 1996, TIG., 12, 510).

In one aspect the invention features, an expression vector comprising nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features, the expression vector comprises: a transcription initiation region (e.g., eukaryotic pol I, II or III initiation region); b) a transcription termination region (e.g., eukaryotic pol I, II or III termination region); c) a gene encoding at least one of the nucleic acid catalyst of the instant invention; and wherein said gene is operably linked to said initiation region and said termination region, in a manner which allows expression and/or delivery of said nucleic acid molecule. The vector may optionally include an open reading frame (ORF) for a protein operably linked on the 5′ side or the 3′-side of the gene encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990 Proc. Natl. Acad. Sci. USA, 87, 6743-7; Gao and Huang 1993 Nucleic Acids Res., 21, 2867-72; Lieber et al., 1993 Methods Enzymol., 217, 47-66; Zhou et al., 1990 Mol. Cell. Biol., 10, 4529-37). Several investigators have demonstrated that nucleic acid molecules, such as ribozymes expressed from such promoters can function in mammalian cells (e.g. Kashani-Sabet et al., 1992 Antisense Res. Dev., 2, 3-15; Ojwang et al., 1992 Proc. Natl. Acad. Sci. USA, 89, 10802-6; Chen et al., 1992 Nucleic Acids Res., 20, 4581-9; Yu et al., 1993 Proc. Natl. Acad. Sci. USA, 90, 6340-4; L'Huillier et al., 1992 EMBO J. 11, 4411-8; Lisziewicz et al., 1993 Proc. Natl. Acad. Sci. U.S.A., 90, 80004; Thompson et al., 1995 Nucleic Acids Res. 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther. 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a gene encoding at least one said nucleic acid molecule; and wherein said gene is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a gene encoding at least one said nucleic acid molecule, wherein said gene is operably linked to the 3′-end of said open reading frame; and wherein said gene is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a gene encoding at least one said nucleic acid molecule; and wherein said gene is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a gene encoding at least one said nucleic acid molecule, wherein said gene is operably linked to the 3′-end of said open reading frame; and wherein said gene is operably linked to said initiation region, said intron, said open reading frame and said termination region, in a manner which allows expression and/or delivery of said nucleic acid molecule.

### EXAMPLE

The following are non-limiting examples showing the selection, isolation, synthesis and activity of nucleic acids of the instant invention.

The following examples demonstrate the selection and design of Antisense, hammerhead, DNAzyme, NCH, or G-Cleaver ribozyme molecules and binding/cleavage sites within TERT RNA.

### Example 1

Identification of Potential Target Sites in Human TERT RNA

The sequence of human TERT was screened for accessible sites using a computer folding algorithm. Regions of the RNA that did not form secondary folding structures and contained potential ribozyme and/or antisense binding/cleavage sites were identified. The sequences of these cleavage sites are shown in tables III-VII.

### Example 2

Selection of Enzymatic Nucleic Acid Cleavage Sites in Human TERT RNA

To test whether the sites predicted by the computer-based RNA folding algorithm corresponded to accessible sites in TERT RNA, 10 hammerhead ribozyme and three G-Cleaver ribozyme sites were selected for further analysis (Table VI). Ribozyme target sites were chosen by analyzing sequences of Human TERT (Nakamura et al., 1997 Science 277, 955-959; Genbank sequence accession number: NM_{—}003219) and prioritizing the sites on the basis of folding. Ribozymes were designed that could bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

### Example 3

Chemical Synthesis and Purification of Ribozymes for Efficient Cleavage of TERT RNA

Ribozymes were designed to anneal to various sites in the RNA message. The binding arms are complementary to the target site sequences described above. The ribozymes were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were >98%.

Ribozymes were also synthesized from DNA templates using bacteriophage T7 RNA polymerase (Milligan and Uhlenbeck, 1989, Methods Enzymol. 180, 51). Ribozymes were purified by gel electrophoresis using general methods or were purified by high pressure liquid chromatography (HPLC; See Wincott et al., supra; the totality of which is hereby incorporated herein by reference) and were resuspended in water. The sequences of the chemically synthesized ribozymes used in this study are shown below in Table III-VII.

### Example 4

Ribozyme Cleavage of TERT RNA Target In Vitro

Ribozymes targeted to the human TERT RNA are designed and synthesized as described above. These ribozymes can be tested for cleavage activity in vitro, for example using the following procedure. The target sequences and the nucleotide location within the TERT RNA are given in Tables III-VII.

Cleavage Reactions: Full-length or partially full-length, internally-labeled target RNA for ribozyme cleavage assay is prepared by in vitro transcription in the presence of [a-³²p] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming 15 μl of a 2× concentration of purified ribozyme in ribozyme cleavage buffer (50 mM Tris-HCl, pH 7.5 at 37° C., 10 mM MgCl₂) and the cleavage reaction was initiated by adding the 2× ribozyme mix to an equal volume (15 μl) of substrate RNA (maximum of 1-5 nM; 5×10⁵ to 1×10⁷ cpm) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume (30 μl) of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

Cell Culture Models

Various methods have been developed to assay telomerase activity in vitro. The most widely used method to characterize telomerase activity is the telomeric repeat amplification protocol (TRAP). TRAP utilizes RT-PCR of cellular extracts to measure telomerase activity by making the amount of PCR target dependant upon the biochemical activity of the enzyme (Kim, N. W., 1997, Nucleic Acids Research, 25, 2595-2597).

Human cell culture studies have been established to assay inhibition of telomerase activity in human carcinomas responding to various therapeutics. A human breast cancer model for studying telomerase inhibitors is described (Raymond, E., 1999, Br. J. Cancer, 80, 1332-1341). Human studies of telomerase expression as related to various other cancers are described including cervical cancer (Nakano, K., 1998, Am. J. Pathol, 153, 857-864), endometrial cancer (Kyo, S., 1999, Int. J. Cancer, 80, 60-63), meningeal carcinoma (Kleinschmidt-DeMasters, B. K., 1998, J. Neurol. Sci., 161, 124-134), lung carcinoma (Yashima, K., 1997, Cancer Reseach, 57, 2372-2377), testicular cancer in response to cisplatin (Burger, A. M., 1997, Eur. J. Cancer, 33, 638-644), and ovarian carcinoma (Counter, C. M., 1994, Proc. Natl. Acad. Sci., 91, 2900-2904).

Animal Models

A variety of animal models have been designed to assay telomerase activity in vivo. Inhibition of telomerase activity has been analyzed in rats via cell proliferation studies with MNU (N-methyl-N-nitosurea) induced mammary carcinomas in response to treatment with 4-(hydroxyphenyl)retinamide (4-HPR), a known inhibitor of mammary carcinogenesis in animal models and premenopausal women (Bednarek, A., 1999, Carcinogenesis, 20, 879-883). The method of Bednarek et al. uses N-methyl-N-nitrosourea (MNU)-induced mammary carcinomas in rats to analyze the effect of telomerase inhibitors in vivo. MNU-induced tumors express high telomerase activity. Female virgin Sprague-Dawley rats are injected twice with MNU (50 mg/kg body weight) at days 43 and 50 days of age. Mammary tumors are allowed to grow to 4-8 mm before commencing treatment with an agent, such as 4-(hydroxyphenyl) retinamide (used by Bednarek et al.) or a nucleic acid of the invention being tested as a modulator of telomerase activity. Following treatment with an agent for 0 to 6 weeks, telomerase activity is assayed using the TRAP method on CHAPS-extracted tumor-cell protein samples. A decrease of 10% or more in telomerase activity relative to the level in tumors of untreated animals indicates an agent is a telomerase inhibitor. Additional studies have focused on the up-regulation of telomerase in transformed cell lines from animal and human model systems (Zhang, P. B., 1998, Leuk. Res., 22, 509-516), (Chadeneau, C., 1995, Oncogene, 11, 893-898), (Greenberg, R., 1999, Oncogene, 18, 1219-1226).

Indications

Particular degenerative and disease states that can be associated with telomerase expression modulation include but are not limited to:

Cancer: Almost all human tumors have detectable telomerase activity (Shay, J. W., 1997, Eur. J. Cancer, 33, 787-791). Treatment with telomerase inhibitors may provide effective cancer therapy with minimal side effects in normal somatic cells that lack telomerase activity. The therapeutic potential exists for the treatment of a wide variety of cancer types.

Restinosis: Telomerase inhibition in vascular smooth muscle cells may inhibit restinosis by limiting proliferation of these cells.

Infectious disease: Telomerase inhibition in infectious cell types that express telomerase activity may provide selective antibiotic activity. Such treatment may prove especially effective in protozoan-based infection such as Giardia and Leishmaniasis.

Transplant rejection: Telomerase inhibition in endothelial cell types may demonstrate selective immunnosuppressant activity. Activation of telomerase in transplant cells could benefit grafting success through increased proliferative potential.

Autoimmune disease: Telomerase modulation in various immune cells may prove beneficial in treating diseases such as multiple sclerosis, lupus, and AIDS.

Age related disease: Activation of telomerase expression in cells at or nearing senescence as a result of advanced age or premature aging could benefit conditions such as macular degeneration, skin ulceration, and rheumatoid arthritis.

The present body of knowledge in telomerase research indicates the need for methods to assay telomerase activity and for compounds that can regulate telomerase expression for research, diagnostic, and therapeutic use.

Gemcytabine and cyclophosphamide are non-limiting examples of chemotherapeutic agents that can be combined with or used in conjunction with the nucleic acid molecules (e.g. ribozymes and antisense molecules) of the instant invention. Those skilled in the art will recognize that other drugs such as anti-cancer compounds and therapies can be similarly be readily combined with the nucleic acid molecules of the instant invention (e.g. ribozymes and antisense molecules) and are hence within the scope of the instant invention. Such compounds and therapies are well known in the art (see for example Cancer: Principles and Pranctice of Oncology, Volumes 1 and 2, eds Devita, V. T., Hellman, S., and Rosenberg, S. A., J. B. Lippincott Company, Philadelphia, USA; incorporated herein by reference) and include, without limitations, antifolates; fluoropyrimidines; cytarabine; purine analogs; adenosine analogs; amsacrine; topoisomerase I inhibitors; anthrapyrazoles; retinoids; antibiotics such as bleomycin, anthacyclins, mitomycin C, dactinomycin, and mithramycin; hexamethylmelamine; dacarbazine; I-asperginase; platinum analogs; alkylating agents such as nitrogen mustard, melphalan, chlorambucil, busulfan, ifosfamide, 4-hydroperoxycyclophosphamide, nitrosoureas, thiotepa; plant derived compounds such as vinca alkaloids, epipodophyllotoxins, taxol; Tomaxifen; radiation therapy; surgery; nutritional supplements; gene therapy; radiotherapy such as 3D-CRT; immunotoxin therapy such as ricin, monoclonal antibodies herceptin; and the like. For combination therapy, the nucleic acids of the invention are prepared in one of two ways. First, the agents are physically combined in a preparation of nucleic acid and chemotherapeutic agent, such as a mixture of a nucleic acid of the invention encapsulated in liposomes and ifosfamide in a solution for intravenous administration, wherein both agents are present in a therapeutically effective concentration (e.g., ifosfamide in solution to deliver 1000-1250 mg/m2/day and liposome-associated nucleic acid of the invention in the same solution to deliver 0.1-100 mg/kg/day). Alternatively, the agents are administered separately but simultaneously in their respective effective doses (e.g., 1000-1250 mg/m2/d ifosfamide and 0.1 to 100 mg/kg/day nucleic acid of the invention).

Diagnostic Uses

The nucleic acid molecules of this invention (e.g., ribozymes) may be used as diagnostic tools to examine genetic drift and mutations within diseased cells or to detect the presence of TERT RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These experiments will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with TERT-related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme will be used to identify mutant RNA in the sample. As reaction controls, synthetic substrates of both wild-type and mutant RNA will be cleaved by both ribozymes to demonstrate the relative ribozyme efficiencies in the reactions and the absence of cleavage of the “non-targeted” RNA species. The cleavage products from the synthetic substrates will also serve to generate size markers for the analysis of wild-type and mutant RNAs in the sample population. Thus each analysis will require two ribozymes, two substrates and one unknown sample which will be combined into six reactions. The presence of cleavage products will be determined using an RNAse protection assay so that full-length and cleavage fragments of each RNA can be analyzed in one lane of a polyacrylamide gel. For example, the cleavage reactions are performed in ribozyme cleavage buffer with a final reaction volume of 30 μl per reaction as follows: 1) ribozyme specific for (i.e., that specifically cleaves) wild-type RNA (wt ribozyme; 40 nM final reaction concentration) is incubated with wild type substrate (1-5 nM final reaction concentration) at 37° C. for one hour; 2) wt ribozyme is incubated with mutant substrate (same conditions); 3) wt ribozyme (40 nM final concentration) is incubated with 50 μg of total RNA from the individual being tested, at 37° C. for one hour; 4) same as (1), only with 40 nM final concentration of ribozyme specific for mutant RNA; 5) same as (2), only with ribozyme specific for mutant RNA; and 6) same as (3), only with ribozyme specific for mutant RNA. Cleavage products are precipitated with ethanol and resuspended in 20 μl of hybridization buffer for RNAse protection with 5×10⁵ to 1×10⁷ cpm of ³²P-labeled RNA probe. Hybridization buffer consists of the following (per reaction): 24 μl Formamide, 2 μl 0.6M PIPES, 2.4 μl 5M NaCl, 0.3 μl 0.1M EDTA, and DEPC-treated water to 30 μl. Samples are heated at 95° C. for 10 minutes, then incubated 4 hours at 55° C. (hybridization temperatures may be estimated by one of skill in the art and optimized empirically for a given probe:target combination without undue experimentation). Following hybridization, hybridized sequences are digested with ribonucleases by the addition of 350 μl of RNase digestion buffer (300 mM NaOAc, 10 mM Tris, 5 mM EDTA) followed by addition of 1 μl of 4 mg/ml RNase A and 0.4 μl of 10 u/μl RNase T1. Digestion is carried out for 45 minutes to 1 hour at 30° C., followed by the addition of 10 μl of 20% SDS and 2.5 μl of 10mg/ml Proteinase K. Samples are incubated at 37° C. for 15-20 minutes followed by phenol/chloroform/isoamyl alcohol (25:24:1) extraction and precipitation with ethanol. Samples are resuspended in formamide loading buffer, heat denatured and electrophoresed on a denaturing polyacrylamide gel. Protected cleavage products are visualized by autoradiography and quantitated by phosphorimager analysis. It is not absolutely required to quantify the results to gain insight into the expression of mutant RNAs and putative risk of the desired phenotypic changes in target cells. The expression of mRNA whose protein product is implicated in the development of the phenotype (i.e., TERT) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to
generate cleavage products with 3′-OH and 5′-guanosine.
Additional protein cofactors required in some cases to help folding and
maintainance of the active structure.
Over 300 known members of this class. Found as an intervening sequence
in Tetrahymena thermophila rRNA, fungal mitochondria, chloroplasts,
phage T4, blue-green algae, and others.
Major structural features largely established through phylogenetic
comparisons, mutagenesis, and biochemical studies [^{i,ii}].
Complete kinetic framework established for one ribozyme [^{iii,iv,v,vi}].
Studies of ribozyme folding and substrate docking underway [^{vii,viii,ix}].
Chemical modification investigation of important residues well
established [^{x,xi}].
The small (4-6 nt) binding site may make this ribozyme too non-specific
for targeted RNA cleavage, however, the Tetrahymena group I intron has
been used to repair a “defective” β-galactosidase message by the ligation
of new β-galactosidase sequences onto the defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate
cleavage products with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is
possible through hybridization of an External Guide Sequence (EGS) to
the target RNA [^{xiv,xv}]
Important phosphate and 2′ OH contacts recently identified [^{xvi,xvii}]
Group II Introns
Size: >1000 nucleotides.
Trans cleavage of target RNAs recently demonstrated [^{xviii,xix}].
Sequence requirements not fully determined.
Reaction mechanism: 2′-OH of an internal adenosine generates cleavage
products with 3′-OH and a “lariat” RNA containing a 3-5′ and a 2′-5′
branch point.
Only natural ribozyme with demonstrated participation in DNA
cleavage [^{xx,xxi}] in addition to RNA cleavage and ligation.
Major structural features largely established through phylogenetic
comparisons [^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal
structures [^{xxvi,xxvii}]
Minimal ligation activity demonstrated (for engineering through in vitro
selection) [^{xxviii}]
Complete kinetic framework established for two or more ribozymes [^{xxix}].
Chemical modification investigation of important residues well
established [^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi,xxxii,xxxiii,xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues
begun [^{xxxvii,xxxviii}].
Hepatitis Delta Virus (HDV) Ribozym
Size: ˜60 nucleotides.
Trans cleavage of target RNAs demonstrated [^{xxxix}].
Binding sites and structural requirements not fully determined, although
no sequences 5′ of cleavage site are required. Folded ribozyme contains a
pseudoknot structure [^{xl}].
Reaction mechanism: attack by 2′-OH 5′ to the scissile bond to generate
cleavage products with 2′,3′-cyclic phosphate and 5′-OH ends.
Only 2 known members of this class. Found in human HDV.
Circular form of HDV is active and shows increased nuclease stability [^{xli}]
[ⁱ] Michel, Francois; Westhof, Eric. Slippery substrates. Nat. Struct. Biol. (1994), 1(1), 5-7.
[ⁱⁱ] Lisacek, Frederique; Diaz, Yalande; Michel, Francois. Automatic identification of group I intron cores in genomic DNA sequences. J. Mol. Biol. (1994), 235(4), 1206-17.
[ⁱⁱⁱ] Herschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
[^{iv}] Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
[^{v}] Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
[^{vi}] Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
[^{vii}] Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
[^{viii}] Banerjee, Aloke Raj; Turner, Douglas H.. The time dependence of chemical modification reveals slow steps in the folding of a group I ribozyme. Biochemistry (1995), 34(19), 6504-12.
[^{ix}] Zarrinkar, Patrick P.; Williamson, James R.. The P9.1-P9.2 peripheral extension helps guide folding of the Tetrahymena ribozyme. Nucleic Acids Res. (1996), 24(5), 854-8.
[^{x}] Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
[^{xi}] Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
[^{xii}] Sullenger, Bruce A.; Cech, Thomas R.. Ribozyme-mediated repair of defective mRNA by targeted trans-splicing. Nature (London) (1994), 371(6498), 619-22.
[^{xiii}] Robertson, H. D.; Altman, S.; Smith, J. D. J. Biol Chem., 247, 5243-5251 (1972).
[^{xiv}] Forster, Anthony C.; Altman, Sidney. External guide sequences for an RNA enzyme. Science (Washington, D.C., 1883-) (1990), 249(4970), 783-6.
[^{xv}] Yuan, Y.; Hwang, E. S.; Altman, S. Targeted cleavage of mRNA by human RNase P. Proc. Natl. Acad. Sci. USA (1992) 89, 8006-10.
[^{xvi}] Harris, Michael E.; Pace, Norman R.. Identification of phosphates involved in catalysis by the ribozyme RNase P RNA. RNA (1995), 1(2), 210-18.
[^{xvii}] Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
[^{xviii}] Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
[^{xix}] Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
[^{xx}] Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
[^{xxi}] Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
[^{xxii}] Michel, Francois; Ferat, Jean Luc. Structure and activities of group II introns. Annu. Rev. Biochem. (1995), 64, 435-61.
[^{xxiii}] Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
[^{xxiv}] Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
[^{xxv}] Guo, Hans C. T.; Collins, Richard A.. Efficient trans-cleavage of a stem-loop RNA substrate by a ribozyme derived from Neurospora VS RNA. EMBO J. (1995), 14(2), 368-76.
[^{xxvi}] Scott, W. G., Finch, J. T., Aaron, K. The crystal structure of an all RNA hammerhead ribozyme: A proposed mechanism for RNA catalytic cleavage. Cell, (1995), 81, 991-1002.
[^{xxvii}] McKay, Structure and function of the hammerhead ribozyme: an unfinished story. RNA, (1996), 2, 395-403.
[^{xxviii}] Long, D., Uhlenbeck, O., Hertel, K. Ligation with hammerhead ribozymes. U.S. Pat. No. 5,633,133.
[^{xxix}] Hertel, K. J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
[^{xxx}] Beigelman, L., et al., Chemical modifications of hammerhead ribozymes. J. Biol. Chem., (1995) 270, 25702-25708.
[^{xxxi}] Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.
[^{xxxii}] Chowrira, Bharat M.; Berzal-Herranz, Alfredo; Burke, John M.. Novel guanosine requirement for catalysis by the hairpin ribozyme. Nature (London) (1991), 354(6351), 320-2.
[^{xxxiii}] Berzal-Herranz, Alfredo; Joseph, Simpson: Chowrira, Bharat M.; Butcher, Samuel E.; Burke, John M.. Essential nucleotide sequences and secondary structure elements of the hairpin ribozyme. EMBO J. (1993), 12(6), 2567-73.
[^{xxxiv}] Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
[^{xxxv}] Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
[^{xxxvi}] Hegg, Lisa A.; Fedor, Martha J.. Kinetics and Thermodynamics of Intermolecular Catalysis by Hairpin Ribozymes. Biochemistry (1995), 34(48), 15813-28.
[^{xxxvii}] Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
[^{xxxviii}] Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
[^{xxix}] Perrotta, Anne T.; Been, Michael D.. Cleavage of oligoribonucleotides by a ribozyme derived from the hepatitis delta. virus RNA sequence. Biochemistry (1992), 31(1), 16-21.
[^{xl}] Perrotta, Anne T.; Been, Michael D.. A pseudoknot-like structure required for efficient self-cleavage of hepatitis delta virus RNA. Nature (London) (1991), 350(6317), 434-6.
[^{xli}] Puttaraju, M.; Perrotta, Anne T.; Been, Michael D.. A circular trans-acting hepatitis delta virus ribozyme. Nucleic Acids Res. (1993), 21(18), 4253-8.

TABLE II
0.2 μmol RNA Synthesis Cycle

| Reagents | Equivalents | Amounts (microL) | Wait time (sec) |
|---|---|---|---|
| Phosphoramidites | 15 | 31 | 465 |
| SET | 38.7 | 31 | 465 |
| Acetic anhydride | 655 | 124 | 5 |
| N-methyl-imidazole | 1245 | 124 | 5 |
| TCA | 700 | 732 | 10 |
| Iodine | 20.6 | 244 | 15 |

* Wait time does not include contact time during delivery.

TABLE III
Human telomerase reverse transcriptase (TERT) Hammerhead Ribozyme and Target Sequence

| nt. | | Seq ID | Substrate | Seq ID |
|---|---|---|---|---|
| Position | Ribozyme Sequence | Nos. | Sequence | Nos. |
| 13 | CGCAGCAG CUGAUGAG GCCGUUAGGC CGAA ACGCAGCG | 2780 | CGCUGCGU C CUGCUGCG | 1 |
| 68 | GCAGCGGG CUGAUGAG GCCGUUAGGC CGAA AGCGCGCG | 2781 | CGCGCGCU C CCCGCUGC | 2 |
| 90 | GCAGCAGG CUGAUGAG GCCGUUAGGC CGAA AGCGCACG | 2782 | CGUGCGCU C CCUGCUGC | 3 |
| 108 | CCUCGCGG CUGAUGAG GCCGUUAGGC CGAA AGUGGCUG | 2783 | CAGCCACU A CCGCGAGG | 4 |
| 135 | GCCGCACG CUGAUGAG GCCGUUAGGC CGAA ACGUGGCC | 2784 | GGCCACGU U CGUGCGGC | 5 |
| 136 | CGCCGCAC CUGAUGAG GCCGUUAGGC CGAA AACGUGGC | 2785 | GCCACGUU C GUGCGGCG | 6 |
| 194 | CGCGCGGA CUGAUGAG GCCGUUAGGC CGAA AGCCGCCG | 2786 | CGGCGGCU U UCCGCGCG | 7 |
| 195 | GCGCGCGG CUGAUGAG GCCGUUAGGC CGAA AAGCCGCC | 2787 | GGCGGCUU U CCGCGCGC | 8 |
| 196 | AGCGCGCG CUGAUGAG GCCGUUAGGC CGAA AAAGCCGC | 2788 | GCGGCUUU C CGCGCGCU | 9 |
| 264 | GGCGGAAG CUGAUGAG GCCGUUAGGC CGAA AGGGGGCG | 2789 | CGCCCCCU C CUUCCGCC | 10 |
| 267 | CCUGGCGG CUGAUGAG GCCGUUAGGC CGAA AGGAGGGG | 2790 | CCCCUCCU U CCGCCAGG | 11 |
| 268 | ACCUGGCG CUGAUGAG GCCGUUAGGC CGAA AAGGAGGG | 2791 | CCCUCCUU C CGCCAGGU | 12 |
| 279 | UCAGGCAG CUGAUGAG GCCGUUAGGC CGAA ACACCUGG | 2792 | CCAGGUGU C CUGCCUGA | 13 |
| 351 | CGAAGCCG CUGAUGAG GCCGUUAGGC CGAA AGGCCAGC | 2793 | GCUGGCCU U CGGCUUCG | 14 |
| 352 | GCGAAGCC CUGAUGAG GCCGUUAGGC CGAA AAGGCCAG | 2794 | CUGGCCUU C GGCUUCGC | 15 |
| 357 | GCAGCGCG CUGAUGAG GCCGUUAGGC CGAA AGCCGAAG | 2795 | CUUCGGCU U CGCGCUGC | 16 |
| 358 | AGCAGCGC CUGAUGAG GCCGUUAGGC CGAA AAGCCGAA | 2796 | UUCGGCUU C GCGCUGCU | 17 |
| 399 | UGGUGGUG CUGAUGAG GCCGUUAGGC CGAA AGGCCUCG | 2797 | CGAGGCCU U CACCACCA | 18 |
| 400 | CUGGUGGU CUGAUGAG GCCGUUAGGC CGAA AAGGCCUC | 2798 | GAGGCCUU C ACCACCAG | 19 |
| 420 | UGGGCAGG CUGAUGAG GCCGUUAGGC CGAA AGCUGCGC | 2799 | GCGCAGCU A CCUGCCCA | 20 |
| 505 | AGCAGGUG CUGAUGAG GCCGUUAGGC CGAA ACCAGCAC | 2800 | GUGCUGGU U CACCUGCU | 21 |
| 506 | CAGCAGGU CUGAUGAG GCCGUUAGGC CGAA AACCAGCA | 2801 | UGCUGGUU C ACCUGCUG | 22 |
| 529 | AGCACAAA CUGAUGAG GCCGUUAGGC CGAA AGCGCGCA | 2802 | UGCGCGCU C UUUGUGCU | 23 |
| 531 | CCAGCACA CUGAUGAG GCCGUUAGGC CGAA AGAGCGCG | 2803 | CGCGCUCU U UGUGCUGG | 24 |
| 532 | ACCAGCAC CUGAUGAG GCCGUUAGGC CGAA AAGAGCGC | 2804 | GCGCUCUU U GUGCUGGU | 25 |
| 545 | GCAGCUGG CUGAUGAG GCCGUUAGGC CGAA AGCCACCA | 2805 | UGGUGGCU C CCAGCUGC | 26 |
| 558 | ACACCUGG CUGAUGAG GCCGUUAGGC CGAA AGGCGCAG | 2806 | CUGCGCCU A CCAGGUGU | 27 |
| 582 | CGAGCUGG CUGAUGAG GCCGUUAGGC CGAA ACAGCGGC | 2807 | GCCGCUGU A CCAGCUCG | 28 |
| 589 | GCAGCGCC CUGAUGAG GCCGUUAGGC CGAA AGCUGGUA | 2808 | UACCAGCU C GGCGCUGC | 29 |
| 602 | CCGGGCCU CUGAUGAG GCCGUUAGGC CGAA AGUGGCAG | 2809 | CUGCCACU C AGGCCCGG | 30 |
| 626 | GGGUCCAC CUGAUGAG GCCGUUAGGC CGAA AGCGUGUG | 2810 | CACACGCU A GUGGACCC | 31 |
| 644 | GCAUCCCA CUGAUGAG GCCGUUAGGC CGAA ACGCCUUC | 2811 | GAAGGCGU C UGGGAUGC | 32 |
| 671 | CCUGACGC CUGAUGAG GCCGUUAGGC CGAA AUGGUUCC | 2812 | GGAACCAU A GCGUCAGG | 33 |
| 676 | GCCUCCCU CUGAUGAG GCCGUUAGGC CGAA ACGCUAUG | 2813 | CAUAGCGU C AGGGAGGC | 34 |
| 691 | CCCAGGGG CUGAUGAG GCCGUUAGGC CGAA ACCCCGGC | 2814 | GCCGGGGU C CCCCUGGG | 35 |
| 749 | CAACGGCA CUGAUGAG GCCGUUAGGC CGAA ACUUCGGC | 2815 | GCCGAAGU C UGCCGUUG | 36 |
| 756 | UCUUGGGC CUGAUGAG GCCGUUAGGC CGAA ACGGCAGA | 2816 | UCUGCCGU U GCCCAAGA | 37 |
| 808 | CCCUGCCC CUGAUGAG GCCGUUAGGC CGAA ACGGGCGU | 2817 | ACGCCCGU U GGGCAGGG | 38 |
| 819 | GGGCCCAG CUGAUGAG GCCGUUAGGC CGAA ACCCCUGC | 2818 | GCAGGGGU C CUGGGCCC | 39 |
| 863 | CACACAGA CUGAUGAG GCCGUUAGGC CGAA ACCACGGU | 2819 | ACCGUGGU U UCUGUGUG | 40 |
| 864 | CCACACAG CUGAUGAG GCCGUUAGGC CGAA AACCACGG | 2820 | CCGUGGUU U CUGUGUGG | 41 |
| 865 | ACCACACA CUGAUGAG GCCGUUAGGC CGAA AAACCACG | 2821 | CGUGGUUU C UGUGUGGU | 42 |
| 876 | UGGCAGGU CUGAUGAG GCCGUUAGGC CGAA ACACCACA | 2822 | UGUGGUGU C ACCUGCCA | 43 |
| 906 | CCUCCAAA CUGAUGAG GCCGUUAGGC CGAA AGGUGGCU | 2823 | AGCCACCU C UUUGGAGG | 44 |
| 908 | ACCCUCCA CUGAUGAG GCCGUUAGGC CGAA AGAGGUGG | 2824 | CCACCUCU U UGGAGGGU | 45 |
| 909 | CACCCUCC CUGAUGAG GCCGUUAGGC CGAA AAGAGGUG | 2825 | CACCUCUU U GGAGGGUG | 46 |
| 922 | GUGCCAGA CUGAUGAG GCCGUUAGGC CGAA AGCGCACC | 2826 | GGUGCGCU C UCUGGCAC | 47 |
| 924 | GCGUGCCA CUGAUGAG GCCGUUAGGC CGAA AGAGCGCA | 2827 | UGCGCUCU C UGGCACGC | 48 |
| 939 | AUGGGUGG CUGAUGAG GCCGUUAGGC CGAA AGUGGCGC | 2828 | GCGCCACU C CCACCCAU | 49 |
| 948 | GGCCCACG CUGAUGAG GCCGUUAGGC CGAA AUGGGUGG | 2829 | CCACCCAU C CGUGGGCC | 50 |
| 981 | GCGAUGUG CUGAUGAG GCCGUUAGGC CGAA AUGGGGGG | 2830 | CCCCCCAU C CACAUCGC | 51 |
| 987 | GUGGCCGC CUGAUGAG GCCGUUAGGC CGAA AUGUGGAU | 2831 | AUCCACAU C GCGGCCAC | 52 |
| 1001 | GUCCCAGG CUGAUGAG GCCGUUAGGC CGAA ACGUGGUG | 2832 | CACCACGU C CCUGGGAC | 53 |
| 1016 | CGGGGGAC CUGAUGAG GCCGUUAGGC CGAA AGGCGUGU | 2833 | ACACGCCU U GUCCCCCG | 54 |
| 1019 | CACCGGGG CUGAUGAG GCCGUUAGGC CGAA ACAAGGCG | 2834 | CGCCUUGU C CCCCGGUG | 55 |
| 1029 | UCUCGGCG CUGAUGAG GCCGUUAGGC CGAA ACACCGGG | 2835 | CCCGGUGU A CGCCGAGA | 56 |
| 1047 | AGUAGAGG CUGAUGAG GCCGUUAGGC CGAA AGUGCUUG | 2836 | CAAGCACU U CCUCUACU | 57 |
| 1048 | GAGUAGAG CUGAUGAG GCCGUUAGGC CGAA AAGUGCUU | 2837 | AAGCACUU C CUCUACUC | 58 |
| 1051 | GAGGAGUA CUGAUGAG GCCGUUAGGC CGAA AGGAAGUG | 2838 | CACUUCCU C UACUCCUC | 59 |
| 1053 | CUGAGGAG CUGAUGAG GCCGUUAGGC CGAA AGAGGAAG | 2839 | CUUCCUCU A CUCCUCAG | 60 |
| 1056 | CGCCUGAG CUGAUGAG GCCGUUAGGC CGAA AGUAGAGG | 2840 | CCUCUACU C CUCAGGCG | 61 |
| 1059 | UGUCGCCU CUGAUGAG GCCGUUAGGC CGAA AGGAGUAG | 2841 | CUACUCCU C AGGCGACA | 62 |
| 1086 | GUAGGAAG CUGAUGAG GCCGUUAGGC CGAA AGGGCCGC | 2842 | GCGGCCCU C CUUCCUAC | 63 |
| 1089 | UGAGUAGG CUGAUGAG GCCGUUAGGC CGAA AGGAGGGC | 2843 | GCCCUCCU U CCUACUCA | 64 |
| 1090 | CUGAGUAG CUGAUGAG GCCGUUAGGC CGAA AAGGAGGG | 2844 | CCCUCCUU C CUACUCAG | 65 |
| 1093 | GAGCUGAG CUGAUGAG GCCGUUAGGC CGAA AGGAAGGA | 2845 | UCCUUCCU A CUCAGCUC | 66 |
| 1096 | AGAGAGCU CUGAUGAG GCCGUUAGGC CGAA AGUAGGAA | 2846 | UUCCUACU C AGCUCUCU | 67 |
| 1101 | GCCUCAGA CUGAUGAG GCCGUUAGGC CGAA AGCUGAGU | 2847 | ACUCAGCU C UCUGAGGC | 68 |
| 1103 | GGGCCUCA CUGAUGAG GCCGUUAGGC CGAA AGAGCUGA | 2848 | UCAGCUCU C UGAGGCCC | 69 |
| 1127 | GAGCCUCC CUGAUGAG GCCGUUAGGC CGAA AGCGCCAG | 2849 | CUGGCGCU C GGAGGCUC | 70 |
| 1135 | GUCUCCAC CUGAUGAG GCCGUUAGGC CGAA AGCCUCCG | 2850 | CGGAGGCU C GUGGAGAC | 71 |
| 1147 | CCCAGAAA CUGAUGAG GCCGUUAGGC CGAA AUGGUCUC | 2851 | GAGACCAU C UUUCUGGG | 72 |
| 1149 | AACCCAGA CUGAUGAG GCCGUUAGGC CGAA AGAUGGUC | 2852 | GACCAUCU U UCUGGGUU | 73 |
| 1150 | GAACCCAG CUGAUGAG GCCGUUAGGC CGAA AAGAUGGU | 2853 | ACCAUCUU U CUGGGUUC | 74 |
| 1151 | GGAACCCA CUGAUGAG GCCGUUAGGC CGAA AAAGAUGG | 2854 | CCAUCUUU C UGGGUUCC | 75 |
| 1157 | GGGCCUGG CUGAUGAG GCCGUUAGGC CGAA ACCCAGAA | 2855 | UUCUGGGU U CCAGGCCC | 76 |
| 1158 | AGGGCCUG CUGAUGAG GCCGUUAGGC CGAA AACCCAGA | 2856 | UCUGGGUU C CAGGCCCU | 77 |
| 1181 | CCUGCGGG CUGAUGAG GCCGUUAGGC CGAA AGUCCCUG | 2857 | CAGGGACU C CCCGCAGG | 78 |
| 1191 | GGCGGGGC CUGAUGAG GCCGUUAGGC CGAA ACCUGCGG | 2858 | CCGCAGGU U GCCCCGCC | 79 |
| 1212 | UUUGCCAG CUGAUGAG GCCGUUAGGC CGAA AGCGCUGG | 2859 | CCAGCGCU A CUGGCAAA | 80 |
| 1233 | GCUCCAGA CUGAUGAG GCCGUUAGGC CGAA ACAGGGGC | 2860 | GCCCCUGU U UCUGGAGC | 81 |
| 1234 | AGCUCCAG CUGAUGAG GCCGUUAGGC CGAA AACAGGGG | 2861 | CCCCUGUU U CUGGAGCU | 82 |
| 1235 | CAGCUCCA CUGAUGAG GCCGUUAGGC CGAA AAACAGGG | 2862 | CCCUGUUU C UGGAGCUG | 83 |
| 1246 | UGGUUCCC CUGAUGAG GCCGUUAGGC CGAA AGCAGCUC | 2863 | GAGCUGCU U GGGAACCA | 84 |
| 1269 | GCACCCCG CUGAUGAG GCCGUUAGGC CGAA AGGGGCAC | 2864 | GUGCCCCU A CGGGGUGC | 85 |
| 1279 | GUCUUGAG CUGAUGAG GCCGUUAGGC CGAA AGCACCCC | 2865 | GGGGUGCU C CUCAAGAC | 86 |
| 1282 | UGCGUCUU CUGAUGAG GCCGUUAGGC CGAA AGGAGCAC | 2866 | GUGCUCCU C AAGACGCA | 87 |
| 1312 | GCUGGGGU CUGAUGAG GCCGUUAGGC CGAA ACCGCAGC | 2867 | GCUGCGGU C ACCCCAGC | 88 |
| 1330 | CGGGCACA CUGAUGAG GCCGUUAGGC CGAA ACACCGGC | 2868 | GCCGGUGU C UGUGCCCG | 89 |
| 1356 | CCGCCACA CUGAUGAG GCCGUUAGGC CGAA AGCCCUGG | 2869 | CCAGGGCU C UGUGGCGG | 90 |
| 1394 | CACCAGGC CUGAUGAG GCCGUUAGGC CGAA ACGGGGGU | 2870 | ACCCCCGU C GCCUGGUG | 91 |
| 1411 | UGCUGGCG CUGAUGAG GCCGUUAGGC CGAA AGCAGCUG | 2871 | CAGCUGCU C CGCCAGCA | 92 |
| 1440 | CGAAGCCG CUGAUGAG GCCGUUAGGC CGAA ACACCUGC | 2872 | GCAGGUGU A CGGCUUCG | 93 |
| 1446 | CCCGCACG CUGAUGAG GCCGUUAGGC CGAA AGCCGUAC | 2873 | GUACGGCU U CGUGCGGG | 94 |
| 1447 | GCCCGCAC CUGAUGAG GCCGUUAGGC CGAA AAGCCGUA | 2874 | UACGGCUU C GUGCGGGC | 95 |
| 1486 | GAGCCCCA CUGAUGAG GCCGUUAGGC CGAA AGGCCUGG | 2875 | CCAGGCCU C UGGGGCUC | 96 |
| 1494 | UGUGCCUG CUGAUGAG GCCGUUAGGC CGAA AGCCCCAG | 2876 | CUGGGGCU C CAGGCACA | 97 |
| 1515 | UCCUGAGG CUGAUGAG GCCGUUAGGC CGAA AGCGGCGU | 2877 | ACGCCGCU U CCUCAGGA | 98 |
| 1516 | UUCCUGAG CUGAUGAG GCCGUUAGGC CGAA AAGCGGCG | 2878 | CGCCGCUU C CUCAGGAA | 99 |
| 1519 | GUGUUCCU CUGAUGAG GCCGUUAGGC CGAA AGGAAGCG | 2879 | CGCUUCCU C AGGAACAC | 100 |
| 1536 | GGGAGAUG CUGAUGAG GCCGUUAGGC CGAA ACUUCUUG | 2880 | CAAGAAGU U CAUCUCCC | 101 |
| 1537 | AGGGAGAU CUGAUGAG GCCGUUAGGC CGAA AACUUCUU | 2881 | AAGAAGUU C AUCUCCCU | 102 |
| 1540 | CCCAGGGA CUGAUGAG GCCGUUAGGC CGAA AUGAACUU | 2882 | AAGUUCAU C UCCCUGGG | 103 |
| 1542 | UCCCCAGG CUGAUGAG GCCGUUAGGC CGAA AGAUGAAC | 2883 | GUUCAUCU C CCUGGGGA | 104 |
| 1564 | UGCAGCGA CUGAUGAG GCCGUUAGGC CGAA AGCUUGGC | 2884 | GCCAAGCU C UCGCUGCA | 105 |
| 1566 | CCUGCAGC CUGAUGAG GCCGUUAGGC CGAA AGAGCUUG | 2885 | CAAGCUCU C GCUGCAGG | 106 |
| 1610 | GCGCAGCC CUGAUGAG GCCGUUAGGC CGAA AGCGCAGU | 2886 | ACUGCGCU U GGCUGCGC | 107 |
| 1633 | ACACAGCC CUGAUGAG GCCGUUAGGC CGAA ACCCCUGG | 2887 | CCAGGGGU U GGCUGUGU | 108 |
| 1642 | GCGGCCGG CUGAUGAG GCCGUUAGGC CGAA ACACAGCC | 2888 | GGCUGUGU U CCGGCCGC | 109 |
| 1643 | UGCGGCCG CUGAUGAG GCCGUUAGGC CGAA AACACAGC | 2889 | GCUGUGUU C CGGCCGCA | 110 |
| 1661 | GUCACGCA CUGAUGAG GCCGUUAGGC CGAA ACGGUGCU | 2890 | AGCACCGU C UGCGUGAG | 111 |
| 1675 | UUGGCCAG CUGAUGAG GCCGUUAGGC CGAA AUCUCCUC | 2891 | GAGGAGAU C CUGGCCAA | 112 |
| 1686 | AGUGCAGG CUGAUGAG GCCGUUAGGC CGAA ACUUGGCC | 2892 | GGCCAAGU U CCUGCACU | 113 |
| 1687 | CAGUGCAG CUGAUGAG GCCGUUAGGC CGAA AACUUGGC | 2893 | GCCAAGUU C CUGCACUG | 114 |
| 1710 | CGACGACG CUGAUGAG GCCGUUAGGC CGAA ACACACUC | 2894 | GAGUGUGU A CGUCGUCG | 115 |
| 1714 | AGCUCGAC CUGAUGAG GCCGUUAGGC CGAA ACGUACAC | 2895 | GUGUACGU C GUCGAGCU | 116 |
| 1717 | AGCAGCUC CUGAUGAG GCCGUUAGGC CGAA ACGACGUA | 2896 | UACGUCGU C GAGCUGCU | 117 |
| 1726 | AAAGACCU CUGAUGAG GCCGUUAGGC CGAA AGCAGCUC | 2897 | GAGCUGCU C AGGUCUUU | 118 |
| 1731 | AAAAGAAA CUGAUGAG GCCGUUAGGC CGAA ACCUGAGC | 2898 | GCUCAGGU C UUUCUUUU | 119 |
| 1733 | AUAAAAGA CUGAUGAG GCCGUUAGGC CGAA AGACCUGA | 2899 | UCAGGUCU U UCUUUUAU | 120 |
| 1734 | CAUAAAAG CUGAUGAG GCCGUUAGGC CGAA AAGACCUG | 2900 | CAGGUCUU U CUUUUAUG | 121 |
| 1735 | ACAUAAAA CUGAUGAG GCCGUUAGGC CGAA AAAGACCU | 2901 | AGGUCUUU C UUUUAUGU | 122 |
| 1737 | UGACAUAA CUGAUGAG GCCGUUAGGC CGAA AGAAAGAC | 2902 | GUCUUUCU U UUAUGUCA | 123 |
| 1738 | GUGACAUA CUGAUGAG GCCGUUAGGC CGAA AAGAAAGA | 2903 | UCUUUCUU U UAUGUCAC | 124 |
| 1739 | CGUGACAU CUGAUGAG GCCGUUAGGC CGAA AAAGAAAG | 2904 | CUUUCUUU U AUGUCACG | 125 |
| 1740 | CCGUGACA CUGAUGAG GCCGUUAGGC CGAA AAAAGAAA | 2905 | UUUCUUUU A UGUCACGG | 126 |
| 1744 | GUCUCCGU CUGAUGAG GCCGUUAGGC CGAA ACAUAAAA | 2906 | UUUUAUGU C ACGGAGAC | 127 |
| 1758 | UCUUUUGA CUGAUGAG GCCGUUAGGC CGAA ACGUGGUC | 2907 | GACCACGU U UCAAAAGA | 128 |
| 1759 | UUCUUUUG CUGAUGAG GCCGUUAGGC CGAA AACGUGGU | 2908 | ACCACGUU U CAAAAGAA | 129 |
| 1760 | GUUCUUUU CUGAUGAG GCCGUUAGGC CGAA AAACGUGG | 2909 | CCACGUUU C AAAAGAAC | 130 |
| 1774 | UAGAAAAA CUGAUGAG GCCGUUAGGC CGAA AGCCUGUU | 2910 | AACAGGCU C UUUUUCUA | 131 |
| 1776 | GGUAGAAA CUGAUGAG GCCGUUAGGC CGAA AGAGCCUG | 2911 | CAGGCUCU U UUUCUACC | 122 |
| 1777 | CGGUAGAA CUGAUGAG GCCGUUAGGC CGAA AAGAGCCU | 2912 | AGGCUCUU U UUCUACCG | 133 |
| 1778 | CCGGUAGA CUGAUGAG GCCGUUAGGC CGAA AAAGAGCC | 2913 | GGCUCUUU U UCUACCGG | 134 |
| 1779 | UCCGGUAG CUGAUGAG GCCGUUAGGC CGAA AAAAGAGC | 2914 | GCUCUUUU U CUACCGGA | 135 |
| 1780 | UUCCGGUA CUGAUGAG GCCGUUAGGC CGAA AAAAAGAG | 2915 | CUCUUUUU C UACCGGAA | 136 |
| 1782 | UCUUCCGG CUGAUGAG GCCGUUAGGC CGAA AGAAAAAG | 2916 | CUUUUUCU A CCGGAAGA | 137 |
| 1795 | UUGCUCCA CUGAUGAG GCCGUUAGGC CGAA ACACUCUU | 2917 | AAGAGUGU C UGGAGCAA | 138 |
| 1806 | UGCUUUGC CUGAUGAG GCCGUUAGGC CGAA ACUUGCUC | 2918 | GAGCAAGU U GCAAAGCA | 139 |
| 1816 | CUGAUUCC CUGAUGAG GCCGUUAGGC CGAA AUGCUUUG | 2919 | CAAAGCAU U GGAAUCAG | 140 |
| 1822 | UGCUGUCU CUGAUGAG GCCGUUAGGC CGAA AUUCCAAU | 2920 | AUUGGAAU C AGACAGCA | 141 |
| 1833 | CCCUCUUC CUGAUGAG GCCGUUAGGC CGAA AGUGCUGU | 2921 | ACAGCACU U GAAGAGGG | 142 |
| 1860 | CUGCUUCC CUGAUGAG GCCGUUAGGC CGAA ACAGCUCC | 2922 | GGAGCUGU C GGAAGCAG | 143 |
| 1873 | UGCUGCCU CUGAUGAG GCCGUUAGGC CGAA ACCUCUGC | 2923 | GCAGAGGU C AGGCAGCA | 144 |
| 1883 | GGCUUCCC CUGAUGAG GCCGUUAGGC CGAA AUGCUGCC | 2924 | GGCAGCAU C GGGAAGCC | 145 |
| 1911 | GGAGUCUG CUGAUGAG GCCGUUAGGC CGAA ACGUCAGC | 2925 | GCUGACGU C CAGACUCC | 146 |
| 1918 | AUGAAGCG CUGAUGAG GCCGUUAGGC CGAA AGUCUGGA | 2926 | UCCAGACU C CGCUUCAU | 147 |
| 1923 | UGGGGAUG CUGAUGAG GCCGUUAGGC CGAA AGCGGAGU | 2927 | ACUCCGCU U CAUCCCCA | 148 |
| 1924 | UUGGGGAU CUGAUGAG GCCGUUAGGC CGAA AAGCGGAG | 2928 | CUCCGCUU C AUCCCCAA | 149 |
| 1927 | GGCUUGGG CUGAUGAG GCCGUUAGGC CGAA AUGAAGCG | 2929 | CGCUUCAU C CCCAAGCC | 150 |
| 1954 | AUGUUCAC CUGAUGAG GCCGUUAGGC CGAA AUCGGCCG | 2930 | CGGCCGAU U GUGAACAU | 151 |
| 1968 | CCACGACG CUGAUGAG GCCGUUAGGC CGAA AGUCCAUG | 2931 | CAUGGACU A CGUCGUGG | 152 |
| 1972 | GCUCCCAC CUGAUGAG GCCGUUAGGC CGAA ACGUAGUC | 2932 | GACUACGU C GUGGGAGC | 153 |
| 1989 | CUCUGCGG CUGAUGAG GCCGUUAGGC CGAA ACGUUCUG | 2933 | CAGAACGU U CCGCAGAG | 154 |
| 1990 | UCUCUGCG CUGAUGAG GCCGUUAGGC CGAA AACGUUCU | 2934 | AGAACGUU C CGCAGAGA | 155 |
| 2015 | CGAGGUGA CUGAUGAG GCCGUUAGGC CGAA ACGCUCGG | 2935 | CCGAGCGU C UCACCUCG | 156 |
| 2017 | CUCGAGGU CUGAUGAG GCCGUUAGGC CGAA AGACGCUC | 2936 | GAGCGUCU C ACCUCGAG | 157 |
| 2022 | UCACCCUC CUGAUGAG GCCGUUAGGC CGAA AGGUGAGA | 2937 | UCUCACCU C GAGGGUGA | 158 |
| 2040 | GCACGCUG CUGAUGAG GCCGUUAGGC CGAA ACAGUGCC | 2938 | GGCACUGU U CAGCGUGC | 159 |
| 2041 | AGCACGCU CUGAUGAG GCCGUUAGGC CGAA AACAGUGC | 2939 | GCACUGUU C AGCGUGCU | 160 |
| 2050 | UCGUAGUU CUGAUGAG GCCGUUAGGC CGAA AGCACGCU | 2940 | AGCGUGCU C AACUACGA | 161 |
| 2055 | CCCGCUCG CUGAUGAG GCCGUUAGGC CGAA AGUUGAGC | 2941 | GCUCAACU A CGAGCGGG | 162 |
| 2080 | GCGCCCAG CUGAUGAG GCCGUUAGGC CGAA AGGCCGGG | 2942 | CCCGGCCU C CUGGGCGC | 163 |
| 2091 | CCAGCACA CUGAUGAG GCCGUUAGGC CGAA AGGCGCCC | 2943 | GGGCGCCU C UGUGCUGG | 164 |
| 2111 | CCUGUGGA CUGAUGAG GCCGUUAGGC CGAA AUCGUCCA | 2944 | UGGACGAU A UCCACAGG | 165 |
| 2113 | GCCCUGUG CUGAUGAG GCCGUUAGGC CGAA AUAUCGUC | 2945 | GACGAUAU C CACAGGGC | 166 |
| 2133 | GCAGCACG CUGAUGAG GCCGUUAGGC CGAA AGGUGCGC | 2946 | GCGCACCU U CGUGCUGC | 167 |
| 2134 | CGCAGCAC CUGAUGAG GCCGUUAGGC CGAA AAGGUGCG | 2947 | CGCACCUU C GUGCUGCG | 168 |
| 2175 | UGACAAAG CUGAUGAG GCCGUUAGGC CGAA ACAGCUCA | 2948 | UGAGCUGU A CUUUGUCA | 169 |
| 2178 | CCUUGACA CUGAUGAG GCCGUUAGGC CGAA AGUACAGC | 2949 | GCUGUACU U UGUCAAGG | 170 |
| 2179 | ACCUUGAC CUGAUGAG GCCGUUAGGC CGAA AAGUACAG | 2950 | CUGUACUU U GUCAAGGU | 171 |
| 2182 | UCCACCUU CUGAUGAG GCCGUUAGGC CGAA ACAAAGUA | 2951 | UACUUUGU C AAGGUGGA | 172 |
| 2205 | UGGUGUCG CUGAUGAG GCCGUUAGGC CGAA ACGCGCCC | 2952 | GGGCGCGU A CGACACCA | 173 |
| 2215 | UCCUGGGG CUGAUGAG GCCGUUAGGC CGAA AUGGUGUC | 2953 | GACACCAU C CCCCAGGA | 174 |
| 2230 | ACCUCCGU CUGAUGAG GCCGUUAGGC CGAA AGCCUGUC | 2954 | GACAGGCU C ACGGAGGU | 175 |
| 2239 | CUGGCGAU CUGAUGAG GCCGUUAGGC CGAA ACCUCCGU | 2955 | ACGGAGGU C AUCGCCAG | 176 |
| 2242 | AUGCUGGC CUGAUGAG GCCGUUAGGC CGAA AUGACCUC | 2956 | GAGGUCAU C GCCAGCAU | 177 |
| 2251 | GGUUUGAU CUGAUGAG GCCGUUAGGC CGAA AUGCUGGC | 2957 | GCCAGCAU C AUCAAACC | 178 |
| 2254 | UGGGGUUU CUGAUGAG GCCGUUAGGC CGAA AUGAUGCU | 2958 | AGCAUCAU C AAACCCCA | 179 |
| 2271 | GCACGCAG CUGAUGAG GCCGUUAGGC CGAA ACGUGUUC | 2959 | GAACACGU A CUGCGUGC | 180 |
| 2282 | GGCAUACC CUGAUGAG GCCGUUAGGC CGAA ACGCACGC | 2960 | GCGUGCGU C GGUAUGCC | 181 |
| 2286 | CCACGGCA CUGAUGAG GCCGUUAGGC CGAA ACCGACGC | 2961 | GCGUCGGU A UGCCGUGG | 182 |
| 2296 | GCCUUCUG CUGAUGAG GCCGUUAGGC CGAA ACCACGGC | 2962 | GCCGUGGU C CAGAAGGC | 183 |
| 2320 | GCCUUGCG CUGAUGAG GCCGUUAGGC CGAA ACGUGCCC | 2963 | GGGCACGU C CGCAAGGC | 184 |
| 2331 | GGCUCUUG CUGAUGAG GCCGUUAGGC CGAA AGGCCUUG | 2964 | CAAGGCCU U CAAGAGCC | 185 |
| 2332 | UGGCUCUU CUGAUGAG GCCGUUAGGC CGAA AAGGCCUU | 2965 | AAGGCCUU C AAGAGCCA | 186 |
| 2344 | AAGGUAGA CUGAUGAG GCCGUUAGGC CGAA ACGUGGCU | 2966 | AGCCACGU C UCUACCUU | 187 |
| 2346 | UCAAGGUA CUGAUGAG GCCGUUAGGC CGAA AGACGUGG | 2967 | CCACGUCU C UACCUUGA | 188 |
| 2348 | UGUCAAGG CUGAUGAG GCCGUUAGGC CGAA AGAGACGU | 2968 | ACGUCUCU A CCUUGACA | 189 |
| 2352 | GGUCUGUC CUGAUGAG GCCGUUAGGC CGAA AGGUAGAG | 2969 | CUCUACCU U GACAGACC | 190 |
| 2362 | UACGGCUG CUGAUGAG GCCGUUAGGC CGAA AGGUCUGU | 2970 | ACAGACCU C CAGCCGUA | 191 |
| 2370 | GUCGCAUG CUGAUGAG GCCGUUAGGC CGAA ACGGCUGG | 2971 | CCAGCCGU A CAUGCGAC | 192 |
| 2382 | GAGCCACG CUGAUGAG GCCGUUAGGC CGAA ACUGUCGC | 2972 | GCGACAGU U CGUGGCUC | 193 |
| 2383 | UGAGCCAC CUGAUGAG GCCGUUAGGC CGAA AACUGUCG | 2973 | CGACAGUU C GUGGCUCA | 194 |
| 2390 | CUGCAGGU CUGAUGAG GCCGUUAGGC CGAA AGCCACGA | 2974 | UCGUGGCU C ACCUGCAG | 195 |
| 2425 | UCGAUGAC CUGAUGAG GCCGUUAGGC CGAA ACGGCAUC | 2975 | GAUGCCGU C GUCAUCGA | 196 |
| 2428 | UGCUCGAU CUGAUGAG GCCGUUAGGC CGAA ACGACGGC | 2976 | GCCGUCGU C AUCGAGCA | 197 |
| 2431 | CUCUGCUC CUGAUGAG GCCGUUAGGC CGAA AUGACGAC | 2977 | GUCGUCAU C GAGCAGAG | 198 |
| 2442 | UCAGGGAG CUGAUGAG GCCGUUAGGC CGAA AGCUCUGC | 2978 | GCAGAGCU C CUCCCUGA | 199 |
| 2445 | CAUUCAGG CUGAUGAG GCCGUUAGGC CGAA AGGAGCUC | 2979 | GAGCUCCU C CCUGAAUG | 200 |
| 2470 | ACGUCGAA CUGAUGAG GCCGUUAGGC CGAA AGGCCACU | 2980 | AGUGGCCU C UUCGACGU | 201 |
| 2472 | AGACGUCG CUGAUGAG GCCGUUAGGC CGAA AGAGGCCA | 2981 | UGGCCUCU U CGACGUCU | 202 |
| 2473 | AAGACGUC CUGAUGAG GCCGUUAGGC CGAA AAGAGGCC | 2982 | GGCCUCUU C GACGUCUU | 203 |
| 2479 | CGUAGGAA CUGAUGAG GCCGUUAGGC CGAA ACGUCGAA | 2983 | UUCGACGU C UUCCUACG | 204 |
| 2481 | AGCGUAGG CUGAUGAG GCCGUUAGGC CGAA AGACGUCG | 2984 | CGACGUCU U CCUACGCU | 205 |
| 2482 | AAGCGUAG CUGAUGAG GCCGUUAGGC CGAA AAGACGUC | 2985 | GACGUCUU C CUACGCUU | 206 |
| 2485 | AUGAAGCG CUGAUGAG GCCGUUAGGC CGAA AGGAAGAC | 2986 | GUCUUCCU A CGCUUCAU | 207 |
| 2490 | GGCACAUG CUGAUGAG GCCGUUAGGC CGAA AGCGUAGG | 2987 | CCUACGCU U CAUGUGCC | 208 |
| 2491 | UGGCACAU CUGAUGAG GCCGUUAGGC CGAA AAGCGUAG | 2988 | CUACGCUU C AUGUGCCA | 209 |
| 2515 | UUGCCCCU CUGAUGAG GCCGUUAGGC CGAA AUGCGCAC | 2989 | GUGCGCAU C AGGGGCAA | 210 |
| 2526 | GGACGUAG CUGAUGAG GCCGUUAGGC CGAA ACUUGCCC | 2990 | GGGCAAGU C CUACGUCC | 211 |
| 2529 | ACUGGACG CUGAUGAG GCCGUUAGGC CGAA AGGACUUG | 2991 | CAAGUCCU A CGUCCAGU | 212 |
| 2533 | UGGCACUG CUGAUGAG GCCGUUAGGC CGAA ACGUAGGA | 2992 | UCCUACGU C CAGUGCCA | 213 |
| 2548 | CCCUGCGG CUGAUGAG GCCGUUAGGC CGAA AUCCCCUG | 2993 | CAGGGGAU C CCGCAGGG | 214 |
| 2559 | AGAGGAUG CUGAUGAG GCCGUUAGGC CGAA AGCCCUGC | 2994 | GCAGGGCU C CAUCCUCU | 215 |
| 2563 | GUGGAGAG CUGAUGAG GCCGUUAGGC CGAA AUGGAGCC | 2995 | GGCUCCAU C CUCUCCAC | 216 |
| 2566 | AGCGUGGA CUGAUGAG GCCGUUAGGC CGAA AGGAUGGA | 2996 | UCCAUCCU C UCCACGCU | 217 |
| 2568 | GCAGCGUG CUGAUGAG GCCGUUAGGC CGAA AGAGGAUG | 2997 | CAUCCUCU C CACGCUGC | 218 |
| 2578 | AGGCUGCA CUGAUGAG GCCGUUAGGC CGAA AGCAGCGU | 2998 | ACGCUGCU C UGCAGCCU | 219 |
| 2592 | UGUCGCCG CUGAUGAG GCCGUUAGGC CGAA AGCACAGG | 2999 | CCUGUGCU A CGGCGACA | 220 |
| 2616 | UCCCCGCA CUGAUGAG GCCGUUAGGC CGAA ACAGCUUG | 3000 | CAAGCUGU U UGCGGGGA | 221 |
| 2617 | AUCCCCGC CUGAUGAG GCCGUUAGGC CGAA AACAGCUU | 3001 | AAGCUGUU U GCGGGGAU | 222 |
| 2626 | UCCCGCCG CUGAUGAG GCCGUUAGGC CGAA AUCCCCGC | 3002 | GCGGGGAU U CGGCGGGA | 223 |
| 2627 | GUCCCGCC CUGAUGAG GCCGUUAGGC CGAA AAUCCCCG | 3003 | CGGGGAUU C GGCGGGAC | 224 |
| 2644 | AAACGCAG CUGAUGAG GCCGUUAGGC CGAA AGCAGCCC | 3004 | GGGCUGCU C CUGCGUUU | 225 |
| 2651 | AUCCACCA CUGAUGAG GCCGUUAGGC CGAA ACGCAGGA | 3005 | UCCUGCGU U UGGUGGAU | 226 |
| 2652 | CAUCCACC CUGAUGAG GCCGUUAGGC CGAA AACGCAGG | 3006 | CCUGCGUU U GGUGGAUG | 227 |
| 2663 | CAACAAGA CUGAUGAG GCCGUUAGGC CGAA AUCAUCCA | 3007 | UGGAUGAU U UCUUGUUG | 228 |
| 2664 | CCAACAAG CUGAUGAG GCCGUUAGGC CGAA AAUCAUCC | 3008 | GGAUGAUU U CUUGUUGG | 229 |
| 2665 | ACCAACAA CUGAUGAG GCCGUUAGGC CGAA AAAUCAUC | 3009 | GAUGAUUU C UUGUUGGU | 230 |
| 2667 | UCACCAAC CUGAUGAG GCCGUUAGGC CGAA AGAAAUCA | 3010 | UGAUUUCU U GUUGGUGA | 231 |
| 2670 | GUGUCACC CUGAUGAG GCCGUUAGGC CGAA ACAAGAAA | 3011 | UUUCUUGU U GGUGACAC | 232 |
| 2681 | GGUGAGGU CUGAUGAG GCCGUUAGGC CGAA AGGUGUCA | 3012 | UGACACCU C ACCUCACC | 233 |
| 2686 | GCGUGGGU CUGAUGAG GCCGUUAGGC CGAA AGGUGAGG | 3013 | CCUCACCU C ACCCACGC | 234 |
| 2703 | UCCUGAGG CUGAUGAG GCCGUUAGGC CGAA AGGUUUUC | 3014 | GAAAACCU U CCUCAGGA | 235 |
| 2704 | GUCCUGAG CUGAUGAG GCCGUUAGGC CGAA AAGGUUUU | 3015 | AAAACCUU C CUCAGGAC | 236 |
| 2707 | AGGGUCCU CUGAUGAG GCCGUUAGGC CGAA AGGAAGGU | 3016 | ACCUUCCU C AGGACCCU | 237 |
| 2719 | ACACCUCG CUGAUGAG GCCGUUAGGC CGAA ACCAGGGU | 3017 | ACCCUGGU C CGAGGUGU | 238 |
| 2728 | UACUCAGG CUGAUGAG GCCGUUAGGC CGAA ACACCUCG | 3018 | CGAGGUGU C CCUGAGUA | 239 |
| 2736 | CGCAGCCA CUGAUGAG GCCGUUAGGC CGAA ACUCAGGG | 3019 | CCCUGAGU A UGGCUGCG | 240 |
| 2754 | UCUUCCGC CUGAUGAG GCCGUUAGGC CGAA AGUUCACC | 3020 | GGUGAACU U GCGGAAGA | 241 |
| 2775 | CUACAGGG CUGAUGAG GCCGUUAGGC CGAA AGUUCACC | 3021 | GGUGAACU U CCCUGUAG | 242 |
| 2776 | UCUACAGG CUGAUGAG GCCGUUAGGC CGAA AAGUUCAC | 3022 | GUGAACUU C CCUGUAGA | 243 |
| 2782 | UCGUCUUC CUGAUGAG GCCGUUAGGC CGAA ACAGGGAA | 3023 | UUCCCUGU A GAAGACGA | 244 |
| 2810 | CUGAACAA CUGAUGAG GCCGUUAGGC CGAA AGCCGUGC | 3024 | GCACGGCU U UUGUUCAG | 245 |
| 2811 | UCUGAACA CUGAUGAG GCCGUUAGGC CGAA AAGCCGUG | 3025 | CACGGCUU U UGUUCAGA | 246 |
| 2812 | AUCUGAAC CUGAUGAG GCCGUUAGGC CGAA AAAGCCGU | 3026 | ACGGCUUU U GUUCAGAU | 247 |
| 2815 | GGCAUCUG CUGAUGAG GCCGUUAGGC CGAA ACAAAAGC | 3027 | GCUUUUGU U CAGAUGCC | 248 |
| 2816 | CGGCAUCU CUGAUGAG GCCGUUAGGC CGAA AACAAAAG | 3028 | CUUUUGUU C AGAUGCCG | 249 |
| 2836 | CAGGGGAA CUGAUGAG GCCGUUAGGC CGAA AGGCCGUG | 3029 | CACGGCCU A UUCCCCUG | 250 |
| 2838 | ACCAGGGG CUGAUGAG GCCGUUAGGC CGAA AUAGGCCG | 3030 | CGGCCUAU U CCCCUGGU | 251 |
| 2839 | CACCAGGG CUGAUGAG GCCGUUAGGC CGAA AAUAGGCC | 3031 | GGCCUAUU C CCCUGGUG | 252 |
| 2864 | GGUCCGGG CUGAUGAG GCCGUUAGGC CGAA AUCCAGCA | 3032 | UGCUGGAU A CCCGGACC | 253 |
| 2892 | AGCUGGAG CUGAUGAG GCCGUUAGGC CGAA AGUCGCUC | 3033 | GAGCGACU A CUCCAGCU | 254 |
| 2895 | CAUAGCUG CUGAUGAG GCCGUUAGGC CGAA AGUAGUCG | 3034 | CGACUACU C CAGCUAUG | 255 |
| 2901 | UCCGGGCA CUGAUGAG GCCGUUAGGC CGAA AGCUGGAG | 3035 | CUCCAGCU A UGCCCGGA | 256 |
| 2913 | CUCUGAUG CUGAUGAG GCCGUUAGGC CGAA AGGUCCGG | 3036 | CCGGACCU C CAUCAGAG | 257 |
| 2917 | CUGGCUCU CUGAUGAG GCCGUUAGGC CGAA AUGGAGGU | 3037 | ACCUCCAU C AGAGCCAG | 258 |
| 2927 | GAAGGUGA CUGAUGAG GCCGUUAGGC CGAA ACUGGCUC | 3038 | GAGCCAGU C UCACCUUC | 259 |
| 2929 | UUGAAGGU CUGAUGAG GCCGUUAGGC CGAA AGACUGGC | 3039 | GCCAGUCU C ACCUUCAA | 260 |
| 2934 | CGCGGUUG CUGAUGAG GCCGUUAGGC CGAA AGGUGAGA | 3040 | UCUCACCU U CAACCGCG | 261 |
| 2935 | CCGCGGUU CUGAUGAG GCCGUUAGGC CGAA AAGGUGAG | 3041 | CUCACCUU C AACCGCGG | 262 |
| 2946 | CAGCCUUG CUGAUGAG GCCGUUAGGC CGAA AGCCGCGG | 3042 | CCGCGGCU U CAAGGCUG | 263 |
| 2947 | CCAGCCUU CUGAUGAG GCCGUUAGGC CGAA AAGCCGCG | 3043 | CGCGGCUU C AAGGCUGG | 264 |
| 2969 | GAGUUUGC CUGAUGAG GCCGUUAGGC CGAA ACGCAUGU | 3044 | ACAUGCGU C GCAAACUC | 265 |
| 2977 | ACCCCAAA CUGAUGAG GCCGUUAGGC CGAA AGUUUGCG | 3045 | CGCAAACU C UUUGGGGU | 266 |
| 2979 | AGACCCCA CUGAUGAG GCCGUUAGGC CGAA AGAGUUUG | 3046 | CAAACUCU U UGGGGUCU | 267 |
| 2980 | AAGACCCC CUGAUGAG GCCGUUAGGC CGAA AAGAGUUU | 3047 | AAACUCUU U GGGGUCUU | 268 |
| 2986 | AGCCGCAA CUGAUGAG GCCGUUAGGC CGAA ACCCCAAA | 3048 | UUUGGGGU C UUGCGGCU | 269 |
| 2988 | UCAGCCGC CUGAUGAG GCCGUUAGGC CGAA AGACCCCA | 3049 | UGGGGUCU U GCGGCUGA | 270 |
| 3002 | CAGGCUGU CUGAUGAG GCCGUUAGGC CGAA ACACUUCA | 3050 | UGAAGUGU C ACAGCCUG | 271 |
| 3012 | AAUCCAGA CUGAUGAG GCCGUUAGGC CGAA ACAGGCUG | 3051 | CAGCCUGU U UCUGGAUU | 272 |
| 3013 | AAAUCCAG CUGAUGAG GCCGUUAGGC CGAA AACAGGCU | 3052 | AGCCUGUU U CUGGAUUU | 273 |
| 3014 | CAAAUCCA CUGAUGAG GCCGUUAGGC CGAA AAACAGGC | 3053 | GCCUGUUU C UGGAUUUG | 274 |
| 3020 | CACCUGCA CUGAUGAG GCCGUUAGGC CGAA AUCCAGAA | 3054 | UUCUGGAU U UGCAGGUG | 275 |
| 3021 | UCACCUGC CUGAUGAG GCCGUUAGGC CGAA AAUCCAGA | 3055 | UCUGGAUU U GCAGGUGA | 276 |
| 3037 | ACCGUCUG CUGAUGAG GCCGUUAGGC CGAA AGGCUGUU | 3056 | AACAGCCU C CAGACGGU | 277 |
| 3058 | AUCUUGUA CUGAUGAG GCCGUUAGGC CGAA AUGUUGGU | 3057 | ACCAACAU C UACAAGAU | 278 |
| 3060 | GGAUCUUG CUGAUGAG GCCGUUAGGC CGAA AGAUGUUG | 3058 | CAACAUCU A CAAGAUCC | 279 |
| 3067 | AGCAGGAG CUGAUGAG GCCGUUAGGC CGAA AUCUUGUA | 3059 | UACAAGAU C CUCCUGCU | 280 |
| 3070 | UGCAGCAG CUGAUGAG GCCGUUAGGC CGAA AGGAUCUU | 3060 | AAGAUCCU C CUGCUGCA | 281 |
| 3084 | GAAACCUG CUGAUGAG GCCGUUAGGC CGAA ACGCCUGC | 3061 | GCAGGCGU A CAGGUUUC | 282 |
| 3090 | AUGCGUGA CUGAUGAG GCCGUUAGGC CGAA ACCUGUAC | 3062 | GUACAGGU U UCACGCAU | 283 |
| 3091 | CAUGCGUG CUGAUGAG GCCGUUAGGC CGAA AACCUGUA | 3063 | UACAGGUU U CACGCAUG | 284 |
| 3092 | ACAUGCGU CUGAUGAG GCCGUUAGGC CGAA AAACCUGU | 3064 | ACAGGUUU C ACGCAUGU | 285 |
| 3112 | UGAAAUGG CUGAUGAG GCCGUUAGGC CGAA AGCUGCAG | 3065 | CUGCAGCU C CCAUUUCA | 286 |
| 3117 | GCUGAUGA CUGAUGAG GCCGUUAGGC CGAA AUGGGAGC | 3066 | GCUCCCAU U UCAUCAGC | 287 |
| 3118 | UGCUGAUG CUGAUGAG GCCGUUAGGC CGAA AAUGGGAG | 3067 | CUCCCAUU U CAUCAGCA | 288 |
| 3119 | UUGCUGAU CUGAUGAG GCCGUUAGGC CGAA AAAUGGGA | 3068 | UCCCAUUU C AUCAGCAA | 289 |
| 3122 | AACUUGCU CUGAUGAG GCCGUUAGGC CGAA AUGAAAUG | 3069 | CAUUUCAU C AGCAAGUU | 290 |
| 3130 | UUCUUCCA CUGAUGAG GCCGUUAGGC CGAA ACUUGCUG | 3070 | CAGCAAGU U UGGAAGAA | 291 |
| 3131 | GUUCUUCC CUGAUGAG GCCGUUAGGC CGAA AACUUGCU | 3071 | AGCAAGUU U GGAAGAAC | 292 |
| 3147 | GCAGGAAA CUGAUGAG GCCGUUAGGC CGAA AUGUGGGG | 3072 | CCCCACAU U UUUCCUGC | 293 |
| 3148 | CGCAGGAA CUGAUGAG GCCGUUAGGC CGAA AAUGUGGG | 3073 | CCCACAUU U UUCCUGCG | 294 |
| 3149 | GCGCAGGA CUGAUGAG GCCGUUAGGC CGAA AAAUGUGG | 3074 | CCACAUUU U UCCUGCGC | 295 |
| 3150 | CGCGCAGG CUGAUGAG GCCGUUAGGC CGAA AAAAUGUG | 3075 | CACAUUUU U CCUGCGCG | 296 |
| 3151 | ACGCGCAG CUGAUGAG GCCGUUAGGC CGAA AAAAAUGU | 3076 | ACAUUUUU C CUGCGCGU | 297 |
| 3160 | UCAGAGAU CUGAUGAG GCCGUUAGGC CGAA ACGCGCAG | 3077 | CUGCGCGU C AUCUCUGA | 298 |
| 3163 | GUGUCAGA CUGAUGAG GCCGUUAGGC CGAA AUGACGCG | 3078 | CGCGUCAU C UCUGACAC | 299 |
| 3165 | CCGUGUCA CUGAUGAG GCCGUUAGGC CGAA AGAUGACG | 3079 | CGUCAUCU C UGACACGG | 300 |
| 3177 | AGCAGAGG CUGAUGAG GCCGUUAGGC CGAA AGGCCGUG | 3080 | CACGGCCU C CCUCUGCU | 301 |
| 3181 | GAGUAGCA CUGAUGAG GCCGUUAGGC CGAA AGGGAGGC | 3081 | GCCUCCCU C UGCUACUC | 302 |
| 3186 | GGAUGGAG CUGAUGAG GCCGUUAGGC CGAA AGCAGAGG | 3082 | CCUCUGCU A CUCCAUCC | 303 |
| 3189 | UCAGGAUG CUGAUGAG GCCGUUAGGC CGAA AGUAGCAG | 3083 | CUGCUACU C CAUCCUGA | 304 |
| 3193 | GCUUUCAG CUGAUGAG GCCGUUAGGC CGAA AUGGAGUA | 3084 | UACUCCAU C CUGAAAGC | 305 |
| 3219 | CCCCCAGC CUGAUGAG GCCGUUAGGC CGAA ACAUCCCU | 3085 | AGGGAUGU C GCUGGGGG | 306 |
| 3248 | GGAGGGCA CUGAUGAG GCCGUUAGGC CGAA AGGGCCGG | 3086 | CCGGCCCU C UGCCCUCC | 307 |
| 3255 | CGGCCUCG CUGAUGAG GCCGUUAGGC CGAA AGGGCAGA | 3087 | UCUGCCCU C CGAGGCCG | 308 |
| 3288 | UGAGCAGG CUGAUGAG GCCGUUAGGC CGAA AUGCUUGG | 3088 | CCAAGCAU U CCUGCUCA | 309 |
| 3289 | UUGAGCAG CUGAUGAG GCCGUUAGGC CGAA AAUGCUUG | 3089 | CAAGCAUU C CUGCUCAA | 310 |
| 3295 | GUCAGCUU CUGAUGAG GCCGUUAGGC CGAA AGCAGGAA | 3090 | UUCCUGCU C AAGCUGAC | 311 |
| 3305 | ACGGUGUC CUGAUGAG GCCGUUAGGC CGAA AGUCAGCU | 3091 | AGCUGACU C GACACCGU | 312 |
| 3316 | ACGUAGGU CUGAUGAG GCCGUUAGGC CGAA ACACGGUG | 3092 | CACCGUGU C ACCUACGU | 313 |
| 3321 | GUGGCACG CUGAUGAG GCCGUUAGGC CGAA AGGUGACA | 3093 | UGUCACCU A CGUGCCAC | 314 |
| 3331 | GACCCCAG CUGAUGAG GCCGUUAGGC CGAA AGUGGCAC | 3094 | GUGCCACU C CUGGGGUC | 315 |
| 3339 | UCCUGAGU CUGAUGAG GCCGUUAGGC CGAA ACCCCAGG | 3095 | CCUGGGGU C ACUCAGGA | 316 |
| 3343 | GCUGUCCU CUGAUGAG GCCGUUAGGC CGAA AGUGACCC | 3096 | GGGUCACU C AGGACAGC | 317 |
| 3368 | GAGCUUCC CUGAUGAG GCCGUUAGGC CGAA ACUCAGCU | 3097 | AGCUGAGU C GGAAGCUC | 318 |
| 3376 | GUCCCCGG CUGAUGAG GCCGUUAGGC CGAA AGCUUCCG | 3098 | CGGAAGCU C CCGGGGAC | 319 |
| 3429 | UGAAGUCU CUGAUGAG GCCGUUAGGC CGAA AGGGCAGU | 3099 | ACUGCCCU C AGACUUCA | 320 |
| 3435 | UGGUCUUG CUGAUGAG GCCGUUAGGC CGAA AGUCUGAG | 3100 | CUCAGACU U CAAGACCA | 321 |
| 3436 | AUGGUCUU CUGAUGAG GCCGUUAGGC CGAA AAGUCUGA | 3101 | UCAGACUU C AAGACCAU | 322 |
| 3445 | CAGUCCAG CUGAUGAG GCCGUUAGGC CGAA AUGGUCUU | 3102 | AAGACCAU C CUGGACUG | 323 |
| 3503 | CCCGGCGU CUGAUGAG GCCGUUAGGC CGAA ACAGGGCU | 3103 | AGCCCUGU C ACGCCGGG | 324 |
| 3514 | GGGACGUA CUGAUGAG GCCGUUAGGC CGAA AGCCCGGC | 3104 | GCCGGGCU C UACGUCCC | 325 |
| 3516 | CUGGGACG CUGAUGAG GCCGUUAGGC CGAA AGAGCCCG | 3105 | CGGGCUCU A CGUCCCAG | 326 |
| 3520 | CUCCCUGG CUGAUGAG GCCGUUAGGC CGAA ACGUAGAG | 3106 | CUCUACGU C CCAGGGAG | 327 |
| 3568 | AGGCCUCA CUGAUGAG GCCGUUAGGC CGAA ACUCCCAG | 3107 | CUGGGAGU C UGAGGCCU | 328 |
| 3587 | CUCGGCCA CUGAUGAG GCCGUUAGGC CGAA ACACUCAC | 3108 | GUGAGUGU U UGGCCGAG | 329 |
| 3588 | CCUCGGCC CUGAUGAG GCCGUUAGGC CGAA AACACUCA | 3109 | UGAGUGUU U GGCCGAGG | 330 |
| 3606 | UUCAGCCG CUGAUGAG GCCGUUAGGC CGAA ACAUGCAG | 3110 | CUGCAUGU C CGGCUGAA | 331 |
| 3625 | CUCAGCCG CUGAUGAG GCCGUUAGGC CGAA ACACUCAG | 3111 | CUGAGUGU C CGGCUGAG | 332 |
| 3648 | CUUGGCUG CUGAUGAG GCCGUUAGGC CGAA ACACUCGC | 3112 | GCGAGUGU C CAGCCAAG | 333 |
| 3667 | GUGUGCUG CUGAUGAG GCCGUUAGGC CGAA ACACUCAG | 3113 | CUGAGUGU C CAGCACAC | 334 |
| 3683 | GAAGUGAA CUGAUGAG GCCGUUAGGC CGAA ACGGCAGG | 3114 | CCUGCCGU C UUCACUUC | 335 |
| 3685 | GCGAAGUG CUGAUGAG GCCGUUAGGC CGAA AGACGGCA | 3115 | UGCCGUCU U CACUUCCC | 336 |
| 3686 | GGGGAAGU CUGAUGAG GCCGUUAGGC CGAA AAGACGGC | 3116 | GCCGUCUU C ACUUCCCC | 337 |
| 3690 | CUGUGGGG CUGAUGAG GCCGUUAGGC CGAA AGUGAAGA | 3117 | UCUUCACU U CCCCACAG | 338 |
| 3691 | CCUGUGGG CUGAUGAG GCCGUUAGGC CGAA AAGUGAAG | 3118 | CUUCACUU C CCCACAGG | 339 |
| 3708 | GUGGAGCC CUGAUGAG GCCGUUAGGC CGAA AGCGCCAG | 3119 | CUGGCGCU C GGCUCCAC | 340 |
| 3713 | CUGGGGUG CUGAUGAG GCCGUUAGGC CGAA AGCCGAGC | 3120 | GCUCGGCU C CACCCCAG | 341 |
| 3730 | GUGAGGAA CUGAUGAG GCCGUUAGGC CGAA AGCUGGCC | 3121 | GGCCAGCU U UUCCUCAC | 342 |
| 3731 | GGUGAGGA CUGAUGAG GCCGUUAGGC CGAA AAGCUGGC | 3122 | GCCAGCUU U UCCUCACC | 343 |
| 3732 | UGGUGAGG CUGAUGAG GCCGUUAGGC CGAA AAAGCUGG | 3123 | CCAGCUUU U CCUCACCA | 344 |
| 3733 | CUGGUGAG CUGAUGAG GCCGUUAGGC CGAA AAAAGCUG | 3124 | CAGCUUUU C CUCACCAG | 345 |
| 3736 | CUCCUGGU CUGAUGAG GCCGUUAGGC CGAA AGGAAAAG | 3125 | CUUUUCCU C ACCAGGAG | 346 |
| 3752 | GGGAGUGG CUGAUGAG GCCGUUAGGC CGAA AGCCGGGC | 3126 | GCCCGGCU U CCACUCCC | 347 |
| 3753 | GGGGAGUG CUGAUGAG GCCGUUAGGC CGAA AAGCCGGG | 3127 | CCCGGCUU C CACUCCCC | 348 |
| 3758 | UAUGUGGG CUGAUGAG GCCGUUAGGC CGAA AGUGGAAG | 3128 | CUUCCACU C CCCACAUA | 349 |
| 3766 | ACUAUUCC CUGAUGAG GCCGUUAGGC CGAA AUGUGGGG | 3129 | CCCCACAU A GGAAUAGU | 350 |
| 3772 | GGAUGGAC CUGAUGAG GCCGUUAGGC CGAA AUUCCUAU | 3130 | AUAGGAAU A GUCCAUCC | 351 |
| 3775 | UGGGGAUG CUGAUGAG GCCGUUAGGC CGAA ACUAUUCC | 3131 | GGAAUAGU C CAUCCCCA | 352 |
| 3779 | AAUCUGGG CUGAUGAG GCCGUUAGGC CGAA AUGGACUA | 3132 | UAGUCCAU C CCCAGAUU | 353 |
| 3787 | CAAUGGCG CUGAUGAG GCCGUUAGGC CGAA AUCUGGGG | 3133 | CCCCAGAU U CGCCAUUG | 354 |
| 3788 | ACAAUGGC CUGAUGAG GCCGUUAGGC CGAA AAUCUGGG | 3134 | CCCAGAUU C GCCAUUGU | 355 |
| 3794 | GGGUGAAC CUGAUGAG GCCGUUAGGC CGAA AUGGCGAA | 3135 | UUCGCCAU U GUUCACCC | 356 |
| 3797 | GAGGGGUG CUGAUGAG GCCGUUAGGC CGAA ACAAUGGC | 3136 | GCCAUUGU U CACCCCUC | 357 |
| 3798 | CGAGGGGU CUGAUGAG GCCGUUAGGC CGAA AACAAUGG | 3137 | CCAUUGUU C ACCCCUCG | 358 |
| 3805 | GGCAGGGC CUGAUGAG GCCGUUAGGC CGAA AGGGGUGA | 3138 | UCACCCCU C GCCCUGCC | 359 |
| 3816 | AGGCAAAG CUGAUGAG GCCGUUAGGC CGAA AGGGCAGG | 3139 | CCUGCCCU C CUUUGCCU | 360 |
| 3819 | GGAAGGCA CUGAUGAG GCCGUUAGGC CGAA AGGAGGGC | 3140 | GCCCUCCU U UGCCUUCC | 361 |
| 3820 | UGGAAGGC CUGAUGAG GCCGUUAGGC CGAA AAGGAGGG | 3141 | CCCUCCUU U GCCUUCCA | 362 |
| 3825 | GGGGGUGG CUGAUGAG GCCGUUAGGC CGAA AGGCAAAG | 3142 | CUUUGCCU U CCACCCCC | 363 |
| 3626 | UGGGGGUG CUGAUGAG GCCGUUAGGC CGAA AAGGCAAA | 3143 | UUUGCCUU C CACCCCCA | 364 |
| 3839 | UCCACCUG CUGAUGAG GCCGUUAGGC CGAA AUGGUGGG | 3144 | CCCACCAU C CAGGUGGA | 365 |
| 3873 | AAUUCCCA CUGAUGAG GCCGUUAGGC CGAA AGCUCCCA | 3145 | UGGGAGCU C UGGGAAUU | 366 |
| 3881 | UCACUCCA CUGAUGAG GCCGUUAGGC CGAA AUUCCCAG | 3146 | CUGGGAAU U UGGAGUGA | 367 |
| 3882 | GUCACUCC CUGAUGAG GCCGUUAGGC CGAA AAUUCCCA | 3147 | UGGGAAUU U GGAGUGAC | 368 |
| 3907 | CGCCUGUG CUGAUGAG GCCGUUAGGC CGAA ACAGGGCA | 3148 | UGCCCUGU A CACAGGCG | 369 |
| 3940 | CCCACAGG CUGAUGAG GCCGUUAGGC CGAA ACCCCCAU | 3149 | AUGGGGGU C CCUGUGGG | 370 |
| 3950 | CCCAAUUU CUGAUGAG GCCGUUAGGC CGAA ACCCACAG | 3150 | CUGUGGGU C AAAUUGGG | 371 |
| 3955 | CUCCCCCC CUGAUGAG GCCGUUAGGC CGAA AUUUGACC | 3151 | GGUCAAAU U GGGGGGAG | 372 |
| 3977 | CAGUAUUU CUGAUGAG GCCGUUAGGC CGAA ACUCCCAC | 3152 | GUGGGAGU A AAAUACUG | 373 |
| 3982 | AUAUUCAG CUGAUGAG GCCGUUAGGC CGAA AUUUUACU | 3153 | AGUAAAAU A CUGAAUAU | 374 |
| 3989 | AACUCAUA CUGAUGAG GCCGUUAGGC CGAA AUUCAGUA | 3154 | UACUGAAU A UAUGAGUU | 375 |
| 3991 | AAAACUCA CUGAUGAG GCCGUUAGGC CGAA AUAUUCAG | 3155 | CUGAAUAU A UGAGUUUU | 376 |
| 3997 | AACUGAAA CUGAUGAG GCCGUUAGGC CGAA ACUCAUAU | 3156 | AUAUGAGU U UUUCAGUU | 377 |
| 3998 | AAACUGAA CUGAUGAG GCCGUUAGGC CGAA AACUCAUA | 3157 | UAUGAGUU U UUCAGUUU | 378 |
| 3999 | AAAACUGA CUGAUGAG GCCGUUAGGC CGAA AAACUCAU | 3158 | AUGAGUUU U UCAGUUUU | 379 |
| 4000 | CAAAACUG CUGAUGAG GCCGUUAGGC CGAA AAAACUCA | 3159 | UGAGUUUU U CAGUUUUG | 380 |
| 4001 | UCAAAACU CUGAUGAG GCCGUUAGGC CGAA AAAAACUC | 3160 | GAGUUUUU C AGUUUUGA | 381 |
| 4005 | UUUUUCAA CUGAUGAG GCCGUUAGGC CGAA ACUGAAAA | 3161 | UUUUCAGU U UUGAAAAA | 382 |
| 4006 | UUUUUUCA CUGAUGAG GCCGUUAGGC CGAA AACUGAAA | 3162 | UUUCAGUU U UGAAAAAA | 383 |
| 4007 | UUUUUUUC CUGAUGAG GCCGUUAGGC CGAA AAACUGAA | 3163 | UUCAGUUU U GAAAAAAA | 384 |

Stem Length = 8.
Core Sequence = CUGAUGAG GCCGUUAGGC CGAA
Seq1 = TERT (Homo sapiens telomerase reverse transcriptase (TERT) mRNA, 4015 bp); Nakamura et al., Science 277 (5328), 955-959 (1997)

TABLE IV
Human telomerase reverse transcriptase (TERT) NCH Ribozyme and Target Sequence

| nt. | | Seq ID | Substrate | Seq ID |
|---|---|---|---|---|
| Position | Ribozyme Sequence | Nos | Sequence | Nos |
| 14 | GCGCAGCA CUGAUGAG GCCGUUAGGC CGAA IACGCAGC | 385 | GCUGCGUC C UGCUGCGC | 3164 |
| 15 | UGCGCAGC CUGAUGAG GCCGUUAGGC CGAA IGACGCAG | 386 | CUGCGUCC U GCUGCGCA | 3165 |
| 18 | ACGUGCGC CUGAUGAG GCCGUUAGGC CGAA ICAGGACG | 387 | CGUCCUGC U GCGCACGU | 3166 |
| 23 | UUCCCACG CUGAUGAG GCCGUUAGGC CGAA ICGCAGCA | 388 | UGCUGCGC A CGUGGGAA | 3167 |
| 34 | GGGGCCAG CUGAUGAG GCCGUUAGGC CGAA ICUUCCCA | 389 | UGGGAAGC C CUGGCCCC | 3168 |
| 35 | CGGGGCCA CUGAUGAG GCCGUUAGGC CGAA IGCUUCCC | 390 | GGGAAGCC C UGGCCCCG | 3169 |
| 36 | CCGGGGCC CUGAUGAG GCCGUUAGGC CGAA IGGCUUCC | 391 | GGAAGCCC U GGCCCCGG | 3170 |
| 40 | GUGGCCGG CUGAUGAG GCCGUUAGGC CGAA ICCAGGGC | 392 | GCCCUGGC C CCGGCCAC | 3171 |
| 41 | GGUGGCCG CUGAUGAG GCCGUUAGGC CGAA IGCCAGGG | 393 | CCCUGGCC C CGGCCACC | 3172 |
| 42 | GGGUGGCC CUGAUGAG GCCGUUAGGC CGAA IGGCCAGG | 394 | CCUGGCCC C GGCCACCC | 3173 |
| 46 | GCGGGGGU CUGAUGAG GCCGUUAGGC CGAA ICCGGGGC | 395 | GCCCCGGC C ACCCCCGC | 3174 |
| 47 | CGCGGGGG CUGAUGAG GCCGUUAGGC CGAA IGCCGGGG | 396 | CCCCGGCC A CCCCCGCG | 3175 |
| 49 | AUCGCGGG CUGAUGAG GCCGUUAGGC CGAA IUGGCCGG | 397 | CCGGCCAC C CCCGCGAU | 3176 |
| 50 | CAUCGCGG CUGAUGAG GCCGUUAGGC CGAA IGUGGCCG | 398 | CGGCCACC C CCGCGAUG | 3177 |
| 51 | GCAUCGCG CUGAUGAG GCCGUUAGGC CGAA IGGUGGCC | 399 | GGCCACCC C CGCGAUGC | 3178 |
| 52 | GGCAUCGC CUGAUGAG GCCGUUAGGC CGAA IGGGUGGC | 400 | GCCACCCC C GCGAUGCC | 3179 |
| 60 | GAGCGCGC CUGAUGAG GCCGUUAGGC CGAA ICAUCGCG | 401 | CGCGAUGC C GCGCGCUC | 3180 |
| 67 | CAGCGGGG CUGAUGAG GCCGUUAGGC CGAA ICGCGCGG | 402 | CCGCGCGC U CCCCGCUG | 3181 |
| 69 | GGCAGCGG CUGAUGAG GCCGUUAGGC CGAA IAGCGCGC | 403 | GCGCGCUC C CCGCUGCC | 3182 |
| 70 | CGGCAGCG CUGAUGAG GCCGUUAGGC CGAA IGAGCGCG | 404 | CGCGCUCC C CGCUGCCG | 3183 |
| 71 | UCGGCAGC CUGAUGAG GCCGUUAGGC CGAA IGGAGCGC | 405 | GCGCUCCC C GCUGCCGA | 3184 |
| 74 | GGCUCGGC CUGAUGAG GCCGUUAGGC CGAA ICGGGGAG | 406 | CUCCCCGC U GCCGAGCC | 3185 |
| 77 | CACGGCUC CUGAUGAG GCCGUUAGGC CGAA ICAGCGGG | 407 | CCCGCUGC C GAGCCGUG | 3186 |
| 82 | GAGCGCAC CUGAUGAG GCCGUUAGGC CGAA ICUCGGCA | 408 | UGCCGAGC C GUGCGCUC | 3187 |
| 89 | CAGCAGGG CUGAUGAG GCCGUUAGGC CGAA ICGCACGG | 409 | CCGUGCGC U CCCUGCUG | 3188 |
| 91 | CGCAGCAG CUGAUGAG GCCGUUAGGC CGAA IAGCGCAC | 410 | GUGCGCUC C CUGCUGCG | 3189 |
| 92 | GCGCAGCA CUGAUGAG GCCGUUAGGC CGAA IGAGCGCA | 411 | UGCGCUCC C UGCUGCGC | 3190 |
| 93 | UGCGCAGC CUGAUGAG GCCGUUAGGC CGAA IGGAGCGC | 412 | GCGCUCCC U GCUGCGCA | 3191 |
| 96 | GGCUGCGC CUGAUGAG GCCGUUAGGC CGAA ICAGGGAG | 413 | CUCCCUGC U GCGCAGCC | 3192 |
| 101 | GUAGUGGC CUGAUGAG GCCGUUAGGC CGAA ICGCAGCA | 414 | UGCUGCGC A GCCACUAC | 3193 |
| 104 | GCGGUAGU CUGAUGAG GCCGUUAGGC CGAA ICUGCGCA | 415 | UGCGCAGC C ACUACCGC | 3194 |
| 105 | CGCGGUAG CUGAUGAG GCCGUUAGGC CGAA IGCUGCGC | 416 | GCGCAGCC A CUACCGCG | 3195 |
| 107 | CUCGCGGU CUGAUGAG GCCGUUAGGC CGAA IUGGCUGC | 417 | GCAGCCAC U ACCGCGAG | 3196 |
| 110 | CACCUCGC CUGAUGAG GCCGUUAGGC CGAA IUAGUGGC | 418 | GCCACUAC C GCGAGGUG | 3197 |
| 120 | CCAGCGGC CUGAUGAG GCCGUUAGGC CGAA ICACCUCG | 419 | CGAGGUGC U GCCGCUGG | 3198 |
| 123 | UGGCCAGC CUGAUGAG GCCGUUAGGC CGAA ICAGCACC | 420 | GGUGCUGC C GCUGGCCA | 3199 |
| 126 | ACGUGGCC CUGAUGAG GCCGUUAGGC CGAA ICGGCAGC | 421 | GCUGCCGC U GGCCACGU | 3200 |
| 130 | ACGAACGU CUGAUGAG GCCGUUAGGC CGAA ICCAGCGG | 422 | CCGCUGGC C ACGUUCGU | 3201 |
| 131 | CACGAACG CUGAUGAG GCCGUUAGGC CGAA IGCCAGCG | 423 | CGCUGGCC A CGUUCGUG | 3202 |
| 146 | GGGCCCCA CUGAUGAG GCCGUUAGGC CGAA ICGCCGCA | 424 | UGCGGCGC C UGGGGCCC | 3203 |
| 147 | GGGGCCCC CUGAUGAG GCCGUUAGGC CGAA IGCGCCGC | 425 | GCGGCGCC U GGGGCCCC | 3204 |
| 153 | AGCCCUGG CUGAUGAG GCCGUUAGGC CGAA ICCCCAGG | 426 | CCUGGGGC C CCAGGGCU | 3205 |
| 154 | CAGCCCUG CUGAUGAG GCCGUUAGGC CGAA IGCCCCAG | 427 | CUGGGGCC C CAGGGCUG | 3206 |
| 155 | CCAGCCCU CUGAUGAG GCCGUUAGGC CGAA IGGCCCCA | 428 | UGGGGCCC C AGGGCUGG | 3207 |
| 156 | GCCAGCCC CUGAUGAG GCCGUUAGGC CGAA IGGGCCCC | 429 | GGGGCCCC A GGGCUGGC | 3208 |
| 161 | CAGCCGCC CUGAUGAG GCCGUUAGGC CGAA ICCCUGGG | 430 | CCCAGGGC U GGCGGCUG | 3209 |
| 168 | GCUGCACC CUGAUGAG GCCGUUAGGC CGAA ICCGCCAG | 431 | CUGGCGGC U GGUGCAGC | 3210 |
| 174 | CCCCGCGC CUGAUGAG GCCGUUAGGC CGAA ICACCAGC | 432 | GCUGGUGC A GCGCGGGG | 3211 |
| 185 | AGCCGCCG CUGAUGAG GCCGUUAGGC CGAA IUCCCCGC | 433 | GCGGGGAC C CGGCGGCU | 3212 |
| 186 | AAGCCGCC CUGAUGAG GCCGUUAGGC CGAA IGUCCCCG | 434 | CGGGGACC C GGCGGCUU | 3213 |
| 193 | GCGCGGAA CUGAUGAG GCCGUUAGGC CGAA ICCGCCGG | 435 | CCGGCGGC U UUCCGCGC | 3214 |
| 197 | CAGCGCGC CUGAUGAG GCCGUUAGGC CGAA IAAAGCCG | 436 | CGGCUUUC C GCGCGCUG | 3215 |
| 204 | GGGCCACC CUGAUGAG GCCGUUAGGC CGAA ICGCGCGG | 437 | CCGCGCGC U GGUGGCCC | 3216 |
| 211 | AGGCACUG CUGAUGAG GCCGUUAGGC CGAA ICCACCAG | 438 | CUGGUGGC C CAGUGCCU | 3217 |
| 212 | CAGGCACU CUGAUGAG GCCGUUAGGC CGAA IGCCACCA | 439 | UGGUGGCC C AGUGCCUG | 3218 |
| 213 | CCAGGCAC CUGAUGAG GCCGUUAGGC CGAA IGGCCACC | 440 | GGUGGCCC A GUGCCUGG | 3219 |
| 218 | GCACACCA CUGAUGAG GCCGUUAGGC CGAA ICACUGGG | 441 | CCCAGUGC C UGGUGUGC | 3220 |
| 219 | CGCACACC CUGAUGAG GCCGUUAGGC CGAA IGCACUGG | 442 | CCAGUGCC U GGUGUGCG | 3221 |
| 231 | CGUCCCAG CUGAUGAG GCCGUUAGGC CGAA ICACGCAC | 443 | GUGCGUGC C CUGGGACG | 3222 |
| 232 | GCGUCCCA CUGAUGAG GCCGUUAGGC CGAA IGCACGCA | 444 | UGCGUGCC C UGGGACGC | 3223 |
| 233 | UGCGUCCC CUGAUGAG GCCGUUAGGC CGAA IGGCACGC | 445 | GCGUGCCC U GGGACGCA | 3224 |
| 241 | GGCGGCCG CUGAUGAG GCCGUUAGGC CGAA ICGUCCCA | 446 | UGGGACGC A CGGCCGCC | 3225 |
| 246 | CGGGGGGC CUGAUGAG GCCGUUAGGC CGAA ICCGUGCG | 447 | CGCACGGC C GCCCCCCG | 3226 |
| 249 | CGGCGGGG CUGAUGAG GCCGUUAGGC CGAA ICGGCCGU | 448 | ACGGCCGC C CCCCGCCG | 3227 |
| 250 | GCGGCGGG CUGAUGAG GCCGUUAGGC CGAA IGCGGCCG | 449 | CGGCCGCC C CCCGCCGC | 3228 |
| 251 | GGCGGCGG CUGAUGAG GCCGUUAGGC CGAA IGGCGGCC | 450 | GGCCGCCC C CCGCCGCC | 3229 |
| 252 | GGGCGGCG CUGAUGAG GCCGUUAGGC CGAA IGGGCGGC | 451 | GCCGCCCC C CGCCGCCC | 3230 |
| 253 | GGGGCGGC CUGAUGAG GCCGUUAGGC CGAA IGGGGCGG | 452 | CCGCCCCC C GCCGCCCC | 3231 |
| 256 | GAGGGGGC CUGAUGAG GCCGUUAGGC CGAA ICGGGGGG | 453 | CCCCCCGC C GCCCCCUC | 3232 |
| 259 | AAGGAGGG CUGAUGAG GCCGUUAGGC CGAA ICGGCGGG | 454 | CCCGCCGC C CCCUCCUU | 3233 |
| 260 | GAAGGAGG CUGAUGAG GCCGUUAGGC CGAA IGCGGCGG | 455 | CCGCCGCC C CCUCCUUC | 3234 |
| 261 | GGAAGGAG CUGAUGAG GCCGUUAGGC CGAA IGGCGGCG | 456 | CGCCGCCC C CUCCUUCC | 3235 |
| 262 | CGGAAGGA CUGAUGAG GCCGUUAGGC CGAA IGGGCGGC | 457 | GCCGCCCC C UCCUUCCG | 3236 |
| 263 | GCGGAAGG CUGAUGAG GCCGUUAGGC CGAA IGGGGCGG | 458 | CCGCCCCC U CCUUCCGC | 3237 |
| 265 | UGGCGGAA CUGAUGAG GCCGUUAGGC CGAA IAGGGGGC | 459 | GCCCCCUC C UUCCGCCA | 3238 |
| 266 | CUGGCGGA CUGAUGAG GCCGUUAGGC CGAA IGAGGGGG | 460 | CCCCCUCC U UCCGCCAG | 3239 |
| 269 | CACCUGGC CUGAUGAG GCCGUUAGGC CGAA IAAGGAGG | 461 | CCUCCUUC C GCCAGGUG | 3240 |
| 272 | GGACACCU CUGAUGAG GCCGUUAGGC CGAA ICGGAAGG | 462 | CCUUCCGC C AGGUGUCC | 3241 |
| 273 | AGGACACC CUGAUGAG GCCGUUAGGC CGAA IGCGGAAG | 463 | CUUCCGCC A GGUGUCCU | 3242 |
| 280 | UUCAGGCA CUGAUGAG GCCGUUAGGC CGAA IACACCUG | 464 | CAGGUGUC C UGCCUGAA | 3243 |
| 281 | CUUCAGGC CUGAUGAG GCCGUUAGGC CGAA IGACACCU | 465 | AGGUGUCC U GCCUGAAG | 3244 |
| 284 | CUCCUUCA CUGAUGAG GCCGUUAGGC CGAA ICAGGACA | 466 | UGUCCUGC C UGAAGGAG | 3245 |
| 285 | GCUCCUUC CUGAUGAG GCCGUUAGGC CGAA IGCAGGAC | 467 | GUCCUGCC U GAAGGAGC | 3246 |
| 294 | GGGCCACC CUGAUGAG GCCGUUAGGC CGAA ICUCCUUC | 468 | GAAGGAGC U GGUGGCCC | 3247 |
| 301 | AGCACUCG CUGAUGAG GCCGUUAGGC CGAA ICCACCAG | 469 | CUGGUGGC C CGAGUGCU | 3248 |
| 302 | CAGCACUC CUGAUGAG GCCGUUAGGC CGAA IGCCACCA | 470 | UGGUGGCC C GAGUGCUG | 3249 |
| 309 | GCCUCUGC CUGAUGAG GCCGUUAGGC CGAA ICACUCGG | 471 | CCGAGUGC U GCAGAGGC | 3250 |
| 312 | ACAGCCUC CUGAUGAG GCCGUUAGGC CGAA ICAGCACU | 472 | AGUGCUGC A GAGGCUGU | 3251 |
| 318 | GCUCGCAC CUGAUGAG GCCGUUAGGC CGAA ICCUCUGC | 473 | GCAGAGGC U GUGCGAGC | 3252 |
| 345 | CGAAGGCC CUGAUGAG GCCGUUAGGC CGAA ICACGUUC | 474 | GAACGUGC U GGCCUUCG | 3253 |
| 349 | AAGCCGAA CUGAUGAG GCCGUUAGGC CGAA ICCAGCAC | 475 | GUGCUGGC C UUCGGCUU | 3254 |
| 350 | GAAGCCGA CUGAUGAG GCCGUUAGGC CGAA IGCCAGCA | 476 | UGCUGGCC U UCGGCUUC | 3255 |
| 356 | CAGCGCGA CUGAUGAG GCCGUUAGGC CGAA ICCGAAGG | 477 | CCUUCGGC U UCGCGCUG | 3256 |
| 363 | CGUCCAGC CUGAUGAG GCCGUUAGGC CGAA ICGCGAAG | 478 | CUUCGCGC U GCUGGACG | 3257 |
| 366 | CCCCGUCC CUGAUGAG GCCGUUAGGC CGAA ICAGCGCG | 479 | CGCGCUGC U GGACGGGG | 3258 |
| 376 | CCCCCGCG CUGAUGAG GCCGUUAGGC CGAA ICCCCGUC | 480 | GACGGGGC C CGCGGGGG | 3259 |
| 377 | GCCCCCGC CUGAUGAG GCCGUUAGGC CGAA IGCCCCGU | 481 | ACGGGGCC C GCGGGGGC | 3260 |
| 386 | CUCGGGGG CUGAUGAG GCCGUUAGGC CGAA ICCCCCGC | 482 | GCGGGGGC C CCCCCGAG | 3261 |
| 387 | CCUCGGGG CUGAUGAG GCCGUUAGGC CGAA IGCCCCCG | 483 | CGGGGGCC C CCCCGAGG | 3262 |
| 388 | GCCUCGGG CUGAUGAG GCCGUUAGGC CGAA IGGCCCCC | 484 | GGGGGCCC C CCCGAGGC | 3263 |
| 389 | GGCCUCGG CUGAUGAG GCCGUUAGGC CGAA IGGGCCCC | 485 | GGGGCCCC C CCGAGGCC | 3264 |
| 390 | AGGCCUCG CUGAUGAG GCCGUUAGGC CGAA IGGGGCCC | 486 | GGGCCCCC C CGAGGCCU | 3265 |
| 391 | AAGGCCUC CUGAUGAG GCCGUUAGGC CGAA IGGGGGCC | 487 | GGCCCCCC C GAGGCCUU | 3266 |
| 397 | GUGGUGAA CUGAUGAG GCCGUUAGGC CGAA ICCUCGGG | 488 | CCCGAGGC C UUCACCAC | 3267 |
| 398 | GGUGGUGA CUGAUGAG GCCGUUAGGC CGAA IGCCUCGG | 489 | CCGAGGCC U UCACCACC | 3268 |
| 401 | GCUGGUGG CUGAUGAG GCCGUUAGGC CGAA IAAGGCCU | 490 | AGGCCUUC A CCACCAGC | 3269 |
| 403 | ACGCUGGU CUGAUGAG GCCGUUAGGC CGAA IUGAAGGC | 491 | GCCUUCAC C ACCAGCGU | 3270 |
| 404 | CACGCUGG CUGAUGAG GCCGUUAGGC CGAA IGUGAAGG | 492 | CCUUCACC A CCAGCGUG | 3271 |
| 406 | CGCACGCU CUGAUGAG GCCGUUAGGC CGAA IUGGUGAA | 493 | UUCACCAC C AGCGUGCG | 3272 |
| 407 | GCGCACGC CUGAUGAG GCCGUUAGGC CGAA IGUGGUGA | 494 | UCACCACC A GCGUGCGC | 3273 |
| 416 | CAGGUAGC CUGAUGAG GCCGUUAGGC CGAA ICGCACGC | 495 | GCGUGCGC A GCUACCUG | 3274 |
| 419 | GGGCAGGU CUGAUGAG GCCGUUAGGC CGAA ICUGCGCA | 496 | UGCGCAGC U ACCUGCCC | 3275 |
| 422 | GUUGGGCA CUGAUGAG GCCGUUAGGC CGAA IUAGCUGC | 497 | GCAGCUAC C UGCCCAAC | 3276 |
| 423 | UGUUGGGC CUGAUGAG GCCGUUAGGC CGAA IGUAGCUG | 498 | CAGCUACC U GCCCAACA | 3277 |
| 426 | CCGUGUUG CUGAUGAG GCCGUUAGGC CGAA ICAGGUAG | 499 | CUACCUGC C CAACACGG | 3278 |
| 427 | ACCGUGUU CUGAUGAG GCCGUUAGGC CGAA IGCAGGUA | 500 | UACCUGCC C AACACGGU | 3279 |
| 428 | CACCGUGU CUGAUGAG GCCGUUAGGC CGAA IGGCAGGU | 501 | ACCUGCCC A ACACGGUG | 3280 |
| 431 | GGUCACCG CUGAUGAG GCCGUUAGGC CGAA IUUGGGCA | 502 | UGCCCAAC A CGGUGACC | 3281 |
| 439 | AGUGCGUC CUGAUGAG GCCGUUAGGC CGAA IUCACCGU | 503 | ACGGUGAC C GACGCACU | 3282 |
| 445 | CCCCGCAG CUGAUGAG GCCGUUAGGC CGAA ICGUCGGU | 504 | ACCGACGC A CUGCGGGG | 3283 |
| 447 | UCCCCCGC CUGAUGAG GCCGUUAGGC CGAA IUGCGUCG | 505 | CGACGCAC U GCGGGGGA | 3284 |
| 471 | GCAGCAGC CUGAUGAG GCCGUUAGGC CGAA ICCCCCAC | 506 | GUGGGGGC U GCUGCUGC | 3285 |
| 474 | GGCGCAGC CUGAUGAG GCCGUUAGGC CGAA ICAGCCCC | 507 | GGGGCUGC U GCUGCGCC | 3286 |
| 477 | CGCGGCGC CUGAUGAG GCCGUUAGGC CGAA ICAGCAGC | 508 | GCUGCUGC U GCGCCGCG | 3287 |
| 482 | GCCCACGC CUGAUGAG GCCGUUAGGC CGAA ICGCAGCA | 509 | UGCUGCGC C GCGUGGGC | 3288 |
| 501 | GGUGAACC CUGAUGAG GCCGUUAGGC CGAA ICACGUCG | 510 | CGACGUGC U GGUUCACC | 3289 |
| 507 | CCAGCAGG CUGAUGAG GCCGUUAGGC CGAA IAACCAGC | 511 | GCUGGUUC A CCUGCUGG | 3290 |
| 509 | UGCCAGCA CUGAUGAG GCCGUUAGGC CGAA IUGAACCA | 512 | UGGUUCAC C UGCUGGCA | 3291 |
| 510 | GUGCCAGC CUGAUGAG GCCGUUAGGC CGAA IGUGAACC | 513 | GGUUCACC U GCUGGCAC | 3292 |
| 513 | AGCGUGCC CUGAUGAG GCCGUUAGGC CGAA ICAGGUGA | 514 | UCACCUGC U GGCACGCU | 3293 |
| 517 | GCGCAGCG CUGAUGAG GCCGUUAGGC CGAA ICCAGCAG | 515 | CUGCUGGC A CGCUGCGC | 3294 |
| 521 | GAGCGCGC CUGAUGAG GCCGUUAGGC CGAA ICGUGCCA | 516 | UGGCACGC U GCGCGCUC | 3295 |
| 528 | GCACAAAG CUGAUGAG GCCGUUAGGC CGAA ICGCGCAG | 517 | CUGCGCGC U CUUUGUGC | 3296 |
| 530 | CAGCACAA CUGAUGAG GCCGUUAGGC CGAA IAGCGCGC | 518 | GCGCGCUC U UUGUGCUG | 3297 |
| 537 | GAGCCACC CUGAUGAG GCCGUUAGGC CGAA ICACAAAG | 519 | CUUUGUGC U GGUGGCUC | 3298 |
| 544 | CAGCUGGG CUGAUGAG GCCGUUAGGC CGAA ICCACCAG | 520 | CUGGUGGC U CCCAGCUG | 3299 |
| 546 | CGCAGCUG CUGAUGAG GCCGUUAGGC CGAA IAGCCACC | 521 | GGUGGCUC C CAGCUGCG | 3300 |
| 547 | GCGCAGCU CUGAUGAG GCCGUUAGGC CGAA IGAGCCAC | 522 | GUGGCUCC C AGCUGCGC | 3301 |
| 548 | GGCGCAGC CUGAUGAG GCCGUUAGGC CGAA IGGAGCCA | 523 | UGGCUCCC A GCUGCGCC | 3302 |
| 551 | GUAGGCGC CUGAUGAG GCCGUUAGGC CGAA ICUGGGAG | 524 | CUCCCAGC U GCGCCUAC | 3303 |
| 556 | ACCUGGUA CUGAUGAG GCCGUUAGGC CGAA ICGCAGCU | 525 | AGCUGCGC C UACCAGGU | 3304 |
| 557 | CACCUGGU CUGAUGAG GCCGUUAGGC CGAA IGCGCAGC | 526 | GCUGCGCC U ACCAGGUG | 3305 |
| 560 | GCACACCU CUGAUGAG GCCGUUAGGC CGAA IUAGGCGC | 527 | GCGCCUAC C AGGUGUGC | 3306 |
| 561 | CGCACACC CUGAUGAG GCCGUUAGGC CGAA IGUAGGCG | 528 | CGCCUACC A GGUGUGCG | 3307 |
| 573 | ACAGCGGC CUGAUGAG GCCGUUAGGC CGAA ICCCGCAC | 529 | GUGCGGGC C GCCGCUGU | 3308 |
| 576 | GGUACAGC CUGAUGAG GCCGUUAGGC CGAA ICGGCCCG | 530 | CGGGCCGC C GCUGUACC | 3309 |
| 579 | GCUGGUAC CUGAUGAG GCCGUUAGGC CGAA ICGGCGGC | 531 | GCCGCCGC U GUACCAGC | 3310 |
| 584 | GCCGAGCU CUGAUGAG GCCGUUAGGC CGAA IUACAGCG | 532 | CGCUGUAC C AGCUCGGC | 3311 |
| 585 | CGCCGAGC CUGAUGAG GCCGUUAGGC CGAA IGUACAGC | 533 | GCUGUACC A GCUCGGCG | 3312 |
| 588 | CAGCGCCG CUGAUGAG GCCGUUAGGC CGAA ICUGGUAC | 534 | GUACCAGC U CGGCGCUG | 3313 |
| 595 | UGAGUGGC CUGAUGAG GCCGUUAGGC CGAA ICGCCGAG | 535 | CUCGGCGC U GCCACUCA | 3314 |
| 598 | GCCUGAGU CUGAUGAG GCCGUUAGGC CGAA ICAGCGCC | 536 | GGCGCUGC C ACUCAGGC | 3315 |
| 599 | GGCCUGAG CUGAUGAG GCCGUUAGGC CGAA IGCAGCGC | 537 | GCGCUGCC A CUCAGGCC | 3316 |
| 601 | CGGGCCUG CUGAUGAG GCCGUUAGGC CGAA IUGGCAGC | 538 | GCUGCCAC U CAGGCCCG | 3317 |
| 603 | GCCGGGCC CUGAUGAG GCCGUUAGGC CGAA IAGUGGCA | 539 | UGCCACUC A GGCCCGGC | 3318 |
| 607 | GGGGGCCG CUGAUGAG GCCGUUAGGC CGAA ICCUGAGU | 540 | ACUCAGGC C CGGCCCCC | 3319 |
| 608 | CGGGGGCC CUGAUGAG GCCGUUAGGC CGAA IGCCUGAG | 541 | CUCAGGCC C GGCCCCCG | 3320 |
| 612 | GUGGCGGG CUGAUGAG GCCGUUAGGC CGAA ICCGGGCC | 542 | GGCCCGGC C CCCGCCAC | 3321 |
| 613 | UGUGGCGG CUGAUGAG GCCGUUAGGC CGAA IGCCGGGC | 543 | GCCCGGCC C CCGCCACA | 3322 |
| 614 | GUGUGGCG CUGAUGAG GCCGUUAGGC CGAA IGGCCGGG | 544 | CCCGGCCC C CGCCACAC | 3323 |
| 615 | CGUGUGGC CUGAUGAG GCCGUUAGGC CGAA IGGGCCGG | 545 | CCGGCCCC C GCCACACG | 3324 |
| 618 | UAGCGUGU CUGAUGAG GCCGUUAGGC CGAA ICGGGGGC | 546 | GCCCCCGC C ACACGCUA | 3325 |
| 619 | CUAGCGUG CUGAUGAG GCCGUUAGGC CGAA IGCGGGGG | 547 | CCCCCGCC A CACGCUAG | 3326 |
| 621 | CACUAGCG CUGAUGAG GCCGUUAGGC CGAA IUGGCGGG | 548 | CCCGCCAC A CGCUAGUG | 3327 |
| 625 | GGUCCACU CUGAUGAG GCCGUUAGGC CGAA ICGUGUGG | 549 | CCACACGC U AGUGGACC | 3328 |
| 633 | GCCUUCGG CUGAUGAG GCCGUUAGGC CGAA IUCCACUA | 550 | UAGUGGAC C CCGAAGGC | 3329 |
| 634 | CGCCUUCG CUGAUGAG GCCGUUAGGC CGAA IGUCCACU | 551 | AGUGGACC C CGAAGGCG | 3330 |
| 635 | ACGCCUUC CUGAUGAG GCCGUUAGGC CGAA IGGUCCAC | 552 | GUGGACCC C GAAGGCGU | 3331 |
| 645 | CGCAUCCC CUGAUGAG GCCGUUAGGC CGAA IACGCCUU | 553 | AAGGCGUC U GGGAUGCG | 3332 |
| 661 | UGGUUCCA CUGAUGAG GCCGUUAGGC CGAA ICCCGUUC | 554 | GAACGGGC C UGGAACCA | 3333 |
| 662 | AUGGUUCC CUGAUGAG GCCGUUAGGC CGAA IGCCCGUU | 555 | AACGGGCC U GGAACCAU | 3334 |
| 668 | GACGCUAU CUGAUGAG GCCGUUAGGC CGAA IUUCCAGG | 556 | CCUGGAAC C AUAGCGUC | 3335 |
| 669 | UGACGCUA CUGAUGAG GCCGUUAGGC CGAA IGUUCCAG | 557 | CUGGAACC A UAGCGUCA | 3336 |
| 677 | GGCCUCCC CUGAUGAG GCCGUUAGGC CGAA IACGCUAU | 558 | AUAGCGUC A GGGAGGCC | 3337 |
| 685 | GGGACCCC CUGAUGAG GCCGUUAGGC CGAA ICCUCCCU | 559 | AGGGAGGC C GGGGUCCC | 3338 |
| 692 | GCCCAGGG CUGAUGAG GCCGUUAGGC CGAA IACCCCGG | 560 | CCGGGGUC C CCCUGGGC | 3339 |
| 693 | GGCCCAGG CUGAUGAG GCCGUUAGGC CGAA IGACCCCG | 561 | CGGGGUCC C CCUGGGCC | 3340 |
| 694 | AGGCCCAG CUGAUGAG GCCGUUAGGC CGAA IGGACCCC | 562 | GGGGUCCC C CUGGGCCU | 3341 |
| 695 | CAGGCCCA CUGAUGAG GCCGUUAGGC CGAA IGGGACCC | 563 | GGGUCCCC C UGGGCCUG | 3342 |
| 696 | GCAGGCCC CUGAUGAG GCCGUUAGGC CGAA IGGGGACC | 564 | GGUCCCCC U GGGCCUGC | 3343 |
| 701 | GGCUGGCA CUGAUGAG GCCGUUAGGC CGAA ICCCAGGG | 565 | CCCUGGGC C UGCCAGCC | 3344 |
| 702 | GGGCUGGC CUGAUGAG GCCGUUAGGC CGAA IGCCCAGG | 566 | CCUGGGCC U GCCAGCCC | 3345 |
| 705 | CCGGGGCU CUGAUGAG GCCGUUAGGC CGAA ICAGGCCC | 567 | GGGCCUGC C AGCCCCGG | 3346 |
| 706 | CCCGGGGC CUGAUGAG GCCGUUAGGC CGAA IGCAGGCC | 568 | GGCCUGCC A GCCCCGGG | 3347 |
| 709 | GCACCCGG CUGAUGAG GCCGUUAGGC CGAA ICUGGCAG | 569 | CUGCCAGC C CCGGGUGC | 3348 |
| 710 | CGCACCCG CUGAUGAG GCCGUUAGGC CGAA IGCUGGCA | 570 | UGCCAGCC C CGGGUGCG | 3349 |
| 711 | UCGCACCC CUGAUGAG GCCGUUAGGC CGAA IGGCUGGC | 571 | GCCAGCCC C GGGUGCGA | 3350 |
| 734 | GCUGGCAC CUGAUGAG GCCGUUAGGC CGAA ICCCCCGC | 572 | GCGGGGGC A GUGCCAGC | 3351 |
| 739 | CUUCGGCU CUGAUGAG GCCGUUAGGC CGAA ICACUGCC | 573 | GGCAGUGC C AGCCGAAG | 3352 |
| 740 | ACUUCGGC CUGAUGAG GCCGUUAGGC CGAA IGCACUGC | 574 | GCAGUGCC A GCCGAAGU | 3353 |
| 743 | CAGACUUC CUGAUGAG GCCGUUAGGC CGAA ICUGGCAC | 575 | GUGCCAGC C GAAGUCUG | 3354 |
| 750 | GCAACGGC CUGAUGAG GCCGUUAGGC CGAA IACUUCGG | 576 | CCGAAGUC U GCCGUUGC | 3355 |
| 753 | UGGGCAAC CUGAUGAG GCCGUUAGGC CGAA ICAGACUU | 577 | AAGUCUGC C GUUGCCCA | 3356 |
| 759 | GCCUCUUG CUGAUGAG GCCGUUAGGC CGAA ICAACGGC | 578 | GCCGUUGC C CAAGAGGC | 3357 |
| 760 | GGCCUCUU CUGAUGAG GCCGUUAGGC CGAA IGCAACGG | 579 | CCGUUGCC C AAGAGGCC | 3358 |
| 761 | GGGCCUCU CUGAUGAG GCCGUUAGGC CGAA IGGCAACG | 580 | CGUUGCCC A AGAGGCCC | 3359 |
| 768 | CACGCCUG CUGAUGAG GCCGUUAGGC CGAA ICCUCUUG | 581 | CAAGAGGC C CAGGCGUG | 3360 |
| 769 | CCACGCCU CUGAUGAG GCCGUUAGGC CGAA IGCCUCUU | 582 | AAGAGGCC C AGGCGUGG | 3361 |
| 770 | GCCACGCC CUGAUGAG GCCGUUAGGC CGAA IGGCCUCU | 583 | AGAGGCCC A GGCGUGGC | 3362 |
| 781 | UCAGGGGC CUGAUGAG GCCGUUAGGC CGAA ICGCCACG | 584 | CGUGGCGC U GCCCCUGA | 3363 |
| 784 | GGCUCAGG CUGAUGAG GCCGUUAGGC CGAA ICAGCGCC | 585 | GGCGCUGC C CCUGAGCC | 3364 |
| 785 | CGGCUCAG CUGAUGAG GCCGUUAGGC CGAA IGCAGCGC | 586 | GCGCUGCC C CUGAGCCG | 3365 |
| 786 | CCGGCUCA CUGAUGAG GCCGUUAGGC CGAA IGGCAGCG | 587 | CGCUGCCC C UGAGCCGG | 3366 |
| 787 | UCCGGCUC CUGAUGAG GCCGUUAGGC CGAA IGGGCAGC | 588 | GCUGCCCC U GAGCCGGA | 3367 |
| 792 | UCCGCUCC CUGAUGAG GCCGUUAGGC CGAA ICUCAGGG | 589 | CCCUGAGC C GGAGCGGA | 3368 |
| 804 | GCCCAACG CUGAUGAG GCCGUUAGGC CGAA ICGUCCGC | 590 | GCGGACGC C CGUUGGGC | 3369 |
| 805 | UGCCCAAC CUGAUGAG GCCGUUAGGC CGAA IGCGUCCG | 591 | CGGACGCC C GUUGGGCA | 3370 |
| 813 | AGGACCCC CUGAUGAG GCCGUUAGGC CGAA ICCCAACG | 592 | CGUUGGGC A GGGGUCCU | 3371 |
| 820 | UGGGCCCA CUGAUGAG GCCGUUAGGC CGAA IACCCCUG | 593 | CAGGGGUC C UGGGCCCA | 3372 |
| 821 | GUGGGCCC CUGAUGAG GCCGUUAGGC CGAA IGACCCCU | 594 | AGGGGUCC U GGGCCCAC | 3373 |
| 826 | CCCGGGUG CUGAUGAG GCCGUUAGGC CGAA ICCCAGGA | 595 | UCCUGGOC C CACCCGGG | 3374 |
| 827 | GCCCGGGU CUGAUGAG GCCGUUAGGC CGAA IGCCCAGG | 596 | CCUGGGCC C ACCCGGGC | 3375 |
| 828 | UGCCCGGG CUGAUGAG GCCGUUAGGC CGAA IGGCCCAG | 597 | CUGGGCCC A CCCGGGCA | 3376 |
| 830 | CCUGCCCG CUGAUGAG GCCGUUAGGC CGAA IUGGGCCC | 598 | GGGCCCAC C CGGGCAGG | 3377 |
| 831 | UCCUGCCC CUGAUGAG GCCGUUAGGC CGAA IGUGGGCC | 599 | GGCCCACC C GGGCAGGA | 3378 |
| 836 | ACGCGUCC CUGAUGAG GCCGUUAGGC CGAA ICCCGGGU | 600 | ACCCGGGC A GGACGCGU | 3379 |
| 849 | GGUCACUC CUGAUGAG GCCGUUAGGC CGAA IUCCACGC | 601 | GCGUGGAC C GAGUGACC | 3380 |
| 857 | GAAACCAC CUGAUGAG GCCGUUAGGC CGAA IUCACUCG | 602 | CGAGUGAC C GUGGUUUC | 3381 |
| 866 | CACCACAC CUGAUGAG GCCGUUAGGC CGAA IAAACCAC | 603 | GUGGUUUC U GUGUGGUG | 3382 |
| 877 | CUGGCAGG CUGAUGAG GCCGUUAGGC CGAA IACACCAC | 604 | GUGGUGUC A CCUGCCAG | 3383 |
| 879 | GUCUGGCA CUGAUGAG GCCGUUAGGC CGAA IUGACACC | 605 | GGUGUCAC C UGCCAGAC | 3384 |
| 880 | GGUCUGGC CUGAUGAG GCCGUUAGGC CGAA IGUGACAC | 606 | GUGUCACC U GCCAGACC | 3385 |
| 883 | GCGGGUCU CUGAUGAG GCCGUUAGGC CGAA ICAGGUGA | 607 | UCACCUGC C AGACCCGC | 3386 |
| 884 | GGCGGGUC CUGAUGAG GCCGUUAGGC CGAA IGCAGGUG | 608 | CACCUGCC A GACCCGCC | 3387 |
| 888 | CUUCGGCG CUGAUGAG GCCGUUAGGC CGAA IUCUGGCA | 609 | UGCCAGAC C CGCCGAAG | 3388 |
| 889 | UCUUCGGC CUGAUGAG GCCGUUAGGC CGAA IGUCUGGC | 610 | GCCAGACC C GCCGAAGA | 3389 |
| 892 | GCUUCUUC CUGAUGAG GCCGUUAGGC CGAA ICGGGUCU | 611 | AGACCCGC C GAAGAAGC | 3390 |
| 901 | AAAGAGGU CUGAUGAG GCCGUUAGGC CGAA ICUUCUUC | 612 | GAAGAAGC C ACCUCUUU | 3391 |
| 902 | CAAAGAGG CUGAUGAG GCCGUUAGGC CGAA IGCUUCUU | 613 | AAGAAGCC A CCUCUUUG | 3392 |
| 904 | UCCAAAGA CUGAUGAG GCCGUUAGGC CGAA IUGGCUUC | 614 | GAAGCCAC C UCUUUGGA | 3393 |
| 905 | CUCCAAAG CUGAUGAG GCCGUUAGGC CGAA IGUGGCUU | 615 | AAGCCACC U CUUUGGAG | 3394 |
| 907 | CCCUCCAA CUGAUGAG GCCGUUAGGC CGAA IAGGUGGC | 616 | GCCACCUC U UUGGAGGG | 3395 |
| 921 | UGCCAGAG CUGAUGAG GCCGUUAGGC CGAA ICGCACCC | 617 | GGGUGCGC U CUCUGGCA | 3396 |
| 923 | CGUGCCAG CUGAUGAG GCCGUUAGGC CGAA IAGCGCAC | 618 | GUGCGCUC U CUGGCACG | 3397 |
| 925 | CGCGUGCC CUGAUGAG GCCGUUAGGC CGAA IAGAGCGC | 619 | GCGCUCUC U GGCACGCG | 3398 |
| 929 | GUGGCGCG CUGAUGAG GCCGUUAGGC CGAA ICCAGAGA | 620 | UCUCUGGC A CGCGCCAC | 3399 |
| 935 | GUGGGAGU CUGAUGAG GCCGUUAGGC CGAA ICGCGUGC | 621 | GCACGCGC C ACUCCCAC | 3400 |
| 936 | GGUGGGAG CUGAUGAG GCCGUUAGGC CGAA IGCGCGUG | 622 | CACGCGCC A CUCCCACC | 3401 |
| 938 | UGGGUGGG CUGAUGAG GCCGUUAGGC CGAA IUGGCGCG | 623 | CGCGCCAC U CCCACCCA | 3402 |
| 940 | GAUGGGUG CUGAUGAG GCCGUUAGGC CGAA IAGUGGCG | 624 | CGCCACUC C CACCCAUC | 3403 |
| 941 | GGAUGGGU CUGAUGAG GCCGUUAGGC CGAA IGAGUGGC | 625 | GCCACUCC C ACCCAUCC | 3404 |
| 942 | CGGAUGGG CUGAUGAG GCCGUUAGGC CGAA IGGAGUGG | 626 | CCACUCCC A CCCAUCCG | 3405 |
| 944 | CACGGAUG CUGAUGAG GCCGUUAGGC CGAA IUGGGAGU | 627 | ACUCCCAC C CAUCCGUG | 3406 |
| 945 | CCACGGAU CUGAUGAG GCCGUUAGGC CGAA IGUGGGAG | 628 | CUCCCACC C AUCCGUGG | 3407 |
| 946 | CCCACGGA CUGAUGAG GCCGUUAGGC CGAA IGGUGGGA | 629 | UCCCACCC A UCCGUGGG | 3408 |
| 949 | CGGCCCAC CUGAUGAG GCCGUUAGGC CGAA IAUGGGUG | 630 | CACCCAUC C GUGGGCCG | 3409 |
| 956 | GUGCUGGC CUGAUGAG GCCGUUAGGC CGAA ICCCACGG | 631 | CCGUGGGC C GCCAGCAC | 3410 |
| 959 | GUGGUGCU CUGAUGAG GCCGUUAGGC CGAA ICGGCCCA | 632 | UGGGCCGC C AGCACCAC | 3411 |
| 960 | CGUGGUGC CUGAUGAG GCCGUUAGGC CGAA IGCGGCCC | 633 | GGGCCGCC A GCACCACG | 3412 |
| 963 | CCGCGUGG CUGAUGAG GCCGUUAGGC CGAA ICUGGCGG | 634 | CCGCCAGC A CCACGCGG | 3413 |
| 965 | GCCCGCGU CUGAUGAG GCCGUUAGGC CGAA IUGCUGGC | 635 | GCCAGCAC C ACGCGGGC | 3414 |
| 966 | GGCCCGCG CUGAUGAG GCCGUUAGGC CGAA IGUGCUGG | 636 | CCAGCACC A CGCGGGCC | 3415 |
| 974 | GGAUGGGG CUGAUGAG GCCGUUAGGC CGAA ICCCGCGU | 637 | ACGCGGGC C CCCCAUCC | 3416 |
| 975 | UGGAUGGG CUGAUGAG GCCGUUAGGC CGAA IGCCCGCG | 638 | CGCGGGCC C CCCAUCCA | 3417 |
| 976 | GUGGAUGG CUGAUGAG GCCGUUAGGC CGAA IGGCCCGC | 639 | GCGGGCCC C CCAUCCAC | 3418 |
| 977 | UGUGGAUG CUGAUGAG GCCGUUAGGC CGAA IGGGCCCG | 640 | CGGGCCCC C CAUCCACA | 3419 |
| 978 | AUGUGGAU CUGAUGAG GCCGUUAGGC CGAA IGGGGCCC | 641 | GGGCCCCC C AUCCACAU | 3420 |
| 979 | GAUGUGGA CUGAUGAG GCCGUUAGGC CGAA IGGGGGCC | 642 | GGCCCCCC A UCCACAUC | 3421 |
| 982 | CGCGAUGU CUGAUGAG GCCGUUAGGC CGAA IAUGGGGG | 643 | CCCCCAUC C ACAUCGCG | 3422 |
| 983 | CCGCGAUG CUGAUGAG GCCGUUAGGC CGAA IGAUGGGG | 644 | CCCCAUCC A CAUCGCGG | 3423 |
| 985 | GGCCGCGA CUGAUGAG GCCGUUAGGC CGAA IUGGAUGG | 645 | CCAUCCAC A UCGCGGCC | 3424 |
| 993 | GACGUGGU CUGAUGAG GCCGUUAGGC CGAA ICCGCGAU | 646 | AUCGCGGC C ACCACGUC | 3425 |
| 994 | GGACGUGG CUGAUGAG GCCGUUAGGC CGAA IGCCGCGA | 647 | UCGCGGCC A CCACGUCC | 3426 |
| 996 | AGGGACGU CUGAUGAG GCCGUUAGGC CGAA IUGGCCGC | 648 | GCGGCCAC C ACGUCCCU | 3427 |
| 997 | CAGGGACG CUGAUGAG GCCGUUAGGC CGAA IGUGGCCG | 649 | CGGCCACC A CGUCCCUG | 3428 |
| 1002 | UGUCCCAG CUGAUGAG GCCGUUAGGC CGAA IACGUGGU | 650 | ACCACGUC C CUGGGACA | 3429 |
| 1003 | GUGUCCCA CUGAUGAG GCCGUUAGGC CGAA IGACGUGG | 651 | CCACGUCC C UGGGACAC | 3430 |
| 1004 | CGUGUCCC CUGAUGAG GCCGUUAGGC CGAA IGGACGUG | 652 | CACGUCCC U GGGACACG | 3431 |
| 1010 | ACAAGGCG CUGAUGAG GCCGUUAGGC CGAA IUCCCAGG | 653 | CCUGGGAC A CGCCUUGU | 3432 |
| 1014 | GGGGACAA CUGAUGAG GCCGUUAGGC CGAA ICGUGUCC | 654 | GGACACGC C UUGUCCCC | 3433 |
| 1015 | GGGGGACA CUGAUGAG GCCGUUAGGC CGAA IGCGUGUC | 655 | GACACGCC U UGUCCCCC | 3434 |
| 1020 | ACACCGGG CUGAUGAG GCCGUUAGGC CGAA IACAAGGC | 656 | GCCUUGUC C CCCGGUGU | 3435 |
| 1021 | UACACCGG CUGAUGAG GCCGUUAGGC CGAA IGACAAGG | 657 | CCUUGUCC C CCGGUGUA | 3436 |
| 1022 | GUACACCG CUGAUGAG GCCGUUAGGC CGAA IGGACAAG | 658 | CUUGUCCC C CGGUGUAC | 3437 |
| 1023 | CGUACACC CUGAUGAG GCCGUUAGGC CGAA IGGGACAA | 659 | UUGUCCCC C GGUGUACG | 3438 |
| 1033 | UUGGUCUC CUGAUGAG GCCGUUAGGC CGAA ICGUACAC | 660 | GUGUACGC C GAGACCAA | 3439 |
| 1039 | AAGUGCUU CUGAUGAG GCCGUUAGGC CGAA IUCUCGGC | 661 | GCCGAGAC C AAGCACUU | 3440 |
| 1040 | GAAGUGCU CUGAUGAG GCCGUUAGGC CGAA IGUCUCGG | 662 | CCGAGACC A AGCACUUC | 3441 |
| 1044 | AGAGGAAG CUGAUGAG GCCGUUAGGC CGAA ICUUGGUC | 663 | GACCAAGC A CUUCCUCU | 3442 |
| 1046 | GUAGAGGA CUGAUGAG GCCGUUAGGC CGAA IUGCUUGG | 664 | CCAAGCAC U UCCUCUAC | 3443 |
| 1049 | GGAGUAGA CUGAUGAG GCCGUUAGGC CGAA IAAGUGCU | 665 | AGCACUUC C UCUACUCC | 3444 |
| 1050 | AGGAGUAG CUGAUGAG GCCGUUAGGC CGAA IGAAGUGC | 666 | GCACUUCC U CUACUCCU | 3445 |
| 1052 | UGAGGAGU CUGAUGAG GCCGUUAGGC CGAA IAGGAAGU | 667 | ACUUCCUC U ACUCCUCA | 3446 |
| 1055 | GCCUGAGG CUGAUGAG GCCGUUAGGC CGAA IUAGAGGA | 668 | UCCUCUAC U CCUCAGGC | 3447 |
| 1057 | UCGCCUGA CUGAUGAG GCCGUUAGGC CGAA IAGUAGAG | 669 | CUCUACUC C UCAGGCGA | 3448 |
| 1058 | GUCGCCUG CUGAUGAG GCCGUUAGGC CGAA IGAGUAGA | 670 | UCUACUCC U CAGGCGAC | 3449 |
| 1060 | UUGUCGCC CUGAUGAG GCCGUUAGGC CGAA IAGGAGUA | 671 | UACUCCUC A GGCGACAA | 3450 |
| 1067 | CUGCUCCU CUGAUGAG GCCGUUAGGC CGAA IUCGCCUG | 672 | CAGGCGAC A AGGAGCAG | 3451 |
| 1074 | GCCGCAGC CUGAUGAG GCCGUUAGGC CGAA ICUCCUUG | 673 | CAAGGAGC A GCUGCGGC | 3452 |
| 1077 | AGGGCCGC CUGAUGAG GCCGUUAGGC CGAA ICUGCUCC | 674 | GGAGCAGC U GCGGCCCU | 3453 |
| 1083 | GGAAGGAG CUGAUGAG GCCGUUAGGC CGAA ICCGCAGC | 675 | GCUGCGGC C CUCCUUCC | 3454 |
| 1084 | AGGAAGGA CUGAUGAG GCCGUUAGGC CGAA IGCCGCAG | 676 | CUGCGGCC C UCCUUCCU | 3455 |
| 1085 | UAGGAAGG CUGAUGAG GCCGUUAGGC CGAA IGGCCGCA | 677 | UGCGGCCC U CCUUCCUA | 3456 |
| 1087 | AGUAGGAA CUGAUGAG GCCGUUAGGC CGAA IAGGGCCG | 678 | CGGCCCUC C UUCCUACU | 3457 |
| 1088 | GAGUAGGA CUGAUGAG GCCGUUAGGC CGAA IGAGGGCC | 679 | GGCCCUCC U UCCUACUC | 3458 |
| 1091 | GCUGAGUA CUGAUGAG GCCGUUAGGC CGAA IAAGGAGG | 680 | CCUCCUUC C UACUCAGC | 3459 |
| 1092 | AGCUGAGU CUGAUGAG GCCGUUAGGC CGAA IGAAGGAG | 681 | CUCCUUCC U ACUCAGCU | 3460 |
| 1095 | GAGAGCUG CUGAUGAG GCCGUUAGGC CGAA IUAGGAAG | 682 | CUUCCUAC U CAGCUCUC | 3461 |
| 1097 | CAGAGAGC CUGAUGAG GCCGUUAGGC CGAA IAGUAGGA | 683 | UCCUACUC A GCUCUCUG | 3462 |
| 1100 | CCUCAGAG CUGAUGAG GCCGUUAGGC CGAA ICUGAGUA | 684 | UACUCAGC U CUCUGAGG | 3463 |
| 1102 | GGCCUCAG CUGAUGAG GCCGUUAGGC CGAA IAGCUGAG | 685 | CUCAGCUC U CUGAGGCC | 3464 |
| 1104 | UGGGCCUC CUGAUGAG GCCGUUAGGC CGAA IAGAGCUG | 686 | CAGCUCUC U GAGGCCCA | 3465 |
| 1110 | UCAGGCUG CUGAUGAG GCCGUUAGGC CGAA ICCUCAGA | 687 | UCUGAGGC C CAGCCUGA | 3466 |
| 1111 | GUCAGGCU CUGAUGAG GCCGUUAGGC CGAA IGCCUCAG | 688 | CUGAGGCC C AGCCUGAC | 3467 |
| 1112 | AGUCAGGC CUGAUGAG GCCGUUAGGC CGAA IGGCCUCA | 689 | UGAGGCCC A GCCUGACU | 3468 |
| 1115 | GCCAGUCA CUGAUGAG GCCGUUAGGC CGAA ICUGGGCC | 690 | GGCCCAGC C UGACUGOC | 3469 |
| 1116 | COCCAGUC CUGAUGAG GCCGUUAGGC CGAA IGCUGGGC | 691 | GCCCAGCC U GACUGGCG | 3470 |
| 1120 | CGAGCGCC CUGAUGAG GCCGUUAGGC CGAA IUCAGGCU | 692 | AGCCUGAC U GGCGCUCG | 3471 |
| 1126 | AGCCUCCG CUGAUGAG GCCGUUAGGC CGAA ICGCCAGU | 693 | ACUGGCGC U CGGAGGCU | 3472 |
| 1134 | UCUCCACG CUGAUGAG GCCGUUAGGC CGAA ICCUCCGA | 694 | UCGGAGGC U CGUGGAGA | 3473 |
| 1144 | AGAAAGAU CUGAUGAG GCCGUUAGGC CGAA IUCUCCAC | 695 | GUGGAGAC C AUCUUUCU | 3474 |
| 1145 | CAGAAAGA CUGAUGAG GCCGUUAGGC CGAA IGUCUCCA | 696 | UGGAGACC A UCUUUCUG | 3475 |
| 1148 | ACCCAGAA CUGAUGAG GCCGUUAGGC CGAA IAUGGUCU | 697 | AGACCAUC U UUCUGGGU | 3476 |
| 1152 | UGGAACCC CUGAUGAG GCCGUUAGGC CGAA IAAAGAUG | 698 | CAUCUUUC U GGGUUCCA | 3477 |
| 1159 | CAGGGCCU CUGAUGAG GCCGUUAGGC CGAA IAACCCAG | 699 | CUGGGUUC C AGGCCCUG | 3478 |
| 1160 | CCAGGGCC CUGAUGAG GCCGUUAGGC CGAA IGAACCCA | 700 | UGGGUUCC A GGCCCUGG | 3479 |
| 1164 | GCAUCCAG CUGAUGAG GCCGUUAGGC CGAA ICCUGGAA | 701 | UUCCAGGC C CUGGAUGC | 3480 |
| 1165 | GGCAUCCA CUGAUGAG GCCGUUAGGC CGAA IGCCUGGA | 702 | UCCAGGCC C UGGAUGCC | 3481 |
| 1166 | UGGCAUCC CUGAUGAG GCCGUUAGGC CGAA IGGCCUGG | 703 | CCAGGCCC U GGAUGCCA | 3482 |
| 1173 | GAGUCCCU CUGAUGAG GCCGUUAGGC CGAA ICAUCCAG | 704 | CUGGAUGC C AGGGACUC | 3483 |
| 1174 | GGAGUCCC CUGAUGAG GCCGUUAGGC CGAA IGCAUCCA | 705 | UGGAUGCC A GGGACUCC | 3484 |
| 1180 | CUGCGGGG CUGAUGAG GCCGUUAGGC CGAA IUCCCUGG | 706 | CCAGGGAC U CCCCGCAG | 3485 |
| 1182 | ACCUGCGG CUGAUGAG GCCGUUAGGC CGAA IAGUCCCU | 707 | AGGGACUC C CCGCAGGU | 3486 |
| 1183 | AACCUGCG CUGAUGAG GCCGUUAGGC CGAA IGAGUCCC | 708 | GGGACUCC C CGCAGGUU | 3487 |
| 1184 | CAACCUGC CUGAUGAG GCCGUUAGGC CGAA IGGAGUCC | 709 | GGACUCCC C GCAGGUUG | 3488 |
| 1187 | GGGCAACC CUGAUGAG GCCGUUAGGC CGAA ICGGGGAG | 710 | CUCCCCGC A GGUUGCCC | 3489 |
| 1194 | GCAGGCGG CUGAUGAG GCCGUUAGGC CGAA ICAACCUG | 711 | CAGGUUGC C CCGCCUGC | 3490 |
| 1195 | GGCAGGCG CUGAUGAG GCCGUUAGGC CGAA IGCAACCU | 712 | AGGUUGCC C CGCCUGCC | 3491 |
| 1196 | GGGCAGGC CUGAUGAG GCCGUUAGGC CGAA IGGCAACC | 713 | GGUUGCCC C GCCUGCCC | 3492 |
| 1199 | CUGGGGCA CUGAUGAG GCCGUUAGGC CGAA ICGGGGCA | 714 | UGCCCCGC C UGCCCCAG | 3493 |
| 1200 | GCUGGGGC CUGAUGAG GCCGUUAGGC CGAA IGCGGGGC | 715 | GCCCCGCC U GCCCCAGC | 3494 |
| 1203 | AGCGCUGG CUGAUGAG GCCGUUAGGC CGAA ICAGGCGG | 716 | CCGCCUGC C CCAGCGCU | 3495 |
| 1204 | UAGCGCUG CUGAUGAG GCCGUUAGGC CGAA IGCAGGCG | 717 | CGCCUGCC C CAGCGCUA | 3496 |
| 1205 | GUAGCGCU CUGAUGAG GCCGUUAGGC CGAA IGGCAGGC | 718 | GCCUGCCC C AGCGCUAC | 3497 |
| 1206 | AGUAGCGC CUGAUGAG GCCGUUAGGC CGAA IGGGCAGG | 719 | CCUGCCCC A GCGCUACU | 3498 |
| 1211 | UUGCCAGU CUGAUGAG GCCGUUAGGC CGAA ICGCUGGG | 720 | CCCAGCGC U ACUGGCAA | 3499 |
| 1214 | CAUUUGCC CUGAUGAG GCCGUUAGGC CGAA IUAGCGCU | 721 | AGCGCUAC U GGCAAAUG | 3500 |
| 1218 | GCCGCAUU CUGAUGAG GCCGUUAGGC CGAA ICCAGUAG | 722 | CUACUGGC A AAUGCGGC | 3501 |
| 1227 | GAAACAGG CUGAUGAG GCCGUUAGGC CGAA ICCGCAUU | 723 | AAUGCGGC C CCUGUUUC | 3502 |
| 1228 | AGAAACAG CUGAUGAG GCCGUUAGGC CGAA IGCCGCAU | 724 | AUGCGGCC C CUGUUUCU | 3503 |
| 1229 | CAGAAACA CUGAUGAG GCCGUUAGGC CGAA IGGCCGCA | 725 | UGCGGCCC C UGUUUCUG | 3504 |
| 1230 | CCAGAAAC CUGAUGAG GCCGUUAGGC CGAA IGGGCCGC | 726 | GCGGCCCC U GUUUCUGG | 3505 |
| 1236 | GCAGCUCC CUGAUGAG GCCGUUAGGC CGAA IAAACAGG | 727 | CCUGUUUC U GGAGCUGC | 3506 |
| 1242 | UCCCAAGC CUGAUGAG GCCGUUAGGC CGAA ICUCCAGA | 728 | UCUGGAGC U GCUUGGGA | 3507 |
| 1245 | GGUUCCCA CUGAUGAG GCCGUUAGGC CGAA ICAGCUCC | 729 | GGAGCUGC U UGGGAACC | 3508 |
| 1253 | CUGCGCGU CUGAUGAG GCCGUUAGGC CGAA IUUCCCAA | 730 | UUGGGAAC C ACGCGCAG | 3509 |
| 1254 | ACUGCGCG CUGAUGAG GCCGUUAGGC CGAA IGUUCCCA | 731 | UGGGAACC A CGCGCAGU | 3510 |
| 1260 | AGGGGCAC CUGAUGAG GCCGUUAGGC CGAA ICGCGUGG | 732 | CCACGCGC A GUGCCCCU | 3511 |
| 1265 | CCCGUAGG CUGAUGAG GCCGUUAGGC CGAA ICACUGCG | 733 | CGCAGUGC C CCUACGGG | 3512 |
| 1266 | CCCCGUAG CUGAUGAG GCCGUUAGGC CGAA IGCACUGC | 734 | GCAGUGCC C CUACGGGG | 3513 |
| 1267 | ACCCCGUA CUGAUGAG GCCGUUAGGC CGAA IGGCACUG | 735 | CAGUGCCC C UACGGGGU | 3514 |
| 1268 | CACCCCGU CUGAUGAG GCCGUUAGGC CGAA IGGGCACU | 736 | AGUGCCCC U ACGGGGUG | 3515 |
| 1278 | UCUUGAGG CUGAUGAG GCCGUUAGGC CGAA ICACCCCG | 737 | CGGGGUGC U CCUCAAGA | 3516 |
| 1280 | CGUCUUGA CUGAUGAG GCCGUUAGGC CGAA IAGCACCC | 738 | GGGUGCUC C UCAAGACG | 3517 |
| 1281 | GCGUCUUG CUGAUGAG GCCGUUAGGC CGAA IGAGCACC | 739 | GGUGCUCC U CAAGACGC | 3518 |
| 1283 | GUGCGUCU CUGAUGAG GCCGUUAGGC CGAA IAGGAGCA | 740 | UGCUCCUC A AGACGCAC | 3519 |
| 1290 | GCGGGCAG CUGAUGAG GCCGUUAGGC CGAA ICGUCUUG | 741 | CAAGACGC A CUGCCCGC | 3520 |
| 1292 | CAGCGGGC CUGAUGAG GCCGUUAGGC CGAA IUGCGUCU | 742 | AGACGCAC U GCCCGCUG | 3521 |
| 1295 | UCGCAGCG CUGAUGAG GCCGUUAGGC CGAA ICAGUGCG | 743 | CGCACUGC C CGCUGCGA | 3522 |
| 1296 | CUCGCAGC CUGAUGAG GCCGUUAGGC CGAA IGCAGUGC | 744 | GCACUGCC C GCUGCGAG | 3523 |
| 1299 | CAGCUCGC CUGAUGAG GCCGUUAGGC CGAA ICGGGCAG | 745 | CUGCCCGC U GCGAGCUG | 3524 |
| 1306 | GUGACCGC CUGAUGAG GCCGUUAGGC CGAA ICUCGCAG | 746 | CUGCGAGC U GCGGUCAC | 3525 |
| 1313 | UGCUGGGG CUGAUGAG GCCGUUAGGC CGAA IACCGCAG | 747 | CUGCGGUC A CCCCAGCA | 3526 |
| 1315 | GCUGCUGG CUGAUGAG GCCGUUAGGC CGAA IUGACCGC | 748 | GCGGUCAC C CCAGCAGC | 3527 |
| 1316 | GGCUGCUG CUGAUGAG GCCGUUAGGC CGAA IGUGACCG | 749 | CGGUCACC C CAGCAGCC | 3528 |
| 1317 | CGGCUGCU CUGAUGAG GCCGUUAGGC CGAA IGGUGACC | 750 | GGUCACCC C AGCAGCCG | 3529 |
| 1318 | CCGGCUGC CUGAUGAG GCCGUUAGGC CGAA IGGGUGAC | 751 | GUCACCCC A GCAGCCGG | 3530 |
| 1321 | ACACCGGC CUGAUGAG GCCGUUAGGC CGAA ICUGGGGU | 752 | ACCCCAGC A GCCGGUGU | 3531 |
| 1324 | CAGACACC CUGAUGAG GCCGUUAGGC CGAA ICUGCUGG | 753 | CCAGCAGC C GGUGUCUG | 3532 |
| 1331 | CCGGGCAC CUGAUGAG GCCGUUAGGC CGAA IACACCGG | 754 | CCGGUGUC U GUGCCCGG | 3533 |
| 1236 | UUCUCCCG CUGAUGAG GCCGUUAGGC CGAA ICACAGAC | 755 | GUCUGUGC C CGGGAGAA | 3534 |
| 1337 | CUUCUCCC CUGAUGAG GCCGUUAGGC CGAA IGCACAGA | 756 | UCUGUGCC C GGGAGAAG | 3535 |
| 1347 | AGCCCUGG CUGAUGAG GCCGUUAGGC CGAA ICUUCUCC | 757 | GGAGAAGC C CCAGGGCU | 3536 |
| 1348 | GAGCCCUG CUGAUGAG GCCGUUAGGC CGAA IGCUUCUC | 758 | GAGAAGCC C CAGGGCUC | 3537 |
| 1349 | AGAGCCCU CUGAUGAG GCCGUUAGGC CGAA IGGCUUCU | 759 | AGAAGCCC C AGGGCUCU | 3538 |
| 1350 | CAGAGCCC CUGAUGAG GCCGUUAGGC CGAA IGGGCUUC | 760 | GAAGCCCC A GGGCUCUG | 3539 |
| 1355 | CGCCACAG CUGAUGAG GCCGUUAGGC CGAA ICCCUGGG | 761 | CCCAGGGC U CUGUGGCG | 3540 |
| 1357 | GCCGCCAC CUGAUGAG GCCGUUAGGC CGAA IAGCCCUG | 762 | CAGGGCUC U GUGGCGGC | 3541 |
| 1366 | UCCUCGGG CUGAUGAG GCCGUUAGGC CGAA ICCGCCAC | 763 | GUGGCGGC C CCCGAGGA | 3542 |
| 1367 | CUCCUCGG CUGAUGAG GCCGUUAGGC CGAA IGCCGCCA | 764 | UGGCGGCC C CCGAGGAG | 3543 |
| 1368 | CCUCCUCG CUGAUGAG GCCGUUAGGC CGAA IGGCCGCC | 765 | GGCGGCCC C CGAGGAGG | 3544 |
| 1369 | UCCUCCUC CUGAUGAG GCCGUUAGGC CGAA IGGGCCGC | 766 | GCGGCCCC C GAGGAGGA | 3545 |
| 1382 | GGGGUCUG CUGAUGAG GCCGUUAGGC CGAA IUCCUCCU | 767 | AGGAGGAC A CAGACCCC | 3546 |
| 1384 | CGGGGGUC CUGAUGAG GCCGUUAGGC CGAA IUGUCCUC | 768 | GAGGACAC A GACCCCCG | 3547 |
| 1388 | GCGACGGG CUGAUGAG GCCGUUAGGC CGAA IUCUGUGU | 769 | ACACAGAC C CCCGUCGC | 3548 |
| 1389 | GGCGACGG CUGAUGAG GCCGUUAGGC CGAA IGUCUGUG | 770 | CACAGACC C CCGUCGCC | 3549 |
| 1390 | AGGCGACG CUGAUGAG GCCGUUAGGC CGAA IGGUCUGU | 771 | ACAGACCC C CGUCGCCU | 3550 |
| 1391 | CAGGCGAC CUGAUGAG GCCGUUAGGC CGAA IGGGUCUG | 772 | CAGACCCC C GUCGCCUG | 3551 |
| 1397 | CUGCACCA CUGAUGAG GCCGUUAGGC CGAA ICGACGGG | 773 | CCCGUCGC C UGGUGCAG | 3552 |
| 1398 | GCUGCACC CUGAUGAG GCCGUUAGGC CGAA IGCGACGG | 774 | CCGUCGCC U GGUGCAGC | 3553 |
| 1404 | GGAGCAGC CUGAUGAG GCCGUUAGGC CGAA ICACCAGG | 775 | CCUGGUGC A GCUGCUCC | 3554 |
| 1407 | GGCGGAGC CUGAUGAG GCCGUUAGGC CGAA ICUGCACC | 776 | GGUGCAGC U GCUCCGCC | 3555 |
| 1410 | GCUGGCGG CUGAUGAG GCCGUUAGGC CGAA ICAGCUGC | 777 | GCAGCUGC U CCGCCAGC | 3556 |
| 1412 | GUGCUGGC CUGAUGAG GCCGUUAGGC CGAA IAGCAGCU | 778 | AGCUGCUC C GCCAGCAC | 3557 |
| 1415 | GCUGUGCU CUGAUGAG GCCGUUAGGC CGAA ICGGAGCA | 779 | UGCUCCGC C AGCACAGC | 3558 |
| 1416 | UGCUGUGC CUGAUGAG GCCGUUAGGC CGAA IGCGGAGC | 780 | GCUCCGCC A GCACAGCA | 3559 |
| 1419 | GGCUGCUG CUGAUGAG GCCGUUAGGC CGAA ICUGGCGG | 781 | CCGCCAGC A CAGCAGCC | 3560 |
| 1421 | GGGGCUGC CUGAUGAG GCCGUUAGGC CGAA IUGCUGGC | 782 | GCCAGCAC A GCAGCCCC | 3561 |
| 1424 | CCAGGGGC CUGAUGAG GCCGUUAGGC CGAA ICUGUGCU | 783 | AGCACAGC A GCCCCUGG | 3562 |
| 1427 | CUGCCAGG CUGAUGAG GCCGUUAGGC CGAA ICUGCUGU | 784 | ACAGCAGC C CCUGGCAG | 3563 |
| 1428 | CCUGCCAG CUGAUGAG GCCGUUAGGC CGAA IGCUGCUG | 785 | CAGCAGCC C CUGGCAGG | 3564 |
| 1429 | ACCUGCCA CUGAUGAG GCCGUUAGGC CGAA IGGCUGCU | 786 | AGCAGCCC C UGGCAGGU | 3565 |
| 1430 | CACCUGCC CUGAUGAG GCCGUUAGGC CGAA IGGGCUGC | 787 | GCAGCCCC U GGCAGGUG | 3566 |
| 1434 | CGUACACC CUGAUGAG GCCGUUAGGC CGAA ICCAGGGG | 788 | CCCCUGGC A GGUGUACG | 3567 |
| 1445 | CCGCACGA CUGAUGAG GCCGUUAGGC CGAA ICOGUACA | 789 | UGUACGGC U UCGUGCGG | 3568 |
| 1456 | CGCAGGCA CUGAUGAG GCCGUUAGGC CGAA ICCCGCAC | 790 | GUGCGGGC C UGCCUGCG | 3569 |
| 1457 | GCGCAGGC CUGAUGAG GCCGUUAGGC CGAA IGCCCGCA | 791 | UGCGGGCC U GCCUGCGC | 3570 |
| 1460 | CCGGCGCA CUGAUGAG GCCGUUAGGC CGAA ICAGGCCC | 792 | GGGCCUGC C UGCGCCGG | 3571 |
| 1461 | GCCGGCGC CUGAUGAG GCCGUUAGGC CGAA IGCAGGCC | 793 | GGCCUGCC U GCGCCGGC | 3572 |
| 1466 | CACCAGCC CUGAUGAG GCCGUUAGGC CGAA ICGCAGGC | 794 | GCCUGCGC C GGCUGGUG | 3573 |
| 1470 | GGGGCACC CUGAUGAG GCCGUUAGGC CGAA ICCGGCGC | 795 | GCGCCGGC U GGUGCCCC | 3574 |
| 1476 | GGCCUGGG CUGAUGAG GCCGUUAGGC CGAA ICACCAGC | 796 | GCUGGUGC C CCCAGGCC | 3575 |
| 1477 | AGGCCUGG CUGAUGAG GCCGUUAGGC CGAA IGCACCAG | 797 | CUGGUGCC C CCAGGCCU | 3576 |
| 1478 | GAGGCCUG CUGAUGAG GCCGUUAGGC CGAA IGGCACCA | 798 | UGGUGCCC C CAGGCCUC | 3577 |
| 1479 | AGAGGCCU CUGAUGAG GCCGUUAGGC CGAA IGGGCACC | 799 | GGUGCCCC C AGGCCUCU | 3578 |
| 1480 | CAGAGGCC CUGAUGAG GCCGUUAGGC CGAA IGGGGCAC | 800 | GUGCCCCC A GGCCUCUG | 3579 |
| 1484 | GCCCCAGA CUGAUGAG GCCGUUAGGC CGAA ICCUGGGG | 801 | CCCCAGGC C UCUGGGGC | 3580 |
| 1485 | AGCCCCAG CUGAUGAG GCCGUUAGGC CGAA IGCCUGGG | 802 | CCCAGGCC U CUGGGGCU | 3581 |
| 1487 | GGAGCCCC CUGAUGAG GCCGUUAGGC CGAA IAGGCCUG | 803 | CAGGCCUC U GGGGCUCC | 3582 |
| 1493 | GUGCCUGG CUGAUGAG GCCGUUAGGC CGAA ICCCCAGA | 804 | UCUGGGGC U CCAGGCAC | 3583 |
| 1495 | UUGUGCCU CUGAUGAG GCCGUUAGGC CGAA IAGCCCCA | 805 | UGGGGCUC C AGGCACAA | 3584 |
| 1496 | GUUGUGCC CUGAUGAG GCCGUUAGGC CGAA IGAGCCCC | 806 | GGGGCUCC A GGCACAAC | 3585 |
| 1500 | GUUCGUUG CUGAUGAG GCCGUUAGGC CGAA ICCUGGAG | 807 | CUCCAGGC A CAACGAAC | 3586 |
| 1502 | GCGUUCGU CUGAUGAG GCCGUUAGGC CGAA IUGCCUGG | 808 | CCAGGCAC A ACGAACGC | 3587 |
| 1511 | GAGGAAGC CUGAUGAG GCCGUUAGGC CGAA ICGUUCGU | 809 | ACGAACGC C GCUUCCUC | 3588 |
| 1514 | CCUGAGGA CUGAUGAG GCCGUUAGGC CGAA ICGGCGUU | 810 | AACGCCGC U UCCUCAGG | 3589 |
| 1517 | GUUCCUGA CUGAUGAG GCCGUUAGGC CGAA IAAGCGGC | 811 | GCCGCUUC C UCAGGAAC | 3590 |
| 1518 | UGUUCCUG CUGAUGAG GCCGUUAGGC CGAA IGAAGCGG | 812 | CCGCUUCC U CAGGAACA | 3591 |
| 1520 | GGUGUUCC CUGAUGAG GCCGUUAGGC CGAA IAGGAAGC | 813 | GCUUCCUC A GGAACACC | 3592 |
| 1526 | CUUCUUGG CUGAUGAG GCCGUUAGGC CGAA IUUCCUGA | 814 | UCAGGAAC A CCAAGAAG | 3593 |
| 1528 | AACUUCUU CUGAUGAG GCCGUUAGGC CGAA IUGUUCCU | 815 | AGGAACAC C AAGAAGUU | 3594 |
| 1529 | GAACUUCU CUGAUGAG GCCGUUAGGC CGAA IGUGUUCC | 816 | GGAACACC A AGAAGUUC | 3595 |
| 1538 | CAGGGAGA CUGAUGAG GCCGUUAGGC CGAA IAACUUCU | 817 | AGAAGUUC A UCUCCCUG | 3596 |
| 1541 | CCCCAGGG CUGAUGAG GCCGUUAGGC CGAA IAUGAACU | 818 | AGUUCAUC U CCCUGGGG | 3597 |
| 1543 | UUCCCCAG CUGAUGAG GCCGUUAGGC CGAA IAGAUGAA | 819 | UUCAUCUC C CUGGGGAA | 3598 |
| 1544 | CUUCCCCA CUGAUGAG GCCGUUAGGC CGAA IGAGAUGA | 820 | UCAUCUCC C UGGGGAAG | 3599 |
| 1545 | GCUUCCCC CUGAUGAG GCCGUUAGGC CGAA IGGAGAUG | 821 | CAUCUCCC U GGGGAAGC | 3600 |
| 1554 | GCUUGGCA CUGAUGAG GCCGUUAGGC CGAA ICUUCCCC | 822 | GGGGAAGC A UGCCAAGC | 3601 |
| 1558 | GAGAGCUU CUGAUGAG GCCGUUAGGC CGAA ICAUGCUU | 823 | AAGCAUGC C AAGCUCUC | 3602 |
| 1559 | CGAGAGCU CUGAUGAG GCCGUUAGGC CGAA IGCAUGCU | 824 | AGCAUGCC A AGCUCUCG | 3603 |
| 1563 | GCAGCGAG CUGAUGAG GCCGUUAGGC CGAA ICUUGGCA | 825 | UGCCAAGC U CUCGCUGC | 3604 |
| 1565 | CUGCAGCG CUGAUGAG GCCGUUAGGC CGAA IAGCUUGG | 826 | CCAAGCUC U CGCUGCAG | 3605 |
| 1569 | GCUCCUGC CUGAUGAG GCCGUUAGGC CGAA ICGAGAGC | 827 | GCUCUCGC U GCAGGAGC | 3606 |
| 1572 | UCAGCUCC CUGAUGAG GCCGUUAGGC CGAA ICAGCGAG | 828 | CUCGCUGC A GGAGCUGA | 3607 |
| 1578 | UCCACGUC CUGAUGAG GCCGUUAGGC CGAA ICUCCUGC | 829 | GCAGGAGC U GACGUGGA | 3608 |
| 1604 | CCAAGCGC CUGAUGAG GCCGUUAGGC CGAA IUCCCGCA | 830 | UGCGGGAC U GCGCUUGG | 3609 |
| 1609 | CGCAGCCA CUGAUGAG GCCGUUAGGC CGAA ICGCAGUC | 831 | GACUGCGC U UGGCUGCG | 3610 |
| 1614 | UCCUGCGC CUGAUGAG GCCGUUAGGC CGAA ICCAAGCG | 832 | CGCUUGGC U GCGCAGGA | 3611 |
| 1619 | UGGGCUCC CUGAUGAG GCCGUUAGGC CGAA ICGCAGCC | 833 | GGCUGCGC A GGAGCCCA | 3612 |
| 1625 | AACCCCUG CUGAUGAG GCCGUUAGGC CGAA ICUCCUGC | 834 | GCAGGAGC C CAGGGGUU | 3613 |
| 1626 | CAACCCCU CUGAUGAG GCCGUUAGGC CGAA IGCUCCUG | 835 | CAGGAGCC C AGGGGUUG | 3614 |
| 1627 | CCAACCCC CUGAUGAG GCCGUUAGGC CGAA IGGCUCCU | 836 | AGGAGCCC A GGGGUUGG | 3615 |
| 1637 | CGGAACAC CUGAUGAG GCCGUUAGGC CGAA ICCAACCC | 837 | GGGUUGGC U GUGUUCCG | 3616 |
| 1644 | CUGCGGCC CUGAUGAG GCCGUUAGGC CGAA IAACACAG | 838 | CUGUGUUC C GGCCGCAG | 3617 |
| 1648 | UGCUCUGC CUGAUGAG GCCGUUAGGC CGAA ICCGGAAC | 839 | GUUCCGGC C GCAGAGCA | 3618 |
| 1651 | CGGUGCUC CUGAUGAG GCCGUUAGGC CGAA ICGGCCGG | 840 | CCGGCCGC A GAGCACCG | 3619 |
| 1656 | GCAGACGG CUGAUGAG GCCGUUAGGC CGAA ICUCUGCG | 841 | CGCAGAGC A CCGUCUGC | 3620 |
| 1658 | ACGCAGAC CUGAUGAG GCCGUUAGGC CGAA IUGCUCUG | 842 | CAGAGCAC C GUCUGCGU | 3621 |
| 1662 | CCUCACGC CUGAUGAG GCCGUUAGGC CGAA IACGGUGC | 843 | GCACCGUC U GCGUGAGG | 3622 |
| 1676 | CUUGGCCA CUGAUGAG GCCGUUAGGC CGAA IAUCUCCU | 844 | AGGAGAUC C UGGCCAAG | 3623 |
| 1677 | ACUUGGCC CUGAUGAG GCCGUUAGGC CGAA IGAUCUCC | 845 | GGAGAUCC U GGCCAAGU | 3624 |
| 1681 | AGGAACUU CUGAUGAG GCCGUUAGGC CGAA ICCAGGAU | 846 | AUCCUGGC C AAGUUCCU | 3625 |
| 1682 | CAGGAACU CUGAUGAG GCCGUUAGGC CGAA IGCCAGGA | 847 | UCCUGGCC A AGUUCCUG | 3626 |
| 1688 | CCAGUGCA CUGAUGAG GCCGUUAGGC CGAA IAACUUGG | 848 | CCAAGUUC C UGCACUGG | 3627 |
| 1689 | GCCAGUGC CUGAUGAG GCCGUUAGGC CGAA IGAACUUG | 849 | CAAGUUCC U GCACUGGC | 3628 |
| 1692 | UCAGCCAG CUGAUGAG GCCGUUAGGC CGAA ICAGGAAC | 850 | GUUCCUGC A CUGGCUGA | 3629 |
| 1694 | CAUCAGCC CUGAUGAG GCCGUUAGGC CGAA IUGCAGGA | 851 | UCCUGCAC U GGCUGAUG | 3630 |
| 1698 | CACUCAUC CUGAUGAG GCCGUUAGGC CGAA ICCAGUGC | 852 | GCACUGGC U GAUGAGUG | 3631 |
| 1722 | ACCUGAGC CUGAUGAG GCCGUUAGGC CGAA ICUCGACG | 853 | CGUCGAGC U GCUCAGGU | 3632 |
| 1725 | AAGACCUG CUGAUGAG GCCGUUAGGC CGAA ICAGCUCG | 854 | CGAGCUGC U CAGGUCUU | 3633 |
| 1727 | GAAAGACC CUGAUGAG GCCGUUAGGC CGAA IAGCAGCU | 855 | AGCUGCUC A GGUCUUUC | 3634 |
| 1732 | UAAAAGAA CUGAUGAG GCCGUUAGGC CGAA IACCUGAG | 856 | CUCAGGUC U UUCUUUUA | 3635 |
| 1736 | GACAUAAA CUGAUGAG GCCGUUAGGC CGAA IAAAGACC | 857 | GGUCUUUC U UUUAUGUC | 3636 |
| 1745 | GGUCUCCG CUGAUGAG GCCGUUAGGC CGAA IACAUAAA | 858 | UUUAUGUC A CGGAGACC | 3637 |
| 1753 | UGAAACGU CUGAUGAG GCCGUUAGGC CGAA IUCUCCGU | 859 | ACGGAGAC C ACGUUUCA | 3638 |
| 1754 | UUGAAACG CUGAUGAG GCCGUUAGGC CGAA IGUCUCCG | 860 | CGGAGACC A CGUUUCAA | 3639 |
| 1761 | UGUUCUUU CUGAUGAG GCCGUUAGGC CGAA IAAACGUG | 861 | CACGUUUC A AAAGAACA | 3640 |
| 1769 | AAAGAGCC CUGAUGAG GCCGUUAGGC CGAA IUUCUUUU | 862 | AAAAGAAC A GGCUCUUU | 3641 |
| 1773 | AGAAAAAG CUGAUGAG GCCGUUAGGC CGAA ICCUGUUC | 863 | GAACAGGC U CUUUUUCU | 3642 |
| 1775 | GUAGAAAA CUGAUGAG GCCGUUAGGC CGAA IAGCCUGU | 864 | ACAGGCUC U UUUUCUAC | 3643 |
| 1781 | CUUCCGGU CUGAUGAG GCCGUUAGGC CGAA IAAAAAGA | 865 | UCUUUUUC U ACCGGAAG | 3644 |
| 1784 | ACUCUUCC CUGAUGAG GCCGUUAGGC CGAA IUAGAAAA | 866 | UUUUCUAC C GGAAGAGU | 3645 |
| 1796 | CUUGCUCC CUGAUGAG GCCGUUAGGC CGAA IACACUCU | 867 | AGAGUGUC U GGAGCAAG | 3646 |
| 1802 | UUGCAACU CUGAUGAG GCCGUUAGGC CGAA ICUCCAGA | 868 | UCUGGAGC A AGUUGCAA | 3647 |
| 1809 | CAAUGCUU CUGAUGAG GCCGUUAGGC CGAA ICAACUUG | 869 | CAAGUUGC A AAGCAUUG | 3648 |
| 1814 | GAUUCCAA CUGAUGAG GCCGUUAGGC CGAA ICUUUGCA | 870 | UGCAAAGC A UUGGAAUC | 3649 |
| 1823 | GUGCUGUC CUGAUGAG GCCGUUAGGC CGAA IAUUCCAA | 871 | UUGGAAUC A GACAGCAC | 3650 |
| 1827 | UCAAGUGC CUGAUGAG GCCGUUAGGC CGAA IUCUGAUU | 872 | AAUCAGAC A GCACUUGA | 3651 |
| 1830 | UCUUCAAG CUGAUGAG GCCGUUAGGC CGAA ICUGUCUG | 873 | CAGACAGC A CUUGAAGA | 3652 |
| 1832 | CCUCUUCA CUGAUGAG GCCGUUAGGC CGAA IUGCUGUC | 874 | GACAGCAC U UGAAGAGG | 3653 |
| 1845 | CCCGCAGC CUGAUGAG GCCGUUAGGC CGAA ICACCCUC | 875 | GAGGGUGC A GCUGCGGG | 3654 |
| 1848 | GCUCCCGC CUGAUGAG GCCGUUAGGC CGAA ICUGCACC | 876 | GGUGCAGC U GCGGGAGC | 3655 |
| 1857 | CUUCCGAC CUGAUGAG GCCGUUAGGC CGAA ICUCCCGC | 877 | GCGGGAGC U GUCGGAAG | 3656 |
| 1867 | CUGACCUC CUGAUGAG GCCGUUAGGC CGAA ICUUCCGA | 878 | UCGGAAGC A GAGGUCAG | 3657 |
| 1874 | AUGCUGCC CUGAUGAG GCCGUUAGGC CGAA IACCUCUG | 879 | CAGAGGUC A GGCAGCAU | 3658 |
| 1878 | CCCGAUGC CUGAUGAG GCCGUUAGGC CGAA ICCUGACC | 880 | GGUCAGGC A GCAUCGGG | 3659 |
| 1881 | CUUCCCGA CUGAUGAG GCCGUUAGGC CGAA ICUGCCUG | 881 | CAGGCAGC A UCGGGAAG | 3660 |
| 1891 | GCGGGCCU CUGAUGAG GCCGUUAGGC CGAA ICUUCCCG | 882 | CGGGAAGC C AGGCCCGC | 3661 |
| 1892 | GGCGGGCC CUGAUGAG GCCGUUAGGC CGAA IGCUUCCC | 883 | GGGAAGCC A GGCCCGCC | 3662 |
| 1896 | GCAGGGCG CUGAUGAG GCCGUUAGGC CGAA ICCUGGCU | 884 | AGCCAGGC C CGCCCUGC | 3663 |
| 1897 | AGCAGGGC CUGAUGAG GCCGUUAGGC CGAA IGCCUGGC | 885 | GCCAGGCC C GCCCUGCU | 3664 |
| 1900 | GUCAGCAG CUGAUGAG GCCGUUAGGC CGAA ICGGGCCU | 886 | AGGCCCGC C CUGCUGAC | 3665 |
| 1901 | CGUCAGCA CUGAUGAG GCCGUUAGGC CGAA IGCGGGCC | 887 | GGCCCGCC C UGCUGACG | 3666 |
| 1902 | ACGUCAGC CUGAUGAG GCCGUUAGGC CGAA IGGCGGGC | 888 | GCCCGCCC U GCUGACGU | 3667 |
| 1905 | UGGACGUC CUGAUGAG GCCGUUAGGC CGAA ICAGGGCG | 889 | CGCCCUGC U GACGUCCA | 3668 |
| 1912 | CGGAGUCU CUGAUGAG GCCGUUAGGC CGAA IACGUCAG | 890 | CUGACGUC C AGACUCCG | 3669 |
| 1913 | GCGGAGUC CUGAUGAG GCCGUUAGGC CGAA IGACGUCA | 891 | UGACGUCC A GACUCCGC | 3670 |
| 1917 | UGAAGCGG CUGAUGAG GCCGUUAGGC CGAA IUCUGGAC | 892 | GUCCAGAC U CCGCUUCA | 3671 |
| 1919 | GAUGAAGC CUGAUGAG GCCGUUAGGC CGAA IAGUCUGG | 893 | CCAGACUC C GCUUCAUC | 3672 |
| 1922 | GGGGAUGA CUGAUGAG GCCGUUAGGC CGAA ICGGAGUC | 894 | GACUCCGC U UCAUCCCC | 3673 |
| 1925 | CUUGGGGA CUGAUGAG GCCGUUAGGC CGAA IAAGCGGA | 895 | UCCGCUUC A UCCCCAAG | 3674 |
| 1928 | AGGCUUGG CUGAUGAG GCCGUUAGGC CGAA IAUGAAGC | 896 | GCUUCAUC C CCAAGCCU | 3675 |
| 1929 | CAGGCUUG CUGAUGAG GCCGUUAGGC CGAA IGAUGAAG | 897 | CUUCAUCC C CAAGCCUG | 3676 |
| 1930 | UCAGGCUU CUGAUGAG GCCGUUAGGC CGAA IGGAUGAA | 898 | UUCAUCCC C AAGCCUGA | 3677 |
| 1931 | GUCAGGCU CUGAUGAG GCCGUUAGGC CGAA IGGGAUGA | 899 | UCAUCCCC A AGCCUGAC | 3678 |
| 1935 | GCCCGUCA CUGAUGAG GCCGUUAGGC CGAA ICUUGGGG | 900 | CCCCAAGC C UGACGGGC | 3679 |
| 1936 | AGCCCGUC CUGAUGAG GCCGUUAGGC CGAA IGCUUGGG | 901 | CCCAAGCC U GACGGGCU | 3680 |
| 1944 | UCGGCCGC CUGAUGAG GCCGUUAGGC CGAA ICCCGUCA | 902 | UGACGGGC U GCGGCCGA | 3681 |
| 1950 | UCACAAUC CUGAUGAG GCCGUUAGGC CGAA ICCGCAGC | 903 | GCUGCGGC C GAUUGUGA | 3682 |
| 1961 | GUAGUCCA CUGAUGAG GCCGUUAGGC CGAA IUUCACAA | 904 | UUGUGAAC A UGGACUAC | 3683 |
| 1967 | CACGACGU CUGAUGAG GCCGUUAGGC CGAA IUCCAUGU | 905 | ACAUGGAC U ACGUCGUG | 3684 |
| 1981 | AACGUUCU CUGAUGAG GCCGUUAGGC CGAA ICUCCCAC | 906 | GUGGGAGC C AGAACGUU | 3685 |
| 1982 | GAACGUUC CUGAUGAG GCCGUUAGGC CGAA IGCUCCCA | 907 | UGGGAGCC A GAACGUUC | 3686 |
| 1991 | UUCUCUGC CUGAUGAG GCCGUUAGGC CGAA IAACGUUC | 908 | GAACGUUC C GCAGAGAA | 3687 |
| 1994 | CUUUUCUC CUGAUGAG GCCGUUAGGC CGAA ICGGAACG | 909 | CGUUCCGC A GAGAAAAG | 3688 |
| 2008 | AGACGCUC CUGAUGAG GCCGUUAGGC CGAA ICCCUCUU | 910 | AAGAGGGC C GAGCGUCU | 3689 |
| 2016 | UCGAGGUG CUGAUGAG GCCGUUAGGC CGAA IACGCUCG | 911 | CGAGCGUC U CACCUCGA | 3690 |
| 2018 | CCUCGAGG CUGAUGAG GCCGUUAGGC CGAA IAGACGCU | 912 | AGCGUCUC A CCUCGAGG | 3691 |
| 2020 | ACCCUCGA CUGAUGAG GCCGUUAGGC CGAA IUGAGACG | 913 | CGUCUCAC C UCGAGGGU | 3692 |
| 2021 | CACCCUCG CUGAUGAG GCCGUUAGGC CGAA IGUGAGAC | 914 | GUCUCACC U CGAGGGUG | 3693 |
| 2035 | CUGAACAG CUGAUGAG GCCGUUAGGC CGAA ICCUUCAC | 915 | GUGAAGGC A CUGUUCAG | 3694 |
| 2037 | CGCUGAAC CUGAUGAG GCCGUUAGGC CGAA IUGCCUUC | 916 | GAAGGCAC U GUUCAGCG | 3695 |
| 2042 | GAGCACGC CUGAUGAG GCCGUUAGGC CGAA IAACAGUG | 917 | CACUGUUC A GCGUGCUC | 3696 |
| 2049 | CGUAGUUG CUGAUGAG GCCGUUAGGC CGAA ICACGCUG | 918 | CAGCGUGC U CAACUACG | 3697 |
| 2051 | CUCGUAGU CUGAUGAG GCCGUUAGGC CGAA IAGCACGC | 919 | GCGUGCUC A ACUACGAG | 3698 |
| 2054 | CCGCUCGU CUGAUGAG GCCGUUAGGC CGAA IUUGAGCA | 920 | UGCUCAAC U ACGAGCGG | 3699 |
| 2072 | GAGGCCGG CUGAUGAG GCCGUUAGGC CGAA ICGCCGCG | 921 | CGCGGCGC C CCGGCCUC | 3700 |
| 2073 | GGAGGCCG CUGAUGAG GCCGUUAGGC CGAA IGCGCCGC | 922 | GCGGCGCC C CGGCCUCC | 3701 |
| 2074 | AGGAGGCC CUGAUGAG GCCGUUAGGC CGAA IGGCGCCG | 923 | CGGCGCCC C GGCCUCCU | 3702 |
| 2078 | GCCCAGGA CUGAUGAG GCCGUUAGGC CGAA ICCGGGGC | 924 | GCCCCGGC C UCCUGGGC | 3703 |
| 2079 | CGCCCAGG CUGAUGAG GCCGUUAGGC CGAA IGCCGGGG | 925 | CCCCGGCC U CCUGGGCG | 3704 |
| 2081 | GGCGCCCA CUGAUGAG GCCGUUAGGC CGAA IAGGCCGG | 926 | CCGGCCUC C UGGGCGCC | 3705 |
| 2082 | AGGCGCCC CUGAUGAG GCCGUUAGGC CGAA IGAGGCCG | 927 | CGGCCUCC U GGGCGCCU | 3706 |
| 2089 | AGCACAGA CUGAUGAG GCCGUUAGGC CGAA ICGCCCAG | 928 | CUGGGCGC C UCUGUGCU | 3707 |
| 2090 | CAGCACAG CUGAUGAG GCCGUUAGGC CGAA IGCGCCCA | 929 | UGGGCGCC U CUGUGCUG | 3708 |
| 2092 | CCCAGCAC CUGAUGAG GCCGUUAGGC CGAA IAGGCGCC | 930 | GGCGCCUC U GUGCUGGG | 3709 |
| 2097 | CCAGGCCC CUGAUGAG GCCGUUAGGC CGAA ICACAGAG | 931 | CUCUGUGC U GGGCCUGG | 3710 |
| 2102 | AUCGUCCA CUGAUGAG GCCGUUAGGC CGAA ICCCAGCA | 932 | UGCUGGGC C UGGACGAU | 3711 |
| 2103 | UAUCGUCC CUGAUGAG GCCGUUAGGC CGAA IGCCCAGC | 933 | GCUGGGCC U GGACGAUA | 3712 |
| 2114 | GGCCCUGU CUGAUGAG GCCGUUAGGC CGAA IAUAUCGU | 934 | ACGAUAUC C ACAGGGCC | 3713 |
| 2115 | AGGCCCUG CUGAUGAG GCCGUUAGGC CGAA IGAUAUCG | 935 | CGAUAUCC A CAGGGCCU | 3714 |
| 2117 | CCAGGCCC CUGAUGAG GCCGUUAGGC CGAA IUGGAUAU | 936 | AUAUCCAC A GGGCCUGG | 3715 |
| 2122 | GUGCGCCA CUGAUGAG GCCGUUAGGC CGAA ICCCUGUG | 937 | CACAGGGC C UGGCGCAC | 3716 |
| 2123 | GGUGCGCC CUGAUGAG GCCGUUAGGC CGAA IGCCCUGU | 938 | ACAGGGCC U GGCGCACC | 3717 |
| 2129 | CACGAAGG CUGAUGAG GCCGUUAGGC CGAA ICGCCAGG | 939 | CCUGGCGC A CCUUCGUG | 3718 |
| 2131 | AGCACGAA CUGAUGAG GCCGUUAGGC CGAA IUGCGCCA | 940 | UGGCGCAC C UUCGUGCU | 3719 |
| 2132 | CAGCACGA CUGAUGAG GCCGUUAGGC CGAA IGUGCGCC | 941 | GGCGCACC U UCGUGCUG | 3720 |
| 2139 | GCACACGC CUGAUGAG GCCGUUAGGC CGAA ICACGAAG | 942 | CUUCGUGC U GCGUGUGC | 3721 |
| 2152 | GGGUCCUG CUGAUGAG GCCGUUAGGC CGAA ICCCGCAC | 943 | GUGCGGGC C CAGGACCC | 3722 |
| 2153 | CGGGUCCU CUGAUGAG GCCGUUAGGC CGAA IGCCCGCA | 944 | UGCGGGCC C AGGACCCG | 3723 |
| 2154 | GCGGGUCC CUGAUGAG GCCGUUAGGC CGAA IGGCCCGC | 945 | GCGGGCCC A GGACCCGC | 3724 |
| 2159 | AGGCGGCG CUGAUGAG GCCGUUAGGC CGAA IUCCUGGG | 946 | CCCAGGAC C CGCCGCCU | 3725 |
| 2160 | CAGGCGGC CUGAUGAG GCCGUUAGGC CGAA IGUCCUGG | 947 | CCAGGACC C GCCGCCUG | 3726 |
| 2163 | GCUCAGGC CUGAUGAG GCCGUUAGGC CGAA ICGGGUCC | 948 | GGACCCGC C GCCUGAGC | 3727 |
| 2166 | ACAGCUCA CUGAUGAG GCCGUUAGGC CGAA ICGGCGGG | 949 | CCCGCCGC C UGAGCUGU | 3728 |
| 2167 | UACAGCUC CUGAUGAG GCCGUUAGGC CGAA IGCGGCGG | 950 | CCGCCGCC U GAGCUGUA | 3729 |
| 2172 | CAAAGUAC CUGAUGAG GCCGUUAGGC CGAA ICUCAGGC | 951 | GCCUGAGC U GUACUUUG | 3730 |
| 2177 | CUUGACAA CUGAUGAG GCCGUUAGGC CGAA IUACAGCU | 952 | AGCUGUAC U UUGUCAAG | 3731 |
| 2183 | AUCCACCU CUGAUGAG GCCGUUAGGC CGAA IACAAAGU | 953 | ACUUUGUC A AGGUGGAU | 3732 |
| 2210 | GGGGAUGG CUGAUGAG GCCGUUAGGC CGAA IUCGUACG | 954 | CGUACGAC A CCAUCCCC | 3733 |
| 2212 | UGGGGGAU CUGAUGAG GCCGUUAGGC CGAA IUGUCGUA | 955 | UACGACAC C AUCCCCCA | 3734 |
| 2213 | CUGGGGGA CUGAUGAG GCCGUUAGGC CGAA IGUGUCGU | 956 | ACGACACC A UCCCCCAG | 3735 |
| 2216 | GUCCUGGG CUGAUGAG GCCGUUAGGC CGAA IAUGGUGU | 957 | ACACCAUC C CCCAGGAC | 3736 |
| 2217 | UGUCCUGG CUGAUGAG GCCGUUAGGC CGAA IGAUGGUG | 958 | CACCAUCC C CCAGGACA | 3737 |
| 2218 | CUGUCCUG CUGAUGAG GCCGUUAGGC CGAA IGGAUGGU | 959 | ACCAUCCC C CAGGACAG | 3738 |
| 2219 | CCUGUCCU CUGAUGAG GCCGUUAGGC CGAA IGGGAUGG | 960 | CCAUCCCC C AGGACAGG | 3739 |
| 2220 | GCCUGUCC CUGAUGAG GCCGUUAGGC CGAA IGGGGAUG | 961 | CAUCCCCC A GGACAGGC | 3740 |
| 2225 | CGUGAGCC CUGAUGAG GCCGUUAGGC CGAA IUCCUGGG | 962 | CCCAGGAC A GGCUCACG | 3741 |
| 2229 | CCUCCGUG CUGAUGAG GCCGUUAGGC CGAA ICCUGUCC | 963 | GGACAGGC U CACGGAGG | 3742 |
| 2231 | GACCUCCG CUGAUGAG GCCGUUAGGC CGAA IAGCCUGU | 964 | ACAGGCUC A CGGAGGUC | 3743 |
| 2240 | GCUGGCGA CUGAUGAG GCCGUUAGGC CGAA IACCUCCG | 965 | CGGAGGUC A UCGCCAGC | 3744 |
| 2245 | AUGAUGCU CUGAUGAG GCCGUUAGGC CGAA ICGAUGAC | 966 | GUCAUCGC C AGCAUCAU | 3745 |
| 2246 | GAUGAUGC CUGAUGAG GCCGUUAGGC CGAA IGCGAUGA | 967 | UCAUCGCC A GCAUCAUC | 3746 |
| 2249 | UUUGAUGA CUGAUGAG GCCGUUAGGC CGAA ICUGGCGA | 968 | UCGCCAGC A UCAUCAAA | 3747 |
| 2252 | GGGUUUGA CUGAUGAG GCCGUUAGGC CGAA IAUGCUGG | 969 | CCAGCAUC A UCAAACCC | 3748 |
| 2255 | CUGGGGUU CUGAUGAG GCCGUUAGGC CGAA IAUGAUGC | 970 | GCAUCAUC A AACCCCAG | 3749 |
| 2259 | UGUUCUGG CUGAUGAG GCCGUUAGGC CGAA IUUUGAUG | 971 | CAUCAAAC C CCAGAACA | 3750 |
| 2260 | GUGUUCUG CUGAUGAG GCCGUUAGGC CGAA IGUUUGAU | 972 | AUCAAACC C CAGAACAC | 3751 |
| 2261 | CGUGUUCU CUGAUGAG GCCGUUAGGC CGAA IGGUUUGA | 973 | UCAAACCC C AGAACACG | 3752 |
| 2262 | ACGUGUUC CUGAUGAG GCCGUUAGGC CGAA IGGGUUUG | 974 | CAAACCCC A GAACACGU | 3753 |
| 2267 | GCAGUACG CUGAUGAG GCCGUUAGGC CGAA IUUCUGGG | 975 | CCCAGAAC A CGUACUGC | 3754 |
| 2273 | ACGCACGC CUGAUGAG GCCGUUAGGC CGAA IUACGUGU | 976 | ACACGUAC U GCGUGCGU | 3755 |
| 2290 | UGGACCAC CUGAUGAG GCCGUUAGGC CGAA ICAUACCG | 977 | CGGUAUGC C GUGGUCCA | 3756 |
| 2297 | GGCCUUCU CUGAUGAG GCCGUUAGGC CGAA IACCACGG | 978 | CCGUGGUC C AGAAGGCC | 3757 |
| 2298 | CGGCCUUC CUGAUGAG GCCGUUAGGC CGAA IGACCACG | 979 | CGUGGUCC A GAAGGCCG | 3758 |
| 2305 | CCAUGGGC CUGAUGAG GCCGUUAGGC CGAA ICCUUCUG | 980 | CAGAAGGC C GCCCAUGG | 3759 |
| 2308 | UGCCCAUG CUGAUGAG GCCGUUAGGC CGAA ICGGCCUU | 981 | AAGGCCGC C CAUGGGCA | 3760 |
| 2309 | GUGCCCAU CUGAUGAG GCCGUUAGGC CGAA IGCGGCCU | 982 | AGGCCGCC C AUGGGCAC | 3761 |
| 2310 | CGUGCCCA CUGAUGAG GCCGUUAGGC CGAA IGGCGGCC | 983 | GGCCGCCC A UGGGCACG | 3762 |
| 2316 | UGCGGACG CUGAUGAG GCCGUUAGGC CGAA ICCCAUGG | 984 | CCAUGGGC A CGUCCGCA | 3763 |
| 2321 | GGCCUUGC CUGAUGAG GCCGUUAGGC CGAA IACGUGCC | 985 | GGCACGUC C GCAAGGCC | 3764 |
| 2324 | GAAGGCCU CUGAUGAG GCCGUUAGGC CGAA ICGGACGU | 986 | ACGUCCGC A AGGCCUUC | 3765 |
| 2329 | CUCUUGAA CUGAUGAG GCCGUUAGGC CGAA ICCUUGCG | 987 | CGCAAGGC C UUCAAGAG | 3766 |
| 2330 | GCUCUUGA CUGAUGAG GCCGUUAGGC CGAA IGCCUUGC | 988 | GCAAGGCC U UCAAGAGC | 3767 |
| 2333 | GUGGCUCU CUGAUGAG GCCGUUAGGC CGAA IAAGGCCU | 989 | AGGCCUUC A AGAGCCAC | 3768 |
| 2339 | AGAGACGU CUGAUGAG GCCGUUAGGC CGAA ICUCUUGA | 990 | UCAAGAGC C ACGUCUCU | 3769 |
| 2340 | UAGAGACG CUGAUGAG GCCGUUAGGC CGAA IGCUCUUG | 991 | CAAGAGCC A CGUCUCUA | 3770 |
| 2345 | CAAGGUAG CUGAUGAG GCCGUUAGGC CGAA IACGUGGC | 992 | GCCACGUC U CUACCUUG | 3771 |
| 2347 | GUCAAGGU CUGAUGAG GCCGUUAGGC CGAA IAGACGUG | 993 | CAGGUCUC U ACCUUGAC | 3772 |
| 2350 | UCUGUCAA CUGAUGAG GCCGUUAGGC CGAA IUAGAGAC | 994 | GUCUCUAC C UUGACAGA | 3773 |
| 2351 | GUCUGUCA CUGAUGAG GCCGUUAGGC CGAA IGUAGAGA | 995 | UCUCUACC U UGACAGAC | 3774 |
| 2356 | UGGAGGUC CUGAUGAG GCCGUUAGGC CGAA IUCAAGGU | 996 | ACCUUGAC A GACCUCCA | 3775 |
| 2360 | CGGCUGGA CUGAUGAG GCCGUUAGGC CGAA IUCUGUCA | 997 | UGACAGAC C UCCAGCCG | 3776 |
| 2361 | ACGGCUGG CUGAUGAG GCCGUUAGGC CGAA IGUCUGUC | 998 | GACAGACC U CCAGCCGU | 3777 |
| 2363 | GUACGGCU CUGAUGAG GCCGUUAGGC CGAA IAGGUCUG | 999 | CAGACCUC C AGCCGUAC | 3778 |
| 2364 | UGUACGGC CUGAUGAG GCCGUUAGGC CGAA IGAGGUCU | 1000 | AGACCUCC A GCCGUACA | 3779 |
| 2367 | GCAUGUAC CUGAUGAG GCCGUUAGGC CGAA ICUGGAGG | 1001 | CCUCCAGC C GUACAUGC | 3780 |
| 2372 | CUGUCGCA CUGAUGAG GCCGUUAGGC CGAA IUACGGCU | 1002 | AGCCGUAC A UGCGACAG | 3781 |
| 2379 | CCACGAAC CUGAUGAG GCCGUUAGGC CGAA IUCGCAUG | 1003 | CAUGCGAC A GUUCGUGG | 3782 |
| 2389 | UGCAGGUG CUGAUGAG GCCGUUAGGC CGAA ICCACGAA | 1004 | UUCGUGGC U CACCUGCA | 3783 |
| 2391 | CCUGCAGG CUGAUGAG GCCGUUAGGC CGAA IAGCCACG | 1005 | CGUGGCUC A CCUGCAGG | 3784 |
| 2393 | CUCCUGCA CUGAUGAG GCCGUUAGGC CGAA IUGAGCCA | 1006 | UGGCUCAC C UGCAGGAG | 3785 |
| 2394 | UCUCCUGC CUGAUGAG GCCGUUAGGC CGAA IGUGAGCC | 1007 | GGCUCACC U GCAGGAGA | 3786 |
| 2397 | UGGUCUCC CUGAUGAG GCCGUUAGGC CGAA ICAGGUGA | 1008 | UCACCUGC A GGAGACCA | 3787 |
| 2404 | AGCGGGCU CUGAUGAG GCCGUUAGGC CGAA IUCUCCUG | 1009 | CAGGAGAC C AGCCCGCU | 3788 |
| 2405 | CAGCGGGC CUGAUGAG GCCGUUAGGC CGAA IGUCUCCU | 1010 | AGGAGACC A GCCCGCUG | 3789 |
| 2408 | CCUCAGCG CUGAUGAG GCCGUUAGGC CGAA ICUGGUCU | 1011 | AGACCAGC C CGCUGAGG | 3790 |
| 2409 | CCCUCAGC CUGAUGAG GCCGUUAGGC CGAA IGCUGGUC | 1012 | GACCAGCC C GCUGAGGG | 3791 |
| 2412 | CAUCCCUC CUGAUGAG GCCGUUAGGC CGAA ICGGGCUG | 1013 | CAGCCCGC U GAGGGAUG | 3792 |
| 2422 | AUGACGAC CUGAUGAG GCCGUUAGGC CGAA ICAUCCCU | 1014 | AGGGAUGC C GUCGUCAU | 3793 |
| 2429 | CUGCUCGA CUGAUGAG GCCGUUAGGC CGAA IACGACGG | 1015 | CCGUCGUC A UCGAGCAG | 3794 |
| 2436 | AGGAGCUC CUGAUGAG GCCGUUAGGC CGAA ICUCGAUG | 1016 | CAUCGAGC A GAGCUCCU | 3795 |
| 2441 | CAGGGAGG CUGAUGAG GCCGUUAGGC CGAA ICUCUGCU | 1017 | AGCAGAGC U CCUCCCUG | 3796 |
| 2443 | UUCAGGGA CUGAUGAG GCCGUUAGGC CGAA IAGCUCUG | 1018 | CAGAGCUC C UCCCUGAA | 3797 |
| 2444 | AUUCAGGG CUGAUGAG GCCGUUAGGC CGAA IGAGCUCU | 1019 | AGAGCUCC U CCCUGAAU | 3798 |
| 2446 | UCAUUCAG CUGAUGAG GCCGUUAGGC CGAA IAGGAGCU | 1020 | AGCUCCUC C CUGAAUGA | 3799 |
| 2447 | CUCAUUCA CUGAUGAG GCCGUUAGGC CGAA IGAGGAGC | 1021 | GCUCCUCC C UGAAUGAG | 3800 |
| 2448 | CCUCAUUC CUGAUGAG GCCGUUAGGC CGAA IGGAGGAG | 1022 | CUCCUCCC U GAAUGAGG | 3801 |
| 2458 | CCACUGCU CUGAUGAG GCCGUUAGGC CGAA ICCUCAUU | 1023 | AAUGAGGC C AGCAGUGG | 3802 |
| 2459 | GCCACUGC CUGAUGAG GCCGUUAGGC CGAA IGCCUCAU | 1024 | AUGAGGCC A GCAGUGGC | 3803 |
| 2462 | GAGGCCAC CUGAUGAG GCCGUUAGGC CGAA ICUGGCCU | 1025 | AGGCCAGC A GUGGCCUC | 3804 |
| 2468 | GUCGAAGA CUGAUGAG GCCGUUAGGC CGAA ICCACUGC | 1026 | GCAGUGGC C UCUUCGAC | 3805 |
| 2469 | CGUCGAAG CUGAUGAG GCCGUUAGGC CGAA IGCCACUG | 1027 | CAGUGGCC U CUUCGACG | 3806 |
| 2471 | GACGUCGA CUGAUGAG GCCGUUAGGC CGAA IAGGCCAC | 1028 | GUGGCCUC U UCGACGUC | 3807 |
| 2480 | GCGUAGGA CUGAUGAG GCCGUUAGGC CGAA IACGUCGA | 1029 | UCGACGUC U UCCUACGC | 3808 |
| 2483 | GAAGCGUA CUGAUGAG GCCGUUAGGC CGAA IAAGACGU | 1030 | ACGUCUUC C UACGCUUC | 3809 |
| 2484 | UGAAGCGU CUGAUGAG GCCGUUAGGC CGAA IGAAGACG | 1031 | CGUCUUCC U ACGCUUCA | 3810 |
| 2489 | GCACAUGA CUGAUGAG GCCGUUAGGC CGAA ICGUAGGA | 1032 | UCCUACGC U UCAUGUGC | 3811 |
| 2492 | GUGGCACA CUGAUGAG GCCGUUAGGC CGAA IAAGCGUA | 1033 | UACGCUUC A UGUGCCAC | 3812 |
| 2498 | GGCGUGGU CUGAUGAG GCCGUUAGGC CGAA ICACAUGA | 1034 | UCAUGUGC C ACCACGCC | 3813 |
| 2499 | CGGCGUGG CUGAUGAG GCCGUUAGGC CGAA IGCACAUG | 1035 | CAUGUGCC A CCACGCCG | 3814 |
| 2501 | CACGGCGU CUGAUGAG GCCGUUAGGC CGAA IUGGCACA | 1036 | UGUGCCAC C ACGCCGUG | 3815 |
| 2502 | GCACGGCG CUGAUGAG GCCGUUAGGC CGAA IGUGGCAC | 1037 | GUGCCACC A CGCCGUGC | 3816 |
| 2506 | AUGCGCAC CUGAUGAG GCCGUUAGGC CGAA ICGUGGUG | 1038 | CACCACGC C GUGCGCAU | 3817 |
| 2513 | GCCCCUGA CUGAUGAG GCCGUUAGGC CGAA ICGCACGG | 1039 | CCGUGCGC A UCAGGGGC | 3818 |
| 2516 | CUUGCCCC CUGAUGAG GCCGUUAGGC CGAA IAUGCGCA | 1040 | UGCGCAUC A GGGGCAAG | 3819 |
| 2522 | GUAGGACU CUGAUGAG GCCGUUAGGC CGAA ICCCCUGA | 1041 | UCAGGGGC A AGUCCUAC | 3820 |
| 2527 | UGGACGUA CUGAUGAG GCCGUUAGGC CGAA IACUUGCC | 1042 | GGCAAGUC C UACGUCCA | 3821 |
| 2528 | CUGGACGU CUGAUGAG GCCGUUAGGC CGAA IGACUUGC | 1043 | GCAAGUCC U ACGUCCAG | 3822 |
| 2534 | CUGGCACU CUGAUGAG GCCGUUAGGC CGAA IACGUAGG | 1044 | CCUACGUC C AGUGCCAG | 3823 |
| 2535 | CCUGGCAC CUGAUGAG GCCGUUAGGC CGAA IGACGUAG | 1045 | CUACGUCC A GUGCCAGG | 3824 |
| 2540 | GAUCCCCU CUGAUGAG GCCGUUAGGC CGAA ICACUGGA | 1046 | UCCAGUGC C AGGGGAUC | 3825 |
| 2541 | GGAUCCCC CUGAUGAG GCCGUUAGGC CGAA IGCACUGG | 1047 | CCAGUGCC A GGGGAUCC | 3826 |
| 2549 | GCCCUGCG CUGAUGAG GCCGUUAGGC CGAA IAUCCCCU | 1048 | AGGGGAUC C CGCAGGGC | 3827 |
| 2550 | AGCCCUGC CUGAUGAG GCCGUUAGGC CGAA IGAUCCCC | 1049 | GGGGAUCC C GCAGGGCU | 3828 |
| 2553 | UGGAGCCC CUGAUGAG GCCGUUAGGC CGAA ICGGGAUC | 1050 | GAUCCCGC A GGGCUCCA | 3829 |
| 2558 | GAGGAUGG CUGAUGAG GCCGUUAGGC CGAA ICCCUGCG | 1051 | CGCAGGGC U CCAUCCUC | 3830 |
| 2560 | GAGAGGAU CUGAUGAG GCCGUUAGGC CGAA IAGCCCUG | 1052 | CAGGGCUC C AUCCUCUC | 3831 |
| 2561 | GGAGAGGA CUGAUGAG GCCGUUAGGC CGAA IGAGCCCU | 1053 | AGGGCUCC A UCCUCUCC | 3832 |
| 2564 | CGUGGAGA CUGAUGAG GCCGUUAGGC CGAA IAUGGAGC | 1054 | GCUCCAUC C UCUCCACG | 3833 |
| 2565 | GCGUGGAG CUGAUGAG GCCGUUAGGC CGAA IGAUGGAG | 1055 | CUCCAUCC U CUCCACGC | 3834 |
| 2567 | CAGCGUGG CUGAUGAG GCCGUUAGGC CGAA IAGGAUGG | 1056 | CCAUCCUC U CCACGCUG | 3835 |
| 2569 | AGCAGCGU CUGAUGAG GCCGUUAGGC CGAA IAGAGGAU | 1057 | AUCCUCUC C ACGCUGCU | 3836 |
| 2570 | GAGCAGCG CUGAUGAG GCCGUUAGGC CGAA IGAGAGGA | 1058 | UCCUCUCC A CGCUGCUC | 3837 |
| 2574 | UGCAGAGC CUGAUGAG GCCGUUAGGC CGAA ICGUGGAG | 1059 | CUCCACGC U GCUCUGCA | 3838 |
| 2577 | GGCUGCAG CUGAUGAG GCCGUUAGGC CGAA ICAGCGUG | 1060 | CACGCUGC U CUGCAGCC | 3839 |
| 2579 | CAGGCUGC CUGAUGAG GCCGUUAGGC CGAA IAGCAGCG | 1061 | CGCUGCUC U GCAGCCUG | 3840 |
| 2582 | GCACAGGC CUGAUGAG GCCGUUAGGC CGAA ICAGAGCA | 1062 | UGCUCUGC A GCCUGUGC | 3841 |
| 2585 | GUAGCACA CUGAUGAG GCCGUUAGGC CGAA ICUGCAGA | 1063 | UCUGCAGC C UGUGCUAC | 3842 |
| 2586 | CGUAGCAC CUGAUGAG GCCGUUAGGC CGAA IGCUGCAG | 1064 | CUGCAGCC U GUGCUACG | 3843 |
| 2591 | GUCGCCGU CUGAUGAG GCCGUUAGGC CGAA ICACAGGC | 1065 | GCCUGUGC U ACGGCGAC | 3844 |
| 2600 | GUUCUCCA CUGAUGAG GCCGUUAGGC CGAA IUCGCCGU | 1066 | ACGGCGAC A UGGAGAAC | 3845 |
| 2609 | AAACAGCU CUGAUGAG GCCGUUAGGC CGAA IUUCUCCA | 1067 | UGGAGAAC A AGCUGUUU | 3846 |
| 2613 | CCGCAAAC CUGAUGAG GCCGUUAGGC CGAA ICUUGUUC | 1068 | GAACAAGC U GUUUGCGG | 3847 |
| 2640 | GCAGGAGC CUGAUGAG GCCGUUAGGC CGAA ICCCGUCC | 1069 | GGACGGGC U GCUCCUGC | 3848 |
| 2643 | AACGCAGG CUGAUGAG GCCGUUAGGC CGAA ICAGCCCG | 1070 | CGGGCUGC U CCUGCGUU | 3849 |
| 2645 | CAAACGCA CUGAUGAG GCCGUUAGGC CGAA IAGCAGCC | 1071 | GGCUGCUC C UGCGUUUG | 3850 |
| 2646 | CCAAACGC CUGAUGAG GCCGUUAGGC CGAA IGAGCAGC | 1072 | GCUGCUCC U GCGUUUGG | 3851 |
| 2666 | CACCAACA CUGAUGAG GCCGUUAGGC CGAA IAAAUCAU | 1073 | AUGAUUUC U UGUUGGUG | 3852 |
| 2677 | AGGUGAGG CUGAUGAG GCCGUUAGGC CGAA IUCACCAA | 1074 | UUGGUGAC A CCUCACCU | 3853 |
| 2679 | UGAGGUGA CUGAUGAG GCCGUUAGGC CGAA IUGUCACC | 1075 | GGUGACAC C UCACCUCA | 3854 |
| 2680 | GUGAGGUG CUGAUGAG GCCGUUAGGC CGAA IGUGUCAC | 1076 | GUGACACC U CACCUCAC | 3855 |
| 2682 | GGGUGAGG CUGAUGAG GCCGUUAGGC CGAA IAGGUGUC | 1077 | GACACCUC A CCUCACCC | 3856 |
| 2684 | GUGGGUGA CUGAUGAG GCCGUUAGGC CGAA IUGAGGUG | 1078 | CACCUCAC C UCACCCAC | 3857 |
| 2685 | CGUGGGUG CUGAUGAG GCCGUUAGGC CGAA IGUGAGGU | 1079 | ACCUCACC U CACCCACG | 3858 |
| 2687 | CGCGUGGG CUGAUGAG GCCGUUAGGC CGAA IAGGUGAG | 1080 | CUCACCUC A CCCACGCG | 3859 |
| 2689 | UUCGCGUG CUGAUGAG GCCGUUAGGC CGAA IUGAGGUG | 1081 | CACCUCAC C CACGCGAA | 3860 |
| 2690 | UUUCGCGU CUGAUGAG GCCGUUAGGC CGAA IGUGAGGU | 1082 | ACCUCACC C ACGCGAAA | 3861 |
| 2691 | UUUUCGCG CUGAUGAG GCCGUUAGGC CGAA IGGUGAGG | 1083 | CCUCACCC A CGCGAAAA | 3862 |
| 2701 | CUGAGGAA CUGAUGAG GCCGUUAGGC CGAA IUUUUCGC | 1084 | GCGAAAAC C UUCCUCAG | 3863 |
| 2702 | CCUGAGGA CUGAUGAG GCCGUUAGGC CGAA IGUUUUCG | 1085 | CGAAAACC U UCCUCAGG | 3864 |
| 2705 | GGUCCUGA CUGAUGAG GCCGUUAGGC CGAA IAAGGUUU | 1086 | AAACCUUC C UCAGGACC | 3865 |
| 2706 | GGGUCCUG CUGAUGAG GCCGUUAGGC CGAA IGAAGGUU | 1087 | AACCUUCC U CAGGACCC | 3866 |
| 2708 | CAGGGUCC CUGAUGAG GCCGUUAGGC CGAA IAGGAAGG | 1088 | CCUUCCUC A GGACCCUG | 3867 |
| 2713 | CGGACCAG CUGAUGAG GCCGUUAGGC CGAA IUCCUGAG | 1089 | CUCAGGAC C CUGGUCCG | 3868 |
| 2714 | UCGGACCA CUGAUGAG GCCGUUAGGC CGAA IGUCCUGA | 1090 | UCAGGACC C UGGUCCGA | 3869 |
| 2715 | CUCGGACC CUGAUGAG GCCGUUAGGC CGAA IGGUCCUG | 1091 | CAGGACCC U GGUCCGAG | 3870 |
| 2720 | GACACCUC CUGAUGAG GCCGUUAGGC CGAA IACCAGGG | 1092 | CCCUGGUC C GAGGUGUC | 3871 |
| 2729 | AUACUCAG CUGAUGAG GCCGUUAGGC CGAA IACACCUC | 1093 | GAGGUGUC C CUGAGUAU | 3872 |
| 2730 | CAUACUCA CUGAUGAG GCCGUUAGGC CGAA IGACACCU | 1094 | AGGUGUCC C UGAGUAUG | 3873 |
| 2731 | CCAUACUC CUGAUGAG GCCGUUAGGC CGAA IGGACACC | 1095 | GGUGUCCC U GAGUAUGG | 3874 |
| 2741 | CACCACGC CUGAUGAG GCCGUUAGGC CGAA ICCAUACU | 1096 | AGUAUGGC U GCGUGGUG | 3875 |
| 2753 | CUUCCGCA CUGAUGAG GCCGUUAGGC CGAA IUUCACCA | 1097 | UGGUGAAC U UGCGGAAG | 3876 |
| 2764 | UUCACCAC CUGAUGAG GCCGUUAGGC CGAA IUCUUCCG | 1098 | CGGAAGAC A GUGGUGAA | 3877 |
| 2774 | UACAGGGA CUGAUGAG GCCGUUAGGC CGAA IUUCACCA | 1099 | UGGUGAAC U UCCCUGUA | 3878 |
| 2777 | UUCUACAG CUGAUGAG GCCGUUAGGC CGAA IAAGUUCA | 1100 | UGAACUUC C CUGUAGAA | 3879 |
| 2778 | CUUCUACA CUGAUGAG GCCGUUAGGC CGAA IGAAGUUC | 1101 | GAACUUCC C UGUAGAAG | 3880 |
| 2779 | UCUUCUAC CUGAUGAG GCCGUUAGGC CGAA IGGAAGUU | 1102 | AACUUCCC U GUAGAAGA | 3881 |
| 2794 | CCACCCAG CUGAUGAG GCCGUUAGGC CGAA ICCUCGUC | 1103 | GACGAGGC C CUGGGUGG | 3882 |
| 2795 | GCCACCCA CUGAUGAG GCCGUUAGGC CGAA IGCCUCGU | 1104 | ACGAGGCC C UGGGUGGC | 3883 |
| 2796 | UGCCACCC CUGAUGAG GCCGUUAGGC CGAA IGGCCUCG | 1105 | CGAGGCCC U GGGUGGCA | 3884 |
| 2804 | AAAAGCCG CUGAUGAG GCCGUUAGGC CGAA ICCACCCA | 1106 | UGGGUGGC A CGGCUUUU | 3885 |
| 2809 | UGAACAAA CUGAUGAG GCCGUUAGGC CGAA ICCGUGCC | 1107 | GGCACGGC U UUUGUUCA | 3886 |
| 2817 | CCGGCAUC CUGAUGAG GCCGUUAGGC CGAA IAACAAAA | 1108 | UUUUGUUC A GAUGCCGG | 3887 |
| 2823 | CGUGGGCC CUGAUGAG GCCGUUAGGC CGAA ICAUCUGA | 1109 | UCAGAUGC C GGCCCACG | 3888 |
| 2827 | AGGCCGUG CUGAUGAG GCCGUUAGGC CGAA ICCGGCAU | 1110 | AUGCCGGC C CACGGCCU | 3889 |
| 2828 | UAGGCCGU CUGAUGAG GCCGUUAGGC CGAA IGCCGGCA | 1111 | UGCCGGCC C ACGGCCUA | 3890 |
| 2829 | AUAGGCCG CUGAUGAG GCCGUUAGGC CGAA IGGCCGGC | 1112 | GCCGGCCC A CGGCCUAU | 3891 |
| 2834 | GGGGAAUA CUGAUGAG GCCGUUAGGC CGAA ICCGUGGG | 1113 | CCCACGGC C UAUUCCCC | 3892 |
| 2835 | AGGGGAAU CUGAUGAG GCCGUUAGGC CGAA IGCCGUGG | 1114 | CCACGGCC U AUUCCCCU | 3893 |
| 2840 | GCACCAGG CUGAUGAG GCCGUUAGGC CGAA IAAUAGGC | 1115 | GCCUAUUC C CCUGGUGC | 3894 |
| 2841 | CGCACCAG CUGAUGAG GCCGUUAGGC CGAA IGAAUAGG | 1116 | CCUAUUCC C CUGGUGCG | 3895 |
| 2842 | CCGCACCA CUGAUGAG GCCGUUAGGC CGAA IGGAAUAG | 1117 | CUAUUCCC C UGGUGCGG | 3896 |
| 2843 | GCCGCACC CUGAUGAG GCCGUUAGGC CGAA IGGGAAUA | 1118 | UAUUCCCC U GGUGCGGC | 3897 |
| 2852 | CAGCAGCA CUGAUGAG GCCGUUAGGC CGAA ICCGCACC | 1119 | GGUGCGGC C UGCUGCUG | 3898 |
| 2853 | CCAGCAGC CUGAUGAG GCCGUUAGGC CGAA IGCCGCAC | 1120 | GUGCGGCC U GCUGCUGG | 3899 |
| 2856 | UAUCCAGC CUGAUGAG GCCGUUAGGC CGAA ICAGGCCG | 1121 | CGGCCUGC U GCUGGAUA | 3900 |
| 2859 | GGGUAUCC CUGAUGAG GCCGUUAGGC CGAA ICAGCAGG | 1122 | CCUGCUGC U GGAUACCC | 3901 |
| 2866 | AGGGUCCG CUGAUGAG GCCGUUAGGC CGAA IUAUCCAG | 1123 | CUGGAUAC C CGGACCCU | 3902 |
| 2867 | CAGGGUCC CUGAUGAG GCCGUUAGGC CGAA IGUAUCCA | 1124 | UGGAUACC C GGACCCUG | 3903 |
| 2872 | ACCUCCAG CUGAUGAG GCCGUUAGGC CGAA IUCCGGGU | 1125 | ACCCGGAC C CUGGAGGU | 3904 |
| 2873 | CACCUCCA CUGAUGAG GCCGUUAGGC CGAA IGUCCGGG | 1126 | CCCGGACC C UGGAGGUG | 3905 |
| 2874 | GCACCUCC CUGAUGAG GCCGUUAGGC CGAA IGGUCCGG | 1127 | CCGGACCC U GGAGGUGC | 3906 |
| 2883 | AGUCGCUC CUGAUGAG GCCGUUAGGC CGAA ICACCUCC | 1128 | GGAGGUGC A GAGCGACU | 3907 |
| 2891 | GCUGGAGU CUGAUGAG GCCGUUAGGC CGAA IUCGCUCU | 1129 | AGAGCGAC U ACUCCAGC | 3908 |
| 2894 | AUAGCUGG CUGAUGAG GCCGUUAGGC CGAA IUAGUCGC | 1130 | GCGACUAC U CCAGCUAU | 3909 |
| 2896 | GCAUAGCU CUGAUGAG GCCGUUAGGC CGAA IAGUAGUC | 1131 | GACUACUC C AGCUAUGC | 3910 |
| 2897 | GGCAUAGC CUGAUGAG GCCGUUAGGC CGAA IGAGUAGU | 1132 | ACUACUCC A GCUAUGCC | 3911 |
| 2900 | CCGGGCAU CUGAUGAG GCCGUUAGGC CGAA ICUGGAGU | 1133 | ACUCCAGC U AUGCCCGG | 3912 |
| 2905 | GAGGUCCG CUGAUGAG GCCGUUAGGC CGAA ICAUAGCU | 1134 | AGCUAUGC C CGGACCUC | 3913 |
| 2906 | GGAGGUCC CUGAUGAG GCCGUUAGGC CGAA IGCAUAGC | 1135 | GCUAUGCC C GGACCUCC | 3914 |
| 2911 | CUGAUGGA CUGAUGAG GCCGUUAGGC CGAA IUCCGGGC | 1136 | GCCCGGAC C UCCAUCAG | 3915 |
| 2912 | UCUGAUGG CUGAUGAG GCCGUUAGGC CGAA IGUCCGGG | 1137 | CCCGGACC U CCAUCAGA | 3916 |
| 2914 | GCUCUGAU CUGAUGAG GCCGUUAGGC CGAA IAGGUCCG | 1138 | CGGACCUC C AUCAGAGC | 3917 |
| 2915 | GGCUCUGA CUGAUGAG GCCGUUAGGC CGAA IGAGGUCC | 1139 | GGACCUCC A UCAGAGCC | 3918 |
| 2918 | ACUGGCUC CUGAUGAG GCCGUUAGGC CGAA IAUGGAGG | 1140 | CCUCCAUC A GAGCCAGU | 3919 |
| 2923 | GUGAGACU CUGAUGAG GCCGUUAGGC CGAA ICUCUGAU | 1141 | AUCAGAGC C AGUCUCAC | 3920 |
| 2924 | GGUGAGAC CUGAUGAG GCCGUUAGGC CGAA IGCUCUGA | 1142 | UCAGAGCC A GUCUCACC | 3921 |
| 2928 | UGAAGGUG CUGAUGAG GCCGUUAGGC CGAA IACUGGCU | 1143 | AGCCAGUC U CACCUUCA | 3922 |
| 2930 | GUUGAAGG CUGAUGAG GCCGUUAGGC CGAA IAGACUGG | 1144 | CCAGUCUC A CCUUCAAC | 3923 |
| 2932 | CGGUUGAA CUGAUGAG GCCGUUAGGC CGAA IUGAGACU | 1145 | AGUCUCAC C UUCAACCG | 3924 |
| 2933 | GCGGUUGA CUGAUGAG GCCGUUAGGC CGAA IGUGAGAC | 1146 | GUCUCACC U UCAACCGC | 3925 |
| 2936 | GCCGCGGU CUGAUGAG GCCGUUAGGC CGAA IAAGGUGA | 1147 | UCACCUUC A ACCGCGGC | 3926 |
| 2939 | GAAGCCGC CUGAUGAG GCCGUUAGGC CGAA IUUGAAGG | 1148 | CCUUCAAC C GCGGCUUC | 3927 |
| 2945 | AGCCUUGA CUGAUGAG GCCGUUAGGC CGAA ICCGCGGU | 1149 | ACCGCGGC U UCAAGGCU | 3928 |
| 2948 | CCCAGCCU CUGAUGAG GCCGUUAGGC CGAA IAAGCCGC | 1150 | GCGGCUUC A AGGCUGGG | 3929 |
| 2953 | UUCCUCCC CUGAUGAG GCCGUUAGGC CGAA ICCUUGAA | 1151 | UUCAAGGC U GGGAGGAA | 3930 |
| 2963 | GCGACGCA CUGAUGAG GCCGUUAGGC CGAA IUUCCUCC | 1152 | GGAGGAAC A UGCGUCGC | 3931 |
| 2972 | AAAGAGUU CUGAUGAG GCCGUUAGGC CGAA ICGACGCA | 1153 | UGCGUCGC A AACUCUUU | 3932 |
| 2976 | CCCCAAAG CUGAUGAG GCCGUUAGGC CGAA IUUUGCGA | 1154 | UCGCAAAC U CUUUGGGG | 3933 |
| 2978 | GACCCCAA CUGAUGAG GCCGUUAGGC CGAA IAGUUUGC | 1155 | GCAAACUC U UUGGGGUC | 3934 |
| 2987 | CAGCCGCA CUGAUGAG GCCGUUAGGC CGAA IACCCCAA | 1156 | UUGGGGUC U UGCGGCUG | 3935 |
| 2994 | GACACUUC CUGAUGAG GCCGUUAGGC CGAA ICCGCAAG | 1157 | CUUGCGGC U GAAGUGUC | 3936 |
| 3003 | ACAGGCUG CUGAUGAG GCCGUUAGGC CGAA IACACUUC | 1158 | GAAGUGUC A CAGCCUGU | 3937 |
| 3005 | AAACAGGC CUGAUGAG GCCGUUAGGC CGAA IUGACACU | 1159 | AGUGUCAC A GCCUGUUU | 3938 |
| 3008 | CAGAAACA CUGAUGAG GCCGUUAGGC CGAA ICUGUGAC | 1160 | GUCACAGC C UGUUUCUG | 3939 |
| 3009 | CCAGAAAC CUGAUGAG GCCGUUAGGC CGAA IGCUGUGA | 1161 | UCACAGCC U GUUUCUGG | 3940 |
| 3015 | GCAAAUCC CUGAUGAG GCCGUUAGGC CGAA IAAACAGG | 1162 | CCUGUUUC U GGAUUUGC | 3941 |
| 3024 | UGUUCACC CUGAUGAG GCCGUUAGGC CGAA ICAAAUCC | 1163 | GGAUUUGC A GGUGAACA | 3942 |
| 3032 | CUGGAGGC CUGAUGAG GCCGUUAGGC CGAA IUUCACCU | 1164 | AGGUGAAC A GCCUCCAG | 3943 |
| 3035 | CGUCUGGA CUGAUGAG GCCGUUAGGC CGAA ICUGUUCA | 1165 | UGAACAGC C UCCAGACG | 3944 |
| 3036 | CCGUCUGG CUGAUGAG GCCGUUAGGC CGAA IGCUGUUC | 1166 | GAACAGCC U CCAGACGG | 3945 |
| 3038 | CACCGUCU CUGAUGAG GCCGUUAGGC CGAA IAGGCUGU | 1167 | ACAGCCUC C AGACGGUG | 3946 |
| 3039 | ACACCGUC CUGAUGAG GCCGUUAGGC CGAA IGAGGCUG | 1168 | CAGCCUCC A GACGGUGU | 3947 |
| 3050 | GAUGUUGG CUGAUGAG GCCGUUAGGC CGAA ICACACCG | 1169 | CGGUGUGC A CCAACAUC | 3948 |
| 3052 | UAGAUGUU CUGAUGAG GCCGUUAGGC CGAA IUGCACAC | 1170 | GUGUGCAC C AACAUCUA | 3949 |
| 3053 | GUAGAUGU CUGAUGAG GCCGUUAGGC CGAA IGUGCACA | 1171 | UGUGCACC A ACAUCUAC | 3950 |
| 3056 | CUUGUAGA CUGAUGAG GCCGUUAGGC CGAA IUUGGUGC | 1172 | GCACCAAC A UCUACAAG | 3951 |
| 3059 | GAUCUUGU CUGAUGAG GCCGUUAGGC CGAA IAUGUUGG | 1173 | CCAACAUC U ACAAGAUC | 3952 |
| 3062 | GAGGAUCU CUGAUGAG GCCGUUAGGC CGAA IUAGAUGU | 1174 | ACAUCUAC A AGAUCCUC | 3953 |
| 3068 | CAGCAGGA CUGAUGAG GCCGUUAGGC CGAA IAUCUUGU | 1175 | ACAAGAUC C UCCUGCUG | 3954 |
| 3069 | GCAGCAGG CUGAUGAG GCCGUUAGGC CGAA IGAUCUUG | 1176 | CAAGAUCC U CCUGCUGC | 3955 |
| 3071 | CUGCAGCA CUGAUGAG GCCGUUAGGC CGAA IAGGAUCU | 1177 | AGAUCCUC C UGCUGCAG | 3956 |
| 3072 | CCUGCAGC CUGAUGAG GCCGUUAGGC CGAA IGAGGAUC | 1178 | GAUCCUCC U GCUGCAGG | 3957 |
| 3075 | ACGCCUGC CUGAUGAG GCCGUUAGGC CGAA ICAGGAGG | 1179 | CCUCCUGC U GCAGGCGU | 3958 |
| 3078 | UGUACGCC CUGAUGAG GCCGUUAGGC CGAA ICAGCAGG | 1180 | CCUGCUGC A GGCGUACA | 3959 |
| 3086 | GUGAAACC CUGAUGAG GCCGUUAGGC CGAA IUACGCCU | 1181 | AGGCGUAC A GGUUUCAC | 3960 |
| 3093 | CACAUGCG CUGAUGAG GCCGUUAGGC CGAA IAAACCUG | 1182 | CAGGUUUC A CGCAUGUG | 3961 |
| 3097 | AGCACACA CUGAUGAG GCCGUUAGGC CGAA ICGUGAAA | 1183 | UUUCACGC A UGUGUGCU | 3962 |
| 3105 | GGAGCUGC CUGAUGAG GCCGUUAGGC CGAA ICACACAU | 1184 | AUGUGUGC U GCAGCUCC | 3963 |
| 3108 | AUGGGAGC CUGAUGAG GCCGUUAGGC CGAA ICAGCACA | 1185 | UGUGCUGC A GCUCCCAU | 3964 |
| 3111 | GAAAUGGG CUGAUGAG GCCGUUAGGC CGAA ICUGCAGC | 1186 | GCUGCAGC U CCCAUUUC | 3965 |
| 3113 | AUGAAAUG CUGAUGAG GCCGUUAGGC CGAA IAGCUGCA | 1187 | UGCAGCUC C CAUUUCAU | 3966 |
| 3114 | GAUGAAAU CUGAUGAG GCCGUUAGGC CGAA IGAGCUGC | 1188 | GCAGCUCC C AUUUCAUC | 3967 |
| 3115 | UGAUGAAA CUGAUGAG GCCGUUAGGC CGAA IGGAGCUG | 1189 | CAGCUCCC A UUUCAUCA | 3968 |
| 3120 | CUUGCUGA CUGAUGAG GCCGUUAGGC CGAA IAAAUGGG | 1190 | CCCAUUUC A UCAGCAAG | 3969 |
| 3123 | AAACUUGC CUGAUGAG GCCGUUAGGC CGAA IAUGAAAU | 1191 | AUUUCAUC A GCAAGUUU | 3970 |
| 3126 | UCCAAACU CUGAUGAG GCCGUUAGGC CGAA ICUGAUGA | 1192 | UCAUCAGC A AGUUUGGA | 3971 |
| 3140 | AAAUGUGG CUGAUGAG GCCGUUAGGC CGAA IUUCUUCC | 1193 | GGAAGAAC C CCACAUUU | 3972 |
| 3141 | AAAAUGUG CUGAUGAG GCCGUUAGGC CGAA IGUUCUUC | 1194 | GAAGAACC C CACAUUUU | 3973 |
| 3142 | AAAAAUGU CUGAUGAG GCCGUUAGGC CGAA IGGUUCUU | 1195 | AAGAACCC C ACAUUUUU | 3974 |
| 3143 | GAAAAAUG CUGAUGAG GCCGUUAGGC CGAA IGGGUUCU | 1196 | AGAACCCC A CAUUUUUC | 3975 |
| 3145 | AGGAAAAA CUGAUGAG GCCGUUAGGC CGAA IUGGGGUU | 1197 | AACCCCAC A UUUUUCCU | 3976 |
| 3152 | GACGCGCA CUGAUGAG GCCGUUAGGC CGAA IAAAAAUG | 1198 | CAUUUUUC C UGCGCGUC | 3977 |
| 3153 | UGACGCGC CUGAUGAG GCCGUUAGGC CGAA IGAAAAAU | 1199 | AUUUUUCC U GCGCGUCA | 3978 |
| 3161 | GUCAGAGA CUGAUGAG GCCGUUAGGC CGAA IACGCGCA | 1200 | UGCGCGUC A UCUCUGAC | 3979 |
| 3164 | CGUGUCAG CUGAUGAG GCCGUUAGGC CGAA IAUGACGC | 1201 | GCGUCAUC U CUGACACG | 3980 |
| 3166 | GCCGUGUC CUGAUGAG GCCGUUAGGC CGAA IAGAUGAC | 1202 | GUCAUCUC U GACACGGC | 3981 |
| 3170 | GGAGGCCG CUGAUGAG GCCGUUAGGC CGAA IUCAGAGA | 1203 | UCUCUGAC A CGGCCUCC | 3982 |
| 3175 | CAGAGGGA CUGAUGAG GCCGUUAGGC CGAA ICCGUGUC | 1204 | GACACGGC C UCCCUCUG | 3983 |
| 3176 | GCAGAGGG CUGAUGAG GCCGUUAGGC CGAA IGCCGUGU | 1205 | ACACGGCC U CCCUCUGC | 3984 |
| 3178 | UAGCAGAG CUGAUGAG GCCGUUAGGC CGAA IAGGCCGU | 1206 | ACGGCCUC C CUCUGCUA | 3985 |
| 3179 | GUAGCAGA CUGAUGAG GCCGUUAGGC CGAA IGAGGCCG | 1207 | CGGCCUCC C UCUGCUAC | 3986 |
| 3180 | AGUAGCAG CUGAUGAG GCCGUUAGGC CGAA IGGAGGCC | 1208 | GGCCUCCC U CUGCUACU | 3987 |
| 3182 | GGAGUAGC CUGAUGAG GCCGUUAGGC CGAA IAGGGAGG | 1209 | CCUCCCUC U GCUACUCC | 3988 |
| 3185 | GAUGGAGU CUGAUGAG GCCGUUAGGC CGAA ICAGAGGG | 1210 | CCCUCUGC U ACUCCAUC | 3989 |
| 3188 | CAGGAUGG CUGAUGAG GCCGUUAGGC CGAA IUAGCAGA | 1211 | UCUGCUAC U CCAUCCUG | 3990 |
| 3190 | UUCAGGAU CUGAUGAG GCCGUUAGGC CGAA IAGUAGCA | 1212 | UGCUACUC C AUCCUGAA | 3991 |
| 3191 | UUUCAGGA CUGAUGAG GCCGUUAGGC CGAA IGAGUAGC | 1213 | GCUACUCC A UCCUGAAA | 3992 |
| 3194 | GGCUUUCA CUGAUGAG GCCGUUAGGC CGAA IAUGGAGU | 1214 | ACUCCAUC C UGAAAGCC | 3993 |
| 3195 | UGGCUUUC CUGAUGAG GCCGUUAGGC CGAA IGAUGGAG | 1215 | CUCCAUCC U GAAAGCCA | 3994 |
| 3202 | GCGUUCUU CUGAUGAG GCCGUUAGGC CGAA ICUUUCAG | 1216 | CUGAAAGC C AAGAACGC | 3995 |
| 3203 | UGCGUUCU CUGAUGAG GCCGUUAGGC CGAA IGCUUUCA | 1217 | UGAAAGCC A AGAACGCA | 3996 |
| 3211 | GACAUCCC CUGAUGAG GCCGUUAGGC CGAA ICGUUCUU | 1218 | AAGAACGC A GGGAUGUC | 3997 |
| 3222 | UGGCCCCC CUGAUGAG GCCGUUAGGC CGAA ICGACAUC | 1219 | GAUGUCGC U GGGGGCCA | 3998 |
| 3229 | GCGCCCUU CUGAUGAG GCCGUUAGGC CGAA ICCCCCAG | 1220 | CUGGGGGC C AAGGGCGC | 3999 |
| 3230 | GGCGCCCU CUGAUGAG GCCGUUAGGC CGAA IGCCCCCA | 1221 | UGGGGGCC A AGGGCGCC | 4000 |
| 3238 | GGGCCGGC CUGAUGAG GCCGUUAGGC CGAA ICGCCCUU | 1222 | AAGGGCGC C GCCGGCCC | 4001 |
| 3241 | AGAGGGCC CUGAUGAG GCCGUUAGGC CGAA ICGGCGCC | 1223 | GGCGCCGC C GGCCCUCU | 4002 |
| 3245 | GGGCAGAG CUGAUGAG GCCGUUAGGC CGAA ICCGGCGG | 1224 | CCGCCGGC C CUCUGCCC | 4003 |
| 3246 | AGGGCAGA CUGAUGAG GCCGUUAGGC CGAA IGCCGGCG | 1225 | CGCCGGCC C UCUGCCCU | 4004 |
| 3247 | GAGGGCAG CUGAUGAG GCCGUUAGGC CGAA IGGCCGGC | 1226 | GCCGGCCC U CUGCCCUC | 4005 |
| 3249 | CGGAGGGC CUGAUGAG GCCGUUAGGC CGAA IAGGGCCG | 1227 | CGGCCCUC U GCCCUCCG | 4006 |
| 3252 | CCUCGGAG CUGAUGAG GCCGUUAGGC CGAA ICAGAGGG | 1228 | CCCUCUGC C CUCCGAGG | 4007 |
| 3253 | GCCUCGGA CUGAUGAG GCCGUUAGGC CGAA IGCAGAGG | 1229 | CCUCUGCC C UCCGAGGC | 4008 |
| 3254 | GGCCUCGG CUGAUGAG GCCGUUAGGC CGAA IGGCAGAG | 1230 | CUCUGCCC U CCGAGGCC | 4009 |
| 3256 | ACGGCCUC CUGAUGAG GCCGUUAGGC CGAA IAGGGCAG | 1231 | CUGCCCUC C GAGGCCGU | 4010 |
| 3262 | CACUGCAC CUGAUGAG GCCGUUAGGC CGAA ICCUCGGA | 1232 | UCCGAGGC C GUGCAGUG | 4011 |
| 3267 | ACAGCCAC CUGAUGAG GCCGUUAGGC CGAA ICACGGCC | 1233 | GGCCGUGC A GUGGCUGU | 4012 |
| 3273 | GGUGGCAC CUGAUGAG GCCGUUAGGC CGAA ICCACUGC | 1234 | GCAGUGGC U GUGCCACC | 4013 |
| 3278 | UGCUUGGU CUGAUGAG GCCGUUAGGC CGAA ICACAGCC | 1235 | GGCUGUGC C ACCAAGCA | 4014 |
| 3279 | AUGCUUGG CUGAUGAG GCCGUUAGGC CGAA IGCACAGC | 1236 | GCUGUGCC A CCAAGCAU | 4015 |
| 3281 | GAAUGCUU CUGAUGAG GCCGUUAGGC CGAA IUGGCACA | 1237 | UGUGCCAC C AAGCAUUC | 4016 |
| 3282 | GGAAUGCU CUGAUGAG GCCGUUAGGC CGAA IGUGGCAC | 1238 | GUGCCACC A AGCAUUCC | 4017 |
| 3286 | AGCAGGAA CUGAUGAG GCCGUUAGGC CGAA ICUUGGUG | 1239 | CACCAAGC A UUCCUGCU | 4018 |
| 3290 | CUUGAGCA CUGAUGAG GCCGUUAGGC CGAA IAAUGCUU | 1240 | AAGCAUUC C UGCUCAAG | 4019 |
| 3291 | GCUUGAGC CUGAUGAG GCCGUUAGGC CGAA IGAAUGCU | 1241 | AGCAUUCC U GCUCAAGC | 4020 |
| 3294 | UCAGCUUG CUGAUGAG GCCGUUAGGC CGAA ICAGGAAU | 1242 | AUUCCUGC U CAAGCUGA | 4021 |
| 3296 | AGUCAGCU CUGAUGAG GCCGUUAGGC CGAA IAGCAGGA | 1243 | UCCUGCUC A AGCUGACU | 4022 |
| 3300 | GUCGAGUC CUGAUGAG GCCGUUAGGC CGAA ICUUGAGC | 1244 | GCUCAAGC U GACUCGAC | 4023 |
| 3304 | CGGUGUCG CUGAUGAG GCCGUUAGGC CGAA IUCAGCUU | 1245 | AAGCUGAC U CGACACCG | 4024 |
| 3309 | UGACACGG CUGAUGAG GCCGUUAGGC CGAA IUCGAGUC | 1246 | GACUCGAC A CCGUGUCA | 4025 |
| 3311 | GGUGACAC CUGAUGAG GCCGUUAGGC CGAA IUGUCGAG | 1247 | CUCGACAC C GUGUCACC | 4026 |
| 3317 | CACGUAGG CUGAUGAG GCCGUUAGGC CGAA IACACGGU | 1248 | ACCGUGUC A CCUACGUG | 4027 |
| 3319 | GGCACGUA CUGAUGAG GCCGUUAGGC CGAA IUGACACG | 1249 | CGUGUCAC C UACGUGCC | 4028 |
| 3320 | UGGCACGU CUGAUGAG GCCGUUAGGC CGAA IGUGACAC | 1250 | GUGUCACC U ACGUGCCA | 4029 |
| 3327 | CCAGGAGU CUGAUGAG GCCGUUAGGC CGAA ICACGUAG | 1251 | CUACGUGC C ACUCCUGG | 4030 |
| 3328 | CCCAGGAG CUGAUGAG GCCGUUAGGC CGAA IGCACGUA | 1252 | UACGUGCC A CUCCUGGG | 4031 |
| 3330 | ACCCCAGG CUGAUGAG GCCGUUAGGC CGAA IUGGCACG | 1253 | CGUGCCAC U CCUGGGGU | 4032 |
| 3332 | UGACCCCA CUGAUGAG GCCGUUAGGC CGAA IAGUGGCA | 1254 | UGCCACUC C UGGGGUCA | 4033 |
| 3333 | GUGACCCC CUGAUGAG GCCGUUAGGC CGAA IGAGUGGC | 1255 | GCCACUCC U GGGGUCAC | 4034 |
| 3340 | GUCCUGAG CUGAUGAG GCCGUUAGGC CGAA IACCCCAG | 1256 | CUGGGGUC A CUCAGGAC | 4035 |
| 3342 | CUGUCCUG CUGAUGAG GCCGUUAGGC CGAA IUGACCCC | 1257 | GGGGUCAC U CAGGACAG | 4036 |
| 3344 | GGCUGUCC CUGAUGAG GCCGUUAGGC CGAA IAGUGACC | 1258 | GGUCACUC A GGACAGCC | 4037 |
| 3349 | GUCUGGGC CUGAUGAG GCCGUUAGGC CGAA IUCCUGAG | 1259 | CUCAGGAC A GCCCAGAC | 4038 |
| 3352 | UGCGUCUG CUGAUGAG GCCGUUAGGC CGAA ICUGUCCU | 1260 | AGGACAGC C CAGACGCA | 4039 |
| 3353 | CUGCGUCU CUGAUGAG GCCGUUAGGC CGAA IGCUGUCC | 1261 | GGACAGCC C AGACGCAG | 4040 |
| 3354 | GCUGCGUC CUGAUGAG GCCGUUAGGC CGAA IGGCUGUC | 1262 | GACAGCCC A GACGCAGC | 4041 |
| 3360 | GACUCAGC CUGAUGAG GCCGUUAGGC CGAA ICGUCUGG | 1263 | CCAGACGC A GCUGAGUC | 4042 |
| 3363 | UCCGACUC CUGAUGAG GCCGUUAGGC CGAA ICUGCGUC | 1264 | GACGCAGC U GAGUCGGA | 4043 |
| 3375 | UCCCCGGG CUGAUGAG GCCGUUAGGC CGAA ICUUCCGA | 1265 | UCGGAAGC U CCCGGGGA | 4044 |
| 3377 | CGUCCCCG CUGAUGAG GCCGUUAGGC CGAA IAGCUUCC | 1266 | GGAAGCUC C CGGGGACG | 4045 |
| 3378 | UCGUCCCC CUGAUGAG GCCGUUAGGC CGAA IGAGCUUC | 1267 | GAAGCUCC C GGGGACGA | 4046 |
| 3390 | GGGCAGUC CUGAUGAG GCCGUUAGGC CGAA ICGUCGUC | 1268 | GACGACGC U GACUGCCC | 4047 |
| 3394 | UCCAGGGC CUGAUGAG GCCGUUAGGC CGAA IUCAGCGU | 1269 | ACGCUGAC U GCCCUGGA | 4048 |
| 3397 | GCCUCCAG CUGAUGAG GCCGUUAGGC CGAA ICAGUCAG | 1270 | CUGACUGC C CUGGAGGC | 4049 |
| 3398 | GOCCUCCA CUGAUGAG GCCGUUAGGC CGAA IGCAGUCA | 1271 | UGACUGCC C UGGAGGCC | 4050 |
| 3399 | CGGCCUCC CUGAUGAG GCCGUUAGGC CGAA IGGCAGUC | 1272 | GACUGCCC U GGAGGCCG | 4051 |
| 3406 | UUGGCUGC CUGAUGAG GCCGUUAGGC CGAA ICCUCCAG | 1273 | CUGGAGGC C GCAGCCAA | 4052 |
| 3409 | GGGUUGGC CUGAUGAG GCCGUUAGGC CGAA ICGGCCUC | 1274 | GAGGCCGC A GCCAACCC | 4053 |
| 3412 | GCCGGGUU CUGAUGAG GCCGUUAGGC CGAA ICUGCGGC | 1275 | GCCGCAGC C AACCCGGC | 4054 |
| 3413 | UGCCGGGU CUGAUGAG GCCGUUAGGC CGAA IGCUGCGG | 1276 | CCGCAGCC A ACCCGGCA | 4055 |
| 3416 | CAGUGCCG CUGAUGAG GCCGUUAGGC CGAA IUUGGCUG | 1277 | CAGCCAAC C CGGCACUG | 4056 |
| 3417 | GCAGUGCC CUGAUGAG GCCGUUAGGC CGAA IGUUGGCU | 1278 | AGCCAACC C GGCACUGC | 4057 |
| 3421 | GAGGGCAG CUGAUGAG GCCGUUAGGC CGAA ICCGGGUU | 1279 | AACCCGGC A CUGCCCUC | 4058 |
| 3423 | CUGAGGGC CUGAUGAG GCCGUUAGGC CGAA IUGCCGGG | 1280 | CCCGGCAC U GCCCUCAG | 4059 |
| 3426 | AGUCUGAG CUGAUGAG GCCGUUAGGC CGAA ICAGUGCC | 1281 | GGCACUGC C CUCAGACU | 4060 |
| 3427 | AAGUCUGA CUGAUGAG GCCGUUAGGC CGAA IGCAGUGC | 1282 | GCACUGCC C UCAGACUU | 4061 |
| 3428 | GAAGUCUG CUGAUGAG GCCGUUAGGC CGAA IGGCAGUG | 1283 | CACUGCCC U CAGACUUC | 4062 |
| 3430 | UUGAAGUC CUGAUGAG GCCGUUAGGC CGAA IAGGGCAG | 1284 | CUGCCCUC A GACUUCAA | 4063 |
| 3434 | GGUCUUGA CUGAUGAG GCCGUUAGGC CGAA IUCUGAGG | 1285 | CCUCAGAC U UCAAGACC | 4064 |
| 3437 | GAUGGUCU CUGAUGAG GCCGUUAGGC CGAA IAAGUCUG | 1286 | CAGACUUC A AGACCAUC | 4065 |
| 3442 | UCCAGGAU CUGAUGAG GCCGUUAGGC CGAA IUCUUGAA | 1287 | UUCAAGAC C AUCCUGGA | 4066 |
| 3443 | GUCCAGGA CUGAUGAG GCCGUUAGGC CGAA IGUCUUGA | 1288 | UCAAGACC A UCCUGGAC | 4067 |
| 3446 | UCAGUCCA CUGAUGAG GCCGUUAGGC CGAA IAUGGUCU | 1289 | AGACCAUC C UGGACUGA | 4068 |
| 3447 | AUCAGUCC CUGAUGAG GCCGUUAGGC CGAA IGAUGGUC | 1290 | GACCAUCC U GGACUGAU | 4069 |
| 3452 | UGGCCAUC CUGAUGAG GCCGUUAGGC CGAA TUCCAGGA | 1291 | UCCUGGAC U GAUGGCCA | 4070 |
| 3459 | GGGCGGGU CUGAUGAG GCCGUUAGGC CGAA ICCAUCAG | 1292 | CUGAUGGC C ACCCGCCC | 4071 |
| 3460 | UGGGCGGG CUGAUGAG GCCGUUAGGC CGAA IGCCAUCA | 1293 | UGAUGGCC A CCCGCCCA | 4072 |
| 3462 | UGUGGGCG CUGAUGAG GCCGUUAGGC CGAA IUGGCCAU | 1294 | AUGGCCAC C CGCCCACA | 4073 |
| 3463 | CUGUGGGC CUGAUGAG GCCGUUAGGC CGAA IGUGGCCA | 1295 | UGGCCACC C GCCCACAG | 4074 |
| 3466 | UGGCUGUG CUGAUGAG GCCGUUAGGC CGAA ICGGGUGG | 1296 | CCACCCGC C CACAGCCA | 4075 |
| 3467 | CUGGCUGU CUGAUGAG GCCGUUAGGC CGAA IGCGGGUG | 1297 | CACCCGCC C ACAGCCAG | 4076 |
| 3468 | CCUGGCUG CUGAUGAG GCCGUUAGGC CGAA IGGCGGGU | 1298 | ACCCGCCC A CAGCCAGG | 4077 |
| 3470 | GGCCUGGC CUGAUGAG GCCGUUAGGC CGAA IUGGGCGG | 1299 | CCGCCCAC A GCCAGGCC | 4078 |
| 3473 | CUCGGCCU CUGAUGAG GCCGUUAGGC CGAA ICUGUGGG | 1300 | CCCACAGC C AGGCCGAG | 4079 |
| 3474 | UCUCGGCC CUGAUGAG GCCGUUAGGC CGAA IGCUGUGG | 1301 | CCACAGCC A GGCCGAGA | 4080 |
| 3478 | CUGCUCUC CUGAUGAG GCCGUUAGGC CGAA ICCUGGCU | 1302 | AGCCAGGC C GAGAGCAG | 4081 |
| 3485 | CUGGUGUC CUGAUGAG GCCGUUAGGC CGAA ICUCUCGG | 1303 | CCGAGAGC A GACACCAG | 4082 |
| 3489 | GCUGCUGG CUGAUGAG GCCGUUAGGC CGAA IUCUGCUC | 1304 | GAGCAGAC A CCAGCAGC | 4083 |
| 3491 | GGGCUGCU CUGAUGAG GCCGUUAGGC CGAA IUGUCUGC | 1305 | GCAGACAC C AGCAGCCC | 4084 |
| 3492 | AGGGCUGC CUGAUGAG GCCGUUAGGC CGAA IGUGUCUG | 1306 | CAGACACC A GCAGCCCU | 4085 |
| 3495 | GACAGGGC CUGAUGAG GCCGUUAGGC CGAA ICUGGUGU | 1307 | ACACCAGC A GCCCUGUC | 4086 |
| 3498 | CGUGACAG CUGAUGAG GCCGUUAGGC CGAA ICUGCUGG | 1308 | CCAGCAGC C CUGUCACG | 4087 |
| 3499 | GCGUGACA CUGAUGAG GCCGUUAGGC CGAA IGCUGCUG | 1309 | CAGCAGCC C UGUCACGC | 4088 |
| 3500 | GGCGUGAC CUGAUGAG GCCGUUAGGC CGAA IGGCUGCU | 1310 | AGCAGCCC U GUCACGCC | 4089 |
| 3504 | GCCCGGCG CUGAUGAG GCCGUUAGGC CGAA IACAGGGC | 1311 | GCCCUGUC A CGCCGGGC | 4090 |
| 3508 | UAGAGCCC CUGAUGAG GCCGUUAGGC CGAA ICGUGACA | 1312 | UGUCACGC C GGGCUCUA | 4091 |
| 3513 | GGACGUAG CUGAUGAG GCCGUUAGGC CGAA ICCCGGCG | 1313 | CGCCGGGC U CUACGUCC | 4092 |
| 3515 | UGGGACGU CUGAUGAG GCCGUUAGGC CGAA IAGCCCGG | 1314 | CCGGGCUC U ACGUCCCA | 4093 |
| 3521 | CCUCCCUG CUGAUGAG GCCGUUAGGC CGAA IACGUAGA | 1315 | UCUACGUC C CAGGGAGG | 4094 |
| 3522 | CCCUCCCU CUGAUGAG GCCGUUAGGC CGAA IGACGUAG | 1316 | CUACGUCC C AGGGAGGG | 4095 |
| 3523 | UCCCUCCC CUGAUGAG GCCGUUAGGC CGAA IGGACGUA | 1317 | UACGUCCC A GGGAGGGA | 4096 |
| 3540 | UGGGUGUG CUGAUGAG GCCGUUAGGC CGAA ICCGCCCC | 1318 | GGGGCGGC C CACACCCA | 4097 |
| 3541 | CUGGGUGU CUGAUGAG GCCGUUAGGC CGAA IGCCGCCC | 1319 | GGGCGGCC C ACACCCAG | 4098 |
| 3542 | CCUGGGUG CUGAUGAG GCCGUUAGGC CGAA IGGCCGCC | 1320 | GGCGGCCC A CACCCAGG | 4099 |
| 3544 | GGCCUGGG CUGAUGAG GCCGUUAGGC CGAA IUGGGCCG | 1321 | CGGCCCAC A CCCAGGCC | 4100 |
| 3546 | CGGGCCUG CUGAUGAG GCCGUUAGGC CGAA IUGUGGGC | 1322 | GCCCACAC C CAGGCCCG | 4101 |
| 3547 | GCGGGCCU CUGAUGAG GCCGUUAGGC CGAA IGUGUGGG | 1323 | CCCACACC C AGGCCCGC | 4102 |
| 3548 | UGCGGGCC CUGAUGAG GCCGUUAGGC CGAA IGGUGUGG | 1324 | CCACACCC A GGCCCGCA | 4103 |
| 3552 | GCGGUGCG CUGAUGAG GCCGUUAGGC CGAA ICCUGGGU | 1325 | ACCCAGGC C CGCACCGC | 4104 |
| 3553 | AGCGGUGC CUGAUGAG GCCGUUAGGC CGAA IGCCUGGG | 1326 | CCCAGGCC C GCACCGCU | 4105 |
| 3556 | CCCAGCGG CUGAUGAG GCCGUUAGGC CGAA ICGGGCCU | 1327 | AGGCCCGC A CCGCUGGG | 4106 |
| 3558 | CUCCCAGC CUGAUGAG GCCGUUAGGC CGAA IUGCGGGC | 1328 | GCCCGCAC C GCUGGGAG | 4107 |
| 3561 | AGACUCCC CUGAUGAG GCCGUUAGGC CGAA ICGGUGCG | 1329 | CGCACCGC U GGGAGUCU | 4108 |
| 3569 | CAGGCCUC CUGAUGAG GCCGUUAGGC CGAA IACUCCCA | 1330 | UGGGAGUC U GAGGCCUG | 4109 |
| 3575 | CUCACUCA CUGAUGAG GCCGUUAGGC CGAA ICCUCAGA | 1331 | UCUGAGGC C UGAGUGAG | 4110 |
| 3576 | ACUCACUC CUGAUGAG GCCGUUAGGC CGAA IGCCUCAG | 1332 | CUGAGGCC U GAGUGAGU | 4111 |
| 3592 | CAGOCCUC CUGAUGAG GCCGUUAGGC CGAA ICCAAACA | 1333 | UGUUUGGC C GAGGCCUG | 4112 |
| 3598 | GACAUGCA CUGAUGAG GCCGUUAGGC CGAA ICCUCGGC | 1334 | GCCGAGGC C UGCAUGUC | 4113 |
| 3599 | GGACAUGC CUGAUGAG GCCGUUAGGC CGAA IGCCUCGG | 1335 | CCGAGGCC U GCAUGUCC | 4114 |
| 3602 | GCCGGACA CUGAUGAG GCCGUUAGGC CGAA ICAGGCCU | 1336 | AGGCCUGC A UGUCCGGC | 4115 |
| 3607 | CUUCAGCC CUGAUGAG GCCGUUAGGC CGAA IACAUGCA | 1337 | UGCAUGUC C GGCUGAAG | 4116 |
| 3611 | CAGCCUUC CUGAUGAG GCCGUUAGGC CGAA ICCGGACA | 1338 | UGUCCGGC U GAAGGCUG | 4117 |
| 3618 | GGACACUC CUGAUGAG GCCGUUAGGC CGAA ICCUUCAG | 1339 | CUGAAGGC U GAGUGUCC | 4118 |
| 3626 | CCUCAGCC CUGAUGAG GCCGUUAGGC CGAA IACACUCA | 1340 | UGAGUGUC C GGCUGAGG | 4119 |
| 3630 | CAGGCCUC CUGAUGAG GCCGUUAGGC CGAA ICCGGACA | 1341 | UGUCCGGC U GAGGCCUG | 4120 |
| 3636 | CUCGCUCA CUGAUGAG GCCGUUAGGC CGAA ICCUCAGC | 1342 | GCUGAGGC C UGAGCGAG | 4121 |
| 3637 | ACUCGCUC CUGAUGAG GCCGUUAGGC CGAA IGCCUCAG | 1343 | CUGAGGCC U GAGCGAGU | 4122 |
| 3649 | CCUUGGCU CUGAUGAG GCCGUUAGGC CGAA IACACUCG | 1344 | CGAGUGUC C AGCCAAGG | 4123 |
| 3650 | CCCUUGGC CUGAUGAG GCCGUUAGGC CGAA IGACACUC | 1345 | GAGUGUCC A GCCAAGGG | 4124 |
| 3653 | CAGCCCUU CUGAUGAG GCCGUUAGGC CGAA ICUGGACA | 1346 | UGUCCAGC C AAGGGCUG | 4125 |
| 3654 | UCAGCCCU CUGAUGAG GCCGUUAGGC CGAA IGCUGGAC | 1347 | GUCCAGCC A AGGGCUGA | 4126 |
| 3660 | GGACACUC CUGAUGAG GCCGUUAGGC CGAA ICCCUUGG | 1348 | CCAAGGGC U GAGUGUCC | 4127 |
| 3668 | GGUGUGCU CUGAUGAG GCCGUUAGGC CGAA IACACUCA | 1349 | UGAGUGUC C AGCACACC | 4128 |
| 3669 | AGGUGUGC CUGAUGAG GCCGUUAGGC CGAA IGACACUC | 1350 | GAGUGUCC A GCACACCU | 4129 |
| 3672 | GGCAGGUG CUGAUGAG GCCGUUAGGC CGAA ICUGGACA | 1351 | UGUCCAGC A CACCUGCC | 4130 |
| 3674 | ACGGCAGG CUGAUGAG GCCGUUAGGC CGAA IUGCUGGA | 1352 | UCCAGCAC A CCUGCCGU | 4131 |
| 3676 | AGACGGCA CUGAUGAG GCCGUUAGGC CGAA IUGUGCUG | 1353 | CAGCACAC C UGCCGUCU | 4132 |
| 3677 | AAGACGGC CUGAUGAG GCCGUUAGGC CGAA IGUGUGCU | 1354 | AGCACACC U GCCGUCUU | 4133 |
| 3680 | GUGAAGAC CUGAUGAG GCCGUUAGGC CGAA ICAGGUGU | 1355 | ACACCUGC C GUCUUCAC | 4134 |
| 3684 | GGAAGUGA CUGAUGAG GCCGUUAGGC CGAA IACGGCAG | 1356 | CUGCCGUC U UCACUUCC | 4135 |
| 3687 | UGGGGAAG CUGAUGAG GCCGUUAGGC CGAA IAAGACGG | 1357 | CCGUCUUC A CUUCCCCA | 4136 |
| 3689 | UGUGGGGA CUGAUGAG GCCGUUAGGC CGAA IUGAAGAC | 1358 | GUCUUCAC U UCCCCACA | 4137 |
| 3692 | GCCUGUGG CUGAUGAG GCCGUUAGGC CGAA IAAGUGAA | 1359 | UUCACUUC C CCACAGGC | 4138 |
| 3693 | AGCCUGUG CUGAUGAG GCCGUUAGGC CGAA IGAAGUGA | 1360 | UCACUUCC C CACAGGCU | 4139 |
| 3694 | CAGCCUGU CUGAUGAG GCCGUUAGGC CGAA IGGAAGUG | 1361 | CACUUCCC C ACAGGCUG | 4140 |
| 3695 | CCAGCCUG CUGAUGAG GCCGUUAGGC CGAA IGGGAAGU | 1362 | ACUUCCCC A CAGGCUGG | 4141 |
| 3697 | CGCCAGCC CUGAUGAG GCCGUUAGGC CGAA IUGGGGAA | 1363 | UUCCCCAC A GGCUGGCG | 4142 |
| 3701 | CGAGCGCC CUGAUGAG GCCGUUAGGC CGAA ICCUGUGG | 1364 | CCACAGGC U GGCGCUCG | 4143 |
| 3707 | UGGAGCCG CUGAUGAG GCCGUUAGGC CGAA ICGCCAGC | 1365 | GCUGGCGC U CGGCUCCA | 4144 |
| 3712 | UGGGGUGG CUGAUGAG GCCGUUAGGC CGAA ICCGAGCG | 1366 | CGCUCGGC U CCACCCCA | 4145 |
| 3714 | CCUGGGGU CUGAUGAG GCCGUUAGGC CGAA IAGCCGAG | 1367 | CUCGGCUC C ACCCCAGG | 4146 |
| 3715 | CCCUGGGG CUGAUGAG GCCGUUAGGC CGAA IGAGCCGA | 1368 | UCGGCUCC A CCCCAGGG | 4147 |
| 3717 | GGCCCUGG CUGAUGAG GCCGUUAGGC CGAA IUGGAGCC | 1369 | GGCUCCAC C CCAGGGCC | 4148 |
| 3718 | UGGCCCUG CUGAUGAG GCCGUUAGGC CGAA IGUGGAGC | 1370 | GCUCCACC C CAGGGCCA | 4149 |
| 3719 | CUGGCCCU CUGAUGAG GCCGUUAGGC CGAA IGGUGGAG | 1371 | CUCCACCC C AGGGCCAG | 4150 |
| 3720 | GCUGGCCC CUGAUGAG GCCGUUAGGC CGAA IGGGUGGA | 1372 | UCCACCCC A GGGCCAGC | 4151 |
| 3725 | GAAAAGCU CUGAUGAG GCCGUUAGGC CGAA ICCCUGGG | 1373 | CCCAGGGC C AGCUUUUC | 4152 |
| 3726 | GGAAAAGC CUGAUGAG GCCGUUAGGC CGAA IGCCCUGG | 1374 | CCAGGGCC A GCUUUUCC | 4153 |
| 3729 | UGAGGAAA CUGAUGAG GCCGUUAGGC CGAA ICUGGCCC | 1375 | GGGCCAGC U UUUCCUCA | 4154 |
| 3734 | CCUGGUGA CUGAUGAG GCCGUUAGGC CGAA IAAAAGCU | 1376 | AGCUUUUC C UCACCAGG | 4155 |
| 3735 | UCCUGGUG CUGAUGAG GCCGUUAGGC CGAA IGAAAAGC | 1377 | GCUUUUCC U CACCAGGA | 4156 |
| 3737 | GCUCCUGG CUGAUGAG GCCGUUAGGC CGAA IAGGAAAA | 1378 | UUUUCCUC A CCAGGAGC | 4157 |
| 3739 | GGGCUCCU CUGAUGAG GCCGUUAGGC CGAA IUGAGGAA | 1379 | UUCCUCAC C AGGAGCCC | 4158 |
| 3740 | CGGGCUCC CUGAUGAG GCCGUUAGGC CGAA IGUGAGGA | 1380 | UCCUCACC A GGAGCCCG | 4159 |
| 3746 | GGAAGCCG CUGAUGAG GCCGUUAGGC CGAA ICUCCUGG | 1381 | CCAGGAGC C CGGCUUCC | 4160 |
| 3747 | UGGAAGCC CUGAUGAG GCCGUUAGGC CGAA IGCUCCUG | 1382 | CAGGAGCC C GGCUUCCA | 4161 |
| 3751 | GGAGUGGA CUGAUGAG GCCGUUAGGC CGAA ICCGGGCU | 1383 | AGCCCGGC U UCCACUCC | 4162 |
| 3754 | UGGGGAGU CUGAUGAG GCCGUUAGGC CGAA IAAGCCGG | 1384 | CCGGCUUC C ACUCCCCA | 4163 |
| 3755 | GUGGGGAG CUGAUGAG GCCGUUAGGC CGAA IGAAGCCG | 1385 | CGGCUUCC A CUCCCCAC | 4164 |
| 3757 | AUGUGGGG CUGAUGAG GCCGUUAGGC CGAA IUGGAAGC | 1386 | GCUUCCAC U CCCCACAU | 4165 |
| 3759 | CUAUGUGG CUGAUGAG GCCGUUAGGC CGAA IAGUGGAA | 1387 | UUCCACUC C CCACAUAG | 4166 |
| 3760 | CCUAUGUG CUGAUGAG GCCGUUAGGC CGAA IGAGUGGA | 1388 | UCCACUCC C CACAUAGG | 4167 |
| 3761 | UCCUAUGU CUGAUGAG GCCGUUAGGC CGAA IGGAGUGG | 1389 | CCACUCCC C ACAUAGGA | 4168 |
| 3762 | UUCCUAUG CUGAUGAG GCCGUUAGGC CGAA IGGGAGUG | 1390 | CACUCCCC A CAUAGGAA | 4169 |
| 3764 | UAUUCCUA CUGAUGAG GCCGUUAGGC CGAA IUGGGGAG | 1391 | CUCCCCAC A UAGGAAUA | 4170 |
| 3776 | CUGGGGAU CUGAUGAG GCCGUUAGGC CGAA IACUAUUC | 1392 | GAAUAGUC C AUCCCCAG | 4171 |
| 3777 | UCUGGGGA CUGAUGAG GCCGUUAGGC CGAA IGACUAUU | 1393 | AAUAGUCC A UCCCCAGA | 4172 |
| 3780 | GAAUCUGG CUGAUGAG GCCGUUAGGC CGAA IAUGGACU | 1394 | AGUCCAUC C CCAGAUUC | 4173 |
| 3781 | CGAAUCUG CUGAUGAG GCCGUUAGGC CGAA IGAUGGAC | 1395 | GUCCAUCC C CAGAUUCG | 4174 |
| 3782 | GCGAAUCU CUGAUGAG GCCGUUAGGC CGAA IGGAUGGA | 1396 | UCCAUCCC C AGAUUCGC | 4175 |
| 3783 | GGCGAAUC CUGAUGAG GCCGUUAGGC CGAA IGGGAUGG | 1397 | CCAUCCCC A GAUUCGCC | 4176 |
| 3791 | UGAACAAU CUGAUGAG GCCGUUAGGC CGAA ICGAAUCU | 1398 | AGAUUCGC C AUUGUUCA | 4177 |
| 3792 | GUGAACAA CUGAUGAG GCCGUUAGGC CGAA IGCGAAUC | 1399 | GAUUCGCC A UUGUUCAC | 4178 |
| 3799 | GCGAGGGG CUGAUGAG GCCGUUAGGC CGAA IAACAAUG | 14P0 | CAUUGUUC A CCCCUCGC | 4179 |
| 3801 | GGGCGAGG CUGAUGAG GCCGUUAGGC CGAA IUGAACAA | 1401 | UUGUUCAC C CCUCGCCC | 4180 |
| 3802 | AGGGCGAG CUGAUGAG GCCGUUAGGC CGAA IGUGAACA | 1402 | UGUUCACC C CUCGCCCU | 4181 |
| 3803 | CAGGGCGA CUGAUGAG GCCGUUAGGC CGAA IGGUGAAC | 1403 | GUUCACCC C UCGCCCUG | 4182 |
| 3804 | GCAGGGCG CUGAUGAG GCCGUUAGGC CGAA IGGGUGAA | 1404 | UUCACCCC U CGCCCUGC | 4183 |
| 3808 | GAGGGCAG CUGAUGAG GCCGUUAGGC CGAA ICGAGGGG | 1405 | CCCCUCGC C CUGCCCUC | 4184 |
| 3809 | GGAGGGCA CUGAUGAG GCCGUUAGGC CGAA IGCGAGGG | 1406 | CCCUCGCC C UGCCCUCC | 4185 |
| 3810 | AGGAGGGC CUGAUGAG GCCGUUAGGC CGAA IGGCGAGG | 1407 | CCUCGCCC U GCCCUCCU | 4186 |
| 3813 | CAAAGGAG CUGAUGAG GCCGUUAGGC CGAA ICAGGGCG | 1408 | CGCCCUGC C CUCCUUUG | 4187 |
| 3814 | GCAAAGGA CUGAUGAG GCCGUUAGGC CGAA IGCAGGGC | 1409 | GCCCUGCC C UCCUUUGC | 4188 |
| 3815 | GGCAAAGG CUGAUGAG GCCGUUAGGC CGAA IGGCAGGG | 1410 | CCCUGCCC U CCUUUGCC | 4189 |
| 3817 | AAGGCAAA CUGAUGAG GCCGUUAGGC CGAA IAGGGCAG | 1411 | CUGCCCUC C UUUGCCUU | 4190 |
| 3818 | GAAGGCAA CUGAUGAG GCCGUUAGGC CGAA IGAGGGCA | 1412 | UGCCCUCC U UUGCCUUC | 4191 |
| 3823 | GGGUGGAA CUGAUGAG GCCGUUAGGC CGAA ICAAAGGA | 1413 | UCCUUUGC C UUCCACCC | 4192 |
| 3824 | GGGGUGGA CUGAUGAG GCCGUUAGGC CGAA IGCAAAGG | 1414 | CCUUUGCC U UCCACCCC | 4193 |
| 3827 | GUGGGGGU CUGAUGAG GCCGUUAGGC CGAA IAAGGCAA | 1415 | UUGCCUUC C ACCCCCAC | 4194 |
| 3828 | GGUGGGGG CUGAUGAG GCCGUUAGGC CGAA IGAAGGCA | 1416 | UGCCUUCC A CCCCCACC | 4195 |
| 3830 | AUGGUGGG CUGAUGAG GCCGUUAGGC CGAA IUGGAAGG | 1417 | CCUUCCAC C CCCACCAU | 4196 |
| 3831 | GAUGGUGG CUGAUGAG GCCGUUAGGC CGAA IGUGGAAG | 1418 | CUUCCACC C CCACCAUC | 4197 |
| 3832 | GGAUGGUG CUGAUGAG GCCGUUAGGC CGAA IGGUGGAA | 1419 | UUCCACCC C CACCAUCC | 4198 |
| 3833 | UGGAUGGU CUGAUGAG GCCGUUAGGC CGAA IGGGUGGA | 1420 | UCCACCCC C ACCAUCCA | 4199 |
| 3834 | CUGGAUGG CUGAUGAG GCCGUUAGGC CGAA IGGGGUGG | 1421 | CCACCCCC A CCAUCCAG | 4200 |
| 3836 | ACCUGGAU CUGAUGAG GCCGUUAGGC CGAA IUGGGGGU | 1422 | ACCCCCAC C AUCCAGGU | 4201 |
| 3837 | CACCUGGA CUGAUGAG GCCGUUAGGC CGAA IGUGGGGG | 1423 | CCCCCACC A UCCAGGUG | 4202 |
| 3840 | CUCCACCU CUGAUGAG GCCGUUAGGC CGAA IAUGGUGG | 1424 | CCACCAUC C AGGUGGAG | 4203 |
| 3841 | UCUCCACC CUGAUGAG GCCGUUAGGC CGAA IGAUGGUG | 1425 | CACCAUCC A GGUGGAGA | 4204 |
| 3851 | CUUCUCAG CUGAUGAG GCCGUUAGGC CGAA IUCUCCAC | 1426 | GUGGAGAC C CUGAGAAG | 4205 |
| 3852 | CCUUCUCA CUGAUGAG GCCGUUAGGC CGAA IGUCUCCA | 1427 | UGGAGACC C UGAGAAGG | 4206 |
| 3853 | UCCUUCUC CUGAUGAG GCCGUUAGGC CGAA IGGUCUCC | 1428 | GGAGACCC U GAGAAGGA | 4207 |
| 3863 | GCUCCCAG CUGAUGAG GCCGUUAGGC CGAA IUCCUUCU | 1429 | AGAAGGAC C CUGGGAGC | 4208 |
| 3864 | AGCUCCCA CUGAUGAG GCCGUUAGGC CGAA IGUCCUUC | 1430 | GAAGGACC C UGGGAGCU | 4209 |
| 3865 | GAGCUCCC CUGAUGAG GCCGUUAGGC CGAA IGGUCCUU | 1431 | AAGGACCC U GGGAGCUC | 4210 |
| 3872 | AUUCCCAG CUGAUGAG GCCGUUAGGC CGAA ICUCCCAG | 1432 | CUGGGAGC U CUGGGAAU | 4211 |
| 3874 | AAAUUCCC CUGAUGAG GCCGUUAGGC CGAA IAGCUCCC | 1433 | GGGAGCUC U GGGAAAUU | 4212 |
| 3891 | ACACCUUU CUGAUGAG GCCGUUAGGC CGAA IUCACUCC | 1434 | GGAGUGAC C AAAGGUGU | 4213 |
| 3892 | CACACCUU CUGAUGAG GCCGUUAGGC CGAA IGUCACUC | 1435 | GAGUGACC A AAGGUGUG | 4214 |
| 3902 | GUGUACAG CUGAUGAG GCCGUUAGGC CGAA ICACACCU | 1436 | AGGUGUGC C CUGUACAC | 4215 |
| 3903 | UGUGUACA CUGAUGAG GCCGUUAGGC CGAA IGCACACC | 1437 | GGUGUGCC C UGUACACA | 4216 |
| 3904 | CUGUGUAC CUGAUGAG GCCGUUAGGC CGAA IGGCACAC | 1438 | GUGUGCCC U GUACACAG | 4217 |
| 3909 | CUCGCCUG CUGAUGAG GCCGUUAGGC CGAA IUACAGGG | 1439 | CCCUGUAC A CAGGCGAG | 4218 |
| 3911 | UCCUCGCC CUGAUGAG GCCGUUAGGC CGAA IUGUACAG | 1440 | CUGUACAC A GGCGAGGA | 4219 |
| 3921 | AGGUGCAG CUGAUGAG GCCGUUAGGC CGAA IUCCUCGC | 1441 | GCGAGGAC C CUGCACCU | 4220 |
| 3922 | CAGGUGCA CUGAUGAG GCCGUUAGGC CGAA IGUCCUCG | 1442 | CGAGGACC C UGCACCUG | 4221 |
| 3923 | CCAGGUGC CUGAUGAG GCCGUUAGGC CGAA IGGUCCUC | 1443 | GAGGACCC U GCACCUGG | 4222 |
| 3926 | CAUCCAGG CUGAUGAG GCCGUUAGGC CGAA ICAGGGUC | 1444 | GACCCUGC A CCUGGAUG | 4223 |
| 3928 | CCCAUCCA CUGAUGAG GCCGUUAGGC CGAA IUGCAGGG | 1445 | CCCUGCAC C UGGAUGGG | 4224 |
| 3929 | CCCCAUCC CUGAUGAG GCCGUUAGGC CGAA IGUGCAGG | 1446 | CCUGCACC U GGAUGGGG | 4225 |
| 3941 | ACCCACAG CUGAUGAG GCCGUUAGGC CGAA IACCCCCA | 1447 | UGGGGGUC C CUGUGGGU | 4226 |
| 3942 | GACCCACA CUGAUGAG GCCGUUAGGC CGAA IGACCCCC | 1448 | GGGGGUCC C UGUGGGUC | 4227 |
| 3943 | UGACCCAC CUGAUGAG GCCGUUAGGC CGAA IGGACCCC | 1449 | GGGGUCCC U GUGGGUCA | 4228 |
| 3951 | CCCCAAUU CUGAUGAG GCCGUUAGGC CGAA IACCCACA | 1450 | UGUGGGUC A AAUUGGGG | 4229 |
| 3968 | ACUCCCAC CUGAUGAG GCCGUUAGGC CGAA ICACCUCC | 1451 | GGAGGUGC U GUGGGAGU | 4230 |
| 3984 | AUAUAUUC CUGAUGAG GCCGUUAGGC CGAA IUAUUUUA | 1452 | UAAAAUAC U GAAUAUAU | 4231 |
| 4002 | UUCAAAAC CUGAUGAG GCCGUUAGGC CGAA IAAAAACU | 1453 | AGUUUUUC A GUUUUGAA | 4232 |

Stem Length = 8.
Core Sequence = CUGAUGAG GCCGUUAGGC CGAA,
I = Inosine nucleotide
Seq1 = TERT (Homo sapiens telomerase reverse transcriptase (TERT) mRNA, 4015 bp); Nakamura et al., Science 277 (5328), 955-959 (1997)

TABLE V

Human telomerase reverse transcriptase (TERT) G-Cleaver Ribozyme and
Target Sequence

| | | | |
|---|---|---|---|
| nt. | | Seq ID | |
| Position | Substrate Sequence | Nos | Ribozyme Sequence |

| | | | | |
|---|---|---|---|---|
| 16 | GCUGCGUCCU G CUGCG | 1454 | CGCAG UGAUGGCAUGCACUAUGCGCG AGGACGCAGC | 4233 |
| 19 | GCGUCCUGCU G CGCAC | 1455 | GUGCG UGAUGGCAUGCACUAUGCGCG AGCAGGACGC | 4234 |
| 21 | GUCCUGCUGC G CACGU | 1456 | ACGUG UGAUGGCAUGCACUAUGCGCG GCAGCAGGAC | 4235 |
| 53 | GGCCACCCCC G CGAUG | 1457 | CAUCG UGAUGGCAUGCACUAUGCGCG GGGGGUGGCC | 4236 |
| 55 | CCACCCCCGC G AUGCC | 1458 | GGCAU UGAUGGCAUGCACUAUGCGCG GCGGGGGUGG | 4237 |
| 58 | CCCCCGCGAU G CCGCG | 1459 | CGCGG UGAUGGCAUGCACUAUGCGCG AUCGCGGGGG | 4238 |
| 61 | CCGCGAUGCC G CGCGC | 1460 | GCGCG UGAUGGCAUGCACUAUGCGCG GGCAUCGCGG | 4239 |
| 63 | GCGAUGCCGC G CGCUC | 1461 | GAGCG UGAUGGCAUGCACUAUGCGCG GCGGCAUCGC | 4240 |
| 65 | GAUGCCGCGC G CUCCC | 1462 | GGGAG UGAUGGCAUGCACUAUGCGCG GCGCGGCAUC | 4241 |
| 72 | CGCGCUCCCC G CUGCC | 1463 | GGCAG UGAUGGCAUGCACUAUGCGCG GGGGAGCGCG | 4242 |
| 75 | GCUCCCCGCU G CCGAG | 1464 | CUCGG UGAUGGCAUGCACUAUGCGCG AGCGGGGAGC | 4243 |
| 78 | CCCCGCUGCC G AGCCG | 1465 | CGGCU UGAUGGCAUGCACUAUGCGCG GGCAGCGGGG | 4244 |
| 85 | GCCGAGCCGU G CGCUC | 1466 | GAGCG UGAUGGCAUGCACUAUGCGCG ACGGCUCGGC | 4245 |
| 87 | CGAGCCGUGC G CUCCC | 1467 | GGGAG UGAUGGCAUGCACUAUGCGCG GCACGGCUCG | 4246 |
| 94 | UGCGCUCCCU G CUGCG | 1468 | CGCAG UGAUGGCAUGCACUAUGCGCG AGGGAGCGCA | 4247 |
| 97 | GCUCCCUGCU G CGCAG | 1469 | CUGCG UGAUGGCAUGCACUAUGCGCG AGCAGGGAGC | 4248 |
| 99 | UCCCUGCUGC G CAGCC | 1470 | GGCUG UGAUGGCAUGCACUAUGCGCG GCAGCAGGGA | 4249 |
| 111 | AGCCACUACC G CGAGG | 1471 | CCUCG UGAUGGCAUGCACUAUGCGCG GGUAGUGGCU | 4250 |
| 113 | CCACUACCGC G AGGUG | 1472 | CACCU UGAUGGCAUGCACUAUGCGCG GCGGUAGUGG | 4251 |
| 118 | ACCGCGAGGU G CUGCC | 1473 | GGCAG UGAUGGCAUGCACUAUGCGCG ACCUCGCGGU | 4252 |
| 121 | GCGAGGUGCU G CCGCU | 1474 | AGCGG UGAUGGCAUGCACUAUGCGCG AGCACCUCGC | 4253 |
| 124 | AGGUGCUGCC G CUGGC | 1475 | GCCAG UGAUGGCAUGCACUAUGCGCG GGCAGCACCU | 4254 |
| 139 | CCACGUUCGU G CGGCG | 1476 | CGCCG UGAUGGCAUGCACUAUGCGCG ACGAACGUGG | 4255 |
| 144 | UUCGUGCGGC G CCUGG | 1477 | CCAGG UGAUGGCAUGCACUAUGCGCG GCCGCACGAA | 4256 |
| 172 | GGCGGCUGGU G CAGCG | 1478 | CGCUG UGAUGGCAUGCACUAUGCGCG ACCAGCCGCC | 4257 |
| 177 | CUGGUGCAGC G CGGGG | 1479 | CCCCG UGAUGGCAUGCACUAUGCGCG GCUGCACCAG | 4258 |
| 198 | GCGGCUUUCC G CGCGC | 1480 | GCGCG UGAUGGCAUGCACUAUGCGCG GGAAAGCCGC | 4259 |
| 200 | GGCUUUCCGC G CGCUG | 1481 | CAGCG UGAUGGCAUGCACUAUGCGCG GCGGAAAGCC | 4260 |
| 202 | CUUUCCGCGC G CUGGU | 1482 | ACCAG UGAUGGCAUGCACUAUGCGCG GCGCGGAAAG | 4261 |
| 216 | GUGGCCCAGU G CCUGG | 1483 | CCAGG UGAUGGCAUGCACUAUGCGCG ACUGGGCCAC | 4262 |
| 223 | AGUGCCUGGU G UGCGU | 1484 | ACGCA UGAUGGCAUGCACUAUGCGCG ACCAGGCACU | 4263 |
| 225 | UGCCUGGUGU G CGUGC | 1485 | GCACG UGAUGGCAUGCACUAUGCGCG ACACCAGGCA | 4264 |
| 229 | UGGUGUGCGU G CCCUG | 1486 | CAGGG UGAUGGCAUGCACUAUGCGCG ACGCACACCA | 4265 |
| 239 | GCCCUGGGAC G CACGG | 1487 | CCGUG UGAUGGCAUGCACUAUGCGCG GUCCCAGGGC | 4266 |
| 247 | ACGCACGGCC G CCCCC | 1488 | GGGGG UGAUGGCAUGCACUAUGCGCG GGCCGUGCGU | 4267 |
| 254 | GCCGCCCCCC G CCGCC | 1489 | GGCGG UGAUGGCAUGCACUAUGCGCG GGGGGGCGGC | 4268 |
| 257 | GCCCCCCGCC G CCCCC | 1490 | GGGGG UGAUGGCAUGCACUAUGCGCG GGCGGGGGGC | 4269 |
| 270 | CCCUCCUUCC G CCAGG | 1491 | CCUGG UGAUGGCAUGCACUAUGCGCG GGAAGGAGGG | 4270 |
| 277 | UCCGCCAGGU G UCCUG | 1492 | CAGGA UGAUGGCAUGCACUAUGCGCG ACCUGGCGGA | 4271 |
| 282 | CAGGUGUCCU G CCUGA | 1493 | UCAGG UGAUGGCAUGCACUAUGCGCG AGGACACCUG | 4272 |
| 286 | UGUCCUGCCU G AAGGA | 1494 | UCCUU UGAUGGCAUGCACUAUGCGCG AGGCAGGACA | 4273 |
| 303 | CUGGUGGCCC G AGUGC | 1495 | GCACU UGAUGGCAUGCACUAUGCGCG GGGCCACCAG | 4274 |
| 307 | UGGCCCGAGU G CUGCA | 1496 | UGCAG UGAUGGCAUGCACUAUGCGCG ACUCGGGCCA | 4275 |
| 310 | CCCGAGUGCU G CAGAG | 1497 | CUCUG UGAUGGCAUGCACUAUGCGCG AGCACUCGGG | 4276 |
| 319 | UGCAGAGGCU G UGCGA | 1498 | UCGCA UGAUGGCAUGCACUAUGCGCG AGCCUCUGCA | 4277 |
| 321 | CAGAGGCUGU G CGAGC | 1499 | GCUCG UGAUGGCAUGCACUAUGCGCG ACAGCCUCUG | 4278 |
| 323 | GAGGCUGUGC G AGCGC | 1500 | GCGCU UGAUGGCAUGCACUAUGCGCG GCACAGCCUC | 4279 |
| 327 | CUGUGCGAGC G CGGCG | 1501 | CGCCG UGAUGGCAUGCACUAUGCGCG GCUCGCACAG | 4280 |
| 332 | CGAGCGCGGC G CGAAG | 1502 | CUUCG UGAUGGCAUGCACUAUGCGCG GCCGCGCUCG | 4281 |
| 334 | AGCGCGGCGC G AAGAA | 1503 | UUCUU UGAUGGCAUGCACUAUGCGCG GCGCCGCGCU | 4282 |
| 343 | CGAAGAACGU G CUGGC | 1504 | GCCAG UGAUGGCAUGCACUAUGCGCG ACGUUCUUCG | 4283 |
| 359 | CUUCGGCUUC G CGCUG | 1505 | CAGCG UGAUGGCAUGCACUAUGCGCG GAAGCCGAAG | 4284 |
| 361 | UCGGCUUCGC G CUGCU | 1506 | AGCAG UGAUGGCAUGCACUAUGCGCG GCGAAGCCGA | 4285 |
| 364 | GCUUCGCGCU G CUGGA | 1507 | UCCAG UGAUGGCAUGCACUAUGCGCG AGCGCGAAGC | 4286 |
| 378 | GACGGGGCCC G CGGGG | 1508 | CCCCG UGAUGGCAUGCACUAUGCGCG GGGCCCCGUC | 4287 |
| 392 | GGGCCCCCCC G AGGCC | 1509 | GGCCU UGAUGGCAUGCACUAUGCGCG GGGGGGGCCC | 4288 |
| 412 | CCACCAGCGU G CGCAG | 1510 | CUGCG UGAUGGCAUGCACUAUGCGCG ACGCUGGUGG | 4289 |
| 414 | ACCAGCGUGC G CAGCU | 1511 | AGCUG UGAUGGCAUGCACUAUGCGCG GCACGCUGGU | 4290 |
| 424 | GCAGCUACCU G CCCAA | 1512 | UUGGG UGAUGGCAUGCACUAUGCGCG AGGUAGCUGC | 4291 |
| 436 | CCAACACGGU G ACCGA | 1513 | UCGGU UGAUGGCAUGCACUAUGCGCG ACCGUGUUGG | 4292 |
| 440 | CACGGUGACC G ACGCA | 1514 | UGCGU UGAUGGCAUGCACUAUGCGCG GGUCACCGUG | 4293 |
| 443 | GGUGACCGAC G CACUG | 1515 | CAGUG UGAUGGCAUGCACUAUGCGCG GUCGGUCACC | 4294 |
| 448 | CCGACGCACU G CGGGG | 1516 | CCCCG UGAUGGCAUGCACUAUGCGCG AGUGCGUCGG | 4295 |
| 472 | CGUGGGGGCU G CUGCU | 1517 | AGCAG UGAUGGCAUGCACUAUGCGCG AGCCCCCACG | 4296 |
| 475 | GGGGGCUGCU G CUGCG | 1518 | CGCAG UGAUGGCAUGCACUAUGCGCG AGCAGCCCCC | 4297 |
| 478 | GGCUGCUGCU G CGCCG | 1519 | CGGCG UGAUGGCAUGCACUAUGCGCG AGCAGCAGCC | 4298 |
| 480 | CUGCUGCUGC G CCGCG | 1520 | CGCGG UGAUGGCAUGCACUAUGCGCG GCAGCAGCAG | 4299 |
| 483 | CUGCUGCGCC G CGUGG | 1521 | CCACG UGAUGGCAUGCACUAUGCGCG GGCGCAGCAG | 4300 |
| 491 | CCGCGUGGGC G ACGAC | 1522 | GUCGU UGAUGGCAUGCACUAUGCGCG GCCCACGCGG | 4301 |
| 494 | CGUGGGCGAC G ACGUG | 1523 | CACGU UGAUGGCAUGCACUAUGCGCG GUCGCCCACG | 4302 |
| 499 | GCGACGACGU G CUGGU | 1524 | ACCAG UGAUGGCAUGCACUAUGCGCG ACGUCGUCGC | 4303 |
| 511 | UGGUUCACCU G CUGGC | 1525 | GCCAG UGAUGGCAUGCACUAUGCGCG AGGUGAACCA | 4304 |
| 519 | CUGCUGGCAC G CUGCG | 1526 | CGCAG UGAUGGCAUGCACUAUGCGCG GUGCCAGCAG | 4305 |
| 522 | CUGGCACGCU G CGCGC | 1527 | GCGCG UGAUGGCAUGCACUAUGCGCG AGCGUGCCAG | 4306 |
| 524 | GGCACGCUGC G CGCUC | 1528 | GAGCG UGAUGGCAUGCACUAUGCGCG GCAGCGUGCC | 4307 |
| 526 | CACGCUGCGC G CUCUU | 1529 | AAGAG UGAUGGCAUGCACUAUGCGCG GCGCAGCGUG | 4308 |
| 533 | CGCGCUCUUU G UGCUG | 1530 | CAGCA UGAUGGCAUGCACUAUGCGCG AAAGAGCGCG | 4309 |
| 535 | CGCUCUUUGU G CUGGU | 1531 | ACCAG UGAUGGCAUGCACUAUGCGCG ACAAAGAGCG | 4310 |
| 552 | GCUCCCAGCU G CGCCU | 1532 | AGGCG UGAUGGCAUGCACUAUGCGCG AGCUGGGAGC | 4311 |
| 554 | UCCCAGCUGC G CCUAC | 1533 | GUAGG UGAUGGCAUGCACUAUGCGCG GCAGCUGGGA | 4312 |
| 565 | CCUACCAGGU G UGCGG | 1534 | CCGCA UGAUGGCAUGCACUAUGCGCG ACCUGGUAGG | 4313 |
| 567 | UACCAGGUGU G CGGGC | 1535 | GCCCG UGAUGGCAUGCACUAUGCGCG ACACCUGGUA | 4314 |
| 574 | UGUGCGGGCC G CCGCU | 1536 | AGCGG UGAUGGCAUGCACUAUGCGCG GGCCCGCACA | 4315 |
| 577 | GCGGGCCGCC G CUGUA | 1537 | UACAG UGAUGGCAUGCACUAUGCGCG GGCGGCCCGC | 4316 |
| 580 | GGCCGCCGCU G UACCA | 1538 | UGGUA UGAUGGCAUGCACUAUGCGCG AGCGGCGGCC | 4317 |
| 593 | CCAGCUCGGC G CUGCC | 1539 | GGCAG UGAUGGCAUGCACUAUGCGCG GCCGAGCUGG | 4318 |
| 596 | GCUCGGCGCU G CCACU | 1540 | AGUGG UGAUGGCAUGCACUAUGCGCG AGCGCCGAGC | 4319 |
| 616 | CCCGGCCCCC G CCACA | 1541 | UGUGG UGAUGGCAUGCACUAUGCGCG GGGGGCCGGG | 4320 |
| 623 | CCCGCCACAC G CUAGU | 1542 | ACUAG UGAUGGCAUGCACUAUGCGCG GUGUGGCGGG | 4321 |
| 636 | AGUGGACCCC G AAGGC | 1543 | GCCUU UGAUGGCAUGCACUAUGCGCG GGGGUCCACU | 4322 |
| 651 | CGUCUGGGAU G CGAAC | 1544 | GUUCG UGAUGGCAUGCACUAUGCGCG AUCCCAGACG | 4323 |
| 653 | UCUGGGAUGC G AACGG | 1545 | CCGUU UGAUGGCAUGCACUAUGCGCG GCAUCCCAGA | 4324 |
| 703 | CCCUGGGCCU G CCAGC | 1546 | GCUGG UGAUGGCAUGCACUAUGCGCG AGGCCCAGGG | 4325 |
| 716 | AGCCCCGGGU G CGAGG | 1547 | CCUCG UGAUGGCAUGCACUAUGCGCG ACCCGGGGCU | 4326 |
| 718 | CCCCGGGUGC G AGGAG | 1548 | CUCCU UGAUGGCAUGCACUAUGCGCG GCACCCGGGG | 4327 |
| 726 | GCGAGGAGGC G CGGGG | 1549 | CCCCG UGAUGGCAUGCACUAUGCGCG GCCUCCUCGC | 4328 |
| 737 | CGGGGGCAGU G CCAGC | 1550 | GCUGG UGAUGGCAUGCACUAUGCGCG ACUGCCCCCG | 4329 |
| 744 | AGUGCCAGCC G AAGUC | 1551 | GACUU UGAUGGCAUGCACUAUGCGCG GGCUGGCACU | 4330 |
| 751 | GCCGAAGUCU G CCGUU | 1552 | AACGG UGAUGGCAUGCACUAUGCGCG AGACUUCGGC | 4331 |
| 757 | GUCUGCCGUU G CCCAA | 1553 | UUGGG UGAUGGCAUGCACUAUGCGCG AACGGCAGAC | 4332 |
| 779 | CAGGCGUGGC G CUGCC | 1554 | GGCAG UGAUGGCAUGCACUAUGCGCG GCCACGCCUG | 4333 |
| 782 | GCGUGGCGCU G CCCCU | 1555 | AGGGG UGAUGGCAUGCACUAUGCGCG AGCGCCACGC | 4334 |
| 788 | CGCUGCCCCU G AGCCG | 1556 | CGGCU UGAUGGCAUGCACUAUGCGCG AGGGGCAGCG | 4335 |
| 802 | CGGAGCGGAC G CCCGU | 1557 | ACGGG UGAUGGCAUGCACUAUGCGCG GUCCGCUCCG | 4336 |
| 841 | CGGGCAGGAC G CGUGG | 1558 | CCACG UGAUGGCAUGCACUAUGCGCG GUCCUGCCCG | 4337 |
| 850 | CGCGUGGACC G AGUGA | 1559 | UCACU UGAUGGCAUGCACUAUGCGCG GGUCCACGCG | 4338 |
| 854 | UGGACCGAGU G ACCGU | 1560 | ACGGU UGAUGGCAUGCACUAUGCGCG ACUCGGUCCA | 4339 |
| 867 | CGUGGUUUCU G UGUGG | 1561 | CCACA UGAUGGCAUGCACUAUGCGCG AGAAACCACG | 4340 |
| 869 | UGGUUUCUGU G UGGUG | 1562 | CACCA UGAUGGCAUGCACUAUGCGCG ACAGAAACCA | 4341 |
| 874 | UCUGUGUGGU G UCACC | 1563 | GGUGA UGAUGGCAUGCACUAUGCGCG ACCACACAGA | 4342 |
| 881 | GGUGUCACCU G CCAGA | 1564 | UCUGG UGAUGGCAUGCACUAUGCGCG AGGUGACACC | 4343 |
| 890 | UGCCAGACCC G CCGAA | 1565 | UUCGG UGAUGGCAUGCACUAUGCGCG GGGUCUGGCA | 4344 |
| 893 | CAGACCCGCC G AAGAA | 1566 | UUCUU UGAUGGCAUGCACUAUGCGCG GGCGGGUCUG | 4345 |
| 917 | UUUGGAGGGU G CGCUC | 1567 | GAGCG UGAUGGCAUGCACUAUGCGCG ACCCUCCAAA | 4346 |
| 919 | UGGAGGGUGC G CUCUC | 1568 | GAGAG UGAUGGCAUGCACUAUGCGCG GCACCCUCCA | 4347 |
| 931 | UCUCUGGCAC G CGCCA | 1569 | UGGCG UGAUGGCAUGCACUAUGCGCG GUGCCAGAGA | 4348 |
| 933 | UCUGGCACGC G CCACU | 1570 | AGUGG UGAUGGCAUGCACUAUGCGCG GCGUGCCAGA | 4349 |
| 957 | UCCGUGGGCC G CCAGC | 1571 | GCUGG UGAUGGCAUGCACUAUGCGCG GGCCCACGGA | 4350 |
| 968 | CCAGCACCAC G CGGGC | 1572 | GCCCG UGAUGGCAUGCACUAUGCGCG GUGGUGCUGG | 4351 |
| 988 | CAUCCACAUC G CGGCC | 1573 | GGCCG UGAUGGCAUGCACUAUGCGCG GAUGUGGAUG | 4352 |
| 1012 | CCUGGGACAC G CCUUG | 1574 | CAAGG UGAUGGCAUGCACUAUGCGCG GUGUCCCAGG | 4353 |
| 1017 | GACACGCCUU G UCCCC | 1575 | GGGGA UGAUGGCAUGCACUAUGCGCG AAGGCGUGUC | 4354 |
| 1027 | GUCCCCCGGU G UACGC | 1576 | GCGUA UGAUGGCAUGCACUAUGCGCG ACCGGGGGAC | 4355 |
| 1031 | CCCGGUGUAC G CCGAG | 1577 | CUCGG UGAUGGCAUGCACUAUGCGCG GUACACCGGG | 4356 |
| 1034 | GGUGUACGCC G AGACC | 1578 | GGUCU UGAUGGCAUGCACUAUGCGCG GGCGUACACC | 4357 |
| 1064 | CUCCUCAGGC G ACAAG | 1579 | CUUGU UGAUGGCAUGCACUAUGCGCG GCCUGAGGAG | 4358 |
| 1078 | AGGAGCAGCU G CGGCC | 1580 | GGCCG UGAUGGCAUGCACUAUGCGCG AGCUGCUCCU | 4359 |
| 1105 | UCAGCUCUCU G AGGCC | 1581 | GGCCU UGAUGGCAUGCACUAUGCGCG AGAGAGCUGA | 4360 |
| 1117 | GGCCCAGCCU G ACUGG | 1582 | CCAGU UGAUGGCAUGCACUAUGCGCG AGGCUGGGCC | 4361 |
| 1124 | CCUGACUGGC G CUCGG | 1583 | CCGAG UGAUGGCAUGCACUAUGCGCG GCCAGUCAGG | 4362 |
| 1171 | GGCCCUGGAU G CCAGG | 1584 | CCUGG UGAUGGCAUGCACUAUGCGCG AUCCAGGGCC | 4363 |
| 1185 | GGGACUCCCC G CAGGU | 1585 | ACCUG UGAUGGCAUGCACUAUGCGCG GGGGAGUCCC | 4364 |
| 1192 | CCCGCAGGUU G CCCCG | 1586 | CGGGG UGAUGGCAUGCACUAUGCGCG AACCUGCGGG | 4365 |
| 1197 | AGGUUGCCCC G CCUGC | 1587 | GCAGG UGAUGGCAUGCACUAUGCGCG GGGGCAACCU | 4366 |
| 1201 | UGCCCCGCCU G CCCCA | 1588 | UGGGG UGAUGGCAUGCACUAUGCGCG AGGCGGGGCA | 4367 |
| 1209 | CUGCCCCAGC G CUACU | 1589 | AGUAG UGAUGGCAUGCACUAUGCGCG GCUGGGGCAG | 4368 |
| 1222 | ACUGGCAAAU G CGGCC | 1590 | GGCCG UGAUGGCAUGCACUAUGCGCG AUUUGCCAGU | 4369 |
| 1231 | UGCGGCCCCU G UUUCU | 1591 | AGAAA UGAUGGCAUGCACUAUGCGCG AGGGGCCGCA | 4370 |
| 1243 | UUCUGGAGCU G CUUGG | 1592 | CCAAG UGAUGGCAUGCACUAUGCGCG AGCUCCAGAA | 4371 |
| 1256 | UGGGAACCAC G CGCAG | 1593 | CUGCG UGAUGGCAUGCACUAUGCGCG GUGGUUCCCA | 4372 |
| 1258 | GGAACCACGC G CAGUG | 1594 | CACUG UGAUGGCAUGCACUAUGCGCG GCGUGGUUCC | 4373 |
| 1263 | CACGCGCAGU G CCCCU | 1595 | AGGGG UGAUGGCAUGCACUAUGCGCG ACUGCGCGUG | 4374 |
| 1276 | CCUACGGGGU G CUCCU | 1596 | AGGAG UGAUGGCAUGCACUAUGCGCG ACCCCGUAGG | 4375 |
| 1288 | UCCUCAAGAC G CACUG | 1597 | CAGUG UGAUGGCAUGCACUAUGCGCG GUCUUGAGGA | 4376 |
| 1293 | AAGACGCACU G CCCGC | 1598 | GCGGG UGAUGGCAUGCACUAUGCGCG AGUGCGUCUU | 4377 |
| 1297 | CGCACUGCCC G CUGCG | 1599 | CGCAG UGAUGGCAUGCACUAUGCGCG GGGCAGUGCG | 4378 |
| 1300 | ACUGCCCGCU G CGAGC | 1600 | GCUCG UGAUGGCAUGCACUAUGCGCG AGCGGGCAGU | 4379 |
| 1302 | UGCCCGCUGC G ACCUG | 1601 | CAGCU UGAUGGCAUGCACUAUGCGCG GCAGCGGGCA | 4380 |
| 1307 | GCUGCGAGCU G CGGUC | 1602 | GACCG UGAUGGCAUGCACUAUGCGCG AGCUCGCAGC | 4381 |
| 1328 | AGCAGCCGGU G UCUGU | 1603 | ACAGA UGAUGGCAUGCACUAUGCGCG ACCGGCUGCU | 4382 |
| 1332 | GCCGGUGUCU G UGCCC | 1604 | GGGCA UGAUGGCAUGCACUAUGCGCG AGACACCGGC | 4383 |
| 1334 | CGGUGUCUGU G CCCGG | 1605 | CCGGG UGAUGGCAUGCACUAUGCGCG ACAGACACCG | 4384 |
| 1358 | CCAGGGCUCU G UGGCG | 1606 | CGCCA UGAUGGCAUGCACUAUGCGCG AGAGCCCUGG | 4385 |
| 1370 | GGCGGCCCCC G AGGAG | 1607 | CUCCU UGAUGGCAUGCACUAUGCGCG GGGGGCCGCC | 4386 |
| 1395 | GACCCCCGUC C CCUGG | 1608 | CCAGG UGAUGGCAUGCACUAUGCGCG GACGGGGGUC | 4387 |
| 1402 | GUCGCCUGGU G CAGCU | 1609 | ACCUG UGAUGGCAUGCACUAUGCGCG ACCAGGCGAC | 4388 |
| 1408 | UGGUGCAGCU G CUCCG | 1610 | CGGAG UGAUGGCAUGCACUAUGCGCG AGCUGCACCA | 4389 |
| 1413 | CAGCUGCUCC G CCAGC | 1611 | GCUGG UGAUGGCAUGCACUAUGCGCG GGAGCAGCUG | 4390 |
| 1438 | CCUGGCAGGU G UACGG | 1612 | CCGUA UGAUGGCAUGCACUAUGCGCG ACCUGCCAGG | 4391 |
| 1450 | ACGGCUUCGU G CGGGC | 1613 | GCCCG UGAUGGCAUGCACUAUGCGCG ACGAAGCCGU | 4392 |
| 1458 | GUGCGGGCCU G CCUGC | 1614 | GCAGG UGAUGGCAUGCACUAUGCGCG AGGCCCGCAC | 4393 |
| 1462 | GGGCCUGCCU G CGCCG | 1615 | CGGCG UGAUGGCAUGCACUAUGCGCG AGGCAGGCCC | 4394 |
| 1464 | GCCUGCCUGC G CCGGC | 1616 | GCCGG UGAUGGCAUGCACUAUGCGCG GCAGGCAGGC | 4395 |
| 1474 | GCCGGCUGGU G CCCCC | 1617 | GGGGG UGAUGGCAUGCACUAUGCGCG ACCAGCCGGC | 4396 |
| 1505 | CAGGCACAAC G AACGC | 1618 | GCGUU UGAUGGCAUGCACUAUGCGCG GUUGUGCCUG | 4397 |
| 1509 | CACAACGAAC G CCGCU | 1619 | AGCGG UGAUGGCAUGCACUAUGCGCG GUUCGUUGUG | 4398 |
| 1512 | AACGAACGCC G CUUCC | 1620 | GGAAG UGAUGGCAUGCACUAUGCGCG GGCGUUCGUU | 4399 |
| 1556 | GGGGAAGCAU G CCAAG | 1621 | CUUGG UGAUGGCAUGCACUAUGCGCG AUGCUUCCCC | 4400 |
| 1567 | CCAAGCUCUC G CUGCA | 1622 | UGCAG UGAUGGCAUGCACUAUGCGCG GAGAGCUUGG | 4401 |
| 1570 | AGCUCUCGCU G CAGGA | 1623 | UCCUG UGAUGGCAUGCACUAUGCGCG AGCGAGAGCU | 4402 |
| 1579 | UGCAGGAGCU G ACGUG | 1624 | CACGU UGAUGGCAUGCACUAUGCGCG AGCUCCUGCA | 4403 |
| 1591 | CGUGGAAGAU G AGCGU | 1625 | ACGCU UGAUGGCAUGCACUAUGCGCG AUCUUCCACG | 4404 |
| 1597 | AGAUGAGCGU G CGGGA | 1626 | UCCCG UGAUGGCAUGCACUAUGCGCG ACGCUCAUCU | 4405 |
| 1605 | GUGCGGGACU G CGCUU | 1627 | AAGCG UGAUGGCAUGCACUAUGCGCG AGUCCCGCAC | 4406 |
| 1607 | GCGGGACUGC G CUUGG | 1628 | CCAAG UGAUGGCAUGCACUAUGCGCG GCAGUCCCGC | 4407 |
| 1615 | GCGCUUGGCU G CGCAG | 1629 | CUGCG UGAUGGCAUGCACUAUGCGCG AGCCAAGCGC | 4408 |
| 1617 | GCUUGGCUGC G CAGGA | 1630 | UCCUG UGAUGGCAUGCACUAUGCGCG GCAGCCAAGC | 4409 |
| 1638 | GGGGUUGGCU G UGUUC | 1631 | GAACA UGAUGGCAUGCACUAUGCGCG AGCCAACCCC | 4410 |
| 1640 | GGUUGGCUGU G UUCCG | 1632 | CGGAA UGAUGGCAUGCACUAUGCGCG ACAGCCAACC | 4411 |
| 1649 | UGUUCCGGCC G CAGAG | 1633 | CUCUG UGAUGGCAUGCACUAUGCGCG GGCCGGAACA | 4412 |
| 1663 | AGCACCGUCU G CGUGA | 1634 | UCACG UGAUGGCAUGCACUAUGCGCG AGACGGUGCU | 4413 |
| 1667 | CCGUCUGCGU G AGGAG | 1635 | CUCCU UGAUGGCAUGCACUAUGCGCG ACGCAGACGG | 4414 |
| 1690 | CCAAGUUCCU G CACUG | 1636 | CAGUG UGAUGGCAUGCACUAUGCGCG AGGAACUUGG | 4415 |
| 1699 | UGCACUGGCU G AUGAG | 1637 | CUCAU UGAUGGCAUGCACUAUGCGCG AGCCAGUGCA | 4416 |
| 1702 | ACUGGCUGAU G AGUGU | 1638 | ACACU UGAUGGCAUGCACUAUGCGCG AUCAGCCAGU | 4417 |
| 1706 | GCUGAUGAGU G UGUAC | 1639 | GUACA UGAUGGCAUGCACUAUGCGCG ACUCAUCAGC | 4418 |
| 1708 | UGAUGAGUGU G UACGU | 1640 | ACGUA UGAUGGCAUGCACUAUGCGCG ACACUCAUCA | 4419 |
| 1718 | GUACGUCGUC G AGCUG | 1641 | CAGCU UGAUGGCAUGCACUAUGCGCG GACGACGUAC | 4420 |
| 1723 | UCGUCGAGCU G CUCAG | 1642 | CUGAG UGAUGGCAUGCACUAUGCGCG AGCUCGACGA | 4421 |
| 1742 | UUUCUUUUAU G UCACG | 1643 | CGUGA UGAUGGCAUGCACUAUGCGCG AUAAAAGAAA | 4422 |
| 1793 | CCGGAAGAGU G UCUGG | 1644 | CCAGA UGAUGGCAUGCACUAUGCGCG ACUCUUCCGG | 4423 |
| 1807 | GGAGCAAGUU G CAAAG | 1645 | CUUUG UGAUGGCAUGCACUAUGCGCG AACUUGCUCC | 4424 |
| 1834 | GACAGCACUU G AAGAG | 1646 | CUCUU UGAUGGCAUGCACUAUGCGCG AAGUGCUGUC | 4425 |
| 1843 | UGAAGAGGGU G CAGCU | 1647 | AGCUG UGAUGGCAUGCACUAUGCGCG ACCCUCUUCA | 4426 |
| 1849 | GGGUGCAGCU G CGGGA | 1648 | UCCCG UGAUGGCAUGCACUAUGCGCG AGCUGCACCC | 4427 |
| 1858 | UGCGGGAGCU G UCGGA | 1649 | UCCGA UGAUGGCAUGCACUAUGCGCG AGCUCCCGCA | 4428 |
| 1898 | AGCCAGGCCC G CCCUG | 1650 | CAGGG UGAUGGCAUGCACUAUGCGCG GGGCCUGGCU | 4429 |
| 1903 | GGCCCGCCCU G CUGAC | 1651 | GUCAG UGAUGGCAUGCACUAUGCGCG AGGGCGGGCC | 4430 |
| 1906 | CCGCCCUGCU G ACGUC | 1652 | GACGU UGAUGGCAUGCACUAUGCGCG AGCAGGGCGG | 4431 |
| 1920 | UCCAGACUCC G CUUCA | 1653 | UGAAG UGAUGGCAUGCACUAUGCGCG GGAGUCUGGA | 4432 |
| 1937 | CCCCAAGCCU G ACGGG | 1654 | CCCGU UGAUGGCAUGCACUAUGCGCG AGGCUUGGGG | 4433 |
| 1945 | CUGACGGGCU G CGGCC | 1655 | GGCCG UGAUGGCAUGCACUAUGCGCG AGCCCGUCAG | 4434 |
| 1951 | GGCUGCGGCC G AUUGU | 1656 | ACAAU UGAUGGCAUGCACUAUGCGCG GGCCGCAGCC | 4435 |
| 1955 | GCGGCCGAUU G UGAAC | 1657 | GUUCA UGAUGGCAUGCACUAUGCGCG AAUCGGCCGC | 4436 |
| 1957 | GGCCGAUUGU G AACAU | 1658 | AUGUU UGAUGGCAUGCACUAUGCGCG ACAAUCGGCC | 4437 |
| 1992 | AGAACGUUCC G CAGAG | 1659 | CUCUG UGAUGGCAUGCACUAUGCGCG GGAACGUUCU | 4438 |
| 2009 | AAAGAGGGCC G AGCGU | 1660 | ACGCU UGAUGGCAUGCACUAUGCGCG GGCCCUCUUU | 4439 |
| 2023 | GUCUCACCUC G AGGGU | 1661 | ACCCU UGAUGGCAUGCACUAUGCGCG GAGGUGAGAC | 4440 |
| 2029 | CCUCGAGGGU G AAGGC | 1662 | GCCUU UGAUGGCAUGCACUAUGCGCG ACCCUCGAGG | 4441 |
| 2038 | UGAAGGCACU G UUCAG | 1663 | CUGAA UGAUGGCAUGCACUAUGCGCG AGUGCCUUCA | 4442 |
| 2047 | UGUUCAGCGU G CUCAA | 1664 | UUGAG UGAUGGCAUGCACUAUGCGCG ACGCUGAACA | 4443 |
| 2057 | GCUCAACUAC G AGCGG | 1665 | CCGCU UGAUGGCAUGCACUAUGCGCG GUAGUUGAGC | 4444 |
| 2065 | ACGAGCGGGC G CGGCG | 1666 | CGCCG UGAUGGCAUGCACUAUGCGCG GCCCGCUCGU | 4445 |
| 2070 | CGGGCGCGGC G CCCCG | 1667 | CGGGG UGAUGGCAUGCACUAUGCGCG GCCGCGCCCG | 4446 |
| 2087 | CCUCCUGGGC G CCUCU | 1668 | AGAGG UGAUGGCAUGCACUAUGCGCG GCCCAGGAGG | 4447 |
| 2093 | GGGCGCCUCU G UGCUG | 1669 | CAGCA UGAUGGCAUGCACUAUGCGCG AGAGGCGCCC | 4448 |
| 2095 | GCGCCUCUGU G CUGGG | 1670 | CCCAG UGAUGGCAUGCACUAUGCGCG ACAGAGGCGC | 4449 |
| 2108 | GGGCCUGGAC G AUAUC | 1671 | GAUAU UGAUGGCAUGCACUAUGCGCG GUCCAGGCCC | 4450 |
| 2127 | AGGGCCUGGC G CACCU | 1672 | AGGUG UGAUGGCAUGCACUAUGCGCG GCCAGGCCCU | 4451 |
| 2137 | GCACCUUCGU G CUGCG | 1673 | CGCAG UGAUGGCAUGCACUAUGCGCG ACGAAGGUGC | 4452 |
| 2140 | CCUUCGUGCU G CGUGU | 1674 | ACACG UGAUGGCAUGCACUAUGCGCG AGCACGAAGG | 4453 |
| 2144 | CGUGCUGCGU G UGCGG | 1675 | CCGCA UGAUGGCAUGCACUAUGCGCG ACGCAGCACG | 4454 |
| 2146 | UGCUGCGUGU G CGGGC | 1676 | GCCCG UGAUGGCAUGCACUAUGCGCG ACACGCAGCA | 4455 |
| 2161 | CCCAGGACCC G CCGCC | 1677 | GGCGG UGAUGGCAUGCACUAUGCGCG GGGUCCUGGG | 4456 |
| 2164 | AGGACCCGCC G CCUGA | 1678 | UCAGG UGAUGGCAUGCACUAUGCGCG GGCGGGUCCU | 4457 |
| 2168 | CCCGCCGCCU G AGCUG | 1679 | CAGCU UGAUGGCAUGCACUAUGCGCG AGGCGGCGGG | 4458 |
| 2173 | CGCCUGAGCU G UACUU | 1680 | AAGUA UGAUGGCAUGCACUAUGCGCG AGCUCAGGCG | 4459 |
| 2180 | GCUGUACUUU G UCAAG | 1681 | CUUGA UGAUGGCAUGCACUAUGCGCG AAAGUACAGC | 4460 |
| 2192 | CAAGGUGGAU G UGACG | 1682 | CGUCA UGAUGGCAUGCACUAUGCGCG AUCCACCUUG | 4461 |
| 2194 | AGGUGGAUGU G ACGGG | 1683 | CCCGU UGAUGGCAUGCACUAUGCGCG ACAUCCACCU | 4462 |
| 2201 | UGUGACGGGC G CGUAC | 1684 | GUACG UGAUGGCAUGCACUAUGCGCG GCCCGUCACA | 4463 |
| 2207 | GGGCGCGUAC G ACACC | 1685 | GGUGU UGAUGGCAUGCACUAUGCGCG GUACGCGCCC | 4464 |
| 2243 | GGAGGUCAUC G CCAGC | 1686 | GCUGG UGAUGGCAUGCACUAUGCGCG GAUGACCUCC | 4465 |
| 2274 | AACACGUACU G CGUGC | 1687 | GCACG UGAUGGCAUGCACUAUGCGCG AGUACGUGUU | 4466 |
| 2278 | CGUACUGCGU G CGUCG | 1688 | CGACG UGAUGGCAUGCACUAUGCGCG ACGCAGUACG | 4467 |
| 2288 | GCGUCGGUAU G CCGUG | 1689 | CACGG UGAUGGCAUGCACUAUGCGCG AUACCGACGC | 4468 |
| 2306 | CCAGAAGGCC G CCCAU | 1690 | AUGGG UGAUGGCAUGCACUAUGCGCG GGCCUUCUGG | 4469 |
| 2322 | GGGCACGUCC G CAAGG | 1691 | CCUUG UGAUGGCAUGCACUAUGCGCG GGACGUGCCC | 4470 |
| 2353 | UCUCUACCUU G ACAGA | 1692 | UCUGU UGAUGGCAUGCACUAUGCGCG AAGGUAGAGA | 4471 |
| 2374 | AGCCGUACAU G CGACA | 1693 | UGUCG UGAUGGCAUGCACUAUGCGCG AUGUACGGCU | 4472 |
| 2376 | CCGUACAUGC G ACAGU | 1694 | ACUGU UGAUGGCAUGCACUAUGCGCG GCAUGUACGG | 4473 |
| 2395 | UGGCUCACCU G CAGGA | 1695 | UCCUG UGAUGGCAUGCACUAUGCGCG AGGUGAGCCA | 4474 |
| 2410 | AGACCAGCCC G CUGAG | 1696 | CUCAG UGAUGGCAUGCACUAUGCGCG GGGCUGGUCU | 4475 |
| 2413 | CCAGCCCGCU G AGGGA | 1697 | UCCCU UGAUGGCAUGCACUAUGCGCG AGCGGGCUGG | 4476 |
| 2420 | GCUGAGGGAU G CCGUC | 1698 | GACGG UGAUGGCAUGCACUAUGCGCG AUCCCUCAGC | 4477 |
| 2432 | CGUCGUCAUC G AGCAG | 1699 | CUGCU UGAUGGCAUGCACUAUGCGCG GAUGACGACG | 4478 |
| 2449 | GCUCCUCCCU G AAUGA | 1700 | UCAUU UGAUGGCAUGCACUAUGCGCG AGGGAGGAGC | 4479 |
| 2453 | CUCCCUGAAU G AGGCC | 1701 | GGCCU UGAUGGCAUGCACUAUGCGCG AUUCAGGGAG | 4480 |
| 2474 | UGGCCUCUUC G ACGUC | 1702 | GACGU UGAUGGCAUGCACUAUGCGCG GAAGAGGCCA | 4481 |
| 2487 | GUCUUCCUAC G CUUCA | 1703 | UGAAG UGAUGGCAUGCACUAUGCGCG GUAGGAAGAC | 4482 |
| 2494 | UACGCUUCAU G UGCCA | 1704 | UGGCA UGAUGGCAUGCACUAUGCGCG AUGAAGCGUA | 4483 |
| 2496 | CGCUUCAUGU G CCACC | 1705 | GGUGG UGAUGGCAUGCACUAUGCGCG ACAUGAAGCG | 4484 |
| 2504 | GUGCCACCAC G CCGUG | 1706 | CACGG UGAUGGCAUGCACUAUGCGCG GUGGUGGCAC | 4485 |
| 2509 | ACCACGCCGU G CGCAU | 1707 | AUGCG UGAUGGCAUGCACUAUGCGCG ACGGCGUGGU | 4486 |
| 2511 | CACGCCGUGC G CAUCA | 1708 | UGAUG UGAUGGCAUGCACUAUGCGCG GCACGGCGUG | 4487 |
| 2538 | UACGUCCAGU G CCAGG | 1709 | CCUGG UGAUGGCAUGCACUAUGCGCG ACUGGACGUA | 4488 |
| 2551 | AGGGGAUCCC G CAGGG | 1710 | CCCUG UGAUGGCAUGCACUAUGCGCG GGGAUCCCCU | 4489 |
| 2572 | UCCUCUCCAC G CUGCU | 1711 | AGCAG UGAUGGCAUGCACUAUGCGCG GUGGAGAGGA | 4490 |
| 2575 | UCUCCACGCU G CUCUG | 1712 | CAGAG UGAUGGCAUGCACUAUGCGCG AGCGUGGAGA | 4491 |
| 2580 | ACGCUGCUCU G CAGCC | 1713 | GGCUG UGAUGGCAUGCACUAUGCGCG AGAGCAGCGU | 4492 |
| 2587 | UCUGCAGCCU G UGCUA | 1714 | UAGCA UGAUGGCAUGCACUAUGCGCG AGGCUGCAGA | 4493 |
| 2589 | UGCAGCCUGU G CUACG | 1715 | CGUAG UGAUGGCAUGCACUAUGCGCG ACAGGCUGCA | 4494 |
| 2597 | GUGCUACGGC G ACAUG | 1716 | CAUGU UGAUGGCAUGCACUAUGCGCG GCCGUAGCAC | 4495 |
| 2614 | AGAACAAGCU G UUUGC | 1717 | GCAAA UGAUGGCAUGCACUAUGCGCG AGCUUGUUCU | 4496 |
| 2618 | CAAGCUGUUU G CGGGG | 1718 | CCCCG UGAUGGCAUGCACUAUGCGCG AAACAGCUUG | 4497 |
| 2641 | GGGACGGGCU G CUCCU | 1719 | AGGAG UGAUGGCAUGCACUAUGCGCG AGCCCGUCCC | 4498 |
| 2647 | GGCUGCUCCU G CGUUU | 1720 | AAACG UGAUGGCAUGCACUAUGCGCG AGGAGCAGCC | 4499 |
| 2660 | UUUGGUGGAU G AUUUC | 1721 | GAAAU UGAUGGCAUGCACUAUGCGCG AUCCACCAAA | 4500 |
| 2668 | AUGAUUUCUU G UUGGU | 1722 | ACCAA UGAUGGCAUGCACUAUGCGCG AAGAAAUCAU | 4501 |
| 2674 | UCUUGUUGGU G ACACC | 1723 | GGUGU UGAUGGCAUGCACUAUGCGCG ACCAACAAGA | 4502 |
| 2693 | CCUCACCCAC G CGAAA | 1724 | UUUCG UGAUGGCAUGCACUAUGCGCG GUGGGUGAGG | 4503 |
| 2695 | UCACCCACGC G AAAAC | 1725 | GUUUU UGAUGGCAUGCACUAUGCGCG GCGUGGGUGA | 4504 |
| 2721 | ACCCUGGUCC G AGGUG | 1726 | CACCU UGAUGGCAUGCACUAUGCGCG GGACCAGGGU | 4505 |
| 2726 | GGUCCGAGGU G UCCCU | 1727 | AGGGA UGAUGGCAUGCACUAUGCGCG ACCUCGGACC | 4506 |
| 2732 | AGGUGUCCCU G AGUAU | 1728 | AUACU UGAUGGCAUGCACUAUGCGCG AGGGACACCU | 4507 |
| 2742 | GAGUAUGGCU G CGUGG | 1729 | CCACG UGAUGGCAUGCACUAUGCGCG AGCCAUACUC | 4508 |
| 2749 | GCUGCGUGGU G AACUU | 1730 | AAGUU UGAUGGCAUGCACUAUGCGCG ACCACGCAGC | 4509 |
| 2755 | UGGUGAACUU G CGGAA | 1731 | UUCCG UGAUGGCAUGCACUAUGCGCG AAGUUCACCA | 4510 |
| 2770 | AGACAGUGGU G AACUU | 1732 | AAGUU UGAUGGCAUGCACUAUGCGCG ACCACUGUCU | 4511 |
| 2780 | GAACUUCCCU G UAGAA | 1733 | UUCUA UGAUGGCAUGCACUAUGCGCG AGGGAAGUUC | 4512 |
| 2789 | UGUAGAAGAC G AGGCC | 1734 | GGCCU UGAUGGCAUGCACUAUGCGCG GUCUUCUACA | 4513 |
| 2813 | CACGGCUUUU G UUCAG | 1735 | CUGAA UGAUGGCAUGCACUAUGCGCG AAAAGCCGUG | 4514 |
| 2821 | UUGUUCAGAU G CCGGC | 1736 | GCCGG UGAUGGCAUGCACUAUGCGCG AUCUGAACAA | 4515 |
| 2847 | UUCCCCUGGU G CGGCC | 1737 | GGCCG UGAUGGCAUGCACUAUGCGCG ACCAGGGGAA | 4516 |
| 2854 | GGUGCGGCCU G CUGCU | 1738 | AGCAG UGAUGGCAUGCACUAUGCGCG AGGCCGCACC | 4517 |
| 2857 | GCGGCCUGCU G CUGGA | 1739 | UCCAG UGAUGGCAUGCACUAUGCGCG AGCAGGCCGC | 4518 |
| 2881 | CCCUGGAGGU G CAGAG | 1740 | CUCUG UGAUGGCAUGCACUAUGCGCG ACCUCCAGGG | 4519 |
| 2888 | GGUGCAGAGC G ACUAC | 1741 | GUAGU UGAUGGCAUGCACUAUGCGCG GCUCUGCACC | 4520 |
| 2903 | CUCCAGCUAU G CCCGG | 1742 | CCGGG UGAUGGCAUGCACUAUGCGCG AUAGCUGGAG | 4521 |
| 2940 | ACCUUCAACC G CGGCU | 1743 | AGCCG UGAUGGCAUGCACUAUGCGCG GGUUGAAGGU | 4522 |
| 2965 | GGAGGAACAU G CGUCG | 1744 | CGACG UGAUGGCAUGCACUAUGCGCG AUGUUCCUCC | 4523 |
| 2970 | AACAUGCGUC G CAAAC | 1745 | GUUUG UGAUGGCAUGCACUAUGCGCG GACGCAUGUU | 4524 |
| 2989 | UUGGGGUCUU G CGGCU | 1746 | AGCCG UGAUGGCAUGCACUAUGCGCG AAGACCCCAA | 4525 |
| 2995 | UCUUGCGGCU G AAGUG | 1747 | CACUU UGAUGGCAUGCACUAUGCGCG AGCCGCAAGA | 4526 |
| 3000 | CGGCUGAAGU G UCACA | 1748 | UGUGA UGAUGGCAUGCACUAUGCGCG ACUUCAGCCG | 4527 |
| 3010 | GUCACAGCCU G UUUCU | 1749 | AGAAA UGAUGGCAUGCACUAUGCGCG AGGCUGUGAC | 4528 |
| 3022 | UUCUGGAUUU G CAGGU | 1750 | ACCUG UGAUGGCAUGCACUAUGCGCG AAAUCCAGAA | 4529 |
| 3028 | AUUUGCAGGU G AACAG | 1751 | CUGUU UGAUGGCAUGCACUAUGCGCG ACCUGCAAAU | 4530 |
| 3046 | UCCAGACGGU G UGCAC | 1752 | GUGCA UGAUGGCAUGCACUAUGCGCG ACCGUCUGGA | 4531 |
| 3048 | CAGACGGUGU G CACCA | 1753 | UGGUG UGAUGGCAUGCACUAUGCGCG ACACCGUCUG | 4532 |
| 3073 | AGAUCCUCCU G CUGCA | 1754 | UGCAG UGAUGGCAUGCACUAUGCGCG AGGAGGAUCU | 4533 |
| 3076 | UCCUCCUGCU G CAGGC | 1755 | GCCUG UGAUGGCAUGCACUAUGCGCG AGCAGGAGGA | 4534 |
| 3095 | CAGGUUUCAC G CAUGU | 1756 | ACAUG UGAUGGCAUGCACUAUGCGCG GUGAAACCUG | 4535 |
| 3099 | UUUCACGCAU G UGUGC | 1757 | GCACA UGAUGGCAUGCACUAUGCGCG AUGCGUGAAA | 4536 |
| 3101 | UCACGCAUGU G UGCUG | 1758 | CAGCA UGAUGGCAUGCACUAUGCGCG ACAUGCGUGA | 4537 |
| 3103 | ACGCAUGUGU G CUGCA | 1759 | UGCAG UGAUGGCAUGCACUAUGCGCG ACACAUGCGU | 4538 |
| 3106 | CAUGUGUGCU G CAGCU | 1760 | AGCUG UGAUGGCAUGCACUAUGCGCG AGCACACAUG | 4539 |
| 3154 | CAUUUUUCCU G CGCGU | 1761 | ACGCG UGAUGGCAUGCACUAUGCGCG AGGAAAAAUG | 4540 |
| 3156 | UUUUUCCUGC G CGUCA | 1762 | UGACG UGAUGGCAUGCACUAUGCGCG GCAGGAAAAA | 4541 |
| 3167 | CGUCAUCUCU G ACACG | 1763 | CGUGU UGAUGGCAUGCACUAUGCGCG AGAGAUGACG | 4542 |
| 3183 | GCCUCCCUCU G CUACU | 1764 | AGUAG UGAUGGCAUGCACUAUGCGCG AGAGGGAGGC | 4543 |
| 3196 | ACUCCAUCCU G AAAGC | 1765 | GCUUU UGAUGGCAUGCACUAUGCGCG AGGAUGGAGU | 4544 |
| 3209 | AGCCAAGAAC G CAGGG | 1766 | CCCUG UGAUGGCAUGCACUAUGCGCG GUUCUUGGCU | 4545 |
| 3217 | ACGCAGGGAU G UCGCU | 1767 | AGCGA UGAUGGCAUGCACUAUGCGCG AUCCCUGCGU | 4546 |
| 3220 | CAGGGAUGUC G CUGGG | 1768 | CCCAG UGAUGGCAUGCACUAUGCGCG GACAUCCCUG | 4547 |
| 3236 | GGCCAAGGGC G CCGCC | 1769 | GGCGG UGAUGGCAUGCACUAUGCGCG GCCCUUGGCC | 4548 |
| 3239 | CAAGGGCGCC G CCGGC | 1770 | GCCGG UGAUGGCAUGCACUAUGCGCG GGCGCCCUUG | 4549 |
| 3250 | CCGGCCCUCU G CCCUC | 1771 | GAGGG UGAUGGCAUGCACUAUGCGCG AGAGGGCCGG | 4550 |
| 3257 | UCUGCCCUCC G AGGCC | 1772 | GGCCU UGAUGGCAUGCACUAUGCGCG GGAGGGCAGA | 4551 |
| 3265 | CCGAGGCCGU G CAGUG | 1773 | CACUG UGAUGGCAUGCACUAUGCGCG ACGGCCUCGG | 4552 |
| 3274 | UGCAGUGGCU G UGCCA | 1774 | UGGCA UGAUGGCAUGCACUAUGCGCG AGCCACUGCA | 4553 |
| 3276 | CAGUGGCUGU G CCACC | 1775 | GGUGG UGAUGGCAUGCACUAUGCGCG ACAGCCACUG | 4554 |
| 3292 | AAGCAUUCCU G CUCAA | 1776 | UUGAG UGAUGGCAUGCACUAUGCGCG AGGAAUGCUU | 4555 |
| 3301 | UGCUCAAGCU G ACUCG | 1777 | CGAGU UGAUGGCAUGCACUAUGCGCG AGCUUGAGCA | 4556 |
| 3306 | AAGCUGACUC G ACACC | 1778 | GGUGU UGAUGGCAUGCACUAUGCGCG GAGUCAGCUU | 4557 |
| 3314 | UCGACACCGU G UCACC | 1779 | GGUGA UGAUGGCAUGCACUAUGCGCG ACGGUGUCGA | 4558 |
| 3325 | UCACCUACGU G CCACU | 1780 | AGUGG UGAUGGCAUGCACUAUGCGCG ACGUAGGUGA | 4559 |
| 3358 | CAGCCCAGAC G CAGCU | 1781 | AGCUG UGAUGGCAUGCACUAUGCGCG GUCUGGGCUG | 4560 |
| 3364 | AGACGCAGCU G AGUCG | 1782 | CGACU UGAUGGCAUGCACUAUGCGCG AGCUGCGUCU | 4561 |
| 3385 | UCCCGGGGAC G ACGCU | 1783 | AGCGU UGAUGGCAUGCACUAUGCGCG GUCCCCGGGA | 4562 |
| 3388 | CGGGGACGAC G CUGAC | 1784 | GUCAG UGAUGGCAUGCACUAUGCGCG GUCGUCCCCG | 4563 |
| 3391 | GGACGACGCU G ACUGC | 1785 | GCAGU UGAUGGCAUGCACUAUGCGCG AGCGUCGUCC | 4564 |
| 3395 | GACGCUGACU G CCCUG | 1786 | CAGGG UGAUGGCAUGCACUAUGCGCG AGUCAGCGUC | 4565 |
| 3407 | CCUGGAGGCC G CAGCC | 1787 | GGCUG UGAUGGCAUGCACUAUGCGCG GGCCUCCAGG | 4566 |
| 3424 | ACCCGGCACU G CCCUC | 1788 | GAGGG UGAUGGCAUGCACUAUGCGCG AGUGCCGGGU | 4567 |
| 3453 | AUCCUGGACU G AUGGC | 1789 | GCCAU UGAUGGCAUGCACUAUGCGCG AGUCCAGGAU | 4568 |
| 3464 | AUGGCCACCC G CCCAC | 1790 | GUGGG UGAUGGCAUGCACUAUGCGCG GGGUGGCCAU | 4569 |
| 3479 | CAGCCAGGCC G AGAGC | 1791 | GCUCU UGAUGGCAUGCACUAUGCGCG GGCCUGGCUG | 4570 |
| 3501 | CAGCAGCCCU G UCACG | 1792 | CGUGA UGAUGGCAUGCACUAUGCGCG AGGGCUGCUG | 4571 |
| 3506 | GCCCUGUCAC G CCGGG | 1793 | CCCGG UGAUGGCAUGCACUAUGCGCG GUGACAGGGC | 4572 |
| 3554 | ACCCAGGCCC G CACCG | 1794 | CGGUG UGAUGGCAUGCACUAUGCGCG GGGCCUGGGU | 4573 |
| 3559 | GGCCCGCACC G CUGGG | 1795 | CCCAG UGAUGGCAUGCACUAUGCGCG GGUGCGGGCC | 4574 |
| 3570 | CUGGGAGUCU G AGGCC | 1796 | GGCCU UGAUGGCAUGCACUAUGCGCG AGACUCCCAG | 4575 |
| 3577 | UCUGAGGCCU G AGUGA | 1797 | UCACU UGAUGGCAUGCACUAUGCGCG AGGCCUCAGA | 4576 |
| 3581 | AGGCCUGAGU G AGUGU | 1798 | ACACU UGAUGGCAUGCACUAUGCGCG ACUCAGGCCU | 4577 |
| 3585 | CUGAGUGAGU G UUUGG | 1799 | CCAAA UGAUGGCAUGCACUAUGCGCG ACUCACUCAG | 4578 |
| 3593 | GUGUUUGGCC G AGGCC | 1800 | GGCCU UGAUGGCAUGCACUAUGCGCG GGCCAAACAC | 4579 |
| 3600 | GCCGAGGCCU G CAUGU | 1801 | ACAUG UGAUGGCAUGCACUAUGCGCG AGGCCUCGGC | 4580 |
| 3604 | AGGCCUGCAU G UCCGG | 1802 | CCGGA UGAUGGCAUGCACUAUGCGCG AUGCAGGCCU | 4581 |
| 3612 | AUGUCCGGCU C AAGGC | 1803 | GCCUU UGAUGGCAUGCACUAUGCGCG AGCCGGACAU | 4582 |
| 3619 | GCUGAAGGCU C AGUGU | 1804 | ACACU UGAUGGCAUGCACUAUGCGCG AGCCUUCAGC | 4583 |
| 3623 | AAGGCUGAGU G UCCGG | 1805 | CCGGA UGAUGGCAUGCACUAUGCGCG ACUCAGCCUU | 4584 |
| 3631 | GUGUCCGGCU G AGGCC | 1806 | GGCCU UGAUGGCAUGCACUAUGCGCG AGCCGGACAC | 4585 |
| 3638 | GCUGAGGCCU G AGCGA | 1807 | UCGCU UGAUGGCAUGCACUAUGCGCG AGGCCUCAGC | 4586 |
| 3642 | AGGCCUGAGC G AGUGU | 1808 | ACACU UGAUGGCAUGCACUAUGCGCG GCUCAGGCCU | 4587 |
| 3646 | CUGAGCGAGU G UCCAG | 1809 | CUGGA UGAUGGCAUGCACUAUGCGCG ACUCGCUCAG | 4588 |
| 3661 | GCCAAGGGCU G AGUGU | 1810 | ACACU UGAUGGCAUGCACUAUGCGCG AGCCCUUGGC | 4589 |
| 3665 | AGGGCUGAGU G UCCAG | 1811 | CUGGA UGAUGGCAUGCACUAUGCGCG ACUCAGCCCU | 4590 |
| 3678 | CAGCACACCU G CCGUC | 1812 | GACGG UGAUGGCAUGCACUAUGCGCG AGGUGUGCUG | 4591 |
| 3705 | ACAGGCUGGC G CUCGG | 1813 | CCGAG UGAUGGCAUGCACUAUGCGCG GCCAGCCUGU | 4592 |
| 3789 | CCCCAGAUUC G CCAUU | 1814 | AAUGG UGAUGGCAUGCACUAUGCGCG GAAUCUGGGG | 4593 |
| 3795 | AUUCGCCAUU G UUCAC | 1815 | GUGAA UGAUGGCAUGCACUAUGCGCG AAUGGCGAAU | 4594 |
| 3806 | UUCACCCCUC G CCCUG | 1816 | CAGGG UGAUGGCAUGCACUAUGCGCG GAGGGGUGAA | 4595 |
| 3811 | CCCUCGCCCU C CCCUC | 1817 | GAGGG UGAUGGCAUGCACUAUGCGCG AGGGCGAGGG | 4596 |
| 3821 | GCCCUCCUUU G CCUUC | 1818 | GAAGG UGAUGGCAUGCACUAUGCGCG AAAGGAGGGC | 4597 |
| 3854 | UGGAGACCCU G AGAAG | 1819 | CUUCU UGAUGGCAUGCACUAUGCGCG AGGGUCUCCA | 4598 |
| 3888 | AAUUUGGAGU G ACCAA | 1820 | UUGGU UGAUGGCAUGCACUAUGCGCG ACUCCAAAUU | 4599 |
| 3898 | GACCAAAGGU G UGCCC | 1821 | GGGCA UGAUGGCAUGCACUAUGCGCG ACCUUUGGUC | 4600 |
| 3900 | CCAAAGGUGU G CCCUG | 1822 | CAGGG UGAUGGCAUGCACUAUGCGCG ACACCUUUGG | 4601 |
| 3905 | GGUGUGCCCU G UACAC | 1823 | GUGUA UGAUGGCAUGCACUAUGCGCG AGGGCACACC | 4602 |
| 3915 | GUACACAGGC G AGGAC | 1824 | GUCCU UGAUGGCAUGCACUAUGCGCG GCCUGUGUAC | 4603 |
| 3924 | CGAGGACCCU G CACCU | 1825 | AGGUG UGAUGGCAUGCACUAUGCGCG AGGGUCCUCG | 4604 |
| 3944 | GGGGGUCCCU G UGGGU | 1826 | ACCCA UGAUGGCAUGCACUAUGCGCG AGGGACCCCC | 4605 |
| 3966 | GGGGGGAGGU G CUGUG | 1827 | CACAG UGAUGGCAUGCACUAUGCGCG ACCUCCCCCC | 4606 |
| 3969 | GGGAGGUGCU G UGGGA | 1828 | UCCCA UGAUGGCAUGCACUAUGCGCG AGCACCUCCC | 4607 |
| 3985 | GUAAAAUACU G AAUAU | 1829 | AUAUU UGAUGGCAUGCACUAUGCGCG AGUAUUUUAC | 4608 |
| 3993 | CUGAAUAUAU G AGUUU | 1830 | AAACU UGAUGGCAUGCACUAUGCGCG AUAUAUUCAG | 4609 |
| 4008 | UUUCAGUUUU G AAAAA | 1831 | UUUUU UGAUGGCAUGCACUAUGCGCG AAAACUGAAA | 4610 |

Seq1 = TERT (Homo sapiens telomerase reverse transcriptase (TERT) mRNA, 4015 bp); Nakamura et al., Science 277 (5328), 955-959 (1997)
Input Sequence = TERT. Cut Site = YG/M or UG/U.
Stem Length = 5/10. Core Sequence = UGAUG GCAUGCACUAUGC GCG

TABLE VI

Human telomerase reverse transcriptase (TERT) DNAzyme and Target
Sequence

| | | | | |
|---|---|---|---|---|
| nt. | | Seq. ID | | Seq. ID |
| Position | DNAzyme Sequence | Nos | Substrate | Nos |

| | | | | |
|---|---|---|---|---|
| 9 | CAGGACGC GGCTAGCTACAACGA AGCGCTGC | 1832 | GCAGCGCU G GCGUCCUG | 4611 |
| 11 | AGCAGGAC GGCTAGCTACAACGA GCAGCGCT | 1833 | AGCGCUGC G GUCCUGCU | 4612 |
| 16 | TGCGCAGC GGCTAGCTACAACGA AGGACGCA | 1834 | UGCGUCCU G GCUGCGCA | 4613 |
| 19 | ACGTGCGC GGCTAGCTACAACGA AGCAGGAC | 1835 | GUCCUGCU G GCGCACGU | 4614 |
| 21 | CCACGTGC GGCTAGCTACAACGA GCAGCAGG | 1836 | CCUGCUGC G GCACGUGG | 4615 |
| 23 | TCCCACGT GGCTAGCTACAACGA GCGCAGCA | 1837 | UGCUGCGC A ACGUGGGA | 4616 |
| 25 | CTTCCCAC GGCTAGCTACAACGA GTGCGCAG | 1838 | CUGCGCAC G GUGGGAAG | 4617 |
| 32 | GCCAGGGC GGCTAGCTACAACGA TTCCCACG | 1839 | CGUGGGAA G GCCCUGGC | 4618 |
| 38 | GCCGGGGC GGCTAGCTACAACGA CAGGGCTT | 1840 | AAGCCCUG G GCCCCGGC | 4619 |
| 44 | GGGGTGGC GGCTAGCTACAACGA CGGGGCCA | 1841 | UGGCCCCG G GCCACCCC | 4620 |
| 47 | GCGGGGGT GGCTAGCTACAACGA GGCCGGGG | 1842 | CCCCGGCC A ACCCCCGC | 4621 |
| 53 | GGCATCGC GGCTAGCTACAACGA GGGGGTGG | 1843 | CCACCCCC G GCGAUGCC | 4622 |
| 56 | CGCGGCAT GGCTAGCTACAACGA CGCGGGGG | 1844 | CCCCCGCG A AUGCCGCG | 4623 |
| 58 | CGCGCGGC GGCTAGCTACAACGA ATCGCGGG | 1845 | CCCGCGAU G GCCGCGCG | 4624 |
| 61 | GAGCGCGC GGCTAGCTACAACGA GGCATCGC | 1846 | GCGAUGCC G GCGCGCUC | 4625 |
| 63 | GGGAGCGC GGCTAGCTACAACGA GCGGCATC | 1847 | GAUGCCGC G GCGCUCCC | 4626 |
| 65 | CGGGGAGC GGCTAGCTACAACGA GCGCGGCA | 1848 | UGCCGCGC G GCUCCCCG | 4627 |
| 72 | TCGGCAGC GGCTAGCTACAACGA GGGGAGCG | 1849 | CGCUCCCC G GCUGCCGA | 4628 |
| 75 | GGCTCGGC GGCTAGCTACAACGA AGCGGGGA | 1850 | UCCCCGCU G GCCGAGCC | 4629 |
| 80 | CGCACGGC GGCTAGCTACAACGA TCGGCAGC | 1851 | GCUGCCGA G GCCGUGCG | 4630 |
| 83 | GAGCGCAC GGCTAGCTACAACGA GGCTCGGC | 1852 | GCCGAGCC G GUGCGCUC | 4631 |
| 85 | GGGAGCGC GGCTAGCTACAACGA ACGGCTCG | 1853 | CGAGCCGU G GCGCUCCC | 4632 |
| 87 | CAGGGAGC GGCTAGCTACAACGA GCACGGCT | 1854 | AGCCGUGC G GCUCCCUG | 4633 |
| 94 | TGCGCAGC GGCTAGCTACAACGA AGGGAGCG | 1855 | CGCUCCCU G GCUGCGCA | 4634 |
| 97 | GGCTGCGC GGCTAGCTACAACGA AGCAGGGA | 1856 | UCCCUGCU G GCGCAGCC | 4635 |
| 99 | GTGGCTGC GGCTAGCTACAACGA GCAGCAGG | 1857 | CCUGCUGC G GCAGCCAC | 4636 |
| 102 | GTAGTGGC GGCTAGCTACAACGA TGCGCAGC | 1858 | GCUGCGCA G GCCACUAC | 4637 |
| 105 | GCGGTAGT GGCTAGCTACAACGA GGCTGCGC | 1859 | GCGCAGCC A ACUACCGC | 4638 |
| 108 | CTCGCGGT GGCTAGCTACAACGA AGTGGCTG | 1860 | CAGCCACU A ACCGCGAG | 4639 |
| 111 | CACCTCGC GGCTAGCTACAACGA GGTAGTGG | 1861 | CCACUACC G GCGAGGUG | 4640 |
| 116 | GGCAGCAC GGCTAGCTACAACGA CTCGCGGT | 1862 | ACCGCGAG G GUGCUGCC | 4641 |
| 118 | GCGGCAGC GGCTAGCTACAACGA ACCTCGCG | 1863 | CGCGAGGU G GCUGCCGC | 4642 |
| 121 | CCAGCGGC GGCTAGCTACAACGA AGCACCTC | 1864 | GAGGUGCU G GCCGCUGG | 4643 |
| 124 | TGGCCAGC GGCTAGCTACAACGA GGCAGCAC | 1865 | GUGCUGCC G GCUGGCCA | 4644 |
| 128 | AACGTGGC GGCTAGCTACAACGA CAGCGGCA | 1866 | UGCCGCUG G GCCACGUU | 4645 |
| 131 | ACGAACGT GGCTAGCTACAACGA GGCCAGCG | 1867 | CGCUGGCC A ACGUUCGU | 4646 |
| 133 | GCACGAAC GGCTAGCTACAACGA GTGGCCAG | 1868 | CUGGCCAC G GUUCGUGC | 4647 |
| 137 | CGCCGCAC GGCTAGCTACAACGA GAACGTGG | 1869 | CCACGUUC G GUGCGGCG | 4648 |
| 139 | GGCGCCGC GGCTAGCTACAACGA ACGAACGT | 1870 | ACGUUCGU G GCGGCGCC | 4649 |
| 142 | CCAGGCGC GGCTAGCTACAACGA CGCACGAA | 1871 | UUCGUGCG G GCGCCUGG | 4650 |
| 144 | CCCCAGGC GGCTAGCTACAACGA GCCGCACG | 1872 | CGUGCGGC G GCCUGGGG | 4651 |
| 151 | CCTGGGGC GGCTAGCTACAACGA CCCAGGCG | 1873 | CGCCUGGG G GCCCCAGG | 4652 |
| 159 | CCGCCAGC GGCTAGCTACAACGA CCTGGGGC | 1874 | GCCCCAGG G GCUGGCGG | 4653 |
| 163 | CCAGCCGC GGCTAGCTACAACGA CAGCCCTG | 1875 | CAGGGCUG G GCGGCUGG | 4654 |
| 166 | GCACCAGC GGCTAGCTACAACGA CGCCAGCC | 1876 | GGCUGGCG G GCUGGUGC | 4655 |
| 170 | CGCTGCAC GGCTAGCTACAACGA CAGCCGCC | 1877 | GGCGGCUG G GUGCAGCG | 4656 |
| 172 | CGCGCTGC GGCTAGCTACAACGA ACCAGCCG | 1878 | CGGCUGGU G GCAGCGCG | 4657 |
| 175 | CCCCGCGC GGCTAGCTACAACGA TGCACCAG | 1879 | CUGGUGCA G GCGCGGGG | 4658 |
| 177 | GTCCCCGC GGCTAGCTACAACGA GCTGCACC | 1880 | GGUGCAGC G GCGGGGAC | 4659 |
| 183 | CGCCGGGT GGCTAGCTACAACGA CCCCGCGC | 1881 | GCGCGGGG A ACCCGGCG | 4660 |
| 188 | AAAGCCGC GGCTAGCTACAACGA CGGGTCCC | 1882 | GGGACCCG G GCGGCUUU | 4661 |
| 191 | CGGAAAGC GGCTAGCTACAACGA CGCCGGGT | 1883 | ACCCGGCG G GCUUUCCG | 4662 |
| 198 | CAGCGCGC GGCTAGCTACAACGA GGAAAGCC | 1884 | GGCUUUCC G GCGCGCUG | 4663 |
| 200 | ACCAGCGC GGCTAGCTACAACGA GCGGAAAG | 1885 | CUUUCCGC G GCGCUGGU | 4664 |
| 202 | CCACCAGC GGCTAGCTACAACGA GCGCGGAA | 1886 | UUCCGCGC G GCUGGUGG | 4665 |
| 206 | TGGGCCAC GGCTAGCTACAACGA CAGCGCGC | 1887 | GCGCGCUG G GUGGCCCA | 4666 |
| 209 | CACTGGGC GGCTAGCTACAACGA CACCAGCG | 1888 | CGCUGGUG G GCCCAGUG | 4667 |
| 214 | CCAGGCAC GGCTAGCTACAACGA TGGGCCAC | 1889 | GUGGCCCA G GUGCCUGG | 4668 |
| 216 | CACCAGGC GGCTAGCTACAACGA ACTGGGCC | 1890 | GGCCCAGU G GCCUGGUG | 4669 |
| 221 | ACGCACAC GGCTAGCTACAACGA CAGGCACT | 1891 | AGUGCCUG G GUGUGCGU | 4670 |
| 223 | GCACGCAC GGCTAGCTACAACGA ACCAGGCA | 1892 | UGCCUGGU G GUGCGUGC | 4671 |
| 225 | GGGCACGC GGCTAGCTACAACGA ACACCAGG | 1893 | CCUGGUGU G GCGUGCCC | 4672 |
| 227 | CAGGGCAC GGCTAGCTACAACGA GCACACCA | 1894 | UGGUGUGC G GUGCCCUG | 4673 |
| 229 | CCCAGGGC GGCTAGCTACAACGA ACGCACAC | 1895 | GUGUGCGU G GCCCUGGG | 4674 |
| 237 | CCGTGCGT GGCTAGCTACAACGA CCCAGGGC | 1896 | GCCCUGGG A ACGCACGG | 4675 |
| 239 | GGCCGTGC GGCTAGCTACAACGA GTCCCAGG | 1897 | CCUGGGAC G GCACGGCC | 4676 |
| 241 | GCGGCCGT GGCTAGCTACAACGA GCGTCCCA | 1898 | UGGGACGC A ACGGCCGC | 4677 |
| 244 | GGGGCGGC GGCTAGCTACAACGA CGTGCGTC | 1899 | GACGCACG G GCCGCCCC | 4678 |
| 247 | CGGGGGGC GGCTAGCTACAACGA GGCCGTGC | 1900 | GCACGGCC G GCCCCCCG | 4679 |
| 254 | GGGGCGGC GGCTAGCTACAACGA GGGGGGCG | 1901 | CGCCCCCC G GCCGCCCC | 4680 |
| 257 | GAGGGGGC GGCTAGCTACAACGA GGCGGGGG | 1902 | CCCCCGCC G GCCCCCUC | 4681 |
| 270 | CACCTGGC GGCTAGCTACAACGA GGAAGGAG | 1903 | CUCCUUCC G GCCAGGUG | 4682 |
| 275 | CAGGACAC GGCTAGCTACAACGA CTGGCGGA | 1904 | UCCGCCAG G GUGUCCUG | 4683 |
| 277 | GGCAGGAC GGCTAGCTACAACGA ACCTGGCG | 1905 | CGCCAGGU G GUCCUGCC | 4684 |
| 282 | CTTCAGGC GGCTAGCTACAACGA AGGACACC | 1906 | GGUGUCCU G GCCUGAAG | 4685 |
| 292 | CCACCAGC GGCTAGCTACAACGA TCCTTCAG | 1907 | CUGAAGGA G GCUGGUGG | 4686 |
| 296 | CGGGCCAC GGCTAGCTACAACGA CAGCTCCT | 1908 | AGGAGCUG G GUGGCCCG | 4687 |
| 299 | ACTCGGGC GGCTAGCTACAACGA CACCAGCT | 1909 | ACCUGGUG G GCCCGAGU | 4688 |
| 305 | TGCAGCAC GGCTAGCTACAACGA TCGGGCCA | 1910 | UGGCCCGA G GUGCUGCA | 4689 |
| 307 | TCTGCAGC GGCTAGCTACAACGA ACTCGGGC | 1911 | GCCCGAGU G GCUGCAGA | 4690 |
| 310 | GCCTCTGC GGCTAGCTACAACGA AGCACTCG | 1912 | CGAGUGCU G GCAGAGGC | 4691 |
| 316 | CGCACAGC GGCTAGCTACAACGA CTCTGCAG | 1913 | CUGCAGAG G GCUGUGCG | 4692 |
| 319 | GCTCGCAC GGCTAGCTACAACGA AGCCTCTG | 1914 | CAGAGGCU G GUGCGAGC | 4693 |
| 321 | GCGCTCGC GGCTAGCTACAACGA ACAGCCTC | 1915 | GAGGCUGU G GCGAGCGC | 4694 |
| 325 | CGCCGCGC GGCTAGCTACAACGA TCGCACAG | 1916 | CUGUGCGA G GCGCGGCG | 4695 |
| 327 | CGCGCCGC GGCTAGCTACAACGA GCTCGCAC | 1917 | GUGCGAGC G GCGGCGCG | 4696 |
| 330 | CTTCGCGC GGCTAGCTACAACGA CGCGCTCG | 1918 | CGAGCGCG G GCGCGAAG | 4697 |
| 332 | TTCTTCGC GGCTAGCTACAACGA GCCGCGCT | 1919 | AGCGCGGC G GCGAAGAA | 4698 |
| 339 | CAGCACGT GGCTAGCTACAACGA TCTTCGCG | 1920 | CGCGAAGA A ACGUGCUG | 4699 |
| 341 | GCCAGCAC GGCTAGCTACAACGA GTTCTTCG | 1921 | CGAAGAAC G GUGCUGGC | 4700 |
| 343 | AGGCCAGC GGCTAGCTACAACGA ACGTTCTT | 1922 | AAGAACGU G GCUGGCCU | 4701 |
| 347 | CCGAAGGC GGCTAGCTACAACGA CAGCACGT | 1923 | ACGUGCUG G GCCUUCGG | 4702 |
| 354 | CGCGAAGC GGCTAGCTACAACGA CGAAGGCC | 1924 | GGCCUUCG G GCUUCGCG | 4703 |
| 359 | AGCAGCGC GGCTAGCTACAACGA GAAGCCGA | 1925 | UCGGCUUC G GCGCUGCU | 4704 |
| 361 | CCAGCAGC GGCTAGCTACAACGA GCGAAGCC | 1926 | GGCUUCGC G GCUGCUGG | 4705 |
| 364 | CGTCCAGC GGCTAGCTACAACGA AGCGCGAA | 1927 | UUCGCGCU G GCUGGACG | 4706 |
| 369 | GGCCCCGT GGCTAGCTACAACGA CCAGCAGC | 1928 | GCUGCUGG A ACGGGGCC | 4707 |
| 374 | CCGCGGGC GGCTAGCTACAACGA CCCGTCCA | 1929 | UGGACGGG G GCCCGCGG | 4708 |
| 378 | GCCCCCGC GGCTAGCTACAACGA GGGCCCCG | 1930 | CGGGGCCC G GCGGGGGC | 4709 |
| 384 | GGGGGGGC GGCTAGCTACAACGA CCCCGCGG | 1931 | CCGCGGGG G GCCCCCCC | 4710 |
| 395 | GTGAAGGC GGCTAGCTACAACGA CTCGGGGG | 1932 | CCCCCGAG G GCCUUCAC | 4711 |
| 401 | CTGGTGGT GGCTAGCTACAACGA GAAGGCCT | 1933 | AGGCCUUC A ACCACCAG | 4712 |
| 404 | ACGCTGGT GGCTAGCTACAACGA GGTGAAGG | 1934 | CCUUCACC A ACCAGCGU | 4713 |
| 408 | GCGCACGC GGCTAGCTACAACGA TGGTGGTG | 1935 | CACCACCA G GCGUGCGC | 4714 |
| 410 | CTGCGCAC GGCTAGCTACAACGA GCTGGTGG | 1936 | CCACCAGC G GUGCGCAG | 4715 |
| 412 | AGCTGCGC GGCTAGCTACAACGA ACGCTGGT | 1937 | ACCAGCGU G GCGCAGCU | 4716 |
| 414 | GTAGCTGC GGCTAGCTACAACGA GCACGCTG | 1938 | CAGCGUGC G GCAGCUAC | 4717 |
| 417 | CAGGTAGC GGCTAGCTACAACGA TGCGCACG | 1939 | CGUGCGCA G GCUACCUG | 4718 |
| 420 | GGGCAGGT GGCTAGCTACAACGA AGCTGCGC | 1940 | GCGCAGCU A ACCUGCCC | 4719 |
| 424 | TGTTGGGC GGCTAGCTACAACGA AGGTAGCT | 1941 | AGCUACCU G GCCCAACA | 4720 |
| 429 | CACCGTGT GGCTAGCTACAACGA TGGGCAGG | 1942 | CCUGCCCA A ACACGGUG | 4721 |
| 431 | GTCACCGT GGCTAGCTACAACGA GTTGGGCA | 1943 | UGCCCAAC A ACGGUGAC | 4722 |
| 434 | TCGGTCAC GGCTAGCTACAACGA CGTGTTGG | 1944 | CCAACACG G GUGACCGA | 4723 |
| 437 | GCGTCGGT GGCTAGCTACAACGA CACCGTGT | 1945 | ACACGGUG A ACCGACGC | 4724 |
| 441 | CAGTGCGT GGCTAGCTACAACGA CGGTCACC | 1946 | GGUGACCG A ACGCACUG | 4725 |
| 443 | CGCAGTGC GGCTAGCTACAACGA GTCGGTCA | 1947 | UGACCGAC G GCACUGCG | 4726 |
| 445 | CCCGCAGT GGCTAGCTACAACGA GCGTCGGT | 1948 | ACCGACGC A ACUGCGGG | 4727 |
| 448 | TCCCCCGC GGCTAGCTACAACGA AGTGCGTC | 1949 | GACGCACU G GCGGGGGA | 4728 |
| 456 | CGCCCCGC GGCTAGCTACAACGA TCCCCCGC | 1950 | GCGGGGGA G GCGGGGCG | 4729 |
| 461 | CCCCACGC GGCTAGCTACAACGA CCCGCTCC | 1951 | GGAGCGGG G GCGUGGGG | 4730 |
| 463 | GCCCCCAC GGCTAGCTACAACGA GCCCCGCT | 1952 | AGCGGGGC G GUGGGGGC | 4731 |
| 469 | GCAGCAGC GGCTAGCTACAACGA CCCCACGC | 1953 | GCGUGGGG G GCUGCUGC | 4732 |
| 472 | GCAGCAGC GGCTAGCTACAACGA AGCCCCCA | 1954 | UGGGGGCU G GCUGCUGC | 4733 |
| 475 | GGCGCAGC GGCTAGCTACAACGA AGCAGCCC | 1955 | GGGCUGCU G GCUGCGCC | 4734 |
| 478 | CGCGGCGC GGCTAGCTACAACGA AGCAGCAG | 1956 | CUGCUGCU G GCGCCGCG | 4735 |
| 480 | CACGCGGC GGCTAGCTACAACGA GCAGCAGC | 1957 | GCUGCUGC G GCCGCGUG | 4736 |
| 483 | GCCCACGC GGCTAGCTACAACGA GGCGCAGC | 1958 | GCUGCGCC G GCGUGGGC | 4737 |
| 485 | TCGCCCAC GGCTAGCTACAACGA GCGGCGCA | 1959 | UGCGCCGC G GUGGGCGA | 4738 |
| 489 | GTCGTCGC GGCTAGCTACAACGA CCACGCGG | 1960 | CCGCGUGG G GCGACGAC | 4739 |
| 492 | CACGTCGT GGCTAGCTACAACGA CGCCCACG | 1961 | CGUGGGCG A ACGACGUG | 4740 |
| 495 | CAGCACGT GGCTAGCTACAACGA CGTCGCCC | 1962 | GGGCGACG A ACGUGCUG | 4741 |
| 497 | ACCAGCAC GGCTAGCTACAACGA GTCGTCGC | 1963 | GCGACGAC C GUGCUGGU | 4742 |
| 499 | GAACCAGC GGCTAGCTACAACGA ACGTCGTC | 1964 | GACGACGU G GCUGGUUC | 4743 |
| 503 | AGGTGAAC GGCTAGCTACAACGA CAGCACGT | 1965 | ACGUGCUG G GUUCACCU | 4744 |
| 507 | CAGCAGGT GGCTAGCTACAACGA GAACCAGC | 1966 | GCUGGUUC A ACCUGCUG | 4745 |
| 511 | GTGCCAGC GGCTAGCTACAACGA AGGTGAAC | 1967 | GUUCACCU C GCUGGCAC | 4746 |
| 515 | CAGCGTGC GGCTAGCTACAACGA CAGCAGGT | 1968 | ACCUCCUG G GCACGCUG | 4747 |
| 517 | CGCAGCGT GGCTAGCTACAACGA GCCAGCAG | 1969 | CUGCUGGC A ACGCUGCG | 4748 |
| 519 | CGCGCAGC GGCTAGCTACAACGA GTGCCAGC | 1970 | GCUGGCAC G GCUGCGCG | 4749 |
| 522 | GAGCGCGC GGCTAGCTACAACGA AGCGTGCC | 1971 | GGCACGCU G GCGCGCUC | 4750 |
| 524 | AAGAGCGC GGCTAGCTACAACGA GCAGCGTG | 1972 | CACGCUGC G GCGCUCUU | 4751 |
| 526 | CAAAGAGC GGCTAGCTACAACGA GCGCAGCG | 1973 | CGCUGCGC C GCUCUUUG | 4752 |
| 533 | ACCAGCAC GGCTAGCTACAACGA AAAGAGCG | 1974 | CGCUCUUU G GUGCUGGU | 4753 |
| 535 | CCACCAGC GGCTAGCTACAACGA ACAAAGAG | 1975 | CUCUUUGU C GCUGGUGG | 4754 |
| 539 | GGAGCCAC GGCTAGCTACAACGA CAGCACAA | 1976 | UUGUGCUG C CUGGCUCC | 4755 |
| 542 | CTGGCAGC GGCTAGCTACAACGA CACCAGCA | 1977 | UCCUGGUG G CCUCCCAG | 4756 |
| 549 | GGCGCAGC GGCTAGCTACAACGA TGGGAGCC | 1978 | GGCUCCCA G GCUGCGCC | 4757 |
| 552 | GTAGGCGC GGCTAGCTACAACGA AGCTGGGA | 1979 | UCCCACCU G GCCCCUAC | 4758 |
| 554 | TGGTAGGC GGCTAGCTACAACGA GCAGCTGG | 1980 | CCAGCUGC G GCCUACCA | 4759 |
| 558 | CACCTGGT GGCTAGCTACAACGA AGGCGCAG | 1981 | CUGCGCCU A ACCAGGUG | 4760 |
| 563 | CCGCACAC GGCTAGCTACAACGA CTGGTAGG | 1982 | CCUACCAG G GUGUGCGG | 4761 |
| 565 | GCCCGCAC GGCTAGCTACAACGA ACCTGGTA | 1983 | UACCAGGU G GUGCGGGC | 4762 |
| 567 | CGGCCCGC GGCTAGCTACAACGA ACACCTGG | 1984 | CCAGGUGU G GCGGGCCG | 4763 |
| 571 | GCGGCGGC GGCTAGCTACAACGA CCGCACAC | 1985 | GUGUGCGG G GCCGCCGC | 4764 |
| 574 | ACAGCGGC GGCTAGCTACAACGA GGCCCGCA | 1986 | UGCGGGCC G GCCGCUGU | 4765 |
| 577 | GGTACAGC GGCTAGCTACAACGA GGCGGCCC | 1987 | GGGCCGCC G GCUGUACC | 4766 |
| 580 | GCTGGTAC GGCTAGCTACAACGA AGCGGCGG | 1988 | CCGCCGCU G GUACCAGC | 4767 |
| 582 | GAGCTGGT GGCTAGCTACAACGA ACAGCGGC | 1989 | GCCGCUGU A ACCAGCUC | 4768 |
| 586 | CGCCGAGC GGCTAGCTACAACGA TGGTACAG | 1990 | CUGUACCA G GCUCGGCG | 4769 |
| 591 | GGCAGCGC GGCTAGCTACAACGA CGAGCTGG | 1991 | CCAGCUCG G GCGCUGCC | 4770 |
| 593 | GTGGCAGC GGCTAGCTACAACGA GCCGAGCT | 1992 | AGCUCGGC G GCUGCCAC | 4771 |
| 596 | TGAGTGGC GGCTAGCTACAACGA AGCGCCGA | 1993 | UCGGCGCU G GCCACUCA | 4772 |
| 599 | GCCTGAGT GGCTAGCTACAACGA GGCAGCGC | 1994 | GCGCUGCC A ACUCAGGC | 4773 |
| 605 | GGCCGGGC GGCTAGCTACAACGA CTGAGTGG | 1995 | CCACUCAG G GCCCGGCC | 4774 |
| 610 | GCGGGGGC GGCTAGCTACAACGA CGGGCCTG | 1996 | CAGGCCCG G GCCCCCGC | 4775 |
| 616 | CGTGTGGC GGCTAGCTACAACGA GGGGGCCG | 1997 | CGGCCCCC G GCCACACG | 4776 |
| 619 | TAGCGTGT GGCTAGCTACAACGA GGCGGGGG | 1998 | CCCCCGCC A ACACGCUA | 4777 |
| 621 | ACTAGCGT GGCTAGCTACAACGA GTGGCGGG | 1999 | CCCGCCAC A ACGCUAGU | 4778 |
| 623 | CCACTAGC GGCTAGCTACAACGA GTGTGGCG | 2000 | CGCCACAC G GCUAGUGG | 4779 |
| 627 | GGGTCCAC GGCTAGCTACAACGA TAGCGTGT | 2001 | ACACGCUA G GUGGACCC | 4780 |
| 631 | TTCGGGGT GGCTAGCTACAACGA CCACTAGC | 2002 | GCUACUGG A ACCCCGAA | 4781 |
| 640 | CCACACGC GGCTAGCTACAACGA CTTCGGGG | 2003 | CCCCGAAG G GCGUCUGG | 4782 |
| 642 | TCCCAGAC GGCTAGCTACAACGA GCCTTCGG | 2004 | CCGAAGGC G GUCUGGGA | 4783 |
| 649 | GTTCGCAT GGCTAGCTACAACGA CCCAGACG | 2005 | CGUCUGGG A AUGCGAAC | 4784 |
| 651 | CCGTTCGC GGCTAGCTACAACGA ATCCCAGA | 2006 | UCUGGGAU G GCGAACGG | 4785 |
| 655 | AGGCCCGT GGCTAGCTACAACGA TCGCATCC | 2007 | GGAUGCGA A ACGGGCCU | 4786 |
| 659 | TTCCAGGC GGCTAGCTACAACGA CCGTTCGC | 2008 | GCGAACGG G GCCUGGAA | 4787 |
| 666 | GCTATGGT GGCTAGCTACAACGA TCCAGGCC | 2009 | GGCCUGGA A ACCAUAGC | 4788 |
| 669 | GACGCTAT GGCTAGCTACAACGA GGTTCCAG | 2010 | CUGGAACC A AUAGCGUC | 4789 |
| 672 | CCTGACGC GGCTAGCTACAACGA TATGGTTC | 2011 | GAACCAUA G GCGUCAGG | 4790 |
| 674 | TCCCTGAC GGCTAGCTACAACGA GCTATGGT | 2012 | ACCAUAGC G GUCAGGGA | 4791 |
| 683 | ACCCCGGC GGCTAGCTACAACGA CTCCCTGA | 2013 | UCAGGGAG G GCCGGGGU | 4792 |
| 689 | AGGGGGAC GGCTAGCTACAACGA CCCGGCCT | 2014 | AGGCCGGG G GUCCCCCU | 4793 |
| 699 | TGGCAGGC GGCTAGCTACAACGA CCAGGGGG | 2015 | CCCCCUGG G GCCUGCCA | 4794 |
| 703 | GGGCTGGC GGCTAGCTACAACGA AGGCCCAG | 2016 | CUGGGCCU G GCCAGCCC | 4795 |
| 707 | CCCGGGGC GGCTAGCTACAACGA TGGCAGGC | 2017 | GCCUGCCA G GCCCCGGG | 4796 |
| 714 | CCTCGCAC GGCTAGCTACAACGA CCGGGGCT | 2018 | AGCCCCGG G GUGCGAGG | 4797 |
| 716 | CTCCTCGC GGCTAGCTACAACGA ACCCGGGG | 2019 | CCCCGGGU G GCGAGGAG | 4798 |
| 724 | CCCCGCGC GGCTAGCTACAACGA CTCCTCGC | 2020 | GCGAGGAG G GCGCGGGG | 4799 |
| 726 | GCCCCCGC GGCTAGCTACAACGA GCCTCCTC | 2021 | GAGGAGGC G GCGGGGGC | 4800 |
| 732 | GGCACTGC GGCTAGCTACAACGA CCCCGCGC | 2022 | GCGCGGGG G GCAGUGCC | 4801 |
| 735 | GCTGGCAC GGCTAGCTACAACGA TGCCCCCG | 2023 | CGGGGGCA G GUGCCAGC | 4802 |
| 737 | CGGCTGGC GGCTAGCTACAACGA ACTGCCCC | 2024 | GGGGCAGU G GCCAGCCG | 4803 |
| 741 | ACTTCGGC GGCTAGCTACAACGA TGGCACTG | 2025 | CAGUGCCA G GCCGAAGU | 4804 |
| 747 | CGGCAGAC GGCTAGCTACAACGA TTCGGCTG | 2026 | CAGCCGAA G GUCUGCCG | 4805 |
| 751 | GCAACGGC GGCTAGCTACAACGA AGACTTCG | 2027 | CGAAGUCU G GCCGUUGC | 4806 |
| 754 | TGGGCAAC GGCTAGCTACAACGA GGCAGACT | 2028 | AGUCUGCC G GUUGCCCA | 4807 |
| 757 | TCTTGGGC GGCTAGCTACAACGA AACGGCAG | 2029 | CUGCCGUU G GCCCAAGA | 4808 |
| 766 | GCCTGGGC GGCTAGCTACAACGA CTCTTGGG | 2030 | CCCAAGAG G GCCCAGGC | 4809 |
| 772 | CGCCACGC GGCTAGCTACAACGA CTGGGCCT | 2031 | AGGCCCAG G GCGUGGCG | 4810 |
| 774 | AGCGCCAC GGCTAGCTACAACGA GCCTGGGC | 2032 | GCCCAGGC G GUGGCGCU | 4811 |
| 777 | GGCAGCGC GGCTAGCTACAACGA CACGCCTG | 2033 | CAGGCGUG G GCGCUGCC | 4812 |
| 779 | GGGGCAGC GGCTAGCTACAACGA GCCACGCC | 2034 | GGCGUGGC G GCUGCCCC | 4813 |
| 782 | TCAGGGGC GGCTAGCTACAACGA AGCGCCAC | 2035 | GUGGCGCU G GCCCCUGA | 4814 |
| 790 | GCTCCGGC GGCTAGCTACAACGA TCAGGGGC | 2036 | GCCCCUGA G GCCGGAGC | 4815 |
| 796 | GCGTCCGC GGCTAGCTACAACGA TCCGGCTC | 2037 | GAGCCGGA G GCGGACGC | 4816 |
| 800 | ACGGGCGT GGCTAGCTACAACGA CCGCTCCG | 2038 | CGGAGCGG A ACGCCCGU | 4817 |
| 802 | CAACGGGC GGCTAGCTACAACGA GTCCGCTC | 2039 | GAGCGGAC G GCCCGUUG | 4818 |
| 806 | TGCCCAAC GGCTAGCTACAACGA GGGCGTCC | 2040 | GGACGCCC G GUUGGGCA | 4819 |
| 811 | ACCCCTGC GGCTAGCTACAACGA CCAACGGG | 2041 | CCCGUUGG G GCAGGGGU | 4820 |
| 817 | CCCAGGAC GGCTAGCTACAACGA CCCTGCCC | 2042 | GGGCAGGG G GUCCUGGG | 4821 |
| 824 | GGGTGGGC GGCTAGCTACAACGA CCAGGACC | 2043 | GGUCCUGG G GCCCACCC | 4822 |
| 828 | GCCCGGGT GGCTAGCTACAACGA GGGCCCAG | 2044 | CUGGGCCC A ACCCGGGC | 4823 |
| 834 | CGTCCTGC GGCTAGCTACAACGA CCGGGTGG | 2045 | CCACCCGG G GCAGGACG | 4824 |
| 839 | CCACGCGT GGCTAGCTACAACGA CCTGCCCG | 2046 | CGGGCAGG A ACGCGUGG | 4825 |
| 841 | GTCCACGC GGCTAGCTACAACGA GTCCTGCC | 2047 | GGCAGGAC G GCGUGGAC | 4826 |
| 843 | CGGTCCAC GGCTAGCTACAACGA GCGTCCTG | 2048 | CAGGACGC G GUGGACCG | 4827 |
| 847 | CACTCGGT GGCTAGCTACAACGA CCACGCGT | 2049 | ACGCGUGG A ACCGAGUG | 4828 |
| 852 | ACGGTCAC GGCTAGCTACAACGA TCGGTCCA | 2050 | UGGACCGA G GUGACCGU | 4829 |
| 855 | ACCACGGT GGCTAGCTACAACGA CACTCGGT | 2051 | ACCGAGUG A ACCGUGGU | 4830 |
| 858 | GAAACCAC GGCTAGCTACAACGA GGTCACTC | 2052 | GAGUGACC G GUGGUUUC | 4831 |
| 861 | ACAGAAAC GGCTAGCTACAACGA CACGGTCA | 2053 | UGACCGUG G GUUUCUGU | 4832 |
| 867 | CACCACAC GGCTAGCTACAACGA AGAAACCA | 2054 | UGGUUUCU G GUGUGGUG | 4833 |
| 869 | GACACCAC GGCTAGCTACAACGA ACAGAAAC | 2055 | GUUUCUGU G GUGGUGUC | 4834 |
| 872 | GGTGACAC GGCTAGCTACAACGA CACACAGA | 2056 | UCUGUGUG G GUGUCACC | 4835 |
| 874 | CAGGTGAC GGCTAGCTACAACGA ACCACACA | 2057 | UGUGUGGU G GUCACCUG | 4836 |
| 877 | TGGCAGGT GGCTAGCTACAACGA GACACCAC | 2058 | GUGGUGUC A ACCUGCCA | 4837 |
| 881 | GGTCTGGC GGCTAGCTACAACGA AGGTGACA | 2059 | UGUCACCU G GCCAGACC | 4838 |
| 886 | CGGCGGGT GGCTAGCTACAACGA CTGGCAGG | 2060 | CCUGCCAG A ACCCGCCG | 4839 |
| 890 | TCTTCGGC GGCTAGCTACAACGA GGGTCTGG | 2061 | CCAGACCC G GCCGAAGA | 4840 |
| 899 | GAGGTGGC GGCTAGCTACAACGA TTCTTCGG | 2062 | CCGAAGAA G GCCACCUC | 4841 |
| 902 | AAAGAGGT GGCTAGCTACAACGA GGCTTCTT | 2063 | AAGAAGCC A ACCUCUUU | 4842 |
| 915 | GAGCGCAC GGCTAGCTACAACGA CCTCCAAA | 2064 | UUUGGAGG G GUGCGCUC | 4843 |
| 917 | GAGAGCGC GGCTAGCTACAACGA ACCCTCCA | 2065 | UGGAGGGU G GCGCUCUC | 4844 |
| 919 | CAGAGAGC GGCTAGCTACAACGA GCACCCTC | 2066 | GAGGGUGC G GCUCUCUG | 4845 |
| 927 | GCGCGTGC GGCTAGCTACAACGA CAGAGAGC | 2067 | GCUCUCUG G GCACGCGC | 4846 |
| 929 | TGGCGCGT GGCTAGCTACAACGA GCCAGAGA | 2068 | UCUCUGGC A ACGCGCCA | 4847 |
| 931 | AGTGGCGC GGCTAGCTACAACGA GTGCCAGA | 2069 | UCUGGCAC G GCGCCACU | 4848 |
| 933 | GGAGTGGC GGCTAGCTACAACGA GCGTGCCA | 2070 | UGGCACGC G GCCACUCC | 4849 |
| 936 | GTGGGAGT GGCTAGCTACAACGA GGCGCGTG | 2071 | CACGCGCC A ACUCCCAC | 4850 |
| 942 | GGATGGGT GGCTAGCTACAACGA GGGAGTGG | 2072 | CCACUCCC A ACCCAUCC | 4851 |
| 946 | CCACGGAT GGCTAGCTACAACGA GGGTGGGA | 2073 | UCCCACCC A AUCCGUGG | 4852 |
| 950 | CGGCCCAC GGCTAGCTACAACGA GGATGGGT | 2074 | ACCCAUCC G GUGGGCCG | 4853 |
| 954 | CTGGCGGC GGCTAGCTACAACGA CCACGGAT | 2075 | AUCCGUGG G GCCGCCAG | 4854 |
| 957 | GTGCTGGC GGCTAGCTACAACGA GGCCCACG | 2076 | CGUGGGCC G GCCAGCAC | 4855 |
| 961 | CGTGGTGC GGCTAGCTACAACGA TGGCGGCC | 2077 | GGCCGCCA G GCACCACG | 4856 |
| 963 | CGCGTGGT GGCTAGCTACAACGA GCTGGCGG | 2078 | CCGCCAGC A ACCACGCG | 4857 |
| 966 | GCCCGCGT GGCTAGCTACAACGA GGTGCTGG | 2079 | CCAGCACC A ACGCGGGC | 4858 |
| 968 | GGGCCCGC GGCTAGCTACAACGA GTGGTGCT | 2080 | AGCACCAC G GCGGGCCC | 4859 |
| 972 | TGGGGGGC GGCTAGCTACAACGA CCGCGTGG | 2081 | CCACGCGG G GCCCCCCA | 4860 |
| 979 | ATGTGGAT GGCTAGCTACAACGA GGGGGGCC | 2082 | GGCCCCCC A AUCCACAU | 4861 |
| 983 | CGCGATGT GGCTAGCTACAACGA GGATGGGG | 2083 | CCCCAUCC A ACAUCGCG | 4862 |
| 985 | GCCGCGAT GGCTAGCTACAACGA GTGGATGG | 2084 | CCAUCCAC A AUCGCGGC | 4863 |
| 988 | GTGGCCGC GGCTAGCTACAACGA GATGTGGA | 2085 | UCCACAUC G GCGGCCAC | 4864 |
| 991 | GTGGTGGC GGCTAGCTACAACGA CGCGATGT | 2086 | ACAUCGCG G GCCACCAC | 4865 |
| 994 | GACGTGGT GGCTAGCTACAACGA GGCCGCGA | 2087 | UCGCGGCC A ACCACGUC | 4866 |
| 997 | AGGGACGT GGCTAGCTACAACGA GGTGGCCG | 2088 | CGGCCACC A ACGUCCCU | 4867 |
| 999 | CCAGGGAC GGCTAGCTACAACGA GTGGTGGC | 2089 | GCCACCAC G GUCCCUGG | 4868 |
| 1008 | AGGCGTGT GGCTAGCTACAACGA CCCAGGGA | 2090 | UCCCUGGG A ACACGCCU | 4869 |
| 1010 | CAAGGCGT GGCTAGCTACAACGA GTCCCAGG | 2091 | CCUGGGAC A ACGCCUUG | 4870 |
| 1012 | GACAAGGC GGCTAGCTACAACGA GTGTCCCA | 2092 | UGGGACAC G GCCUUGUC | 4871 |
| 1017 | CGGGGGAC GGCTAGCTACAACGA AAGGCGTG | 2093 | CACGCCUU G GUCCCCCG | 4872 |
| 1025 | GCGTACAC GGCTAGCTACAACGA CGGGGGAC | 2094 | GUCCCCCG G GUGUACGC | 4873 |
| 1027 | CGGCGTAC GGCTAGCTACAACGA ACCGGGGG | 2095 | CCCCCGGU G GUACGCCG | 4874 |
| 1029 | CTCGGCGT GGCTAGCTACAACGA ACACCGGG | 2096 | CCCGGUGU A ACGCCGAG | 4875 |
| 1031 | GTCTCGGC GGCTAGCTACAACGA GTACACCG | 2097 | CGGUGUAC G GCCGAGAC | 4876 |
| 1037 | TGCTTGGT GGCTAGCTACAACGA CTCGGCGT | 2098 | ACGCCGAG A ACCAAGCA | 4877 |
| 1042 | GGAAGTGC GGCTAGCTACAACGA TTGGTCTC | 2099 | GAGACCAA G GCACUUCC | 4878 |
| 1044 | GAGGAAGT GGCTAGCTACAACGA GCTTGGTC | 2100 | GACCAAGC A ACUUCCUC | 4879 |
| 1053 | TGAGGAGT GGCTAGCTACAACGA AGAGGAAG | 2101 | CUUCCUCU A ACUCCUCA | 4880 |
| 1062 | CTTGTCGC GGCTAGCTACAACGA CTGAGGAG | 2102 | CUCCUCAG G GCGACAAG | 4881 |
| 1065 | CTCCTTGT GGCTAGCTACAACGA CGCCTGAG | 2103 | CUCAGGCG A ACAAGGAG | 4882 |
| 1072 | GCAGCTGC GGCTAGCTACAACGA TCCTTGTC | 2104 | GACAAGGA G GCAGCUGC | 4883 |
| 1075 | GCCGCAGC GGCTAGCTACAACGA TGCTCCTT | 2105 | AAGGAGCA G GCUGCGGC | 4884 |
| 1078 | AGGGCCGC GGCTAGCTACAACGA AGCTGCTC | 2106 | GAGCAGCU G GCGGCCCU | 4885 |
| 1081 | AGGAGGGC GGCTAGCTACAACGA CGCAGCTG | 2107 | CAGCUGCG G GCCCUCCU | 4886 |
| 1093 | AGCTGAGT GGCTAGCTACAACGA AGGAAGGA | 2108 | UCCUUCCU A ACUCAGCU | 4887 |
| 1098 | CAGAGAGC GGCTAGCTACAACGA TGAGTAGG | 2109 | CCUACUCA G GCUCUCUG | 4888 |
| 1108 | GGCTGGGC GGCTAGCTACAACGA CTCAGAGA | 2110 | UCUCUGAG G GCCCAGCC | 4889 |
| 1113 | AGTCAGGC GGCTAGCTACAACGA TGGGCCTC | 2111 | GAGGCCCA G GCCUGACU | 4890 |
| 1118 | GCGCCAGT GGCTAGCTACAACGA CAGGCTGG | 2112 | CCAGCCUG A ACUGGCGC | 4891 |
| 1122 | CCGAGCGC GGCTAGCTACAACGA CAGTCAGG | 2113 | CCUGACUG G GCGCUCGG | 4892 |
| 1124 | CTCCGAGC GGCTAGCTACAACGA GCCAGTCA | 2114 | UGACUGGC G GCUCGGAG | 4893 |
| 1132 | CCACGAGC GGCTAGCTACAACGA CTCCGAGC | 2115 | GCUCGGAG G GCUCGUGG | 4894 |
| 1136 | GTCTCCAC GGCTAGCTACAACGA GAGCCTCC | 2116 | GGAGGCUC G GUGGAGAC | 4895 |
| 1142 | AAGATGGT GGCTAGCTACAACGA CTCCACGA | 2117 | UCGUGGAG A ACCAUCUU | 4896 |
| 1145 | AGAAAGAT GGCTAGCTACAACGA GGTCTCCA | 2118 | UGGAGACC A AUCUUUCU | 4897 |
| 1155 | CCTGGAAC GGCTAGCTACAACGA CCAGAAAG | 2119 | CUUUCUGG G GUUCCAGG | 4898 |
| 1162 | TCCAGGGC GGCTAGCTACAACGA CTGGAACC | 2120 | GGUUCCAG G GCCCUGGA | 4899 |
| 1169 | CCTGGCAT GGCTAGCTACAACGA CCAGGGCC | 2121 | GGCCCUGG A AUGCCAGG | 4900 |
| 1171 | TCCCTGGC GGCTAGCTACAACGA ATCCAGGG | 2122 | CCCUGGAU G GCCAGGGA | 4901 |
| 1178 | CGGGGAGT GGCTAGCTACAACGA CCCTGGCA | 2123 | UGCCAGGG A ACUCCCCG | 4902 |
| 1185 | CAACCTGC GGCTAGCTACAACGA GGGGAGTC | 2124 | GACUCCCC G GCAGGUUG | 4903 |
| 1189 | GGGGCAAC GGCTAGCTACAACGA CTGCGGGG | 2125 | CCCCGCAG G GUUGCCCC | 4904 |
| 1192 | GGCGGGGC GGCTAGCTACAACGA AACCTGCG | 2126 | CGCAGGUU G GCCCCGCC | 4905 |
| 1197 | GGGCAGGC GGCTAGCTACAACGA GGGGCAAC | 2127 | GUUGCCCC G GCCUGCCC | 4906 |
| 1201 | GCTGGGGC GGCTAGCTACAACGA AGGCGGGG | 2128 | CCCCGCCU G GCCCCAGC | 4907 |
| 1207 | AGTAGCGC GGCTAGCTACAACGA TGGGGCAG | 2129 | CUGCCCCA G GCGCUACU | 4908 |
| 1209 | CCAGTAGC GGCTAGCTACAACGA GCTGGGGC | 2130 | GCCCCAGC G GCUACUGG | 4909 |
| 1212 | TTGCCAGT GGCTAGCTACAACGA AGCGCTGG | 2131 | CCAGCGCU A ACUGGCAA | 4910 |
| 1216 | GCATTTGC GGCTAGCTACAACGA CAGTAGCG | 2132 | CGCUACUG G GCAAAUGC | 4911 |
| 1220 | GGCCGCAT GGCTAGCTACAACGA TTGCCAGT | 2133 | ACUGGCAA A AUGCGGCC | 4912 |
| 1222 | GGGGCCGC GGCTAGCTACAACGA ATTTGCCA | 2134 | UGGCAAAU G GCGGCCCC | 4913 |
| 1225 | ACAGGGGC GGCTAGCTACAACGA CGCATTTG | 2135 | CAAAUGCG G GCCCCUGU | 4914 |
| 1231 | CCAGAAAC GGCTAGCTACAACGA AGGGGCCG | 2136 | CGGCCCCU G GUUUCUGG | 4915 |
| 1240 | CAAGCAGC GGCTAGCTACAACGA TCCAGAAA | 2137 | UUUCUGGA G GCUGCUUG | 4916 |
| 1243 | TCCCAAGC GGCTAGCTACAACGA AGCTCCAG | 2138 | CUGGAGCU G GCUUGGGA | 4917 |
| 1251 | CGCGTGGT GGCTAGCTACAACGA TCCCAAGC | 2139 | GCUUGGGA A ACCACGCG | 4918 |
| 1254 | CTGCGCGT GGCTAGCTACAACGA GGTTCCCA | 2140 | UGGGAACC A ACGCGCAG | 4919 |
| 1256 | CACTGCGC GGCTAGCTACAACGA GTGGTTCC | 2141 | GGAACCAC G GCGCAGUG | 4920 |
| 1258 | GGCACTGC GGCTAGCTACAACGA GCGTGGTT | 2142 | AACCACGC G GCAGUGCC | 4921 |
| 1261 | AGGGGCAC GGCTAGCTACAACGA TGCGCGTG | 2143 | CACGCGCA G GUGCCCCU | 4922 |
| 1263 | GTAGGGGC GGCTAGCTACAACGA ACTGCGCG | 2144 | CGCGCAGU G GCCCCUAC | 4923 |
| 1269 | CACCCCGT GGCTAGCTACAACGA AGGGGCAC | 2145 | GUGCCCCU A ACGGGGUG | 4924 |
| 1274 | AGGAGCAC GGCTAGCTACAACGA CCCGTAGG | 2146 | CCUACGGG G GUGCUCCU | 4925 |
| 1276 | TGAGGAGC GGCTAGCTACAACGA ACCCCGTA | 2147 | UACGGGGU G GCUCCUCA | 4926 |
| 1286 | CAGTGCGT GGCTAGCTACAACGA CTTGAGGA | 2148 | UCCUCAAG A ACGCACUG | 4927 |
| 1288 | GGCAGTGC GGCTAGCTACAACGA GTCTTGAG | 2149 | CUCAAGAC G GCACUGCC | 4928 |
| 1290 | CGGGCAGT GGCTAGCTACAACGA GCGTCTTG | 2150 | CAAGACGC A ACUGCCCG | 4929 |
| 1293 | CAGCGGGC GGCTAGCTACAACGA AGTGCGTC | 2151 | GACGCACU G GCCCGCUG | 4930 |
| 1297 | CTCGCAGC GGCTAGCTACAACGA GGGCAGTG | 2152 | CACUGCCC G GCUGCGAG | 4931 |
| 1300 | CAGCTCGC GGCTAGCTACAACGA AGCGGGCA | 2153 | UGCCCGCU G GCGAGCUG | 4932 |
| 1304 | ACCGCAGC GGCTAGCTACAACGA TCGCAGCG | 2154 | CGCUGCGA G GCUGCGGU | 4933 |
| 1307 | GTGACCGC GGCTAGCTACAACGA AGCTCGCA | 2155 | UGCGAGCU G GCGGUCAC | 4934 |
| 1310 | GGGGTGAC GGCTAGCTACAACGA CGCAGCTC | 2156 | GAGCUGCG G GUCACCCC | 4935 |
| 1313 | GCTGGGGT GGCTAGCTACAACGA GACCGCAG | 2157 | CUGCGGUC A ACCCCAGC | 4936 |
| 1319 | CCGGCTGC GGCTAGCTACAACGA TGGGGTGA | 2158 | UCACCCCA G GCAGCCGG | 4937 |
| 1322 | ACACCGGC GGCTAGCTACAACGA TGCTGGGG | 2159 | CCCCAGCA G GCCGGUGU | 4938 |
| 1326 | ACAGACAC GGCTAGCTACAACGA CGGCTGCT | 2160 | AGCAGCCG G GUGUCUGU | 4939 |
| 1328 | GCACAGAC GGCTAGCTACAACGA ACCGGCTG | 2161 | CAGCCGGU G GUCUGUGC | 4940 |
| 1332 | CCGGGCAC GGCTAGCTACAACGA AGACACCG | 2162 | CGGUGUCU G GUGCCCGG | 4941 |
| 1334 | TCCCGGGC GGCTAGCTACAACGA ACAGACAC | 2163 | GUGUCUGU G GCCCGGGA | 4942 |
| 1345 | CCTGGGGC GGCTAGCTACAACGA TTCTCCCG | 2164 | CGGGAGAA G GCCCCAGG | 4943 |
| 1353 | CACAGAGC GGCTAGCTACAACGA CCTGGGGC | 2165 | GCCCCAGG G GCUCUGUG | 4944 |
| 1358 | GCCGCCAC GGCTAGCTACAACGA AGAGCCCT | 2166 | AGGGCUCU G GUGGCGGC | 4945 |
| 1361 | GGGGCCGC GGCTAGCTACAACGA CACAGAGC | 2167 | GCUCUGUG G GCGGCCCC | 4946 |
| 1364 | TCGGGGGC GGCTAGCTACAACGA CGCCACAG | 2168 | CUGUGGCG G GCCCCCGA | 4947 |
| 1380 | GTCTGTGT GGCTAGCTACAACGA CCTCCTCC | 2169 | GGAGGAGG A ACACAGAC | 4948 |
| 1382 | GGGTCTGT GGCTAGCTACAACGA GTCCTCCT | 2170 | AGGAGGAC A ACAGACCC | 4949 |
| 1386 | ACGGGGGT GGCTAGCTACAACGA CTGTGTCC | 2171 | GGACACAG A ACCCCCGU | 4950 |
| 1392 | CAGGCGAC GGCTAGCTACAACGA GGGGGTCT | 2172 | AGACCCCC G GUCGCCUG | 4951 |
| 1395 | CACCAGGC GGCTAGCTACAACGA GACGGGGG | 2173 | CCCCCGUC G GCCUGGUG | 4952 |
| 1400 | AGCTGCAC GGCTAGCTACAACGA CAGGCGAC | 2174 | GUCGCCUG G GUGCAGCU | 4953 |
| 1402 | GCAGCTGC GGCTAGCTACAACGA ACCAGGCG | 2175 | CGCCUGGU G GCAGCUGC | 4954 |
| 1405 | GGAGCAGC GGCTAGCTACAACGA TGCACCAG | 2176 | CUGGUGCA G GCUGCUCC | 4955 |
| 1408 | GGCGGAGC GGCTAGCTACAACGA AGCTGCAC | 2177 | GUGCAGCU G GCUCCGCC | 4956 |
| 1413 | GTGCTGGC GGCTAGCTACAACGA GGAGCAGC | 2178 | GCUGCUCC G GCCAGCAC | 4957 |
| 1417 | TGCTGTGC GGCTAGCTACAACGA TGGCGGAG | 2179 | CUCCGCCA G GCACAGCA | 4958 |
| 1419 | GCTGCTGT GGCTAGCTACAACGA GCTGGCGG | 2180 | CCGCCAGC A ACAGCAGC | 4959 |
| 1422 | GGGGCTGC GGCTAGCTACAACGA TGTGCTGG | 2181 | CCAGCACA G GCAGCCCC | 4960 |
| 1425 | CCAGGGGC GGCTAGCTACAACGA TGCTGTGC | 2182 | GCACAGCA G GCCCCUGG | 4961 |
| 1432 | ACACCTGC GGCTAGCTACAACGA CAGGGGCT | 2183 | AGCCCCUG G GCAGGUGU | 4962 |
| 1436 | CCGTACAC GGCTAGCTACAACGA CTGCCAGG | 2184 | CCUGGCAG G GUGUACGG | 4963 |
| 1438 | AGCCGTAC GGCTAGCTACAACGA ACCTGCCA | 2185 | UGGCAGGU G GUACGGCU | 4964 |
| 1440 | GAAGCCGT GGCTAGCTACAACGA ACACCTGC | 2186 | GCAGGUGU A ACGGCUUC | 4965 |
| 1443 | CACGAAGC GGCTAGCTACAACGA CGTACACC | 2187 | GGUGUACG G GCUUCGUG | 4966 |
| 1448 | GCCCGCAC GGCTAGCTACAACGA GAAGCCGT | 2188 | ACGGCUUC G GUGCGGGC | 4967 |
| 1450 | AGGCCCGC GGCTAGCTACAACGA ACGAAGCC | 2189 | GGCUUCGU G GCGGGCCU | 4968 |
| 1454 | AGGCAGGC GGCTAGCTACAACGA CCGCACGA | 2190 | UCGUGCGG G GCCUGCCU | 4969 |
| 1458 | GCGCAGGC GGCTAGCTACAACGA AGGCCCGC | 2191 | GCGGGCCU G GCCUGCGC | 4970 |
| 1462 | GCCGGCGC GGCTAGCTACAACGA AGGCAGGC | 2192 | GCCUGCCU G GCGCCGGC | 4971 |
| 1464 | CAGCCGGC GGCTAGCTACAACGA GCAGGCAG | 2193 | CUGCCUGC G GCCGGCUG | 4972 |
| 1468 | GCACCAGC GGCTAGCTACAACGA CGGCGCAG | 2194 | CUGCGCCG G GCUGGUGC | 4973 |
| 1472 | GGGGGCAC GGCTAGCTACAACGA CAGCCGGC | 2195 | GCCGGCUG G GUGCCCCC | 4974 |
| 1474 | CTGGGGGC GGCTAGCTACAACGA ACCAGCCG | 2196 | CGGCUGGU G GCCCCCAG | 4975 |
| 1482 | CCAGAGGC GGCTAGCTACAACGA CTGGGGGC | 2197 | GCCCCCAG G GCCUCUGG | 4976 |
| 1491 | CCTGGAGC GGCTAGCTACAACGA CCCAGAGG | 2198 | CCUCUGGG G GCUCCAGG | 4977 |
| 1498 | CGTTGTGC GGCTAGCTACAACGA CTGGAGCC | 2199 | GGCUCCAG G GCACAACG | 4978 |
| 1500 | TTCGTTGT GGCTAGCTACAACGA GCCTGGAG | 2200 | CUCCAGGC A ACAACGAA | 4979 |
| 1503 | GCGTTCGT GGCTAGCTACAACGA TGTGCCTG | 2201 | CAGGCACA A ACGAACGC | 4980 |
| 1507 | AGCGGCGT GGCTAGCTACAACGA TCGTTGTG | 2202 | CACAACGA A ACGCCGCU | 4981 |
| 1509 | GAAGCGGC GGCTAGCTACAACGA GTTCGTTG | 2203 | CAACGAAC G GCCGCUUC | 4982 |
| 1512 | GAGGAAGC GGCTAGCTACAACGA GGCGTTCG | 2204 | CGAACGCC G GCUUCCUC | 4983 |
| 1524 | CTTGGTGT GGCTAGCTACAACGA TCCTGAGG | 2205 | CCUCAGGA A ACACCAAG | 4984 |
| 1526 | TTCTTGGT GGCTAGCTACAACGA GTTCCTGA | 2206 | UCAGGAAC A ACCAAGAA | 4985 |
| 1534 | AGATGAAC GGCTAGCTACAACGA TTCTTGGT | 2207 | ACCAAGAA G GUUCAUCU | 4986 |
| 1538 | AGGGAGAT GGCTAGCTACAACGA GAACTTCT | 2208 | AGAAGUUC A AUCUCCCU | 4987 |
| 1552 | TGGCATGC GGCTAGCTACAACGA TTCCCCAG | 2209 | CUGGGGAA G GCAUGCCA | 4988 |
| 1554 | CTTGGCAT GGCTAGCTACAACGA GCTTCCCC | 2210 | GGGGAAGC A AUGCCAAG | 4989 |
| 1556 | AGCTTGGC GGCTAGCTACAACGA ATGCTTCC | 2211 | GGAAGCAU G GCCAAGCU | 4990 |
| 1561 | GCGAGAGC GGCTAGCTACAACGA TTGGCATG | 2212 | CAUGCCAA G GCUCUCGC | 4991 |
| 1567 | CCTGCAGC GGCTAGCTACAACGA GAGAGCTT | 2213 | AAGCUCUC G GCUGCAGG | 4992 |
| 1570 | GCTCCTGC GGCTAGCTACAACGA AGCGAGAG | 2214 | CUCUCGCU G GCAGGAGC | 4993 |
| 1576 | ACGTCAGC GGCTAGCTACAACGA TCCTGCAG | 2215 | CUGCAGGA G GCUGACCU | 4994 |
| 1580 | TTCCACGT GGCTAGCTACAACGA CAGCTCCT | 2216 | AGGAGCUG A ACGUGGAA | 4995 |
| 1582 | TCTTCCAC GGCTAGCTACAACGA GTCAGCTC | 2217 | GAGCUGAC G GUGGAAGA | 4996 |
| 1589 | ACGCTCAT GGCTAGCTACAACGA CTTCCACG | 2218 | CGUCGAAG A AUGACCGU | 4997 |
| 1593 | CCGCACGC GGCTAGCTACAACGA TCATCTTC | 2219 | GAAGAUGA G GCGUGCGG | 4998 |
| 1595 | TCCCGCAC GGCTAGCTACAACGA GCTCATCT | 2220 | AGAUGAGC G GUGCGGGA | 4999 |
| 1597 | AGTCCCGC GGCTAGCTACAACGA ACGCTCAT | 2221 | AUGAGCGU G GCGGGACU | 5000 |
| 1602 | AGCGCAGT GGCTAGCTACAACGA CCCGCACG | 2222 | CGUGCGGG A ACUGCGCU | 5001 |
| 1605 | CCAAGCGC GGCTAGCTACAACGA AGTCCCGC | 2223 | GCGGGACU G GCGCUUGG | 5002 |
| 1607 | AGCCAAGC GGCTAGCTACAACGA GCAGTCCC | 2224 | GGGACUGC G GCUUGGCU | 5003 |
| 1612 | TGCGCAGC GGCTAGCTACAACGA CAAGCGCA | 2225 | UGCGCUUG G GCUGCGCA | 5004 |
| 1615 | TCCTGCGC GGCTAGCTACAACGA AGCCAAGC | 2226 | GCUUGGCU G GCGCAGGA | 5005 |
| 1617 | GCTCCTGC GGCTAGCTACAACGA GCAGCCAA | 2227 | UUGGCUGC G GCAGGAGC | 5006 |
| 1623 | CCCTGGGC GGCTAGCTACAACGA TCCTGCGC | 2228 | GCGCAGGA G GCCCAGGG | 5007 |
| 1631 | CAGCCAAC GGCTAGCTACAACGA CCCTGGGC | 2229 | GCCCAGGG G GUUGGCUG | 5008 |
| 1635 | AACACACC GGCTAGCTACAACGA CAACCCCT | 2230 | AGGGGUUG G GCUGUGUU | 5009 |
| 1638 | CCGAACAC GGCTAGCTACAACGA AGCCAACC | 2231 | CGUUGGCU G GUGUUCCG | 5010 |
| 1640 | GCCGGAAC GGCTAGCTACAACGA ACAGCCAA | 2232 | UUGGCUGU G GUUCCGGC | 5011 |
| 1646 | TCTGCGGC GGCTAGCTACAACGA CGGAACAC | 2233 | GUGUUCCG G GCCGCAGA | 5012 |
| 1649 | TGCTCTGC GGCTAGCTACAACGA GGCCGGAA | 2234 | UUCCGGCC G GCAGAGCA | 5013 |
| 1654 | GACGGTGC GGCTAGCTACAACGA TCTGCGGC | 2235 | GCCGCAGA G GCACCGUC | 5014 |
| 1656 | CAGACGGT GGCTAGCTACAACGA GCTCTGCG | 2236 | CGCAGAGC A ACCGUCUG | 5015 |
| 1659 | ACGCAGAC GGCTAGCTACAACGA GGTGCTCT | 2237 | AGAGCACC G GUCUGCGU | 5016 |
| 1663 | CCTCACGC GGCTAGCTACAACGA AGACGGTG | 2238 | CACCGUCU G GCGUGAGG | 5017 |
| 1665 | CTCCTCAC GGCTAGCTACAACGA GCAGACGG | 2239 | CCGUCUGC G GUGAGGAG | 5018 |
| 1673 | GCCAGGAT GGCTAGCTACAACGA CTCCTCAC | 2240 | GUGAGGAG A AUCCUGGC | 5019 |
| 1679 | AACTTGGC GGCTAGCTACAACGA CAGGATCT | 2241 | AGAUCCUG G GCCAAGUU | 5020 |
| 1684 | GCAGGAAC GGCTAGCTACAACGA TTGGCCAG | 2242 | CUGGCCAA G GUUCCUGC | 5021 |
| 1690 | GCCAGTGC GGCTAGCTACAACGA AGGAACTT | 2243 | AAGUUCCU G GCACUGGC | 5022 |
| 1692 | CAGCCAGT GGCTAGCTACAACGA GCAGGAAC | 2244 | GUUCCUGC A ACUGGCUG | 5023 |
| 1696 | TCATCAGC GGCTAGCTACAACGA CAGTGCAG | 2245 | CUGCACUG G GCUGAUGA | 5024 |
| 1700 | ACACTCAT GGCTAGCTACAACGA CAGCCAGT | 2246 | ACUGGCUG A AUGAGUGU | 5025 |
| 1704 | GTACACAC GGCTAGCTACAACGA TCATCAGC | 2247 | GCUGAUGA G GUGUGUAC | 5026 |
| 1706 | ACGTACAC GGCTAGCTACAACGA ACTCATCA | 2248 | UGAUGAGU G GUGUACGU | 5027 |
| 1708 | CGACGTAC GGCTAGCTACAACGA ACACTCAT | 2249 | AUGAGUGU G GUACGUCG | 5028 |
| 1710 | GACGACGT GGCTAGCTACAACGA ACACACTC | 2250 | GAGUGUGU A ACGUCGUC | 5029 |
| 1712 | TCGACGAC GGCTAGCTACAACGA GTACACAC | 2251 | GUGUGUAC G GUCGUCGA | 5030 |
| 1715 | AGCTCGAC GGCTAGCTACAACGA GACGTACA | 2252 | UGUACGUC G GUCGAGCU | 5031 |
| 1720 | TGAGCAGC GGCTAGCTACAACGA TCGACGAC | 2253 | GUCGUCGA G GCUGCUCA | 5032 |
| 1723 | ACCTGAGC GGCTAGCTACAACGA AGCTCGAC | 2254 | GUCGAGCU G GCUCAGGU | 5033 |
| 1729 | AGAAAGAC GGCTAGCTACAACGA CTGAGCAG | 2255 | CUGCUCAG G GUCUUUCU | 5034 |
| 1740 | CGTGACAT GGCTAGCTACAACGA AAAAGAAA | 2256 | UUUCUUUU A AUGUCACG | 5035 |
| 1742 | TCCGTGAC GGCTAGCTACAACGA ATAAAAGA | 2257 | UCUUUUAU G GUCACGGA | 5036 |
| 1745 | GTCTCCGT GGCTAGCTACAACGA GACATAAA | 2258 | UUUAUGUC A ACGGAGAC | 5037 |
| 1751 | AACGTGGT GGCTAGCTACAACGA CTCCGTGA | 2259 | UCACGGAG A ACCACGUU | 5038 |
| 1754 | TGAAACGT GGCTAGCTACAACGA GGTCTCCG | 2260 | CGGAGACC A ACGUUUCA | 5039 |
| 1756 | TTTGAAAC GGCTAGCTACAACGA GTGGTCTC | 2261 | GAGACCAC G GUUUCAAA | 5040 |
| 1767 | GAGCCTGT GGCTAGCTACAACGA TCTTTTGA | 2262 | UCAAAAGA A ACAGGCUC | 5041 |
| 1771 | AAAAGAGC GGCTAGCTACAACGA CTGTTCTT | 2263 | AAGAACAG G GCUCUUUU | 5042 |
| 1782 | CTTCCGGT GGCTAGCTACAACGA AGAAAAAG | 2264 | CUUUUUCU A ACCGGAAG | 5043 |
| 1791 | CCAGACAC GGCTAGCTACAACGA TCTTCCGG | 2265 | CCGGAAGA G GUGUCUGG | 5044 |
| 1793 | CTCCAGAC GGCTAGCTACAACGA ACTCTTCC | 2266 | GGAAGAGU G GUCUGGAG | 5045 |
| 1800 | CAACTTGC GGCTAGCTACAACGA TCCAGACA | 2267 | UGUCUGGA G GCAAGUUG | 5046 |
| 1804 | TTTGCAAC GGCTAGCTACAACGA TTGCTCCA | 2268 | UGGAGCAA G GUUGCAAA | 5047 |
| 1807 | TGCTTTGC GGCTAGCTACAACGA AACTTGCT | 2269 | AGCAAGUU G GCAAAGCA | 5048 |
| 1812 | TCCAATGC GGCTAGCTACAACGA TTTGCAAC | 2270 | GUUGCAAA G GCAUUGGA | 5049 |
| 1814 | ATTCCAAT GGCTAGCTACAACGA GCTTTGCA | 2271 | UGCAAAGC A AUUGGAAU | 5050 |
| 1820 | TGTCTGAT GGCTAGCTACAACGA TCCAATGC | 2272 | GCAUUGGA A AUCAGACA | 5051 |
| 1825 | AGTGCTGT GGCTAGCTACAACGA CTGATTCC | 2273 | GGAAUCAG A ACAGCACU | 5052 |
| 1828 | TCAAGTGC GGCTAGCTACAACGA TGTCTGAT | 2274 | AUCAGACA G GCACUUGA | 5053 |
| 1830 | CTTCAAGT GGCTAGCTACAACGA GCTGTCTG | 2275 | CAGACAGC A ACUUGAAG | 5054 |
| 1841 | AGCTGCAC GGCTAGCTACAACGA CCTCTTCA | 2276 | UGAAGAGG G GUGCAGCU | 5055 |
| 1843 | GCAGCTGC GGCTAGCTACAACGA ACCCTCTT | 2277 | AAGAGGGU G GCAGCUGC | 5056 |
| 1846 | CCCGCAGC GGCTAGCTACAACGA TGCACCCT | 2278 | AGGGUGCA G GCUGCGGG | 5057 |
| 1849 | GCTCCCGC GGCTAGCTACAACGA AGCTGCAC | 2279 | GUGCAGCU G GCGGGAGC | 5058 |
| 1855 | CCGACAGC GGCTAGCTACAACGA TCCCGCAG | 2280 | CUGCGGGA G GCUGUCGG | 5059 |
| 1858 | CTTCCGAC GGCTAGCTACAACGA AGCTCCCG | 2281 | CGGGAGCU G GUCGGAAG | 5060 |
| 1865 | ACCTCTGC GGCTAGCTACAACGA TTCCGACA | 2282 | UGUCGGAA G GCAGAGGU | 5061 |
| 1871 | TGCCTGAC GGCTAGCTACAACGA CTCTGCTT | 2283 | AAGCAGAG G GUCAGGCA | 5062 |
| 1876 | GATGCTGC GGCTAGCTACAACGA CTGACCTC | 2284 | GAGGUCAG G GCAGCAUC | 5063 |
| 1879 | CCCGATGC GGCTAGCTACAACGA TGCCTGAC | 2285 | GUCAGGCA G GCAUCGGG | 5064 |
| 1881 | TTCCCGAT GGCTAGCTACAACGA GCTGCCTG | 2286 | CAGGCAGC A AUCGGGAA | 5065 |
| 1889 | GGCCTGGC GGCTAGCTACAACGA TTCCCGAT | 2287 | AUCGGGAA G GCCAGGCC | 5066 |
| 1894 | GGGCGGGC GGCTAGCTACAACGA CTGGCTTC | 2288 | GAAGCCAG G GCCCGCCC | 5067 |
| 1898 | AGCAGGGC GGCTAGCTACAACGA GGGCCTGG | 2289 | CCAGGCCC G GCCCUGCU | 5068 |
| 1903 | ACGTCAGC GGCTAGCTACAACGA AGGGCGGG | 2290 | CCCGCCCU G GCUGACGU | 5069 |
| 1907 | CTGGACGT GGCTAGCTACAACGA CAGCAGGG | 2291 | CCCUGCUG A ACGUCCAG | 5070 |
| 1909 | GTCTGGAC GGCTAGCTACAACGA GTCAGCAG | 2292 | CUGCUGAC G GUCCAGAC | 5071 |
| 1915 | AGCGGAGT GGCTAGCTACAACGA CTGGACGT | 2293 | ACGUCCAG A ACUCCGCU | 5072 |
| 1920 | GATGAAGC GGCTAGCTACAACGA GGAGTCTG | 2294 | CAGACUCC G GCUUCAUC | 5073 |
| 1925 | TTGGGGAT GGCTAGCTACAACGA GAAGCGGA | 2295 | UCCGCUUC A AUCCCCAA | 5074 |
| 1933 | CGTCAGGC GGCTAGCTACAACGA TTGGGGAT | 2296 | AUCCCCAA G GCCUGACG | 5075 |
| 1938 | CAGCCCGT GGCTAGCTACAACGA CAGGCTTG | 2297 | CAAGCCUG A ACGGGCUG | 5076 |
| 1942 | GCCGCAGC GGCTAGCTACAACGA CCGTCAGG | 2298 | CCUGACGG G GCUGCGGC | 5077 |
| 1945 | TCGGCCGC GGCTAGCTACAACGA AGCCCGTC | 2299 | GACGGGCU G GCGGCCGA | 5078 |
| 1948 | CAATCGGC GGCTAGCTACAACGA CGCAGCCC | 2300 | GGGCUGCG G GCCGAUUG | 5079 |
| 1952 | TTCACAAT GGCTAGCTACAACGA CGGCCGCA | 2301 | UGCGGCCG A AUUGUGAA | 5080 |
| 1955 | ATGTTCAC GGCTAGCTACAACGA AATCGGCC | 2302 | GGCCGAUU G GUGAACAU | 5081 |
| 1959 | GTCCATGT GGCTAGCTACAACGA TCACAATC | 2303 | GAUUGUGA A ACAUGGAC | 5082 |
| 1961 | TAGTCCAT GGCTAGCTACAACGA GTTCACAA | 2304 | UUGUGAAC A AUGGACUA | 5083 |
| 1965 | GACGTAGT GGCTAGCTACAACGA CCATGTTC | 2305 | GAACAUGG A ACUACGUC | 5084 |
| 1968 | CACGACGT GGCTAGCTACAACGA AGTCCATG | 2306 | CAUGGACU A ACGUCGUG | 5085 |
| 1970 | CCCACGAC GGCTAGCTACAACGA GTAGTCCA | 2307 | UGGACUAC G GUCGUGGG | 5086 |
| 1973 | GCTCCCAC GGCTAGCTACAACGA GACGTAGT | 2308 | ACUACGUC G GUGGGAGC | 5087 |
| 1979 | GTTCTGGC GGCTAGCTACAACGA TCCCACGA | 2309 | UCGUGGGA G GCCAGAAC | 5088 |
| 1985 | CGGAACGT GGCTAGCTACAACGA TCTGGCTC | 2310 | GAGCCAGA A ACGUUCCG | 5089 |
| 1987 | TGCGGAAC GGCTAGCTACAACGA GTTCTGGC | 2311 | GCCAGAAC G GUUCCGCA | 5090 |
| 1992 | TTCTCTGC GGCTAGCTACAACGA GGAACGTT | 2312 | AACGUUCC G GCAGAGAA | 5091 |
| 2006 | CGCTCGGC GGCTAGCTACAACGA CCTCTTTT | 2313 | AAAAGAGG G GCCGAGCG | 5092 |
| 2011 | TGAGACGC GGCTAGCTACAACGA TCGGCCCT | 2314 | AGGGCCGA G GCGUCUCA | 5093 |
| 2013 | GGTGAGAC GGCTAGCTACAACGA GCTCGGCC | 2315 | GGCCGAGC G GUCUCACC | 5094 |
| 2018 | CTCGAGGT GGCTAGCTACAACGA GAGACGCT | 2316 | AGCGUCUC A ACCUCGAG | 5095 |
| 2027 | GCCTTCAC GGCTAGCTACAACGA CCTCGAGG | 2317 | CCUCGAGG G GUGAAGGC | 5096 |
| 2033 | AACAGTGC GGCTAGCTACAACGA CTTCACCC | 2318 | GGGUGAAG G GCACUGUU | 5097 |
| 2035 | TGAACAGT GGCTAGCTACAACGA GCCTTCAC | 2319 | GUGAAGGC A ACUGUUCA | 5098 |
| 2038 | CGCTGAAC GGCTAGCTACAACGA AGTGCCTT | 2320 | AAGGCACU G GUUCAGCG | 5099 |
| 2043 | GAGCACGC GGCTAGCTACAACGA TGAACAGT | 2321 | ACUGUUCA G GCGUGCUC | 5100 |
| 2045 | TTGAGCAC GGCTAGCTACAACGA GCTGAACA | 2322 | UGUUCAGC G GUGCUCAA | 5101 |
| 2047 | AGTTGAGC GGCTAGCTACAACGA ACGCTGAA | 2323 | UUCAGCGU G GCUCAACU | 5102 |
| 2052 | CTCGTAGT GGCTAGCTACAACGA TGAGCACG | 2324 | CGUGCUCA A ACUACGAG | 5103 |
| 2055 | CCGCTCGT GGCTAGCTACAACGA AGTTGAGC | 2325 | GCUCAACU A ACGAGCGG | 5104 |
| 2059 | GCGCCCGC GGCTAGCTACAACGA TCGTAGTT | 2326 | AACUACGA G GCGGGCGC | 5105 |
| 2063 | CGCCGCGC GGCTAGCTACAACGA CCGCTCGT | 2327 | ACGAGCGG G GCGCGGCG | 5106 |
| 2065 | GGCGCCGC GGCTAGCTACAACGA GCCCGCTC | 2328 | GAGCGGGC G GCGGCGCC | 5107 |
| 2068 | CGGGGCGC GGCTAGCTACAACGA CGCGCCCG | 2329 | CGGGCGCG G GCGCCCCG | 5108 |
| 2070 | GCCGGGGC GGCTAGCTACAACGA GCCGCGCC | 2330 | GGCGCGGC G GCCCCGGC | 5109 |
| 2076 | CAGGAGGC GGCTAGCTACAACGA CGGGGCGC | 2331 | GCGCCCCG G GCCUCCUG | 5110 |
| 2085 | AGAGGCGC GGCTAGCTACAACGA CCAGGAGG | 2332 | CCUCCUGG G GCGCCUCU | 5111 |
| 2087 | ACAGAGGC GGCTAGCTACAACGA GCCCAGGA | 2333 | UCCUGGGC G GCCUCUGU | 5112 |
| 2093 | CCCAGCAC GGCTAGCTACAACGA AGAGGCGC | 2334 | GCGCCUCU G GUGCUGGG | 5113 |
| 2095 | GGCCCAGC GGCTAGCTACAACGA ACAGAGGC | 2335 | GCCUCUGU G GCUGGGCC | 5114 |
| 2100 | GTCCAGGC GGCTAGCTACAACGA CCAGCACA | 2336 | UGUGCUGG G GCCUGGAC | 5115 |
| 2106 | GATATCGT GGCTAGCTACAACGA CCAGGCCC | 2337 | GGGCCUGG A ACGAUAUC | 5116 |
| 2109 | GTGGATAT GGCTAGCTACAACGA CGTCCAGG | 2338 | CCUGGACG A AUAUCCAC | 5117 |
| 2111 | CTGTGGAT GGCTAGCTACAACGA ATCGTCCA | 2339 | UGGACGAU A AUCCACAG | 5118 |
| 2115 | GGCCCTGT GGCTAGCTACAACGA GGATATCG | 2340 | CGAUAUCC A ACAGGGCC | 5119 |
| 2120 | CGCCAGGC GGCTAGCTACAACGA CCTGTGGA | 2341 | UCCACAGG G GCCUGGCG | 5120 |
| 2125 | AGGTGCGC GGCTAGCTACAACGA CAGGCCCT | 2342 | AGGGCCUG G GCGCACCU | 5121 |
| 2127 | GAAGGTGC GGCTAGCTACAACGA GCCAGGCC | 2343 | GGCCUGGC G GCACCUUC | 5122 |
| 2129 | ACGAAGGT GGCTAGCTACAACGA GCGCCAGG | 2344 | CCUGGCGC A ACCUUCGU | 5123 |
| 2135 | CGCAGCAC GGCTAGCTACAACGA GAAGGTGC | 2345 | GCACCUUC G GUGCUGCG | 5124 |
| 2137 | CACGCAGC GGCTAGCTACAACGA ACGAAGGT | 2346 | ACCUUCGU G GCUGCGUG | 5125 |
| 2140 | GCACACGC GGCTAGCTACAACGA AGCACGAA | 2347 | UUCGUGCU G GCGUGUGC | 5126 |
| 2142 | CCGCACAC GGCTAGCTACAACGA GCAGCACG | 2348 | CGUGCUGC G GUGUGCGG | 5127 |
| 2144 | GCCCGCAC GGCTAGCTACAACGA ACGCAGCA | 2349 | UGCUGCGU G GUGCGGGC | 5128 |
| 2146 | GGGCCCGC GGCTAGCTACAACGA ACACGCAG | 2350 | CUGCGUGU G GCGGGCCC | 5129 |
| 2150 | TCCTGGGC GGCTAGCTACAACGA CCGCACAC | 2351 | GUGUGCGG G GCCCAGGA | 5130 |
| 2157 | CGGCGGGT GGCTAGCTACAACGA CCTGGGCC | 2352 | GGCCCAGG A ACCCGCCG | 5131 |
| 2161 | CAGGCGGC GGCTAGCTACAACGA GGGTCCTG | 2353 | CAGGACCC G GCCGCCUG | 5132 |
| 2164 | GCTCAGGC GGCTAGCTACAACGA GGCGGGTC | 2354 | GACCCGCC G GCCUGAGC | 5133 |
| 2170 | AGTACAGC GGCTAGCTACAACGA TCAGGCGG | 2355 | CCGCCUGA G GCUGUACU | 5134 |
| 2173 | CAAAGTAC GGCTAGCTACAACGA AGCTCAGG | 2356 | CCUGAGCU G GUACUUUG | 5135 |
| 2175 | GACAAAGT GGCTAGCTACAACGA ACAGCTCA | 2357 | UGAGCUGU A ACUUUGUC | 5136 |
| 2180 | ACCTTGAC GGCTAGCTACAACGA AAAGTACA | 2358 | UGUACUUU G GUCAAGGU | 5137 |
| 2186 | ACATCCAC GGCTAGCTACAACGA CTTGACAA | 2359 | UUGUCAAG G GUGGAUGU | 5138 |
| 2190 | CGTCACAT GGCTAGCTACAACGA CCACCTTG | 2360 | CAAGGUGG A AUGUGACG | 5139 |
| 2192 | CCCGTCAC GGCTAGCTACAACGA ATCCACCT | 2361 | AGGUGGAU G GUGACGGG | 5140 |
| 2195 | GCGCCCGT GGCTAGCTACAACGA CACATCCA | 2362 | UGGAUGUG A ACGGGCGC | 5141 |
| 2199 | GTACGCGC GGCTAGCTACAACGA CCGTCACA | 2363 | UGUGACGG G GCGCGUAC | 5142 |
| 2201 | TCGTACGC GGCTAGCTACAACGA GCCCGTCA | 2364 | UGACGGGC G GCGUACGA | 5143 |
| 2203 | TGTCGTAC GGCTAGCTACAACGA GCGCCCGT | 2365 | ACGGGCGC G GUACGACA | 5144 |
| 2205 | GGTGTCGT GGCTAGCTACAACGA ACGCGCCC | 2366 | GGGCGCGU A ACGACACC | 5145 |
| 2208 | GATGGTGT GGCTAGCTACAACGA CGTACGCG | 2367 | CGCGUACG A ACACCAUC | 5146 |
| 2210 | GGGATGGT GGCTAGCTACAACGA GTCGTACG | 2368 | CGUACGAC A ACCAUCCC | 5147 |
| 2213 | TGGGGGAT GGCTAGCTACAACGA GGTGTCGT | 2369 | ACGACACC A AUCCCCCA | 5148 |
| 2223 | GAGCCTGT GGCTAGCTACAACGA CCTGGGGG | 2370 | CCCCCAGG A ACAGGCUC | 5149 |
| 2227 | CCGTGAGC GGCTAGCTACAACGA CTGTCCTG | 2371 | CAGGACAG G GCUCACGG | 5150 |
| 2231 | ACCTCCGT GGCTAGCTACAACGA GAGCCTGT | 2372 | ACAGGCUC A ACGGAGGU | 5151 |
| 2237 | GCGATGAC GGCTAGCTACAACGA CTCCGTGA | 2373 | UCACGGAG G GUCAUCGC | 5152 |
| 2240 | CTGGCGAT GGCTAGCTACAACGA GACCTCCG | 2374 | CGGAGGUC A AUCGCCAG | 5153 |
| 2243 | ATGCTGGC GGCTAGCTACAACGA GATGACCT | 2375 | AGGUCAUC G GCCAGCAU | 5154 |
| 2247 | GATUATUC GGCTAGCTACAACGA TGGCGATG | 2376 | CAUCGCCA G GCAUCAUC | 5155 |
| 2249 | TTGATGAT GGCTAGCTACAACGA GCTGGCGA | 2377 | UCGCCAGC A AUCAUCAA | 5156 |
| 2252 | GGTTTGAT GGCTAGCTACAACGA GATGCTGG | 2378 | CCAGCAUC A AUCAAACC | 5157 |
| 2257 | TCTGGGGT GGCTAGCTACAACGA TTGATGAT | 2379 | AUCAUCAA A ACCCCAGA | 5158 |
| 2265 | GTACGTGT GGCTAGCTACAACGA TCTGGGGT | 2380 | ACCCCAGA A ACACGUAC | 5159 |
| 2267 | CAGTACGT GGCTAGCTACAACGA GTTCTGGG | 2381 | CCCAGAAC A ACGUACUG | 5160 |
| 2269 | CGCAGTAC GGCTAGCTACAACGA GTGTTCTG | 2382 | CAGAACAC G GUACUGCG | 5161 |
| 2271 | CACGCAGT GGCTAGCTACAACGA ACGTGTTC | 2383 | GAACACGU A ACUGCGUG | 5162 |
| 2274 | ACUCACUC GUCTAGCTACAACGA AGTACGTG | 2384 | CACGUACU G GCGUGCGU | 5163 |
| 2276 | CGACGCAC GGCTAGCTACAACGA GCAGTACG | 2385 | CGUACUGC G GUGCGUCG | 5164 |
| 2278 | ACCGACGC GGCTAGCTACAACGA ACGCAGTA | 2386 | UACUGCGU G GCUUCGGU | 5165 |
| 2280 | ATACCUAC GGCTAGCTACAACGA GCACGCAG | 2387 | CUGCGUGC G UUCGGUAU | 5166 |
| 2284 | CGUCATAC GGCTAGCTACAACGA CGACGCAC | 2388 | GUGCGUCG G GUAUGCCG | 5167 |
| 2286 | CACUGCAT GGCTAGCTACAACGA ACCGACGC | 2389 | GCGUCGGU A AUGCCGUG | 5168 |
| 2288 | ACCACGGC GGCTAGCTACAACGA ATACCGAC | 2390 | GUCGGUAU G GCCGUGGU | 5169 |
| 2291 | TGGACCAC GGCTAGCTACAACGA GGCATACC | 2391 | GGUAUGCC G GUGGUCCA | 5170 |
| 2294 | TTCTGGAC GGCTAGCTACAACGA CACGGCAT | 2392 | AUGCCGUG G GUCCAGAA | 5171 |
| 2303 | TGGGCGGC GGCTAGCTACAACGA CTTCTGGA | 2393 | UCCAGAAG G GCCGCCCA | 5172 |
| 2306 | CCATGGGC GGCTAGCTACAACGA GGCCTTCT | 2394 | AGAAGGCC G GCCCAUGG | 5173 |
| 2310 | GTGCCCAT GGCTAGCTACAACGA GGGCGGCC | 2395 | GGCCGCCC A AUGGGCAC | 5174 |
| 2314 | GGACGTGC GGCTAGCTACAACGA CCATGGGC | 2396 | GCCCAUGG G GCACGUCC | 5175 |
| 2316 | GCGGACGT GGCTAGCTACAACGA GCCCATGG | 2397 | CCAUGGGC A ACGUCCGC | 5176 |
| 2318 | TTGCGGAC GGCTAGCTACAACGA GTGCCCAT | 2398 | AUGGGCAC G GUCCGCAA | 5177 |
| 2322 | GGCCTTGC GGCTAGCTACAACGA GGACGTGC | 2399 | GCACGUCC G GCAAGGCC | 5178 |
| 2327 | TTGAAGGC GGCTAGCTACAACGA CTTGCGGA | 2400 | UCCGCAAG G GCCUUCAA | 5179 |
| 2337 | GACGTGGC GGCTAGCTACAACGA TCTTGAAG | 2401 | CUUCAAGA G GCCACGUC | 5180 |
| 2340 | AGAGACGT GGCTAGCTACAACGA GGCTCTTG | 2402 | CAAGAGCC A ACGUCUCU | 5181 |
| 2342 | GTAGAGAC GGCTAGCTACAACGA GTGGCTCT | 2403 | AGAGCCAC G GUCUCUAC | 5182 |
| 2348 | GTCAAGGT GGCTAGCTACAACGA AGAGACGT | 2404 | ACGUCUCU A ACCUUGAC | 5183 |
| 2354 | AGGTCTGT GGCTAGCTACAACGA CAAGGTAG | 2405 | CUACCUUG A ACAGACCU | 5184 |
| 2358 | CTGGAGGT GGCTAGCTACAACGA CTGTCAAG | 2406 | CUUGACAG A ACCUCCAG | 5185 |
| 2365 | TGTACGGC GGCTAGCTACAACGA TGGAGGTC | 2407 | GACCUCCA G GCCGUACA | 5186 |
| 2368 | GCATGTAC GGCTAGCTACAACGA GGCTGGAG | 2408 | CUCCAGCC G GUACAUGC | 5187 |
| 2370 | TCGCATGT GGCTAGCTACAACGA ACGGCTGG | 2409 | CCAGCCGU A ACAUGCGA | 5188 |
| 2372 | TGTCGCAT GGCTAGCTACAACGA GTACGGCT | 2410 | AGCCGUAC A AUGCGACA | 5189 |
| 2374 | ACTGTCGC GGCTAGCTACAACGA ATGTACGG | 2411 | CCGUACAU G GCGACAGU | 5190 |
| 2377 | CGAACTGT GGCTAGCTACAACGA CGCATGTA | 2412 | UACAUGCG A ACAGUUCG | 5191 |
| 2380 | CCACGAAC GGCTAGCTACAACGA TGTCGCAT | 2413 | AUGCGACA G GUUCGUGG | 5192 |
| 2384 | TGAGCCAC GGCTAGCTACAACGA GAACTGTC | 2414 | GACAGUUC G GUGGCUCA | 5193 |
| 2387 | AGGTGAGC GGCTAGCTACAACGA CACGAACT | 2415 | AGUUCGUG G GCUCACCU | 5194 |
| 2391 | CTGCAGGT GGCTAGCTACAACGA GAGCCACG | 2416 | CGUGGCUC A ACCUGCAG | 5195 |
| 2395 | TCTCCTGC GGCTAGCTACAACGA AGGTGAGC | 2417 | GCUCACCU G GCAGGAGA | 5196 |
| 2402 | GGGCTGGT GGCTAGCTACAACGA CTCCTGCA | 2418 | UGCAGGAG A ACCAGCCC | 5197 |
| 2406 | CAGCGGGC GGCTAGCTACAACGA TGGTCTCC | 2419 | GGAGACCA G GCCCGCUG | 5198 |
| 2410 | CCCTCAGC GGCTAGCTACAACGA GGGCTGGT | 2420 | ACCAGCCC G GCUGAGGG | 5199 |
| 2418 | GACGGCAT GGCTAGCTACAACGA CCCTCAGC | 2421 | GCUGAGGG A AUGCCGUC | 5200 |
| 2420 | ACGACGGC GGCTAGCTACAACGA ATCCCTCA | 2422 | UGAGGGAU G GCCGUCGU | 5201 |
| 2423 | ATGACGAC GGCTAGCTACAACGA GGCATCCC | 2423 | GGGAUGCC G GUCGUCAU | 5202 |
| 2426 | TCGATGAC GGCTAGCTACAACGA GACGGCAT | 2424 | AUGCCGUC G GUCAUCGA | 5203 |
| 2429 | TGCTCGAT GGCTAGCTACAACGA GACGACGG | 2425 | CCGUCGUC A AUCGAGCA | 5204 |
| 2434 | AGCTCTGC GGCTAGCTACAACGA TCGATGAC | 2426 | GUCAUCGA G GCAGAGCU | 5205 |
| 2439 | GGAGGAGC GGCTAGCTACAACGA TCTGCTCG | 2427 | CGAGCAGA G GCUCCUCC | 5206 |
| 2451 | GGCCTCAT GGCTAGCTACAACGA TCAGGGAG | 2428 | CUCCCUGA A AUGAGGCC | 5207 |
| 2456 | CTGCTGGC GGCTAGCTACAACGA CTCATTCA | 2429 | UGAAUGAG G GCCAGCAG | 5208 |
| 2460 | GCCACTGC GGCTAGCTACAACGA TGGCCTCA | 2430 | UGAGGCCA G GCAGUGGC | 5209 |
| 2463 | GAGGCCAC GGCTAGCTACAACGA TGCTGGCC | 2431 | GGCCAGCA G GUGGCCUC | 5210 |
| 2466 | GAAGAGGC GGCTAGCTACAACGA CACTGCTG | 2432 | CAGCAGUG G GCCUCUUC | 5211 |
| 2475 | GAAGACGT GGCTAGCTACAACGA CGAAGAGG | 2433 | CCUCUUCG A ACGUCUUC | 5212 |
| 2477 | AGGAAGAC GGCTAGCTACAACGA GTCGAAGA | 2434 | UCUUCGAC G GUCUUCCU | 5213 |
| 2485 | TGAAGCGT GGCTAGCTACAACGA AGGAAGAC | 2435 | GUCUUCCU A ACGCUUCA | 5214 |
| 2487 | CATGAAGC GGCTAGCTACAACGA GTAGGAAG | 2436 | CUUCCUAC G GCUUCAUG | 5215 |
| 2492 | TGGCACAT GGCTAGCTACAACGA GAAGCGTA | 2437 | UACGCUUC A AUGUGCCA | 5216 |
| 2494 | GGTGGCAC GGCTAGCTACAACGA ATGAAGCG | 2438 | CGCUUCAU G GUGCCACC | 5217 |
| 2496 | GTGGTGGC GGCTAGCTACAACGA ACATGAAG | 2439 | CUUCAUGU G GCCACCAC | 5218 |
| 2499 | GGCGTGGT GGCTAGCTACAACGA GGCACATG | 2440 | CAUGUGCC A ACCACGCC | 5219 |
| 2502 | CACGGCGT GGCTAGCTACAACGA GGTGGCAC | 2441 | GUGCCACC A ACGCCGUG | 5220 |
| 2504 | CGCACGGC GGCTAGCTACAACGA GTGGTGGC | 2442 | GCCACCAC G GCCGUGCG | 5221 |
| 2507 | ATGCGCAC GGCTAGCTACAACGA GGCGTGGT | 2443 | ACCACGCC G GUGCGCAU | 5222 |
| 2509 | TGATGCGC GGCTAGCTACAACGA ACGGCGTG | 2444 | CACGCCGU G GCGCAUCA | 5223 |
| 2511 | CCTGATGC GGCTAGCTACAACGA GCACGGCG | 2445 | CGCCGUGC G GCAUCAGG | 5224 |
| 2513 | CCCCTGAT GGCTAGCTACAACGA GCGCACGG | 2446 | CCGUGCGC A AUCAGGGG | 5225 |
| 2520 | GGACTTGC GGCTAGCTACAACGA CCCTGATG | 2447 | CAUCAGGG G GCAAGUCC | 5226 |
| 2524 | CGTAGGAC GGCTAGCTACAACGA TTGCCCCT | 2448 | AGGGGCAA G GUCCUACG | 5227 |
| 2529 | CTGGACGT GGCTAGCTACAACGA AGGACTTG | 2449 | CAAGUCCU A ACCUCCAG | 5228 |
| 2531 | CACTGGAC GGCTAGCTACAACGA GTAGGACT | 2450 | AGUCCUAC G GUCCAGUG | 5229 |
| 2536 | CCTGGCAC GGCTAGCTACAACGA TGGACGTA | 2451 | UACGUCCA G GUGCCAGG | 5230 |
| 2538 | CCCCTGGC GGCTAGCTACAACGA ACTGGACG | 2452 | CGUCCAGU G GCCAGGGG | 5231 |
| 2546 | TGCGGGAT GGCTAGCTACAACGA CCCCTGGC | 2453 | GCCAGGGG A AUCCCGCA | 5232 |
| 2551 | AGCCCTGC GGCTAGCTACAACGA GGGATCCC | 2454 | GGGAUCCC G GCAGGGCU | 5233 |
| 2556 | GATGGAGC GGCTAGCTACAACGA CCTGCGGG | 2455 | CCCGCAGG G GCUCCAUC | 5234 |
| 2561 | GAGAGGAT GGCTAGCTACAACGA GGAGCCCT | 2456 | AGGGCUCC A AUCCUCUC | 5235 |
| 2570 | AGCAGCGT GGCTAGCTACAACGA GGAGAGGA | 2457 | UCCUCUCC A ACGCUGCU | 5236 |
| 2572 | AGAGCAGC GGCTAGCTACAACGA GTGGAGAG | 2458 | CUCUCCAC G GCUGCUCU | 5237 |
| 2575 | TGCAGAGC GGCTAGCTACAACGA AGCGTGGA | 2459 | UCCACGCU G GCUCUGCA | 5238 |
| 2580 | CAGGCTGC GGCTAGCTACAACGA AGAGCAGC | 2460 | GCUGCUCU G GCAGCCUG | 5239 |
| 2583 | GCACAGGC GGCTAGCTACAACGA TGCAGAGC | 2461 | GCUCUGCA G GCCUGUGC | 5240 |
| 2587 | CGTAGCAC GGCTAGCTACAACGA AGGCTGCA | 2462 | UGCAGCCU G GUGCUACG | 5241 |
| 2589 | GCCGTAGC GGCTAGCTACAACGA ACAGGCTC | 2463 | CAGCCUGU G GCUACGGC | 5242 |
| 2592 | GTCGCCGT GGCTAGCTACAACGA AGCACAGG | 2464 | CCUGUGCU A ACGGCGAC | 5243 |
| 2595 | CATGTCGC GGCTAGCTACAACGA CGTAGCAC | 2465 | GUGCUACG G GCGACAUG | 5244 |
| 2598 | CTCCATGT GGCTAGCTACAACGA CGCCGTAG | 2466 | CUACGGCG A ACAUGGAG | 5245 |
| 2600 | TTCTCCAT GGCTAGCTACAACGA GTCGCCGT | 2467 | ACGGCGAC A AUGGAGAA | 5246 |
| 2607 | CAGCTTGT GGCTAGCTACAACGA TCTCCATG | 2468 | CAUGGAGA A ACAAGCUG | 5247 |
| 2611 | CAAACAGC GGCTAGCTACAACGA TTGTTCTC | 2469 | GAGAACAA G GCUGUUUG | 5248 |
| 2614 | CCGCAAAC GGCTAGCTACAACGA AGCTTGTT | 2470 | AACAAGCU G GUUUGCGG | 5249 |
| 2618 | ATCCCCGC GGCTAGCTACAACGA AAACAGCT | 2471 | AGCUGUUU G GCGGGGAU | 5250 |
| 2624 | CGCCGAAT GGCTAGCTACAACGA CCCCGCAA | 2472 | UUGCGGGG A AUUCGGCG | 5251 |
| 2629 | CGTCCCGC GGCTAGCTACAACGA CGAATCCC | 2473 | GGGAUUCG G GCGGGACG | 5252 |
| 2634 | CAGCCCGT GGCTAGCTACAACGA CCCGCCCA | 2474 | UCGGCGGG A ACGGGCUG | 5253 |
| 2638 | GGAGCAGC GGCTAGCTACAACGA CCGTCCCG | 2475 | CGGGACGG G GCUGCUCC | 5254 |
| 2641 | GCAGGAGC GGCTAGCTACAACGA AGCCCGTC | 2476 | GACGGGCU G GCUCCUGC | 5255 |
| 2647 | CCAAACGC GGCTAGCTACAACGA AGGAGCAG | 2477 | CUGCUCCU G GCGUUUGG | 5256 |
| 2649 | CACCAAAC GGCTAGCTACAACGA GCAGGAGC | 2478 | GCUCCUGC G GUUUGGUG | 5257 |
| 2654 | TCATCCAC GGCTAGCTACAACGA CAAACGCA | 2479 | UGCGUUUG G GUGGAUGA | 5258 |
| 2658 | GAAATCAT GGCTAGCTACAACGA CCACCAAA | 2480 | UUUGGUGG A AUGAUUUC | 5259 |
| 2661 | CAAGAAAT GGCTAGCTACAACGA CATCCACC | 2481 | GGUGGAUG A AUUUCUUG | 5260 |
| 2668 | TCACCAAC GGCTAGCTACAACGA AAGAAATC | 2482 | GAUUUCUU G GUUGGUGA | 5261 |
| 2672 | GGTGTCAC GGCTAGCTACAACGA CAACAAGA | 2483 | UCUUGUUG G GUGACACC | 5262 |
| 2675 | TGAGGTGT GGCTAGCTACAACGA CACCAACA | 2484 | UGUUGGUG A ACACCUCA | 5263 |
| 2677 | GGTGAGGT GGCTAGCTACAACGA GTCACCAA | 2485 | UUGGUGAC A ACCUCACC | 5264 |
| 2682 | GGTGAGGT GGCTAGCTACAACGA GAGGTGTC | 2486 | GACACCUC A ACCUCACC | 5265 |
| 2687 | GCGTGGGT GGCTAGCTACAACGA GAGGTGAG | 2487 | CUCACCUC A ACCCACGC | 5266 |
| 2691 | TTTCGCGT GGCTAGCTACAACGA GGGTGAGG | 2488 | CCUCACCC A ACGCGAAA | 5267 |
| 2693 | GTTTTCGC GGCTAGCTACAACGA GTGGGTGA | 2489 | UCACCCAC G GCGAAAAC | 5268 |
| 2699 | AGGAAGGT GGCTAGCTACAACGA TTTCGCGT | 2490 | ACGCGAAA A ACCUUCCU | 5269 |
| 2711 | ACCAGGGT GGCTAGCTACAACGA CCTGAGGA | 2491 | UCCUCAGG A ACCCUGGU | 5270 |
| 2717 | CCTCGGAC GGCTAGCTACAACGA CAGGGTCC | 2492 | GGACCCUG G GUCCGAGG | 5271 |
| 2724 | AGGGACAC GGCTAGCTACAACGA CTCGGACC | 2493 | GGUCCGAG G GUGUCCCU | 5272 |
| 2726 | TCAGGGAC GGCTAGCTACAACGA ACCTCGGA | 2494 | UCCGAGGU G GUCCCUGA | 5273 |
| 2734 | AGCCATAC GGCTAGCTACAACGA TCAGGGAC | 2495 | GUCCCUGA G GUAUGGCU | 5274 |
| 2736 | GCAGCCAT GGCTAGCTACAACGA ACTCAGGG | 2496 | CCCUGAGU A AUGGCUGC | 5275 |
| 2739 | CACGCAGC GGCTAGCTACAACGA CATACTCA | 2497 | UGAGUAUG G GCUGCGUG | 5276 |
| 2742 | CACCACGC GGCTAGCTACAACGA AGCCATAC | 2498 | GUAUGGCU G GCGUGGUG | 5277 |
| 2744 | TTCACCAC GGCTAGCTACAACGA GCAGCCAT | 2499 | AUGGCUGC G GUGGUGAA | 5278 |
| 2747 | AAGTTCAC GGCTAGCTACAACGA CACGCAGC | 2500 | GCUGCGUG G GUGAACUU | 5279 |
| 2751 | CCGCAAGT GGCTAGCTACAACGA TCACCACG | 2501 | CGUGGUGA A ACUUGCGG | 5280 |
| 2755 | TCTTCCGC GGCTAGCTACAACGA AAGTTCAC | 2502 | GUGAACUU G GCGGAAGA | 5281 |
| 2762 | ACCACTGT GGCTAGCTACAACGA CTTCCGCA | 2503 | UGCGGAAG A ACAGUGGU | 5282 |
| 2765 | TTCACCAC GGCTAGCTACAACGA TGTCTTCC | 2504 | GGAAGACA G GUGGUGAA | 5283 |
| 2768 | AAGTTCAC GGCTAGCTACAACGA CACTGTCT | 2505 | AGACAGUG G GUGAACUU | 5284 |
| 2772 | AGGGAAGT GGCTAGCTACAACGA TCACCACT | 2506 | AGUGGUGA A ACUUCCCU | 5285 |
| 2780 | TCTTCTAC GGCTAGCTACAACGA AGGGAAGT | 2507 | ACUUCCCU G GUAGAAGA | 5286 |
| 2787 | GGCCTCGT GGCTAGCTACAACGA CTTCTACA | 2508 | UGUAGAAG A ACGAGGCC | 5287 |
| 2792 | CCCAGGGC GGCTAGCTACAACGA CTCGTCTT | 2509 | AAGACGAG G GCCCUGGG | 5288 |
| 2799 | CGTGCCAC GGCTAGCTACAACGA CCAGGGCC | 2510 | GGCCCUGG G GUGGCACG | 5289 |
| 2802 | AGCCGTGC GGCTAGCTACAACGA CACCCAGG | 2511 | CCUGGGUG G GCACGGCU | 5290 |
| 2804 | AAAGCCGT GGCTAGCTACAACGA GCCACCCA | 2512 | UGGGUGGC A ACGGCUUU | 5291 |
| 2807 | ACAAAAGC GGCTAGCTACAACGA CGTGCCAC | 2513 | GUGGCACG G GCUUUUGU | 5292 |
| 2813 | ATCTGAAC GGCTAGCTACAACGA AAAAGCCG | 2514 | CGGCUUUU G GUUCAGAU | 5293 |
| 2819 | GCCGGCAT GGCTAGCTACAACGA CTGAACAA | 2515 | UUGUUCAG A AUGCCGGC | 5294 |
| 2821 | GGGCCGGC GGCTAGCTACAACGA ATCTGAAC | 2516 | GUUCAGAU G GCCGGCCC | 5295 |
| 2825 | CCGTGGGC GGCTAGCTACAACGA CGGCATCT | 2517 | AGAUGCCG G GCCCACGG | 5296 |
| 2829 | TAGGCCGT GGCTAGCTACAACGA GGGCCGGC | 2518 | GCCGGCCC A ACGGCCUA | 5297 |
| 2832 | GAATAGGC GGCTAGCTACAACGA CGTGGGCC | 2519 | GGCCCACG G GCCUAUUC | 5298 |
| 2836 | AGGGGAAT GGCTAGCTACAACGA AGGCCGTG | 2520 | CACGGCCU A AUUCCCCU | 5299 |
| 2845 | GGCCGCAC GGCTAGCTACAACGA CAGGGGAA | 2521 | UUCCCCUG G GUGCGGCC | 5300 |
| 2847 | CAGGCCGC GGCTAGCTACAACGA ACCAGGGG | 2522 | CCCCUGGU G GCGGCCUG | 5301 |
| 2850 | CAGCAGGC GGCTAGCTACAACGA CGCACCAG | 2523 | CUGGUGCG G GCCUGCUG | 5302 |
| 2854 | CCAGCAGC GGCTAGCTACAACGA AGGCCGCA | 2524 | UGCGGCCU G GCUGCUGG | 5303 |
| 2857 | TATCCAGC GGCTAGCTACAACGA AGCAGGCC | 2525 | GGCCUGCU G GCUGGAUA | 5304 |
| 2862 | CCGGGTAT GGCTAGCTACAACGA CCAGCAGC | 2526 | GCUGCUGG A AUACCCGG | 5305 |
| 2864 | GTCCGGGT GGCTAGCTACAACGA ATCCAGCA | 2527 | UGCUGGAU A ACCCGGAC | 5306 |
| 2870 | TCCAGGGT GGCTAGCTACAACGA CCGGGTAT | 2528 | AUACCCGG A ACCCUGGA | 5307 |
| 2879 | CTCTGCAC GGCTAGCTACAACGA CTCCAGGG | 2529 | CCCUGGAG G GUGCAGAG | 5308 |
| 2881 | CGCTCTGC GGCTAGCTACAACGA ACCTCCAG | 2530 | CUGGAGGU G GCAGAGCG | 5309 |
| 2886 | GTAGTCGC GGCTAGCTACAACGA TCTGCACC | 2531 | GGUGCAGA G GCGACUAC | 5310 |
| 2889 | GGAGTAGT GGCTAGCTACAACGA CGCTCTGC | 2532 | GCAGAGCG A ACUACUCC | 5311 |
| 2892 | GCTGGAGT GGCTAGCTACAACGA AGTCGCTC | 2533 | GAGCGACU A ACUCCAGC | 5312 |
| 2898 | GGCATAGC GGCTAGCTACAACGA TGGAGTAG | 2534 | CUACUCCA G GCUAUGCC | 5313 |
| 2901 | CCGGGCAT GGCTAGCTACAACGA AGCTGGAG | 2535 | CUCCAGCU A AUGCCCGG | 5314 |
| 2903 | GTCCGGGC GGCTAGCTACAACGA ATAGCTGG | 2536 | CCAGCUAU G GCCCGGAC | 5315 |
| 2909 | ATGGAGGT GGCTAGCTACAACGA CCGGGCAT | 2537 | AUGCCCGG A ACCUCCAU | 5316 |
| 2915 | GCTCTGAT GGCTAGCTACAACGA GGAGGTCC | 2538 | GGACCUCC A AUCAGAGC | 5317 |
| 2921 | AGACTGGC GGCTAGCTACAACGA TCTGATGG | 2539 | CCAUCAGA G GCCAGUCU | 5318 |
| 2925 | GGTGAGAC GGCTAGCTACAACGA TGGCTCTG | 2540 | CAGAGCCA G GUCUCACC | 5319 |
| 2930 | TTGAAGGT GGCTAGCTACAACGA GAGACTGG | 2541 | CCAGUCUC A ACCUUCAA | 5320 |
| 2937 | GCCGCGGT GGCTAGCTACAACGA TGAAGGTG | 2542 | CACCUUCA A ACCGCGGC | 5321 |
| 2940 | GAAGCCGC GGCTAGCTACAACGA GGTTGAAG | 2543 | CUUCAACC G GCGGCUUC | 5322 |
| 2943 | CTTGAAGC GGCTAGCTACAACGA CGCGGTTG | 2544 | CAACCGCG G GCUUCAAG | 5323 |
| 2951 | CTCCCAGC GGCTAGCTACAACGA CTTGAAGC | 2545 | GCUUCAAG G GCUGGGAG | 5324 |
| 2961 | ACGCATGT GGCTAGCTACAACGA TCCTCCCA | 2546 | UGGGAGGA A ACAUGCGU | 5325 |
| 2963 | CGACGCAT GGCTAGCTACAACGA GTTCCTCC | 2547 | GGAGGAAC A AUGCGUCG | 5326 |
| 2965 | TGCGACGC GGCTAGCTACAACGA ATGTTCCT | 2548 | AGGAACAU G GCGUCGCA | 5327 |
| 2967 | TTTGCGAC GGCTAGCTACAACGA GCATGTTC | 2549 | GAACAUGC G GUCGCAAA | 5328 |
| 2970 | GAGTTTGC GGCTAGCTACAACGA GACGCATG | 2550 | CAUGCGUC G GCAAACUC | 5329 |
| 2974 | CAAAGAGT GGCTAGCTACAACGA TTGCGACG | 2551 | CGUCGCAA A ACUCUUUG | 5330 |
| 2984 | CGCAAGAC GGCTAGCTACAACGA CCCAAAGA | 2552 | UCUUUGGG G GUCUUGCG | 5331 |
| 2989 | TCAGCCGC GGCTAGCTACAACGA AAGACCCC | 2553 | GGGGUCUU G GCGGCUGA | 5332 |
| 2992 | ACTTCAGC GGCTAGCTACAACGA CGCAAGAC | 2554 | GUCUUGCG G GCUGAAGU | 5333 |
| 2998 | TGTGACAC GGCTAGCTACAACGA TTCAGCCG | 2555 | CGGCUGAA G GUGUCACA | 5334 |
| 3000 | GCTGTGAC GGCTAGCTACAACGA ACTTCAGC | 2556 | GCUGAAGU G GUCACAGC | 5335 |
| 3003 | CAGGCTGT GGCTAGCTACAACGA GACACTTC | 2557 | GAAGUGUC A ACAGCCUG | 5336 |
| 3006 | AAACAGGC GGCTAGCTACAACGA TGTGACAC | 2558 | GUGUCACA G GCCUGUUU | 5337 |
| 3010 | CCAGAAAC GGCTAGCTACAACGA AGGCTGTG | 2559 | CACAGCCU G GUUUCUGG | 5338 |
| 3018 | CTGCAAAT GGCTAGCTACAACGA CCAGAAAC | 2560 | GUUUCUGG A AUUUGCAG | 5339 |
| 3022 | TCACCTGC GGCTAGCTACAACGA AAATCCAG | 2561 | CUGGAUUU G GCAGGUGA | 5340 |
| 3026 | CTGTTCAC GGCTAGCTACAACGA CTGCAAAT | 2562 | AUUUGCAG G GUGAACAG | 5341 |
| 3030 | GAGGCTGT GGCTAGCTACAACGA TCACCTGC | 2563 | GCAGGUGA A ACAGCCUC | 5342 |
| 3033 | CTGGAGGC GGCTAGCTACAACGA TGTTCACC | 2564 | GGUGAACA G GCCUCCAG | 5343 |
| 3041 | CACACCGT GGCTAGCTACAACGA CTGGAGGC | 2565 | GCCUCCAG A ACGGUGUG | 5344 |
| 3044 | GTGCACAC GGCTAGCTACAACGA CGTCTGGA | 2566 | UCCAGACG G GUGUGCAC | 5345 |
| 3046 | TGGTGCAC GGCTAGCTACAACGA ACCGTCTG | 2567 | CAGACGGU G GUGCACCA | 5346 |
| 3048 | GTTGGTGC GGCTAGCTACAACGA ACACCGTC | 2568 | GACGGUGU G GCACCAAC | 5347 |
| 3050 | ATGTTGGT GGCTAGCTACAACGA GCACACCG | 2569 | CGGUGUGC A ACCAACAU | 5348 |
| 3054 | GTAGATGT GGCTAGCTACAACGA TGGTGCAC | 2570 | GUGCACCA A ACAUCUAC | 5349 |
| 3056 | TTGTAGAT GGCTAGCTACAACGA GTTGGTGC | 2571 | GCACCAAC A AUCUACAA | 5350 |
| 3060 | GATCTTGT GGCTAGCTACAACGA AGATGTTG | 2572 | CAACAUCU A ACAAGAUC | 5351 |
| 3065 | AGGAGGAT GGCTAGCTACAACGA CTTGTAGA | 2573 | UCUACAAG A AUCCUCCU | 5352 |
| 3073 | CCTGCAGC GGCTAGCTACAACGA AGGAGGAT | 2574 | AUCCUCCU G GCUGCAGG | 5353 |
| 3076 | ACGCCTGC GGCTAGCTACAACGA AGCAGGAG | 2575 | CUCCUGCU G GCAGGCGU | 5354 |
| 3080 | CTGTACGC GGCTAGCTACAACGA CTGCAGCA | 2576 | UGCUGCAG G GCGUACAG | 5355 |
| 3082 | ACCTGTAC GGCTAGCTACAACGA GCCTGCAG | 2577 | CUGCAGGC G GUACAGGU | 5356 |
| 3084 | AAACCTGT GGCTAGCTACAACGA ACGCCTGC | 2578 | GCAGGCGU A ACAGGUUU | 5357 |
| 3088 | CGTGAAAC GGCTAGCTACAACGA CTGTACGC | 2579 | GCGUACAG G GUUUCACG | 5358 |
| 3093 | ACATGCGT GGCTAGCTACAACGA GAAACCTG | 2580 | CAGGUUUC A ACGCAUGU | 5359 |
| 3095 | ACACATGC GGCTAGCTACAACGA GTGAAACC | 2581 | GGUUUCAC G GCAUGUGU | 5360 |
| 3097 | GCACACAT GGCTAGCTACAACGA GCGTGAAA | 2582 | UUUCACGC A AUGUGUGC | 5361 |
| 3099 | CAGCACAC GGCTAGCTACAACGA ATGCGTGA | 2583 | UCACGCAU G GUGUGCUG | 5362 |
| 3101 | TGCAGCAC GGCTAGCTACAACGA ACATGCGT | 2584 | ACGCAUGU G GUGCUGCA | 5363 |
| 3103 | GCTGCAGC GGCTAGCTACAACGA ACACATGC | 2585 | GCAUGUGU G GCUGCAGC | 5364 |
| 3106 | GGAGCTGC GGCTAGCTACAACGA AGCACACA | 2586 | UGUGUGCU G GCAGCUCC | 5365 |
| 3109 | ATGGGAGC GGCTAGCTACAACGA TGCAGCAC | 2587 | GUGCUGCA G GCUCCCAU | 5366 |
| 3115 | GATGAAAT GGCTAGCTACAACGA GGGAGCTG | 2588 | CAGCUCCC A AUUUCAUC | 5367 |
| 3120 | TTGCTGAT GGCTAGCTACAACGA GAAATGGG | 2589 | CCCAUUUC A AUCAGCAA | 5368 |
| 3124 | AAACTTGC GGCTAGCTACAACGA TGATGAAA | 2590 | UUUCAUCA G GCAAGUUU | 5369 |
| 3128 | TTCCAAAC GGCTAGCTACAACGA TTGCTGAT | 2591 | AUCAGCAA G GUUUGGAA | 5370 |
| 3138 | TGTGGGGT GGCTAGCTACAACGA TCTTCCAA | 2592 | UUGGAAGA A ACCCCACA | 5371 |
| 3143 | AAAAATGT GGCTAGCTACAACGA GGGGTTCT | 2593 | AGAACCCC A ACAUUUUU | 5372 |
| 3145 | GGAAAAAT GGCTAGCTACAACGA GTGGGGTT | 2594 | AACCCCAC A AUUUUUCC | 5373 |
| 3154 | TGACGCGC GGCTAGCTACAACGA AGGAAAAA | 2595 | UUUUUCCU G GCGCGUCA | 5374 |
| 3156 | GATGACGC GGCTAGCTACAACGA GCAGGAAA | 2596 | UUUCCUGC G GCGUCAUC | 5375 |
| 3158 | GAGATGAC GGCTAGCTACAACGA GCGCAGGA | 2597 | UCCUGCGC G GUCAUCUC | 5376 |
| 3161 | TCAGAGAT GGCTAGCTACAACGA GACGCGCA | 2598 | UGCGCGUC A AUCUCUGA | 5377 |
| 3168 | GGCCGTGT GGCTAGCTACAACGA CAGAGATG | 2599 | CAUCUCUG A ACACGGCC | 5378 |
| 3170 | GAGGCCGT GGCTAGCTACAACGA GTCAGAGA | 2600 | UCUCUGAC A ACGGCCUC | 5379 |
| 3173 | AGGGAGGC GGCTAGCTACAACGA CGTGTCAG | 2601 | CUGACACG G GCCUCCCU | 5380 |
| 3183 | GGAGTAGC GGCTAGCTACAACGA AGAGGGAG | 2602 | CUCCCUCU G GCUACUCC | 5381 |
| 3186 | GATGGAGT GGCTAGCTACAACGA AGCAGAGG | 2603 | CCUCUGCU A ACUCCAUC | 5382 |
| 3191 | TTCAGGAT GGCTAGCTACAACGA GGAGTAGC | 2604 | GCUACUCC A AUCCUGAA | 5383 |
| 3200 | TTCTTGGC GGCTAGCTACAACGA TTTCAGGA | 2605 | UCCUGAAA G GCCAAGAA | 5384 |
| 3207 | CCCTGCGT GGCTAGCTACAACGA TCTTGGCT | 2606 | AGCCAAGA A ACGCAGGG | 5385 |
| 3209 | ATCCCTGC GGCTAGCTACAACGA GTTCTTGG | 2607 | CCAAGAAC G GCAGGGAU | 5386 |
| 3215 | AGCGACAT GGCTAGCTACAACGA CCCTGCGT | 2608 | ACGCAGGG A AUGUCGCU | 5387 |
| 3217 | CCAGCGAC GGCTAGCTACAACGA ATCCCTGC | 2609 | GCAGGGAU G GUCGCUGG | 5388 |
| 3220 | CCCCCAGC GGCTAGCTACAACGA GACATCCC | 2610 | GGGAUGUC G GCUGGGGG | 5389 |
| 3227 | CCCTTGGC GGCTAGCTACAACGA CCCCAGCG | 2611 | CGCUGGGG G GCCAAGGG | 5390 |
| 3234 | GGCGGCGC GGCTAGCTACAACGA CCTTGGCC | 2612 | GGCCAAGG G GCGCCGCC | 5391 |
| 3236 | CCGGCGGC GGCTAGCTACAACGA GCCCTTGG | 2613 | CCAAGGGC G GCCGCCGG | 5392 |
| 3239 | GGGCCGGC GGCTAGCTACAACGA GGCGCCCT | 2614 | AGGGCGCC G GCCGGCCC | 5393 |
| 3243 | CAGAGGGC GGCTAGCTACAACGA CGGCGGCG | 2615 | CGCCGCCG G GCCCUCUG | 5394 |
| 3250 | CGGAGGGC GGCTAGCTACAACGA AGAGGGCC | 2616 | GGCCCUCU G GCCCUCCG | 5395 |
| 3260 | TGCACGGC GGCTAGCTACAACGA CTCGGAGG | 2617 | CCUCCGAG G GCCGUGCA | 5396 |
| 3263 | CACTGCAC GGCTAGCTACAACGA GGCCTCGG | 2618 | CCGAGGCC G GUGCAGUG | 5397 |
| 3265 | GCCACTGC GGCTAGCTACAACGA ACGGCCTC | 2619 | GAGGCCGU G GCAGUGGC | 5398 |
| 3268 | ACAGCCAC GGCTAGCTACAACGA TGCACGGC | 2620 | GCCGUGCA G GUGGCUGU | 5399 |
| 3271 | GGCACAGG GGCTAGCTACAACGA CACTGCAC | 2621 | GUGCAGUG G GCUGUGCC | 5400 |
| 3274 | GGTGGCAC GGCTAGCTACAACGA AGCCACTG | 2622 | CAGUGGCU G GUGCCACC | 5401 |
| 3276 | TTGGTGGC GGCTAGCTACAACGA ACAGCCAC | 2623 | GUGGCUGU G GCCACCAA | 5402 |
| 3279 | TGCTTGGT GGCTAGCTACAACGA GGCACAGC | 2624 | GCUGUGCC A ACCAAGCA | 5403 |
| 3284 | AGGAATGC GGCTAGCTACAACGA TTGGTGGC | 2625 | GCCACCAA G GCAUUCCU | 5404 |
| 3286 | GCAGGAAT GGCTAGCTACAACGA GCTTGGTG | 2626 | CACCAAGC A AUUCCUGC | 5405 |
| 3292 | GCTTGAGC GGCTAGCTACAACGA AGGAATGC | 2627 | GCAUUCCU G GCUCAAGC | 5406 |
| 3298 | GAGTCAGC GGCTAGCTACAACGA TTGAGCAG | 2628 | CUGCUCAA G GCUGACUC | 5407 |
| 3302 | TGTCGAGT GGCTAGCTACAACGA CAGCTTGA | 2629 | UCAAGCUG A ACUCGACA | 5408 |
| 3307 | CACGGTGT GGCTAGCTACAACGA CGAGTCAG | 2630 | CUGACUCG A ACACCGUG | 5409 |
| 3309 | GACACGGT GGCTAGCTACAACGA GTCGAGTC | 2631 | GACUCGAC A ACCGUGUC | 5410 |
| 3312 | GGTGACAC GGCTAGCTACAACGA GGTGTCGA | 2632 | UCGACACC G GUGUCACC | 5411 |
| 3314 | TAGGTGAC GGCTAGCTACAACGA ACGGTGTC | 2633 | GACACCGU G GUCACCUA | 5412 |
| 3317 | ACGTAGGT GGCTAGCTACAACGA GACACGGT | 2634 | ACCGUGUC A ACCUACGU | 5413 |
| 3321 | TGGCACGT GGCTAGCTACAACGA AGGTGACA | 2635 | UGUCACCU A ACGUGCCA | 5414 |
| 3323 | AGTGGCAC GGCTAGCTACAACGA GTAGGTGA | 2636 | UCACCUAC G GUGCCACU | 5415 |
| 3325 | GGAGTGGC GGCTAGCTACAACGA ACGTAGGT | 2637 | ACCUACGU G GCCACUCC | 5416 |
| 3328 | CCAGGAGT GGCTAGCTACAACGA GGCACGTA | 2638 | UACGUGCC A ACUCCUGG | 5417 |
| 3337 | TGAGTGAC GGCTAGCTACAACGA CCCAGGAG | 2639 | CUCCUGGG G GUCACUCA | 5418 |
| 3340 | TCCTGAGT GGCTAGCTACAACGA GACCCCAG | 2640 | CUGGGGUC A ACUCAGGA | 5419 |
| 3347 | TGGGCTGT GGCTAGCTACAACGA CCTGAGTG | 2641 | CACUCAGG A ACAGCCCA | 5420 |
| 3350 | GTCTGGGC GGCTAGCTACAACGA TGTCCTGA | 2642 | UCAGGACA G GCCCAGAC | 5421 |
| 3356 | AGCTGCGT GGCTAGCTACAACGA CTGGGCTG | 2643 | CAGCCCAG A ACGCAGCU | 5422 |
| 3358 | TCAGCTGC GGCTAGCTACAACGA GTCTGGGC | 2644 | GCCCAGAC G GCAGCUGA | 5423 |
| 3361 | GACTCAGC GGCTAGCTACAACGA TGCGTCTG | 2645 | CAGACGCA G GCUGAGUC | 5424 |
| 3366 | CTTCCGAC GGCTAGCTACAACGA TCAGCTGC | 2646 | GCAGCUGA G GUCGGAAG | 5425 |
| 3373 | CCGGGAGC GGCTAGCTACAACGA TTCCGACT | 2647 | AGUCGGAA G GCUCCCGG | 5426 |
| 3383 | AGCGTCGT GGCTAGCTACAACGA CCCCGGGA | 2648 | UCCCGGGG A ACGACGCU | 5427 |
| 3386 | GTCAGCGT GGCTAGCTACAACGA CGTCCCCG | 2649 | CGGGGACG A ACGCUGAC | 5428 |
| 3388 | CAGTCAGC GGCTAGCTACAACGA GTCGTCCC | 2650 | GGGACGAC G GCUGACUG | 5429 |
| 3392 | AGGGCAGT GGCTAGCTACAACGA CAGCGTCG | 2651 | CGACGCUG A ACUGCCCU | 5430 |
| 3395 | TCCAGGGC GGCTAGCTACAACGA AGTCAGCG | 2652 | CGCUGACU G GCCCUGGA | 5431 |
| 3404 | GCTGCGGC GGCTAGCTACAACGA CTCCAGGG | 2653 | CCCUGGAG G GCCGCAGC | 5432 |
| 3407 | TTGGCTGC GGCTAGCTACAACGA GGCCTCCA | 2654 | UGGAGGCC G GCAGCCAA | 5433 |
| 3410 | GGGTTGGC GGCTAGCTACAACGA TGCGGCCT | 2655 | AGGCCGCA G GCCAACCC | 5434 |
| 3414 | TGCCGGGT GGCTAGCTACAACGA TGGCTGCG | 2656 | CGCAGCCA A ACCCGGCA | 5435 |
| 3419 | GGCAGTGC GGCTAGCTACAACGA CGGGTTGG | 2657 | CCAACCCG G GCACUGCC | 5436 |
| 3421 | AGGGCAGT GGCTAGCTACAACGA GCCGGGTT | 2658 | AACCCGGC A ACUGCCCU | 5437 |
| 3424 | CTGAGGGC GGCTAGCTACAACGA AGTGCCGG | 2659 | CCGGCACU G GCCCUCAG | 5438 |
| 3432 | CTTGAAGT GGCTAGCTACAACGA CTGAGGGC | 2660 | GCCCUCAG A ACUUCAAG | 5439 |
| 3440 | AGGATGGT GGCTAGCTACAACGA CTTGAAGT | 2661 | ACUUCAAG A ACCAUCCU | 5440 |
| 3443 | TCCAGGAT GGCTAGCTACAACGA GGTCTTGA | 2662 | UCAAGACC A AUCCUGGA | 5441 |
| 3450 | CCATCAGT GGCTAGCTACAACGA CCAGGATG | 2663 | CAUCCUGG A ACUGAUGG | 5442 |
| 3454 | GTGGCCAT GGCTAGCTACAACGA CAGTCCAG | 2664 | CUGGACUG A AUGGCCAC | 5443 |
| 3457 | CGGGTGGC GGCTAGCTACAACGA CATCAGTC | 2665 | GACUGAUG G GCCACCCG | 5444 |
| 3460 | GGGCGGGT GGCTAGCTACAACGA GGCCATCA | 2666 | UGAUGGCC A ACCCGCCC | 5445 |
| 3464 | CTGTGGGC GGCTAGCTACAACGA GGGTGGCC | 2667 | GGCCACCC G GCCCACAG | 5446 |
| 3468 | CTGGCTGT GGCTAGCTACAACGA GGGCGGGT | 2668 | ACCCGCCC A ACAGCCAG | 5447 |
| 3471 | GGCCTGGC GGCTAGCTACAACGA TGTGGGCG | 2669 | CGCCCACA G GCCAGGCC | 5448 |
| 3476 | CTCTCGGC GGCTAGCTACAACGA CTGGCTGT | 2670 | ACAGCCAG G GCCGAGAG | 5449 |
| 3483 | GTGTCTGC GGCTAGCTACAACGA TCTCGGCC | 2671 | GGCCGAGA G GCAGACAC | 5450 |
| 3487 | GCTGGTGT GGCTAGCTACAACGA CTGCTCTC | 2672 | GAGAGCAG A ACACCAGC | 5451 |
| 3489 | CTGCTGGT GGCTAGCTACAACGA GTCTGCTC | 2673 | GAGCAGAC A ACCAGCAG | 5452 |
| 3493 | AGGGCTGC GGCTAGCTACAACGA TGGTGTCT | 2674 | AGACACCA G GCAGCCCU | 5453 |
| 3496 | GACAGGGC GGCTAGCTACAACGA TGCTGGTG | 2675 | CACCAGCA G GCCCUGUC | 5454 |
| 3501 | GGCGTGAC GGCTAGCTACAACGA AGGGCTGC | 2676 | GCAGCCCU G GUCACGCC | 5455 |
| 3504 | CCCGGCGT GGCTAGCTACAACGA GACAGGGC | 2677 | GCCCUGUC A ACGCCGGG | 5456 |
| 3506 | AGCCCGGC GGCTAGCTACAACGA GTGACAGG | 2678 | CCUGUCAC G GCCGGGCU | 5457 |
| 3511 | CGTAGAGC GGCTAGCTACAACGA CCGGCGTG | 2679 | CACGCCGG G GCUCUACG | 5458 |
| 3516 | TGGGACGT GGCTAGCTACAACGA AGAGCCCG | 2680 | CGGGCUCU A ACGUCCCA | 5459 |
| 3518 | CCTGGGAC GGCTAGCTACAACGA GTAGAGCC | 2681 | GGCUCUAC G GUCCCAGG | 5460 |
| 3535 | TGGGCCGC GGCTAGCTACAACGA CCCTCCCT | 2682 | AGGGAGGG G GCGGCCCA | 5461 |
| 3538 | GTGTGGGC GGCTAGCTACAACGA CGCCCCTC | 2683 | GAGGGGCG G GCCCACAC | 5462 |
| 3542 | CTGGGTGT GGCTAGCTACAACGA GGGCCGCC | 2684 | GGCGGCCC A ACACCCAG | 5463 |
| 3544 | GCCTGGGT GGCTAGCTACAACGA GTGGGCCG | 2685 | CGGCCCAC A ACCCAGGC | 5464 |
| 3550 | GTGCGGGC GGCTAGCTACAACGA CTGGGTGT | 2686 | ACACCCAG G GCCCGCAC | 5465 |
| 3554 | AGCGGTGC GGCTAGCTACAACGA GGGCCTGG | 2687 | CCAGGCCC G GCACCGCU | 5466 |
| 3556 | CCAGCGGT GGCTAGCTACAACGA GCGGGCCT | 2688 | AGGCCCGC A ACCGCUGG | 5467 |
| 3559 | CTCCCAGC GGCTAGCTACAACGA GGTGCGGG | 2689 | CCCGCACC G GCUGGGAG | 5468 |
| 3566 | CCTCAGAC GGCTAGCTACAACGA TCCCAGCG | 2690 | CGCUGGGA G GUCUGAGG | 5469 |
| 3573 | ACTCAGGC GGCTAGCTACAACGA CTCAGACT | 2691 | AGUCUGAG G GCCUGAGU | 5470 |
| 3579 | ACACTCAC GGCTAGCTACAACGA TCAGGCCT | 2692 | AGGCCUGA G GUGAGUGU | 5471 |
| 3583 | CCAAACAC GGCTAGCTACAACGA TCACTCAG | 2693 | CUGAGUGA G GUGUUUGG | 5472 |
| 3585 | GGCCAAAC GGCTAGCTACAACGA ACTCACTC | 2694 | GAGUGAGU G GUUUGGCC | 5473 |
| 3590 | GCCTCGGC GGCTAGCTACAACGA CAAACACT | 2695 | AGUGUUUG G GCCGAGGC | 5474 |
| 3596 | ATGCAGGC GGCTAGCTACAACGA CTCGGCCA | 2696 | UGGCCGAG G GCCUGCAU | 5475 |
| 3600 | GGACATGC GGCTAGCTACAACGA AGGCCTCG | 2697 | CGAGGCCU G GCAUGUCC | 5476 |
| 3602 | CCGGACAT GGCTAGCTACAACGA GCAGGCCT | 2698 | AGGCCUGC A AUGUCCGG | 5477 |
| 3604 | AGCCGGAC GGCTAGCTACAACGA ATGCAGGC | 2699 | GCCUGCAU G GUCCGGCU | 5478 |
| 3609 | CCTTCAGC GGCTAGCTACAACGA CGGACATG | 2700 | CAUGUCCG G GCUGAAGG | 5479 |
| 3616 | CACTCAGC GGCTAGCTACAACGA CTTCAGCC | 2701 | GGCUGAAG G GCUGAGUG | 5480 |
| 3621 | CCGGACAC GGCTAGCTACAACGA TCAGCCTT | 2702 | AAGGCUGA G GUGUCCGG | 5481 |
| 3623 | AGCCGGAC GGCTAGCTACAACGA ACTCAGCC | 2703 | GGCUGAGU G GUCCGGCU | 5482 |
| 3628 | GCCTCAGC GGCTAGCTACAACGA CGGACACT | 2704 | AGUGUCCG G GCUGAGGC | 5483 |
| 3634 | GCTCAGGC GGCTAGCTACAACGA CTCAGCCG | 2705 | CGGCUGAG G GCCUGAGC | 5484 |
| 3640 | ACACTCGC GGCTAGCTACAACGA TCAGGCCT | 2706 | AGGCCUGA G GCGAGUGU | 5485 |
| 3644 | CTGGACAC GGCTAGCTACAACGA TCGCTCAG | 2707 | CUGAGCGA G GUGUCCAG | 5486 |
| 3646 | GGCTGGAC GGCTAGCTACAACGA ACTCGCTC | 2708 | GAGCGAGU G GUCCAGCC | 5487 |
| 3651 | CCCTTGGC GGCTAGCTACAACGA TGGACACT | 2709 | AGUGUCCA G GCCAAGGG | 5488 |
| 3658 | CACTCAGC GGCTAGCTACAACGA CCTTGGCT | 2710 | AGCCAAGG G GCUGAGUG | 5489 |
| 3663 | CTGGACAC GGCTAGCTACAACGA TCAGCCCT | 2711 | AGGGCUGA G GUGUCCAG | 5490 |
| 3665 | TGCTGGAC GGCTAGCTACAACGA ACTCAGCC | 2712 | GGCUGAGU G GUCCAGCA | 5491 |
| 3670 | AGGTGTGC GGCTAGCTACAACGA TGGACACT | 2713 | AGUGUCCA G GCACACCU | 5492 |
| 3672 | GCAGGTGT GGCTAGCTACAACGA GCTGGACA | 2714 | UGUCCAGC A ACACCUGC | 5493 |
| 3674 | CGGCAGGT GGCTAGCTACAACGA GTGCTGGA | 2715 | UCCAGCAC A ACCUGCCG | 5494 |
| 3678 | AAGACGGC GGCTAGCTACAACGA AGGTGTGC | 2716 | GCACACCU G GCCGUCUU | 5495 |
| 3681 | GTGAAGAC GGCTAGCTACAACGA GGCAGGTG | 2717 | CACCUGCC G GUCUUCAC | 5496 |
| 3687 | GGGGAAGT GGCTAGCTACAACGA GAAGACGG | 2718 | CCGUCUUC A ACUUCCCC | 5497 |
| 3695 | CAGCCTGT GGCTAGCTACAACGA GGGGAAGT | 2719 | ACUUCCCC A ACAGGCUG | 5498 |
| 3699 | GCGCCAGC GGCTAGCTACAACGA CTGTGGGG | 2720 | CCCCACAG G GCUGGCGC | 5499 |
| 3703 | CCGAGCGC GGCTAGCTACAACGA CAGCCTGT | 2721 | ACAGGCUG G GCGCUCGG | 5500 |
| 3705 | AGCCGAGC GGCTAGCTACAACGA GCCAGCCT | 2722 | AGGCUGGC G GCUCGGCU | 5501 |
| 3710 | GGTGGAGC GGCTAGCTACAACGA CGAGCGCC | 2723 | GGCGCUCG G GCUCCACC | 5502 |
| 3715 | CCTGGGGT GGCTAGCTACAACGA GGAGCCGA | 2724 | UCGGCUCC A ACCCCAGG | 5503 |
| 3723 | AAGCTGGC GGCTAGCTACAACGA CCTGGGGT | 2725 | ACCCCAGG G GCCAGCUU | 5504 |
| 3727 | GGAAAAGC GGCTAGCTACAACGA TGGCCCTG | 2726 | CAGGGCCA G GCUUUUCC | 5505 |
| 3737 | CTCCTGGT GGCTAGCTACAACGA GAGGAAAA | 2727 | UUUUCCUC A ACCAGGAG | 5506 |
| 3744 | AGCCGGGC GGCTAGCTACAACGA TCCTGGTG | 2728 | CACCAGGA G GCCCGGCU | 5507 |
| 3749 | GTGGAAGC GGCTAGCTACAACGA CGGGCTCC | 2729 | GGAGCCCG G GCUUCCAC | 5508 |
| 3755 | TGGGGAGT GGCTAGCTACAACGA GGAAGCCG | 2730 | CGGCUUCC A ACUCCCCA | 5509 |
| 3762 | TCCTATGT GGCTAGCTACAACGA GGGGAGTG | 2731 | CACUCCCC A ACAUAGGA | 5510 |
| 3764 | ATTCCTAT GGCTAGCTACAACGA GTGGGGAG | 2732 | CUCCCCAC A AUAGGAAU | 5511 |
| 3770 | TGGACTAT GGCTAGCTACAACGA TCCTATGT | 2733 | ACAUAGGA A AUAGUCCA | 5512 |
| 3773 | GGATGGAC GGCTAGCTACAACGA TATTCCTA | 2734 | UAGGAAUA G GUCCAUCC | 5513 |
| 3777 | CTGGGGAT GGCTAGCTACAACGA GGACTATT | 2735 | AAUAGUCC A AUCCCCAG | 5514 |
| 3785 | TGGCGAAT GGCTAGCTACAACGA CTGGGGAT | 2736 | AUCCCCAG A AUUCGCCA | 5515 |
| 3789 | ACAATGGC GGCTAGCTACAACGA GAATCTGG | 2737 | CCAGAUUC G GCCAUUGU | 5516 |
| 3792 | TGAACAAT GGCTAGCTACAACGA GGCGAATC | 2738 | GAUUCGCC A AUUGUUCA | 5517 |
| 3795 | GGGTGAAC GGCTAGCTACAACGA AATGGCGA | 2739 | UCGCCAUU G GUUCACCC | 5518 |
| 3799 | CGAGGGGT GGCTAGCTACAACGA GAACAATG | 2740 | CAUUGUUC A ACCCCUCG | 5519 |
| 3806 | GGCAGGGC GGCTAGCTACAACGA GAGGGGTG | 2741 | CACCCCUC G GCCCUGCC | 5520 |
| 3811 | AGGAGGGC GGCTAGCTACAACGA AGGGCGAG | 2742 | CUCGCCCU G GCCCUCCU | 5521 |
| 3821 | TGGAAGGC GGCTAGCTACAACGA AAAGGAGG | 2743 | CCUCCUUU G GCCUUCCA | 5522 |
| 3828 | GTGGGGGT GGCTAGCTACAACGA GGAAGGCA | 2744 | UGCCUUCC A ACCCCCAC | 5523 |
| 3834 | TGGATGGT GGCTAGCTACAACGA GGGGGTGG | 2745 | CCACCCCC A ACCAUCCA | 5524 |
| 3837 | ACCTGGAT GGCTAGCTACAACGA GGTGGGGG | 2746 | CCCCCACC A AUCCAGGU | 5525 |
| 3843 | GTCTCCAC GGCTAGCTACAACGA CTGGATGG | 2747 | CCAUCCAG G GUGGAGAC | 5526 |
| 3849 | CTCAGGGT GGCTAGCTACAACGA CTCCACCT | 2748 | AGGUGGAG A ACCCUGAG | 5527 |
| 3861 | CCCAGGGT GGCTAGCTACAACGA CCTTCTCA | 2749 | UGAGAAGG A ACCCUGGG | 5528 |
| 3870 | CCCAGAGC GGCTAGCTACAACGA TCCCAGGG | 2750 | CCCUGGGA G GCUCUGGG | 5529 |
| 3879 | CTCCAAAT GGCTAGCTACAACGA TCCCAGAG | 2751 | CUCUGGGA A AUUUGGAG | 5530 |
| 3886 | TTGGTCAC GGCTAGCTACAACGA TCCAAATT | 2752 | AAUUUGGA G GUGACCAA | 5531 |
| 3889 | CCTTTGGT GGCTAGCTACAACGA CACTCCAA | 2753 | UUGGAGUG A ACCAAAGG | 5532 |
| 3896 | GGGCACAC GGCTAGCTACAACGA CTTTGGTC | 2754 | GACCAAAG G GUGUGCCC | 5533 |
| 3898 | CAGGGCAC GGCTAGCTACAACGA ACCTTTGG | 2755 | CCAAAGGU G GUGCCCUG | 5534 |
| 3900 | TACAGGGC GGCTAGCTACAACGA ACACCTTT | 2756 | AAAGGUGU G GCCCUGUA | 5535 |
| 3905 | CTGTGTAC GGCTAGCTACAACGA AGGGCACA | 2757 | UGUGCCCU G GUACACAG | 5536 |
| 3907 | GCCTGTGT GGCTAGCTACAACGA ACAGGGCA | 2758 | UGCCCUGU A ACACAGGC | 5537 |
| 3909 | TCGCCTGT GGCTAGCTACAACGA GTACAGGG | 2759 | CCCUGUAC A ACAGGCGA | 5538 |
| 3913 | GTCCTCGC GGCTAGCTACAACGA CTGTGTAC | 2760 | GUACACAG G GCGAGGAC | 5539 |
| 3919 | TGCAGGGT GGCTAGCTACAACGA CCTCGCCT | 2761 | AGGCGAGG A ACCCUGCA | 5540 |
| 3924 | CCAGGTGC GGCTAGCTACAACGA AGGGTCCT | 2762 | AGGACCCU G GCACCUGG | 5541 |
| 3926 | ATCCAGGT GGCTAGCTACAACGA GCAGGGTC | 2763 | GACCCUGC A ACCUGGAU | 5542 |
| 3932 | ACCCCCAT GGCTAGCTACAACGA CCAGGTGC | 2764 | GCACCUGG A AUGGGGGU | 5543 |
| 3938 | ACAGGGAC GGCTAGCTACAACGA CCCCATCC | 2765 | GGAUGGGG G GUCCCUGU | 5544 |
| 3944 | TGACCCAC GGCTAGCTACAACGA AGGGACCC | 2766 | GGGUCCCU G GUGGGUCA | 5545 |
| 3948 | AATTTGAC GGCTAGCTACAACGA CCACAGGG | 2767 | CCCUGUGG G GUCAAAUU | 5546 |
| 3953 | CCCCCAAT GGCTAGCTACAACGA TTGACCCA | 2768 | UGGGUCAA A AUUGGGGG | 5547 |
| 3964 | CACAGCAC GGCTAGCTACAACGA CTCCCCCC | 2769 | GGGGGGAG G GUGCUGUG | 5548 |
| 3966 | CCCACAGC GGCTAGCTACAACGA ACCTCCCC | 2770 | GGGGAGGU G GCUGUGGG | 5549 |
| 3969 | ACTCCCAC GGCTAGCTACAACGA AGCACCTC | 2771 | GAGGUGCU G GUGGGAGU | 5550 |
| 3975 | TATTTTAC GGCTAGCTACAACGA TCCCACAG | 2772 | CUGUGGGA G GUAAAAUA | 5551 |
| 3980 | TTCAGTAT GGCTAGCTACAACGA TTTACTCC | 2773 | GGAGUAAA A AUACUGAA | 5552 |
| 3982 | TATTCAGT GGCTAGCTACAACGA ATTTTACT | 2774 | AGUAAAAU A ACUGAAUA | 5553 |
| 3987 | TCATATAT GGCTAGCTACAACGA TCAGTATT | 2775 | AAUACUGA A AUAUAUGA | 5554 |
| 3989 | ACTCATAT GGCTAGCTACAACGA ATTCAGTA | 2776 | UACUGAAU A AUAUGAGU | 5555 |
| 3991 | AAACTCAT GGCTAGCTACAACGA ATATTCAG | 2777 | CUGAAUAU A AUGAGUUU | 5556 |
| 3995 | TGAAAAAC GGCTAGCTACAACGA TCATATAT | 2778 | AUAUAUGA G GUUUUUCA | 5557 |
| 4003 | TTCAAAAC GGCTAGCTACAACGA TGAAAAAC | 2779 | GUUUUUCA G GUUUUGAA | 5558 |

Seq1 = TERT (Homo sapiens telomerase reverse transcriptase (TERT) mRNA, 4015 bp); Nakamura et al., Science 277 (5328), 955-959 (1997)
Cut Site = R/Y (Purine/Pyrimidine)
Stem Length = 8. Core Sequence = GGCTAGCTACAACGA

TABLE VII
Anti-TERT HH and G-Cleaver Ribozymes

| | | Seq ID | Length |
|---|---|---|---|
| Alias | Ribozyme Sequence | Number | (nt) |
| HH | | | |
| TERT-1051 | AGGAGUA CUGAUGAGGCCGUUAGGCCGAA AGGAAGU | 5559 | 36 |
| TERT-1053 | UGAGGAG CUGAUGAGGCCGUUAGGCCGAA AGAGGAA | 5560 | 36 |
| TERT-1918 | UGAAGCG CUGAUGAGGCCGUUAGGCCGAA AGUCUGG | 5561 | 36 |
| TERT-2383 | GAGCCAC CUGAUGAGGCCGUUAGGCCGAA AACUGUC | 5562 | 36 |
| TERT-2485 | UGAAGCG CUGAUGAGGCCGUUAGGCCGAA AGGAAGA | 5563 | 36 |
| TERT-2566 | GCGUGGA CUGAUGAGGCCGUUAGGCCGAA AGGAUGG | 5564 | 36 |
| TERT-3181 | AGUAGCA CUGAUGAGGCCGUUAGGCCGAA AGGGAGG | 5565 | 36 |
| TERT-3691 | CUGUGGG CUGAUGAGGCCGUUAGGCCGAA AAGUGAA | 5566 | 36 |
| TERT-3758 | AUGUGGG CUGAUGAGGCCGUUAGGCCGAA AGUGGAA | 5567 | 36 |
| TERT-3794 | GGUGAAC CUGAUGAGGCCGUUAGGCCGAA AUGGCGA | 5568 | 36 |
| G-Cleaver | | | |
| TERT-757 | UUGGG UGAUGGCAUGCACUAUGCGCG AACGGCAGAC | 4332 | 36 |
| TERT-2353 | UCUGU UGAUGGCAUGCACUAUGCGCG AAGGUAGAGA | 4471 | 36 |
| TERT-3795 | GUGAA UGAUGGCAUGCACUAUGCGCG AAUGGCGAAU | 4594 | 36 |

1. A ribonucleic acid molecule of about 21 nucleotides in length comprising nucleotide sequence complementary to RNA sequence of telomerase reverse transcriptase (TERT) gene, wherein said ribonucleic acid molecule comprises at least one 2′-sugar modification.

2. The ribonucleic acid molecule of claim 1, wherein said ribonucleic acid comprises at least one phosphate backbone modification.

3. The ribonucleic acid molecule of claim 1, wherein said ribonucleic acid comprises a cap structure at the 5′-end, or 3′-end, or both the 5′-end, or 3′-end.

4. The ribonucleic acid molecule of claim 1, wherein said ribonucleic acid molecule is single-stranded.

5. The ribonucleic acid molecule of claim 1, wherein said sugar modification is 2′-amino, 2′-C-allyl, 2′-fluoro, 2′-O-methyl, 2′-H, or any combination thereof.
